(19) United States
(12) Patent Application Publication
(10) Pub. No.: US 2009/0042929 A1
(43) Pub. Date: Feb. 12, 2009

(54) 11-Beta-Hydroxysteroid Dehydrogenase Inhibitors

(75) Inventors: Nigel Vicker, Berkshire (GB); Xiangdong Su, Berkshire (GB); Fabienne Pradaux, Berkshire (GB); Michael John Reed, Berkshire (GB); Barry Victor Lloyd Potter, Berkshire (GB)

Correspondence Address: STEPTOE & JOHNSON LLP, 1330 CONNECTICUT AVENUE, N.W., WASHINGTON, DC, 20036, US

(73) Assignee: Sterix Limited, Slough (GB)

(21) Appl. No.: 11/886,884

(22) PCT Filed: Mar. 23, 2006
(86) PCT No.: PCT/GB2006/001086

§ 371 Date: Jun. 17, 2008

(30) Foreign Application Priority Data
Mar. 24, 2005 (GB) 0506133.8

(51) Int. Cl. A61K 31/437; A61P 3/00; A61P 29/00; A61P 37/00; A61P 19/00; A61P 35/00; C07C 311/20; A61K 31/428; A61K 31/18; C12Q 1/32; A61K 31/381; A61K 31/4439; C07C 311/16; C07D 277/64; C07D 333/24; C07D 213/56; C07D 495/04

(52) U.S. Cl. 514/301; 564/90; 548/160; 549/76; 546/285; 546/114; 514/342; 514/367; 514/438; 514/602; 435/26

(57) ABSTRACT

There is provided a compound having Formula (I) R₁-Z-R₂ wherein R₁ is a group selected from optionally substituted fused polycyclic groups, substituted alkyl groups, branched alkyl groups, and optionally substituted cycloalkyl groups Z is a linker which is or comprises a carbonyl group or a isostere of a carbonyl group R₂ is selected from optionally substituted aromatic rings and optionally substituted heterocyclic rings wherein (a) R₂ is a 2-substituted thiophene group, and/or (b) Z is a group of the formula —C(═O)—CR₃R₄—X—(CR₅R₆)n-, wherein X is selected from NR₇, S, O, S═O, and S(═O)₂, wherein n is 0 or 1 and/or (c) R₁ is an adamantyl group and Z is or comprises an amide group, and/or (d) R₁ is an adamantyl group and Z is or comprises a group of the formula —(CR₈R₉)p-NR₁₀—S(═O)₂—(CR₁₁R₁₂)q-, wherein p is 0 or 1 and q is 0 or 1 and/or (e) R₁ is an adamantyl group and Z is or comprises a group of the formula —(CR₁₃R₁₄)V—Y—(CR₁₅R₁₆)W— where Y is a heteroaryl group in which a bond in the heteroaryl ring is a isostere of a carbonyl group, wherein v is o or 1 and w is 0 or 1; wherein each of R₃, R₄, R₅, R₆, R₈, R₉, R₁₁, R₁₂, R₁₃, R₁₄, R₁₅ and R₁₆, are independently selected from H, hydrocarbyl and halogen, wherein each of R₇ and R₁₀ are independently selected from H and hydrocarbyl.

### FIELD OF INVENTION

The present invention relates to a compound. In particular the present invention provides compounds capable of inhibiting 11β-hydroxysteroid dehydrogenase (11β-HSD).

### INTRODUCTION

### The Role of Glucocorticoids

Glucocorticoids are synthesised in the adrenal cortex from cholesterol. The principle glucocorticoid in the human body is cortisol, this hormone is synthesised and secreted in response to the adrenocortictrophic hormone (ACTH) from the pituitary gland in a circadian, episodic manner, but the secretion of this hormone can also be stimulated by stress, exercise and infection. Cortisol circulates mainly bound to transcortin (cortisol binding protein) or albumin and only a small fraction is free (5-10%) for biological processes [1].

Cortisol has a wide range of physiological effects, including regulation of carbohydrate, protein and lipid metabolism, regulation of normal growth and development, influence on cognitive function, resistance to stress and mineralocorticoid activity. Cortisol works in the opposite direction compared to insulin meaning a stimulation of hepatic gluconeogenesis, inhibition of peripheral glucose uptake and increased blood glucose concentration. Glucocorticoids are also essential in the regulation of the immune response. When circulating at higher concentrations glucocorticoids are immunosuppressive and are used pharmacologically as anti-inflammatory agents.

Glucocorticoids like other steroid hormones are lipophilic and penetrate the cell membrane freely. Cortisol binds, primarily, to the intracellular glucocorticoid receptor (GR) that then acts as a transcription factor to induce the expression of glucocorticoid responsive genes, and as a result of that protein synthesis.

### The Role of the 11β-HSD Enzyme

The conversion of cortisol (F) to its inactive metabolite cortisone (E) by 11β-HSD was first described in the 1950's, however it was not until later that the biological importance for this conversion was suggested [2]. In 1983 Krozowski et al. showed that the mineralocorticoid receptor (MR) has equal binding affinities for glucocorticoids and mineralocorticoids [3]. Because the circulating concentration of cortisol is a 100 times higher than that of aldosterone and during times of stress or high activity even more, it was not clear how the MR remained mineralocorticoid specific and was not constantly occupied by glucocorticoids. Earlier Ulick et al. [4] had described the hypertensive condition known as, “apparent mineralocorticoid excess” (AME), and observed that whilst secretion of aldosterone from the adrenals was in fact low the peripheral metabolism of cortisol was disrupted. These discoveries lead to the suggestion of a protective role for the enzymes. By converting cortisol to cortisone in mineralocorticoid dependent tissues 11β-HSD enzymes protects the MR from occupation by glucocorticoids and allows it to be mineralcorticoid specific. Aldosterone itself is protected from the enzyme by the presence of an aldehyde group at the C-18 position.

Congenital defects in the 11β-HSD enzyme results in over occupation of the MR by cortisol and hypertensive and hypokalemic symptoms seen in AME.

Localisation of the 11β-HSD showed that the enzyme and its activity is highly present in the MR dependent tissues, kidney and parotid. However in tissues where the MR is not mineralocorticoid specific and is normally occupied by glucocorticoids, 11β-HSD is not present in these tissues, for example in the heart and hippocampus [5]. This research also showed that inhibition of 11β-HSD caused a loss of the aldosterone specificity of the MR in these mineralocorticoid dependent tissues.

It has been shown that two iso-enzymes of 11β-HSD exist. Both are members of the short chain alcohol dehydrogenase (SCAD) superfamily which have been widely conserved throughout evolution. 11β-HSD type 2 acts as a dehydrogenase to convert the secondary alcohol group at the C-11 position of cortisol to a secondary ketone, so producing the less active metabolite cortisone. 11β-HSD type 1 is thought to act mainly in vivo as a reductase, that is in the opposite direction to type 2 [6] [see below]. 11β-HSD type 1 and type 2 have only a 30% amino acid homology.

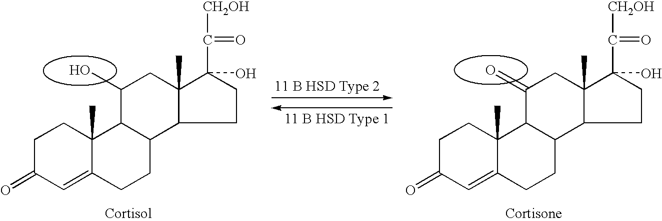

### 11β-HSD Enzyme Activity

The intracellular activity of cortisol is dependent on the concentration of glucocorticoids and can be modified and independently controlled without involving the overall secretion and synthesis of the hormone.

### The Role of 11β-HSD Type 1

The direction of 11β-HSD type 1 reaction in vivo is generally accepted to be opposite to the dehydrogenation of type 2. In vivo homozygous mice with a disrupted type 1 gene are unable to convert cortisone to cortisol, giving further evidence for the reductive activity of the enzyme [7]. 11β-HSD type 1 is expressed in many key glucocorticoid regulated tissues like the liver, pituitary, gonad, brain, adipose and adrenals however, the function of the enzyme in many of these tissues is poorly understood [8].

The concentration of cortisone in the body is higher than that of cortisol, cortisone also binds poorly to binding globulins, making cortisone many times more biologically available. Although cortisol is secreted by the adrenal cortex, there is a growing amount of evidence that the intracellular conversion of E to F may be an important mechanism in regulating the action of glucocorticoids [9].

It may be that 11β-HSD type 1 allows certain tissues to convert cortisone to cortisol to increase local glucocorticoid activity and potentiate adaptive response and counteracting the type 2 activity that could result in a fall in active glucocorticoids [10]. Potentiation of the stress response would be especially important in the brain and high levels of 11β-HSD type 1 are found around the hippocampus, further proving the role of the enzyme. 11β-HSD type 1 also seems to play an important role in hepatocyte maturation [8]. Another emerging role of the 11β-HSD type 1 enzyme is in the detoxification process of many non-steroidal carbonyl compounds, reduction of the carbonyl group of many toxic compounds is a common way to increase solubility and therefore increase their excretion. The 11β-HSD type 1 enzyme has recently been shown to be active in lung tissue [11]. Type 1 activity is not seen until after birth, therefore mothers who smoke during pregnancy expose their children to the harmful effects of tobacco before the child is able to metabolically detoxify this compound.

### The Role of 11β-HSD Type 2

As already stated earlier the 11β-HSD type 2 converts cortisol to cortisone, thus protecting the MR in many key regulatory tissues of the body. The importance of protecting the MR from occupation by glucocorticoids is seen in patients with AME or liquorice intoxification. Defects or inactivity of the type 2 enzyme results in hypertensive syndromes and research has shown that patients with an hypertensive syndrome have an increased urinary excretion ratio of cortisol:cortisone. This along with a reported increase in the half life of radiolabelled cortisol suggests a reduction of 11β-HSD type 2 activity [12].

### Rationale for the Development of 11β-HSD Inhibitors

As said earlier cortisol opposes the action of insulin meaning a stimulation of hepatic gluconeogenesis, inhibition of peripheral glucose uptake and increased blood glucose concentration. The effects of cortisol appear to be enhanced in patients suffering from glucose intolerance or diabetes mellitus. Inhibition of the enzyme 11β-HSD type 1 would increase glucose uptake and inhibit hepatic gluconeogenesis, giving a reduction in circulatory glucose levels. The development of a potent 11β-HSD type 1 inhibitor could therefore have considerable therapeutic potential for conditions associated with elevated blood glucose levels.

An excess in glucocorticoids can result in neuronal dysfunctions and also impair cognitive functions. A specific 11β-HSD type 1 inhibitor might be of some importance by reducing neuronal dysfunctions and the loss of cognitive functions associated with ageing, by blocking the conversion of cortisone to cortisol.

Glucocorticoids also have an important role in regulating part of the immune response [13]. Glucocorticoids can suppress the production of cytokines and regulate the receptor levels. They are also involved in determining whether T-helper (Th) lymphocytes progress into either Th1 or Th2 phenotype. These two different types of Th cells secrete a different profile of cytokines, Th2 is predominant in a glucocorticoid environment. By inhibiting 11β-HSD type 1, Th1 cytokine response would be favoured. It is also possible to inhibit 11β-HSD type 2, thus by inhibiting the inactivation of cortisol, it may be possible to potentiate the anti-inflammatory effects of glucocorticoids.

Aspects of the invention are defined in the appended claims.

### SUMMARY ASPECTS OF THE PRESENT INVENTION

In one aspect the present invention provides a compound having Formula I

R₁-Z-R₂ Formula I

wherein
R₁ is a group selected from optionally substituted fused polycyclic groups, substituted alkyl groups, branched alkyl groups, and optionally substituted cycloalkyl groups
Z is a linker which is or comprises a carbonyl group or a isostere of a carbonyl group
R₂ is selected from optionally substituted aromatic rings and optionally substituted heterocyclic rings
wherein
(a) R₂ is a 2-substituted thiophene group, and/or
(b) Z is a group of the formula —C(═O)—CR₃R₄—X—(CR₅R₆)n-, wherein X is selected from NR₇, S, O, S═O, and S(═O)₂, wherein n is 0 or 1 and/or
(c) R₁ is an adamantyl group and Z is or comprises an amide group, and/or
(d) R₁ is an adamantyl group and Z is or comprises a group of the formula —(CR₈R₉)p-NR₁₀—S(═O)—(CR₁₁R₁₂)q-, wherein p is 0 or 1 and q is 0 or 1 and/or
(e) R₁ is an adamantyl group and Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is o or 1 and w is 0 or 1;
wherein each of R₃, R₄, R₅, R₆, R₈, R₉, R₁₁, R₁₂, R₁₃, R₁₄, R₁₅ and R₁₆, are independently selected from H, hydrocarbyl and halogen,
wherein each of R₇ and R₁₀ are independently selected from H and hydrocarbyl.

In one aspect the present invention provides a pharmaceutical composition comprising (i) a compound having Formula I

R₁-Z-R₂ Formula I

wherein
R₁ is a group selected from optionally substituted fused polycyclic groups, substituted alkyl groups, branched alkyl groups, and optionally substituted cycloalkyl groups
Z is a linker which is or comprises a carbonyl group or a isostere of a carbonyl group
R₂ is selected from optionally substituted aromatic rings and optionally substituted heterocyclic rings
wherein
(a) R₂ is a 2-substituted thiophene group, and/or
(b) Z is a group of the formula —C(═O)—CR₃R₄—X—(CR₅R₆)n-, wherein X is selected from NR₇, S, O, S═O, and S(═O)₂, wherein n is 0 or 1 and/or
(c) R₁ is an adamantyl group and Z is or comprises an amide group, and/or
(d) R₁ is an adamantyl group and Z is or comprises a group of the formula —(CR₈R₉)p-NR₁₀—S(═O)₂—(CR₁₁R₁₂)q-, wherein p is 0 or 1 and q is 0 or 1 and/or
(e) R₁ is an adamantyl group and Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is o or 1 and w is 0 or 1;
wherein each of R₃, R₄, R₅, R₆, R₈, R₉, R₁₁, R₁₂, R₁₃, R₁₄, R₁₅ and R₁₆, are independently selected from H, hydrocarbyl and halogen,
wherein each of R₇ and R₁₀ are independently selected from H and hydrocarbyl.
(ii) optionally admixed with a pharmaceutically acceptable carrier, diluent, excipient or adjuvant.

In one aspect the present invention provides a compound for use in medicine wherein the compound is of Formula I

R₁-Z-R₂ Formula I

wherein
R₁ is a group selected from optionally substituted fused polycyclic groups, substituted alkyl groups, branched alkyl groups, and optionally substituted cycloalkyl groups
Z is a linker which is or comprises a carbonyl group or a isostere of a carbonyl group
R₂ is selected from optionally substituted aromatic rings and optionally substituted heterocyclic rings
wherein
(a) R₂ is a 2-substituted thiophene group, and/or
(b) Z is a group of the formula —C(═O)—CR₃R₄—X—(CR₅R₆)n-, wherein X is selected from NR₇, S, O, S═O, and S(═O)₂, wherein n is 0 or 1 and/or
(c) R₁ is an adamantyl group and Z is or comprises an amide group, and/or
(d) R₁ is an adamantyl group and Z is or comprises a group of the formula —(CR₈R₉)p-NR₁₀—S(═O)₂—(CR₁₁R₁₂)q-, wherein p is 0 or 1 and q is 0 or 1 and/or
(e) R₁ is an adamantyl group and Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is o or 1 and w is 0 or 1;
wherein each of R₃, R₄, R₅, R₆, R₈, R₉, R₁₁, R₁₂, R₁₃, R₁₄, R₁₅ and R₁₆, are independently selected from H, hydrocarbyl and halogen,
wherein each of R₇ and R₁₀ are independently selected from H and hydrocarbyl.

In one aspect the present invention provides a use of a compound in the manufacture of a medicament for use in the therapy of a condition or disease associated with 11β-HSD, wherein the compound has Formula I

R₁-Z-R₂ Formula I

wherein
R₁ is a group selected from optionally substituted fused polycyclic groups, substituted alkyl groups, branched alkyl groups, and optionally substituted cycloalkyl groups
Z is a linker which is or comprises a carbonyl group or a isostere of a carbonyl group
R₂ is selected from optionally substituted aromatic rings and optionally substituted heterocyclic rings
wherein
(a) R₂ is a 2-substituted thiophene group, and/or
(b) Z is a group of the formula —C(═O)—CR₃R₄—X—(CR₅R₆)n-, wherein X is selected from NR₇, S, O, S═O, and S(═O)₂, wherein n is 0 or 1 and/or
(c) R₁ is an adamantyl group and Z is or comprises an amide group, and/or
(d) R₁ is an adamantyl group and Z is or comprises a group of the formula —(CR₈R₉)p-NR₁₀—S(═O)₂—(CR₁₁R₁₂)q-, wherein p is 0 or 1 and q is 0 or 1 and/or
(e) R₁ is an adamantyl group and Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is o or 1 and w is 0 or 1;
wherein each of R₃, R₄, R₅, R₆, R₈, R₉, R₁₁, R₁₂, R₁₃, R₁₄, R₁₅ and R₁₆, are independently selected from H, hydrocarbyl and halogen,
wherein each of R₇ and R₁₀ are independently selected from H and hydrocarbyl.

### SOME ADVANTAGES

One key advantage of the present invention is that the compounds of the present invention can act as 11β-HSD inhibitors. The compounds may inhibit the interconversion of inactive 11-keto steroids with their active hydroxy equivalents. Thus present invention provides methods by which the conversion of the inactive to the active form may be controlled, and to useful therapeutic effects which may be obtained as a result of such control. More specifically, but not exclusively, the invention is concerned with interconversion between cortisone and cortisol in humans.

Another advantage of the compounds of the present invention is that they may be potent 11β-HSD inhibitors in vivo.

Some of the compounds of the present invention are also advantageous in that they may be orally active.

The present invention may provide for a medicament for one or more of (i) regulation of carbohydrate metabolism, (ii) regulation of protein metabolism, (iii) regulation of lipid metabolism, (iv) regulation of normal growth and/or development, (v) influence on cognitive function, (vi) resistance to stress and mineralocorticoid activity.

Some of the compounds of the present invention may also be useful for inhibiting hepatic gluconeogenesis. The present invention may also provide a medicament to relieve the effects of endogenous glucocorticoids in diabetes mellitus, obesity (including centripetal obesity), neuronal loss and/or the cognitive impairment of old age. Thus, in a further aspect, the invention provides the use of an inhibitor of 11113-HSD in the manufacture of a medicament for producing one or more therapeutic effects in a patient to whom the medicament is administered, said therapeutic effects selected from inhibition of hepatic gluconeogenesis, an increase in insulin sensitivity in adipose tissue and muscle, and the prevention of or reduction in neuronal loss/cognitive impairment due to glucocorticoid-potentiated neurotoxicity or neural dysfunction or damage.

From an alternative point of view, the invention provides a method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, and any combination of the aforementioned conditions, the method comprising the step of administering to said patient a medicament comprising a pharmaceutically active amount of a compound in accordance with the present invention.

Some of the compounds of the present invention may be useful for the treatment of cancer, such as breast cancer, as well as (or in the alternative) non-malignant conditions, such as the prevention of auto-immune diseases, particularly when pharmaceuticals may need to be administered from an early age.

### DETAILED ASPECTS OF THE PRESENT INVENTION

As previously mentioned, in one aspect the present invention provides a compound having Formula I defined above.

As previously mentioned, in one aspect the present invention provides a pharmaceutical composition comprising

(i) a compound having Formula I defined above
(ii) optionally admixed with a pharmaceutically acceptable carrier, diluent, excipient or adjuvant.

As previously mentioned, in one aspect the present invention provides a compound having Formula I defined above, for use in medicine.

As previously mentioned, in one aspect the present invention provides a use of a compound having Formula I defined above in the manufacture of a medicament for use in the therapy of a condition or disease associated with 11β-HSD.

In one aspect the present invention provides a use of a compound having Formula I defined above in the manufacture of a medicament for use in the therapy of a condition or disease associated with adverse 11β-HSD levels.

In one aspect the present invention provides a use of a compound having Formula I defined above in the manufacture of a pharmaceutical for modulating 11β-HSD activity.

In one aspect the present invention provides a use of a compound having Formula I defined above in the manufacture of a pharmaceutical for inhibiting 11β-HSD activity.

In one aspect the present invention provides a method comprising (a) performing a 11β-HSD assay with one or more candidate compounds having Formula I defined above; (b) determining whether one or more of said candidate compounds is/are capable of modulating 11β-HSD activity; and (c) selecting one or more of said candidate compounds that is/are capable of modulating 11β-HSD activity.

In one aspect the present invention provides a method comprising (a) performing a 11β-HSD assay with one or more candidate compounds having Formula I defined above; (b) determining whether one or more of said candidate compounds is/are capable of inhibiting 11β-HSD activity; and (c) selecting one or more of said candidate compounds that is/are capable of inhibiting 11β-HSD activity.

In one aspect the present invention provides
a compound identified by the above method,
the use of the said compound in medicine,
a pharmaceutical composition comprising the said compound, optionally admixed with a pharmaceutically acceptable carrier, diluent, excipient or adjuvant,
use of the said compound in the manufacture of a medicament for use in the therapy of a condition or disease associated with 11β-HSD, and
a use of the said compound in the manufacture of a medicament for use in the therapy of a condition or disease associated with adverse 11β-HSD levels.

For ease of reference, these and further aspects of the present invention are now discussed under appropriate section headings. However, the teachings under each section are not necessarily limited to each particular section.

### PREFERABLE ASPECTS

### Compound

As previously mentioned, in one aspect the present invention provides a compound having Formula I

R₁-Z-R₂ Formula I

wherein
R₁ is a group selected from optionally substituted fused polycyclic groups, substituted alkyl groups, branched alkyl groups, and optionally substituted cycloalkyl groups
Z is a linker which is or comprises a carbonyl group or a isostere of a carbonyl group
R₂ is selected from optionally substituted aromatic rings and optionally substituted heterocyclic rings
wherein
(a) R₂ is a 2-substituted thiophene group, and/or
(b) Z is a group of the formula —C(═O)—CR₃R₄—X—(CR₅R₆)n-, wherein X is selected from NR₇, S, O, S═O, and S(═O)₂, wherein n is 0 or 1 and/or
(c) R₁ is an adamantyl group and Z is or comprises an amide group, and/or
(d) R₁ is an adamantyl group and Z is or comprises a group of the formula —(CR₈R₉)p-NR₁₀—S(═O)₂—(CR₁₁R₁₂)q-, wherein p is 0 or 1 and q is 0 or 1 and/or
(e) R₁ is an adamantyl group and Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is o or 1 and w is 0 or 1;
wherein each of R₃, R₄, R₅, R₆, R₈, R₉, R₁₁. R₁₂, R₁₃, R₁₄, R₁₅ and R₁₆, are independently selected from H, hydrocarbyl and halogen,
wherein each of R₇ and R₁₀ are independently selected from H and hydrocarbyl.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In some aspects of the present invention, one or more hydrocarbyl groups is independently selected from optionally substituted alkyl group, optionally substituted haloalkyl group, aryl group, alkylaryl group, alkylarylakyl group, and an alkene group.

In some aspects of the present invention, one or more hydrocarbyl groups is independently selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

In some aspects of the present invention, one or more hydrocarbyl groups is independently selected from aryl groups, alkylaryl groups, alkylarylakyl groups, —(CH₂)₁₋₁₀-aryl, —(CH₂)₁₋₁₀-Ph, (CH₂)₁₋₁₀-Ph-C₁₋₁₀ alkyl, —(CH₂)₁₋₅-Ph, (CH₂)₁₋₅-Ph-C₁₋₅ alkyl, —(CH₂)₁₋₃-Ph, (CH₂)₁₋₃-Ph-C₁₋₃ alkyl, —CH₂-Ph, and —CH₂-Ph-C(CH₃)₃. The aryl groups may contain a hetero atom. Thus the aryl group or one or more of the aryl groups may be carbocyclic or more may heterocyclic. Typical hetero atoms include O, N and S, in particular N.

In some aspects of the present invention, one or more hydrocarbyl groups is independently selected from —(CH₂)₁₋₁₀-cycloalkyl, —(CH₂)₁₋₁₀—C₃₋₁₀cycloalkyl, —(CH₂)₁₋₇—C₃₋₇cycloalkyl, —(CH₂)₁₋₅—C₃₋₅cycloalkyl, —(CH₂)₁₋₃—C₃₋₅cycloalkyl, and —CH₂—C₃cycloalkyl.

In some aspects of the present invention, one or more hydrocarbyl groups is independently selected from alkene groups. Typical alkene groups include C₁-C₁₀ alkene group, C₁-C₆ alkene group, C₁-C₃ alkene group, such as C₁, C₂, C₃, C₄, C₅, C₆, or C₇ alkene group. In a preferred aspect the alkene group contains 1, 2 or 3 C═C bonds. In a preferred aspect the alkene group contains 1 C═C bond. In some preferred aspect at least one C═C bond or the only C═C bond is to the terminal C of the alkene chain, that is the bond is at the distal end of the chain to the ring system.

In some aspects of the present invention, one or more hydrocarbyl groups is independently selected from oxyhydrocarbyl groups.

One particular hydrocarbyl group is an oxyhydrocarbyl group. The term “oxyhydrocarbyl” group as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Typically, the oxyhydrocarbyl group is of the formula C₁₋₆O (such as a C₁₋₃O).

### Group R₁

R₁ is a group selected from optionally substituted fused polycyclic groups, substituted alkyl groups, branched alkyl groups, and optionally substituted cycloalkyl groups

In one preferred aspect R₁ is a group selected from unsubstituted fused polycyclic groups, substituted alkyl groups, branched alkyl groups, and optionally substituted cycloalkyl groups.

### Optionally Substituted Fused Polycyclic Groups

R₁ may be an optionally substituted fused polycyclic group. In one aspect R₁ is an optionally substituted fused polycyclic group.

The optionally substituted fused polycyclic groups may be a substituted fused polycyclic groups or an unsubstituted fused polycyclic groups

The optionally substituted fused polycyclic group may be of any suitable size. For example the optionally substituted fused polycyclic group may be optionally substituted C₇₋₅₀ fused polycyclic group, such as a optionally substituted C₇₋₄ fused polycyclic group, such as a optionally substituted C₇₋₃₀ fused polycyclic group, such as a optionally substituted C₇₋₂₀ fused polycyclic group, such as a optionally substituted C₇₋₁₀ fused polycyclic group, such as a optionally substituted C₇₋₁₀ fused polycyclic group. Examples of optionally substituted fused polycyclic group also include optionally substituted C₈₋₅₀ fused polycyclic group, such as a optionally substituted C₈₋₄₀ fused polycyclic group, such as a optionally substituted C₈₋₃₀ fused polycyclic group, such as a optionally substituted C₈₋₂₀ fused polycyclic group, such as a optionally substituted C₈₋₁₀ fused polycyclic group, such as a optionally substituted C₉₋₁₀ fused polycyclic group.

Particularly preferred are optionally substituted C₇, C₉ and C₁₀ fused polycyclic groups and in particular C₉ and C₁₀ fused polycyclic groups.

If the fused polycyclic group is substituted, the substitution may be at any point on the polycyclic ring. However in one preferred aspect the optionally substituted fused polycyclic group is substituted at the carbon other than the one attaching the fused polycyclic group to Z. In a highly preferred aspect the fused polycyclic group is substituted only at this point, namely only at a carbon not attaching the cycloalkyl group to Z.

If the fused polycyclic group is substituted, it is preferred that the fused polycyclic group is mono-substituted or di-substituted. Thus in one preferred aspect the optionally substituted fused polycyclic group is a mono-substituted fused polycyclic group, a di-substituted fused polycyclic group or an unsubstituted fused polycyclic group

In a further preferred aspect the optionally substituted fused polycyclic group is mono-substituted.

Preferably the or each optional substituent of the optionally substituted fused polycyclic group is independently selected from hydrocarbyl groups, halogens, hydroxyl, amines, and amides. The amines may be unsubstituted, mono-substituted or disubstituted. Preferably the or each optional substituent is selected from hydrocarbon groups, oxyhydrocarbon groups, hydroxyl, halogens, amines and amides. More preferably the or each optional substituent is selected from aromatic hydrocarbon groups, alkyl groups, oxyalkyl groups, halogens, hydroxyl, amines and amides, such as from aromatic hydrocarbon groups, alkyl groups, oxyalkyl groups and halogens. In a preferred aspect the or each optional substituent is selected from phenyl groups, hydroxyl, C₁₋₅alkyl groups, oxy-C₁₋₅-alkyl groups, NH₂, NHC₁₋₅-alkyl and N(C₁₋₅-alkyl)(C₁₋₅-alkyl). More preferably the substituents are selected from hydroxyl, phenyl, methyl, ethyl, propyl, O-methyl, O-ethyl, O-propyl, NH₂, and NHMe. A highly preferred substituent is a methyl group or a hydroxyl.

Preferably the or each optional substituent of the optionally substituted fused polycyclic group is independently selected from hydrocarbon groups, oxyhydrocarbon groups, and halogens.

Preferably the or each optional substituent of the optionally substituted fused polycyclic group is independently selected from alkyl groups.

The alkyl group substituent may be of any suitable length. The alkyl group substituent may straight chain or branched. For example the alkyl group substituent may be a C₁₋₅₀ alkyl group, such as a C₁₋₄₀ alkyl group, such as a C₁₋₃₀ alkyl group, such as a C₁₋₂₀ alkyl group, such as a C₁₋₁₀ alkyl group, such as a C₁₋₅ alkyl group, or such as a C₁₋₃ alkyl group. In one highly preferred aspect the alkyl group is a methyl group.

In one preferred aspect the fused polycyclic group comprises three fused rings. Preferably each ring is fused to each other ring.

The optionally substituted fused polycyclic groups may be carbocyclic or may contain carbon and one or more hetero atoms. Suitable hetero atoms will be apparent to those skilled in the art and include, for instance, sulphur, nitrogen and oxygen. Preferably the fused polycyclic group comprises only carbocyclic fused rings. A suitable and preferred optionally substituted hetero atom containing fused polycyclic group is

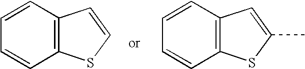

wherein ---- denotes the point of attachment to Z.

A preferred hetero atom containing fused polycyclic group is

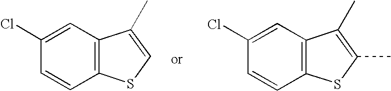

wherein ---- denotes the point of attachment to Z.

In one preferred aspect the fused polycyclic group is non-aromatic.

Particularly preferred fused polycyclic groups are optionally substituted adamantyl groups and optionally substituted noradamantyl groups.

A noradamantyl group is a group of the formula

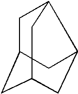

In one highly preferred aspect the fused polycyclic groups are selected from unsubstituted adamantyl group and unsubstituted noradamantyl group.

In one preferred aspect the fused polycyclic groups is an optionally substituted adamantyl group.

In one highly preferred aspect the fused polycyclic groups is an unsubstituted adamantyl group.

In one highly preferred aspect the fused polycyclic groups is a group of the formula

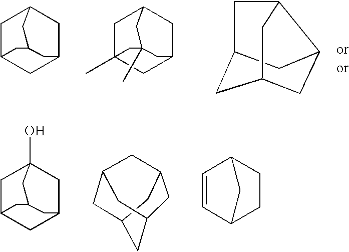

In one highly preferred aspect the fused polycyclic groups is a group of the formula

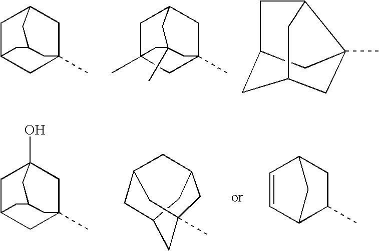

wherein ---- denotes the point of attachment to Z

In one highly preferred aspect the fused polycyclic groups is an adamantyl group of the formula.

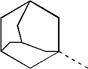

wherein ---- denotes the point of attachment to Z.

### Substituted Alkyl Groups

R₁ may be a substituted alkyl group. In one aspect R₁ is a substituted alkyl group

In a preferred aspect the substituted alkyl group is an alkyl group substituted with at least one substituent independently selected or only with substituents independently selected from hydrocarbyl groups, halogens, hydroxyl, amines, and amides. The amines may be unsubstituted, mono-substituted or disubstituted. Preferably the or each optional substituent is selected from hydrocarbon groups, oxyhydrocarbon groups, hydroxyl, halogens, amines and amides. More preferably the or each optional substituent is selected from aryl groups, aromatic hydrocarbon groups, alkyl groups, oxyalkyl groups, halogens, hydroxyl, amines and amides, such as from aromatic hydrocarbon groups, alkyl groups, oxyalkyl groups and halogens. In a preferred aspect the or each optional substituent is selected from phenyl groups, hydroxyl, C₁₋₅alkyl groups, oxy-C₁₋₅-alkyl groups, NH₂, NHC₁₋₆-alkyl and N(C₁₋₅-alkyl)(C₁₋₆-alkyl). More preferably the substituents are selected from hydroxyl, phenyl, methyl, ethyl, propyl, O-methyl, O-ethyl, O-propyl, NH₂, and NHMe. A highly preferred substituent is a phenyl group.

The alkyl may contain one or more degrees of substitution. If the alkyl group contains more than one substitution, each substitution may be on a different carbon of the alkyl, on the same carbon or if three or more substitutions are present a combination thereof is envisaged. In one preferred aspect the substituted alkyl group is di-substituted. More preferably, both of the substitutions are on the same carbon of the alkyl group.

The alkyl group may be of any suitable length. For example the substituted alkyl group may be a substituted C₁₋₅₀ alkyl group, such as a substituted C₁₋₄₀ alkyl group, such as a substituted C₁₋₃₀ alkyl group, such as a substituted C₁₋₂₀ alkyl group, such as a substituted C₁₋₁₀ alkyl group, or such as a substituted C₁₋₅ alkyl group. In one highly preferred aspect the substituted alkyl group is a substituted ethyl group. In particular a disubstituted ethyl group is preferred.

It will be appreciated by one skilled in the art that any of the above alkyl groups and substituents may be combined. Thus combining two highly preferred aspects it will be appreciated that preferably the substituted alkyl group is —C(Ph)₂-CH₃.

### Branched Alkyl Groups

R₁ may be a branched alkyl group. In one aspect R₁ is a branched alkyl group.

The branched alkyl group may be of any suitable length. For example the branched alkyl group may be a branched C₁₋₅₀ alkyl group, such as a branched C₁₋₄₀ alkyl group, such as a branched C₁₋₃₀ alkyl group, such as a branched C₁₋₂₀ alkyl group, such as a branched C₁₋₁₀ alkyl group, or such as a branched C₁₋₅ alkyl group. In one highly preferred aspect the branched alkyl group is a branched C₄ or C₅ alkyl group.

In a highly preferred aspect the branched alkyl group is or comprises a —C(CH₃)₃ [t-butyl] group. In one preferred aspect the branched alkyl group is a —C(CH₃)₃ [t-butyl] group.

In a highly preferred aspect the branched alkyl group is a —CH₂C(CH₃)₃ group.

### Optionally Substituted Cycloalkyl Groups

R₁ may be an optionally substituted cycloalkyl group. In one aspect R₁ is an optionally substituted cycloalkyl group.

In one aspect the optionally substituted cycloalkyl group is a substituted cycloalkyl group. In one aspect the optionally substituted cycloalkyl group is a unsubstituted cycloalkyl group.

In one aspect R₁ is a substituted cycloalkyl group. In one aspect R₁ is a unsubstituted cycloalkyl group.

The optionally substituted cycloalkyl group may be a single ring system or fused polycyclic ring system. In one aspect the optionally substituted cycloalkyl group contains only a single ring.

The optionally substituted cycloalkyl group may be of any suitable size. For example the optionally substituted cycloalkyl group may be optionally substituted C₃₋₅₀ cycloalkyl group, such as a optionally substituted C₃₋₄₀ cycloalkyl group, such as a optionally substituted C₃₋₃₀ cycloalkyl group, such as a optionally substituted C₃₋₂₀ cycloalkyl group, such as a optionally substituted C₃₋₁₀ cycloalkyl group, such as a optionally substituted C₃₋₆ cycloalkyl group,

Particularly preferred are optionally substituted C₃, C₅ and C₆ cycloalkyl groups.

If the cycloalkyl groups is substituted, the substitution may be at any point on the cycloalkyl ring. However in one preferred aspect the optionally substituted cycloalkyl group is substituted at the carbon attaching the cycloalkyl group to Z. In a highly preferred aspect the cycloalkyl group is substituted only at this point, namely only at the carbon attaching the cycloalkyl group to Z.

If the cycloalkyl groups is substituted, it is preferred that the cycloalkyl group is mono-substituted. Thus in one preferred aspect the optionally substituted cycloalkyl group is a mono-substituted cycloalkyl group or an unsubstituted cycloalkyl group

In a further preferred aspect the optionally substituted cycloalkyl group is mono-substituted.

Preferably the or each optional substituent of the optionally substituted cycloalkyl group is independently selected from hydrocarbyl groups, halogens, hydroxyl, amines, and amides. The amines may be unsubstituted, mono-substituted or disubstituted. Preferably the or each optional substituent is selected from hydrocarbon groups, oxyhydrocarbon groups, hydroxyl, halogens, amines and amides. More preferably the or each optional substituent is selected from aromatic hydrocarbon groups, alkyl groups, oxyalkyl groups, halogens, hydroxyl, amines and amides, such as from aromatic hydrocarbon groups, alkyl groups, oxyalkyl groups and halogens. In a preferred aspect the or each optional substituent is selected from phenyl groups, hydroxyl, C₁₋₅alkyl groups, oxy-C₁₋₅alkyl groups, NH₂, NHC₁₋₅-alkyl and N(C₁₋₅-alkyl)(C₁₋₅-alkyl). More preferably the substituents are selected from hydroxyl, phenyl, methyl, ethyl, propyl, O-methyl, O-ethyl, O-propyl, NH₂, and NHMe. A highly preferred substituent is a methyl group or a hydroxyl.

Preferably the or each optional substituent of the optionally substituted cycloalkyl group is independently selected from hydrocarbon groups, oxyhydrocarbon groups, and halogens.

Preferably the or each optional substituent of the optionally substituted cycloalkyl group is independently selected from alkyl groups and optionally substituted aryl groups.

The alkyl group substituent may be of any suitable length. The alkyl group substituent may straight chain or branched. For example the alkyl group substituent may be a C₁₋₅₀ alkyl group, such as a C₁₋₄₀ alkyl group, such as a C₁₋₃₀ alkyl group, such as a C₁₋₂₀ alkyl group, such as a C₁₋₁₀ alkyl group, such as a C₁₋₅ alkyl group, or such as a C₁₋₃ alkyl group. In one highly preferred aspect the alkyl group is a methyl group.

The aryl group substituent may also be bicyclic such as biphenyl group and/or may heteroaryl, such as a pyridine, thiophene and fused benzo analogues.

The aryl group substituent is an optionally substituted phenyl group. Preferably the optionally substituted aryl group is a substituted phenyl group.

The aryl group substituent may be optionally substituted. Suitable substituents include substituents independently selected from hydrocarbyl groups, halogens, hydroxyl, S═O, S(═O)₂, C≡N, CO₂H, CO₂-hydrocarbyl, amines, and amides. The amines may be unsubstituted, mono-substituted or disubstituted. Preferably the or each optional substituent is selected from hydrocarbon groups, oxyhydrocarbon groups, hydroxyl, halogens, amines and amides. More preferably the or each optional substituent is selected from aromatic hydrocarbon groups, alkyl groups, oxyalkyl groups, halogens, hydroxyl, amines and amides, such as from aromatic hydrocarbon groups, alkyl groups, oxyalkyl groups and halogens. In a preferred aspect the or each optional substituent is selected from halogens, phenyl groups, hydroxyl, C₁₋₅alkyl groups, oxy-C₁₋₅-alkyl groups, NH₂, NHC₁₋₅-alkyl and N(C₁₋₅-alkyl)(C₁₋₅-alkyl). More preferably the substituents are selected from halogens, hydroxyl, phenyl, methyl, ethyl, propyl, O-methyl, O-ethyl, O-propyl, NH₂, and NHMe. A highly preferred substituent is a fluoro or chloro group.

Preferably the aryl group substituent may be selected from halogens, preferably the aryl group substituent(s) is at least one chloro group.

In a highly preferred aspect the optionally substituted cycloalkyl group is selected from

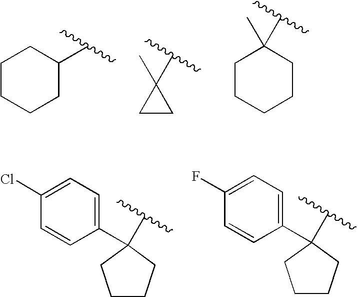

In a highly preferred aspect the optionally substituted cycloalkyl group is

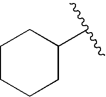

In a highly preferred aspect the optionally substituted cycloalkyl group is

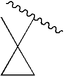

In a highly preferred aspect the optionally substituted cycloalkyl group is

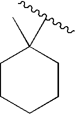

In a highly preferred aspect the optionally substituted cycloalkyl group is

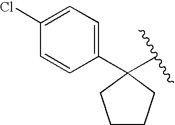

In a highly preferred aspect the optionally substituted cycloalkyl group is

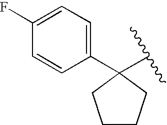

### R₁

It will be appreciated from above that in highly preferred aspects R₁ is selected from an adamantyl group, a noradamantyl group, a —C(Ph)₂-CH₃. group, a —CH₂C(CH₃)₃ group,

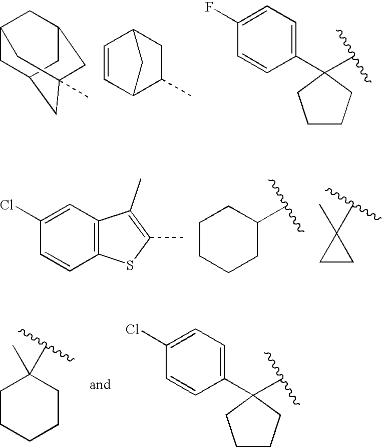

In a further highly preferred R₁ is an adamantyl group

In a further highly preferred R₁ is —C(Ph)₂-CH₃.

In a further highly preferred R₁ is a —CH₂C(CH₃)₃ group.

In a further highly preferred R₁ is a —C(CH₃)₃ group.

In a further highly preferred R₁ is selected from

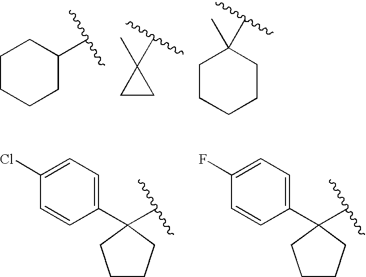

In a further highly preferred R₁ is

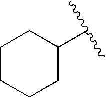

In a further highly preferred R₁ is

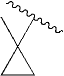

In a further highly preferred R₁ is

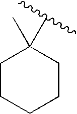

In a further highly preferred R₁ is

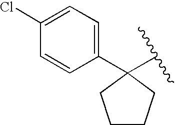

In a further highly preferred R₁ is

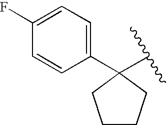

### Group Z

As discussed herein Z is a linker which is or comprises a carbonyl group or an isostere of a carbonyl group

Isosteres and suitable isosteres are discussed in Thomber, C. W., Isosterism and molecular modification in drug design, Chemical Society Reviews (1979), 8(4), 563-80.

It will be appreciated by one skilled in the art that for each of groups Z discussed herein the group may run between R₁ and R₂ either in the direction shown herein or in the reverse direction. In other words if group Z is read from left to right, the left most moiety of group Z is attached to R₁ and the right most moiety of group Z is attached to R₂; or the left most moiety of group Z is attached to R₂ and the right most moiety of group Z is attached to R₁. In a preferred aspect the left most moiety of group Z is attached to R₁ and the right most moiety of group Z is attached to R₂.

In one aspect Z is a linker which is or comprises a carbonyl group. In one aspect Z is a linker which comprises a carbonyl group.

In a further preferred aspect the isostere of a carbonyl group is a group selected from C═N, C—OH, C═C, C═NOH, C═NOC₁₋₅ alkyl, C═NNH₂, C═NNHC₁₋₅ alky, C═NN(C₁₋₅ alky)₂, C≡N, C═NCN, C═NNO₂, C═S, S═O, S(═O)₂, S═NH, S═NC₁₋₅ alky, P═O, P(═O)₂, P—OH, P═S, P—SH, P═NH, and P═NC₁₋₅ alkyl. A preferred isostere of a carbonyl group is —S(═O)₂. When Z comprises an isostere of a carbonyl group, Z is preferably —S(═O)₂—NH—CH₂—.

Preferably Z is a group of the formula —C(═O)—CR₃R₄—X—(CR₅R₆)n-, wherein X is selected from NR₇, S, O, S═O, and S(═O)₂, wherein n is from 0 to 10, such as from 0 to 5, such from 0 to 3, such as 0 or 1, and wherein each of R₃ and R₄, are independently selected from H, hydrocarbyl and halogen, wherein R₇ is selected from H and hydrocarbyl.

Preferably Z is selected from —C(═O)CH₂NH—, —C(═O)CH₂NMe-, —C(═O)CH₂NHCH₂—, —C(═O)CH₂NMeCH₂—(.HCl), —C(═O)CH₂—S—, —C(═O)CH₂—S(—O)₂—, —C(═O)CH₂—S(═O)—, C(═O)CH₂—O, —C(═O)—CH₂—S—CH₂—, —C(═O)CH₂—O—CH₂—, —C(═O)CH₂—S(═O)₂—CH₂—, and —C(═O)CH₂—S(═O)—CH₂—

Preferably Z is selected from —C(═O)CH₂NH—, —C(═O)CH₂—S—, —C(═O)CH₂—S(═O)₂—, —C(═O)CH₂S(═O)—, —C(═O)CH₂—O—, —C(═O)—CH₂—S—CH₂—, —C(═O)CH₂—O—CH₂—, —C(═O)CH₂—S(═O)₂—CH₂—, and —C(═O)CH₂—S(═O)—CH₂—.

In one preferred aspect Z is or comprises an amide group. In one preferred aspect Z is an amide group. Suitable amide groups are of the formula —(CR₁₇R₁₈)₀₋₆—C(═O)NR₁₉—(CR₂₀R₂₁)₀₋₆— wherein each of R₁₇, R₁₈, R₂₀ and R₂₁, is independently selected from H, hydrocarbyl and halogen, wherein R₁₉ is from H and hydrocarbyl. Preferably each of R₁₇, R₁₈, R₁₉, R₂₀ and R₂₁, is independently selected from H and methyl. Further suitable amide groups are of the formula —(CH₂)₀₋₆—C(═O)NH—(CH₂)₀₋₆—.

Preferably Z is selected from —C(═O)NH—, —C(═O)NH—CH₂—, —C(═O)N(CHCH₃CH₃)—CH₂—, —C(═O)NMe-CH₂—, —C(═O)N(CH₂CH₃)—CH₂—, —C(═O)N(CH₂Ph)-CH₂—, —C(═O)N(CH₂-cyclohexane)-CH₂—, —C(═O)NH—(CH₂)₂—, —C(═O)NMe-(CH₂)₂—, —CH₂—C(═O)NH—CH₂—, —CH₂—C(═O)NMe-CH₂—, —CH₂—C(═O)NH—, —CH₂—C(═O)NH—(CH₂)₂—, —C(═O)NMe-CH₂—, —C(═O)NH—(CH₂)₃—, and —CH₂—C(═O)NMe-CH₂—.

Preferably Z is selected from —C(═O)NH—, —C(═O)NH—CH₂—, —C(═O)NH—(CH₂)₂—, —CH₂—C(═O)NH—CH₂—, —CH₂—C(═O)NH—, —CH₂—C(═O)NH—(CH₂)₂—, —C(═O)NMe-CH₂—, —C(═O)NH—(CH₂)₃—, and —CH₂—C(═O)NMe-CH₂—.

It will be understood by one skilled in the art that when Z comprises an amide group (such as —(CR₁₇R₁₈)₀₋₆C(═O)NR₁₉—(CR₂₀R₂₁)₀₋₆—), the entire group Z may contain other atoms such that the group as whole could be described as other than an amide. These other atoms may include groups on the N of the amide moiety which link with other atoms on the compound to form a ring. Provided the group contains the N—C═O moiety it is preferably considered herein to be an amide group. Thus when Z is or comprises an amide group, Z may be of the formula -G-(CR₁₇R₁₈)₀₋₆—C(═O)NR₁₉—(CR₂₀R₂₁)₀₋₆-J-(CR₂₃R₂₄)₀₋₆— wherein each of R₁₇, R₁₈, R₂₀, R₂₁, R₂₃ and R₂₄ is independently selected from H, hydrocarbyl and halogen, wherein R₁₉ is from H and hydrocarbyl, wherein G and J are optional groups independently selected from NR₂, S, O, S═O, and S(═O)₂, wherein R₂ is selected from H and hydrocarbyl. Preferably each of R₁₇, R₁₈, R₁₉, R₂₀, R₂₁ and R₂₂, is independently selected from H and methyl. When Z is or comprises an amide group, Z is preferably selected from groups of the formula
—NR₂—(CR₁₇R₁₈)₀₋₆—C(═O)NR₁₉—(CR₂₀R₂₁)₀₋₆—, such as —NR₂₂—C(═O)NR₁₉—(CR₂₀R₂₁)₀₋₆—, preferably —NH—C(═O)NH—CH₂— or —NH—C(═O)NH—CH₂CH₂—,
—S—(CR₁₇R₁₈)₀₋₆—C(═O)NR₁₉—(CR₂₀R₂₁)₀₋₆—, such as —S—C(═O)NR₁₉—(CR₂₀R₂₁)₀₋₆—, preferably —S—C(═O)NMe-CH₂—,
—S(═O)₂—(CR₁₇R₁₈)₀₋₆—C(═O)NR₁₉—(CR₂₀R₂₁)₀₋₆—, such as —S(═O)₂—(CR₁₇R₁₈)—C(═O)NR₁₉—, preferably —S(═O)₂—CH₂—C(═O)NMe-CH₂— or —S(═O)₂—CH₂—C(═O)NH—.
—(CR₂₀R₂₁)₀₋₆—NR₁₉—C(═O)—(CR₁₇R₁₈)₀₋₆—O—(CR₂₃R₂₄)₀₋₆—, such as —NR₁₉—C(═O)—O—(CR₂₃R₂₄)₀₋₆—, preferably —NH—C(═O)—O—CH₂— or —NH—C(═O)—O—CH₂CH₂—

R₁₉ may together with other members of Z or R₂ form a ring. For example R₁₉ may attach to the R₂ group, such as thiophene or a phenyl group, or other members of the Z group to provide a cyclic structure of which one member is the nitrogen of the amide. For example this may provide a Z group of the formula

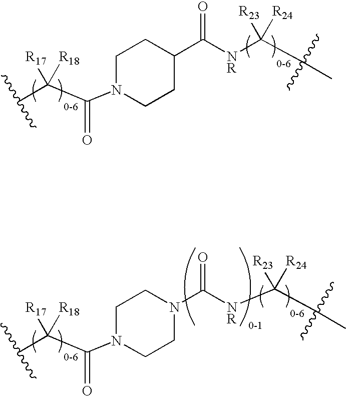

Preferred Z groups wherein R₁₉ may together with other members of Z or R₂ form a ring are selected from

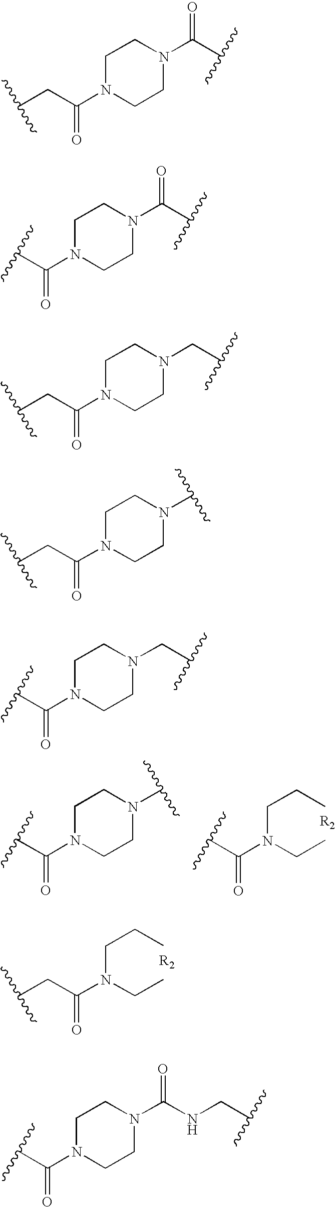

In a preferred aspect Z is or comprises a group of the formula —(CR₈R₉)p-NR₁₀—S(═O)₂—(CR₁₁R₁₂)q-, wherein each of p and q is independently selected from 0 to 10, such as from 0 to 5, such from 0 to 3, such as 0 or 1, wherein each of R₈, R₉, R₁₁, and R₁₂ is independently selected from H, hydrocarbyl and halogen, wherein R₁₀ is selected from H and hydrocarbyl.

In a preferred aspect Z is a group of the formula —(CR₈R₉)p-NR₁₀—S(═O)₂—(CR₁₁R₁₂)q-, wherein each of p and q is independently selected from 0 to 10, such as from 0 to 5, such from 0 to 3, such as 0 or 1, wherein each of R₈, R₉, R₁₁, and R₁₂ is independently selected from H, hydrocarbyl and halogen, wherein R₁₀ is selected from H and hydrocarbyl.

Preferably Z is selected from —NH—S(═O)₂—, —CH₂—NH—S(═O)₂—, and —NH—S(═O)₂—CH₂—.

In a preferred aspect Z is a group of the formula —(CR₁₃R₁₄)v-Y—(CR₁₅R₁₆)w-K— where Y is a heteroaryl group in which a bond in the heteroaryl ring is a isostere of a carbonyl group, wherein each of v and w is independently selected from 0 to 10, such as from 0 to 5, such from 0 to 3, such as 0 or 1 and where K is selected from S, S═O, O, NR₂₅ and S(═O)₂ wherein R₂₅ is selected from H and hydrocarbyl.

In a preferred aspect Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein each of v and w is independently selected from 0 to 10, such as from 0 to 5, such from 0 to 3, such as 0 or 1.

Preferably is selected from groups of the formula —CH₂—Y—CH₂—, —Y—CH₂—, —CH₂—Y— and —Y—, where Y is a heteroaryl group in which a bond in the heteroaryl ring is a isostere of a carbonyl group.

Preferably Y is an oxadiazole group or a triazole (such as 1H-1,2,3-triazole, 1H-1,2,4-triazole, or 4H-1,2,4-triazole).

Preferably Y is an oxadiazole group.

Preferably Y is a triazole group and in particular a 1H-1,2,3-triazole.

In a highly preferred aspect Z is or comprises a group of the formula

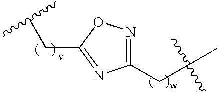

wherein each of v and w is independently selected from 0 to 10, such as from 0 to 5, such from 0 to 3, such as 0 or 1.

In a highly preferred aspect Z is a group of the formula

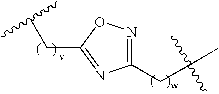

wherein each of v and w is independently selected from 0 to 10, such as from 0 to 5, such from 0 to 3, such as 0 or 1.

In a highly preferred aspect Z is or comprises a group of the formula

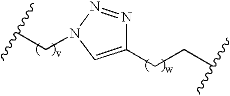

wherein each of v and w is independently selected from 0 to 10, such as from 0 to 5, such from 0 to 3, such as 0 or 1.

In a highly preferred aspect Z is or comprises a group of the formula

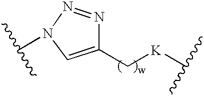

wherein w is selected from 0 to 10, such as from 0 to 5, such from 0 to 3, such as 0 or 1.

In a highly preferred aspect Z is selected from groups of the formulae

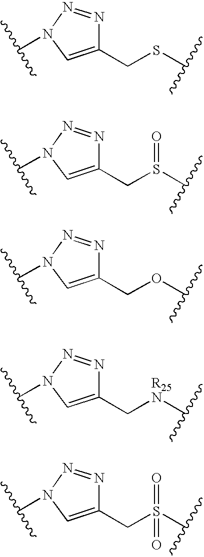

wherein R₂₅ is H or Me

### Group R₂

As discussed herein R₂ is selected from optionally substituted aromatic rings and optionally substituted heterocyclic rings.

In one aspect R₂ is an optionally substituted aromatic ring. The optionally substituted aromatic ring may be a substituted aromatic ring or an unsubstituted aromatic ring.

In one aspect R₂ is an optionally substituted heterocyclic ring. The optionally substituted heterocyclic ring may be a substituted heterocyclic ring or an unsubstituted heterocyclic ring.

Preferably R₂ is selected from substituted carbocyclic aromatic rings and unsubstituted heterocyclic rings.

Preferably the optionally substituted aromatic ring is selected from substituted carbocyclic aromatic rings and unsubstituted heterocyclic aromatic rings.

R₂ may be or comprises an optionally substituted aromatic ring. Preferably the optionally substituted aromatic ring is a five or six membered ring. In one aspect preferably R₂ is an optionally substituted five membered aromatic ring. In another aspect preferably R₂ is an optionally substituted six membered aromatic ring.

In one preferred aspect, the optionally substituted aromatic ring is a heterocyclic ring. Preferably R₂ is an optionally substituted five or six membered aromatic heterocyclic ring.

Preferably the optionally substituted aromatic ring is a heterocyclic ring comprising a carbon and a hetero atom selected from O, S and N. More preferably the optionally substituted aromatic ring is a heterocyclic ring comprising a carbon and a hetero atom selected from O and N.

In a preferred aspect R₂ is selected from optionally substituted rings

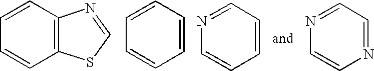

In a preferred aspect R₂ is selected from optionally substituted rings

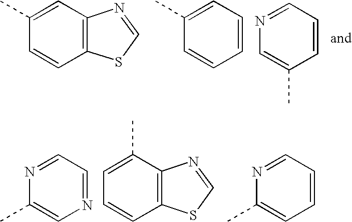

wherein ---- denotes the point of attachment to Z.

In a preferred aspect R₂ is selected from optionally substituted heterocyclic rings

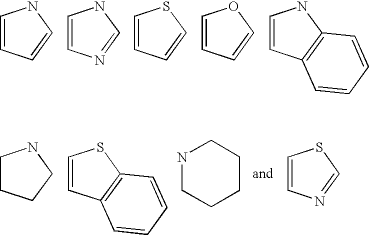

In a preferred aspect R₂ is selected from optionally substituted heterocyclic rings

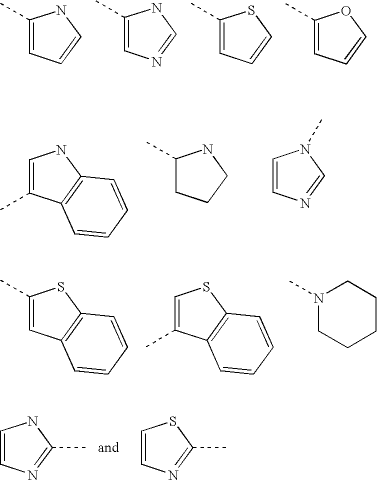

wherein ---- denotes the point of attachment to Z.

In a highly preferred aspect is

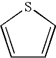

More preferably R₂ is or comprises a group selected from the following wherein ---- indicates the point of attachment to Z.

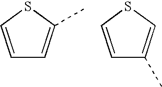

In a highly preferred aspect R₂ is or comprises a group selected from the following wherein ---- indicates the point of attachment to Z.

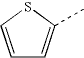

that is R₂ is a 2-thiophenyl group.

When R₂ is a thiophene group and in particular a 2-thiophenyl group, the thiophenyl group may be substituted or unsubstituted. Suitable substituents may be selected from hydrocarbyl groups, oxyhydrocarbon groups, halogens, amines and amides. More preferably the substituents are selected from —Cl, —Br, —CH₂CH₃, —CH₃, —S—CH₃, —S(═O)—CH₃, S(═O)₂—CH₃, —C(═O)—NH—CH₃, and —C(═O)—N(CH₃)—CH₃.

In a highly preferred aspect R₂ is selected from the following groups wherein ---- indicates the point of attachment to Z.

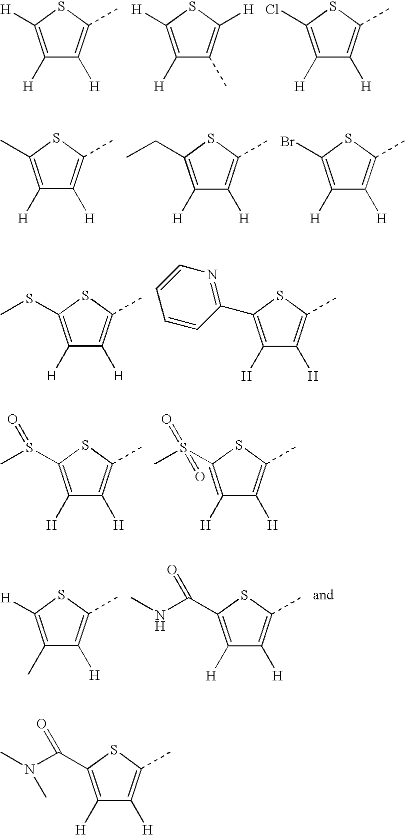

R₂ may be substituted or unsubstituted. Preferably R₂ is substituted.

In one aspect the substituents are selected from hydrocarbon groups, oxyhydrocarbon groups, halogens, amines and amides. More preferably the substituents are selected from aromatic hydrocarbon groups, alkyl groups, oxyalkyl groups, halogens, amines and amides, such as from aromatic hydrocarbon groups, alkyl groups, oxyalkyl groups and halogens.

In one aspect R₂ is substituted with two or more substituents. In one aspect R₂ is substituted with two or more substituents and the two or more substituents together form a ring which is fused to the carbocyclic ring of R₂. The carbocyclic ring may be a five or six membered-aromatic carbocyclic ring. The carbocyclic ring may be a phenyl ring.

In a preferred aspect R₂ is a substituted phenyl ring. Preferably R₂ is substituted with one or more halogen substituents and in particular chloro substituents.

In a preferred aspect R₂ is a substituted phenyl ring wherein the substituents are selected from halogens (preferably chloro and fluoro), amines, amides, optionally substituted (preferably unsubstituted) phenyl groups, alkyl groups, carboxylic acid groups, esters, alkoxy and nitro groups.

In a preferred aspect R₂ is a substituted phenyl ring wherein the substituents are selected from chloro, fluoro unsubstituted phenyl group methyl, tert-butyl, —NO₂, methoxy, —NH—C(═O)-Me, —C(═O)O-Me, —CH₂—C(═O)O-Me, —C(═O)NH—CH-Ph, —C(═O)NH-Ph, —C(═O)NH-Me, —C(═O)NH-Et, —C(═O)NH—CH(CH₃)₂, —C(═O)N-(Me)₂, —C(═O)NH—C(CH₃)₃, —CH₂—C(═O)NH—CH₂Ph, —CH₂—C(═O)NH-Ph, and —C—O—CH₂-Ph.

### FURTHER ASPECTS

For some applications, preferably the compounds have a reversible action.

For some applications, preferably the compounds have an irreversible action.

In one embodiment, the compounds of the present invention are useful for the treatment of breast cancer.

The compounds of the present invention may be in the form of a salt.

The present invention also covers novel intermediates that are useful to prepare the compounds of the present invention. For example, the present invention covers novel alcohol precursors for the compounds. The present invention also encompasses a process comprising precursors for the synthesis of the compounds of the present invention.

The compound of the present invention may have substituents other than those of the ring systems show herein. Furthermore the ring systems herein are given as general formulae and should be interpreted as such. The absence of any specifically shown substituents on a given ring member indicates that the ring member may substituted with any moiety of which H is only one example. Each ring system may contain one or more degrees of unsaturation, for example is some aspects one or more rings of a ring system is aromatic. Each ring system may be carbocyclic or may contain one or more hetero atoms.

The compound of the invention, in particular the ring systems of the compound of the invention may contain substituents other than those show herein. By way of example, these other substituents may be one or more of: one or more halo groups, one or more O groups, one or more hydroxy groups, one or more amino groups, one or more sulphur containing group(s), one or more hydrocarbyl group(s)—such as an oxyhydrocarbyl group.

In general terms the ring systems of the present compounds may contain a variety of non-interfering substituents. In particular, the ring systems may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

In a highly preferred aspect, the compounds of the present invention or for use in the present invention are selected from compounds of the formulae:

| STX | Compound | |
|---|---|---|
| CODE | No. | STRUCTURE |
| 1487 | | 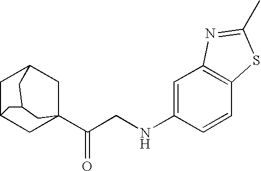 |
| 1535 | | 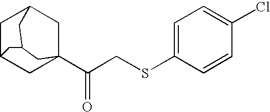 |
| 1537 | | 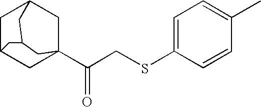 |
| 1538 | | 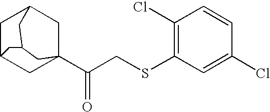 |
| 1539 | | 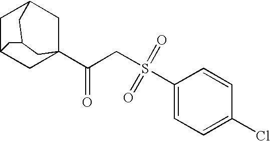 |
| 1562 | | 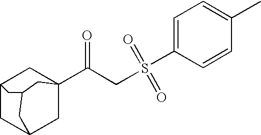 |
| 1563 | | 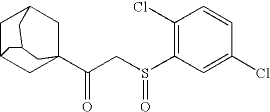 |
| 1567 | | 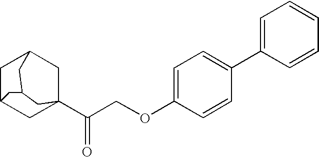 |
| 1568 | | 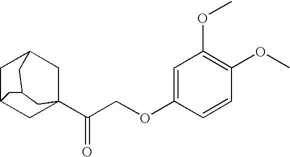 |
| 1569 | | 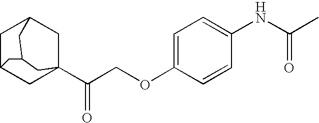 |
| 1610 | | 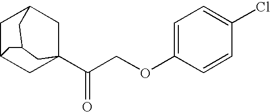 |
| 1618 | | 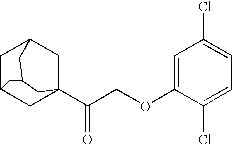 |
| 1619 | | 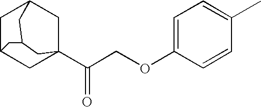 |
| 1621 | | 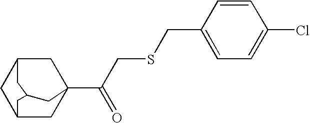 |
| 1626 | | 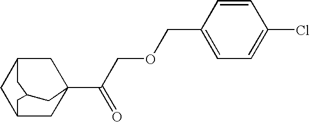 |
| 1628 | | 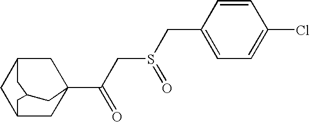 |
| 1632 | | 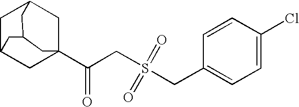 |
| 1372 | | 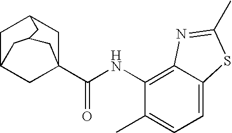 |
| 1373 | | 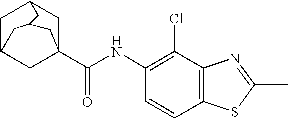 |
| 1374 | | 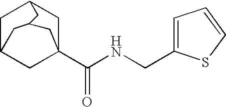 |
| 1375 | | 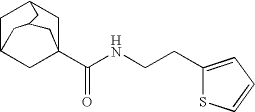 |
| 1540 | | 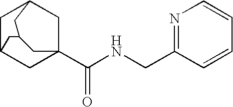 |
| 1541 | | 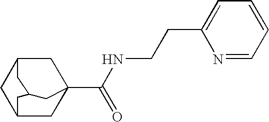 |
| 1542 | | 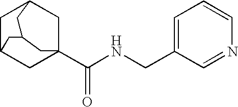 |
| 1543 | | 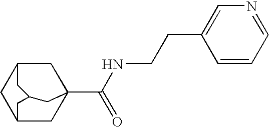 |
| 1544 | | 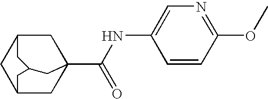 |
| 1545 | | 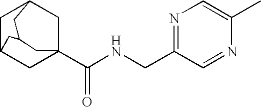 |
| 1564 | | 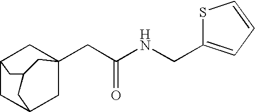 |
| 1565 | | 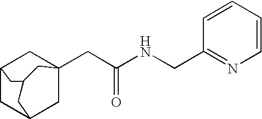 |
| 1566 | | 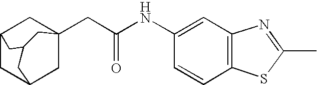 |
| 1570 | | 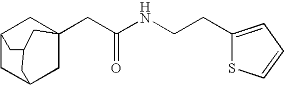 |
| 1571 | | 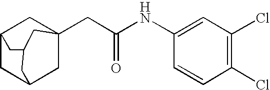 |
| 1572 | | 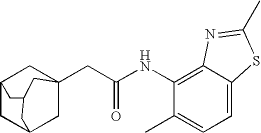 |
| 1576 | | 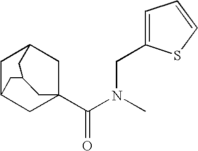 |
| 1577 | | 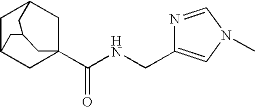 |
| 1578 | | 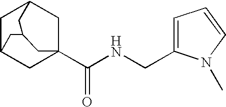 |
| 1579 | | 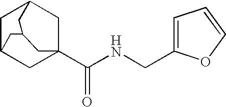 |
| 1580 | | 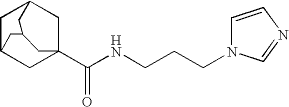 |
| 1581 | | 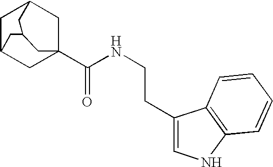 |
| 1597 | | 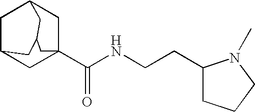 |
| 1598 | | 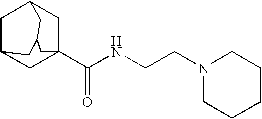 |
| 1661 | | 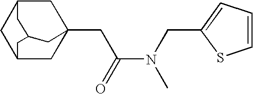 |
| 1662 | | 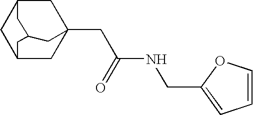 |
| 1663 | | 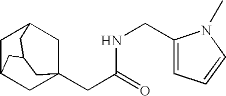 |
| 1688 | | 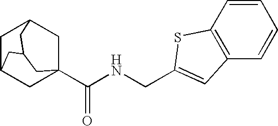 |
| 1689 | | 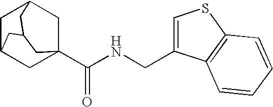 |
| 1690 | | 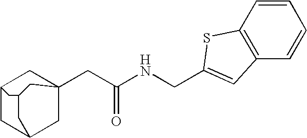 |
| 1691 | | 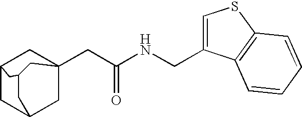 |
| 1341 | | 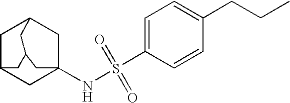 |
| 1342 | | 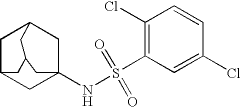 |
| 1343 | | 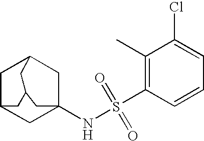 |
| 1344 | | 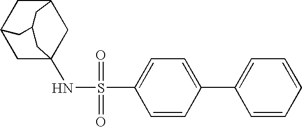 |
| 1368 | | 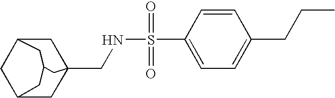 |
| 1369 | | 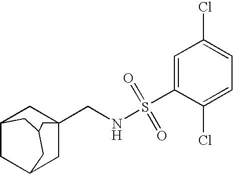 |
| 1370 | | 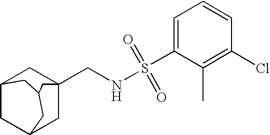 |
| 1371 | | 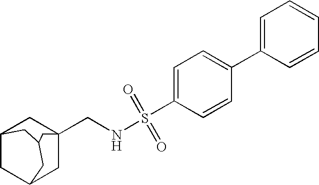 |
| 1499 | | 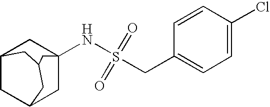 |
| 1500 | | 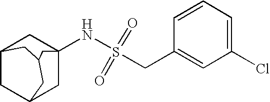 |
| 1573 | | 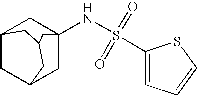 |
| 1574 | | 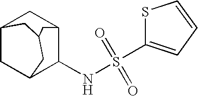 |
| 1575 | | 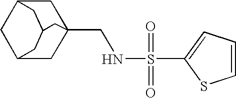 |
| 1599 | | 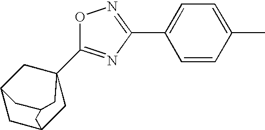 |
| 1620 | | 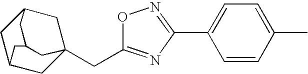 |
| 1627 | | 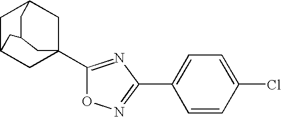 |
| 1659 | | 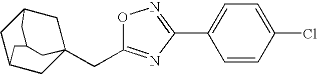 |
| 1660 | | 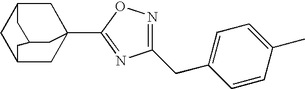 |
| 1664 | | 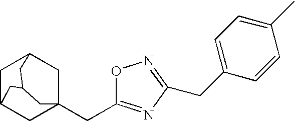 |
| 1687 | | 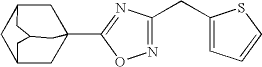 |
| 1638 | | 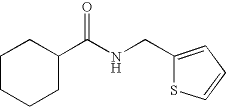 |
| 1639 | | 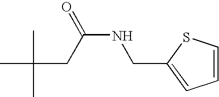 |
| 1640 | | 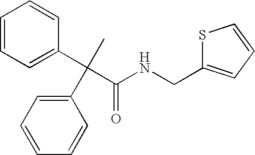 |
| 1641 | | 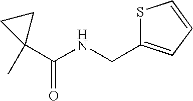 |
| 1642 | | 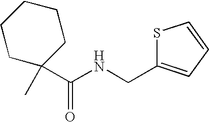 |
| 1648 | | 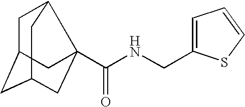 |
| 1649 | | 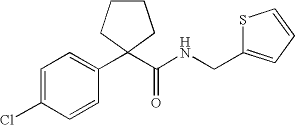 |
| 1671 | | 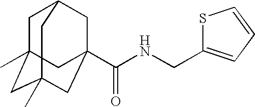 |
| 1673 | | 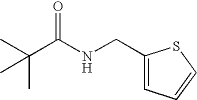 |
| 1675 | | 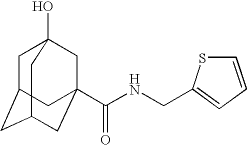 |
| 1692 | | 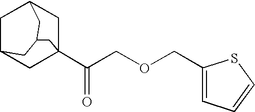 |
| 1693 | | 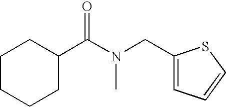 |
| 1694 | | 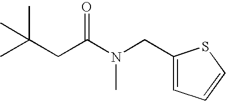 |
| 1695 | | 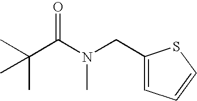 |
| 1696 | | 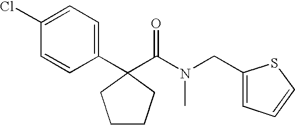 |
| 1697 | | 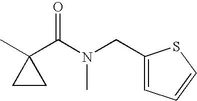 |
| 1698 | | 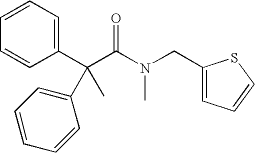 |
| 1699 | | 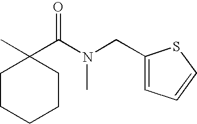 |
| 1700 | | 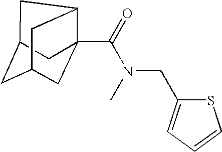 |
| 1650 | | 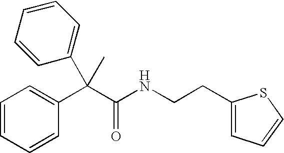 |
| 1651 | | 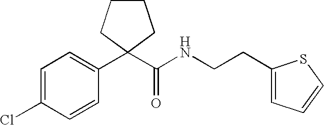 |
| 1652 | | 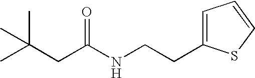 |
| 1653 | | 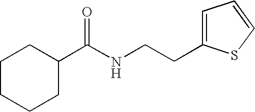 |
| 1654 | | 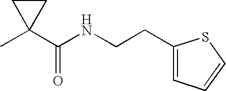 |
| 1655 | | 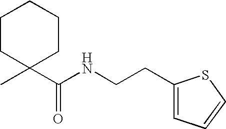 |
| 1656 | | 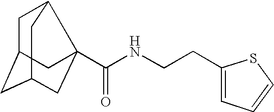 |
| 1672 | | 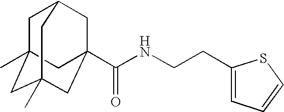 |
| 1674 | | 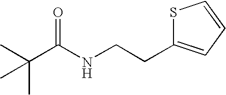 |
| 1676 | | 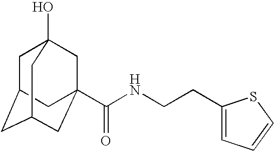 |
| 1706 | CCM01127 | 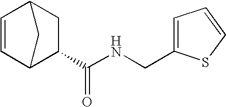 |
| 1707 | CCM01137A | 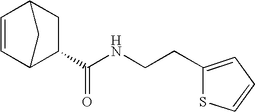 |
| 1708 | CCM01137B | 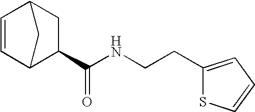 |
| 1709 | CCM01158A | 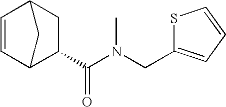 |
| 1710 | CCM01158B | 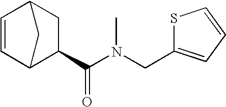 |
| 1711 | XDS04021 | 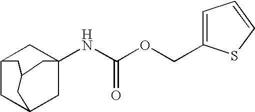 |
| 1712 | XDS04022 | 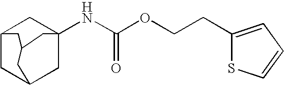 |
| 1713 | XDS04023 | 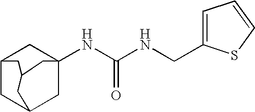 |
| 1714 | XDS04024 | 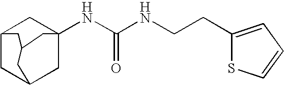 |
| 1721 | XDS04025 | 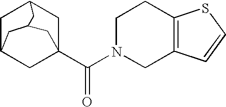 |
| 1722 | XDS04026 | 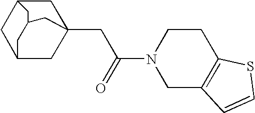 |
| 1737 | CCM01162 | 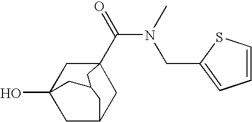 |
| 1740 | CCM01174 | 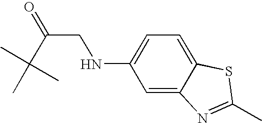 |
| 1780 | CCM01178 | 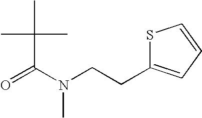 |
| 1781 | CCM01181 | 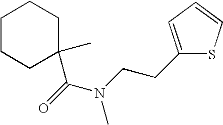 |
| 1782 | CCM01182 | 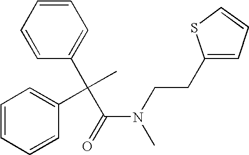 |
| 1887 | | 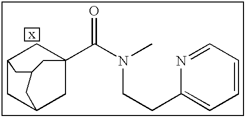 |
| 1888 | XDS04028 | |
| 1889 | XDS04030 | |
| 1890 | XDS04033 | |
| 1891 | XDS04034 | |
| 1892 | XDS04036 | |
| 1893 | XDS04037B | |
| 1894 | XDS04038 | |
| 1895 | XDS04039 | |
| 1896 | XDS04040s | 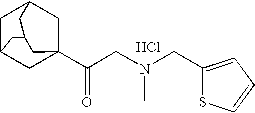 |
| 1897 | XDS04041 | 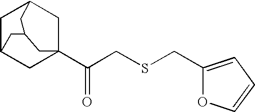 |
| 1898 | XDS04042 | 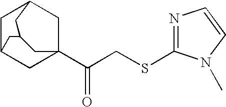 |
| 1899 | XDS04043 | 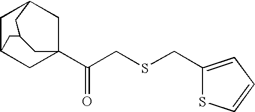 |
| 1900 | XDS04044 | 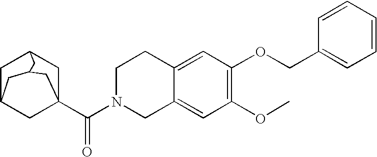 |
| 1901 | XDS04045 | 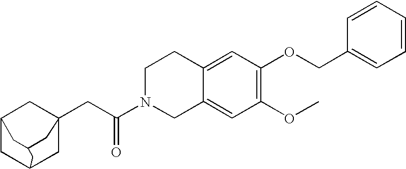 |
| 1902 | XDS04047 | 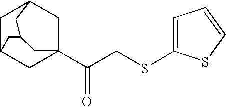 |
| 1903 | XDS04049 | 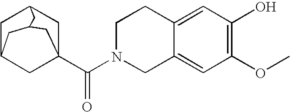 |
| 1904 | XDS04050 | 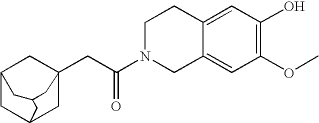 |
| 1905 | XDS04052 | 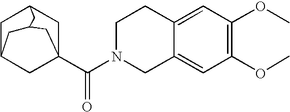 |
| 1906 | XDS04053 | 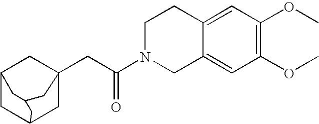 |
| 1908 | XDS04054 | 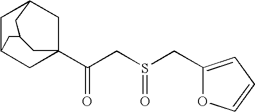 |
| 1910 | CCM02003 | 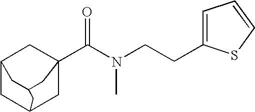 |
| 1911 | CCM02012 | 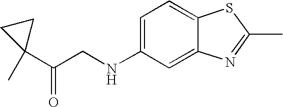 |
| 1924 | XDS04055 | 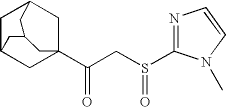 |
| 1925 | XDS04056 | 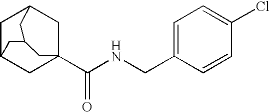 |
| 1926 | XDS02057 | 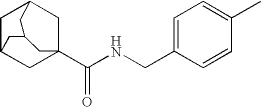 |
| 1927 | XDS04058 | 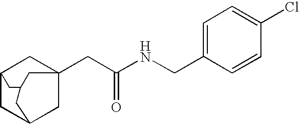 |
| 1928 | XDS04059 | 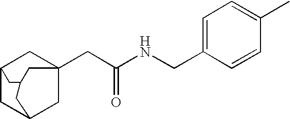 |
| 1929 | XDS04060 | 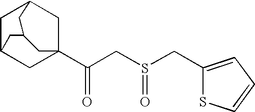 |
| 1930 | XDS04061 | 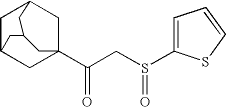 |
| 1945 | CCM02034A | 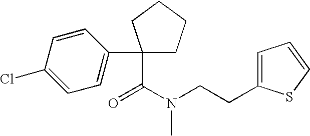 |
| 1956 | CCM02046 | 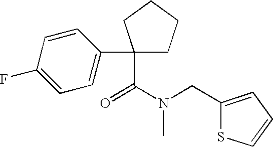 |
| 1957 | CCM02047 | 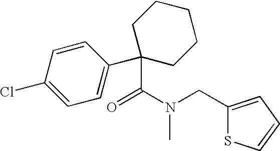 |
| 2002 | XDS04062 | 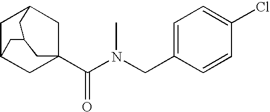 |
| 2003 | XDS04063a | 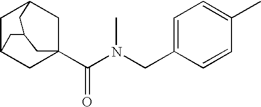 |
| 2004 | XDS04064 | 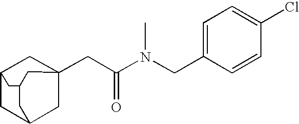 |
| 2005 | XDS04065 | 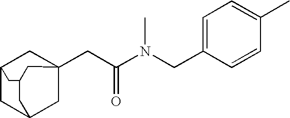 |
| 2062 | XDS04068 | 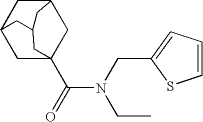 |
| 2063 | XDS04069 | 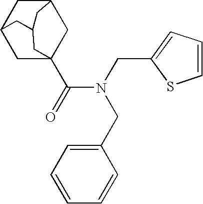 |
| 2064 | XDS04071A | 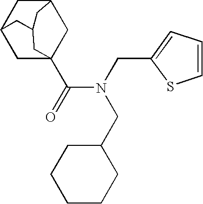 |
| 2065 | XDS04076 | 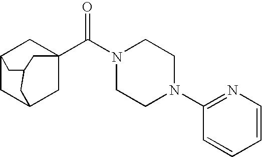 |
| 2066 | XDS04077 | 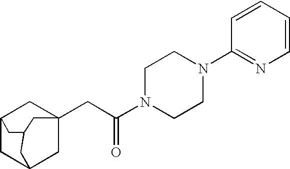 |
| 2067 | XDS04078 | 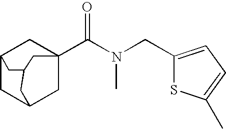 |
| 2068 | XDS04079 | 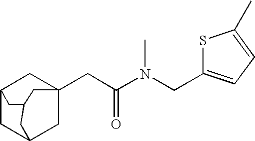 |
| 2073 | FPC01004 | 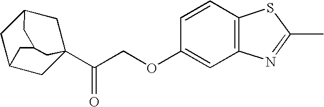 |
| 2081 | FPC01005B | 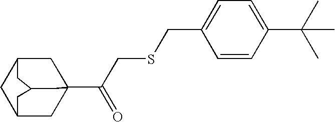 |
| 2082 | FPC01006B | 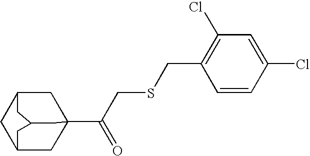 |
| 2083 | FPC01008 | 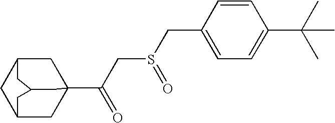 |
| 2084 | FPC01009 | 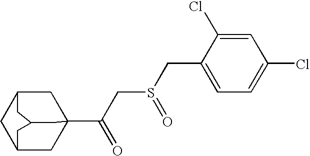 |
| 2094 | FPC01023 | 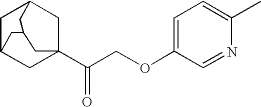 |
| 2095 | FPC01014C | 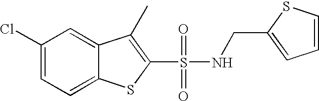 |
| 2115 | XDS04082 | 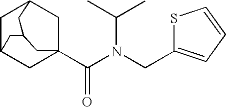 |
| 2116 | XDS04086 | 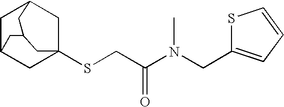 |
| 2118 | XDS04090 | 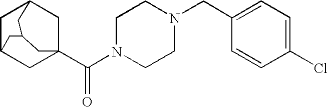 |
| 2119 | XDS04091 | 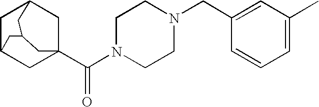 |
| 2120 | XDS04092 | 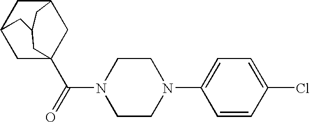 |
| 2121 | XDS04093 | 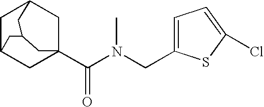 |
| 2122 | XDS04094 | 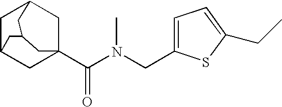 |
| 2123 | XDS04095 | 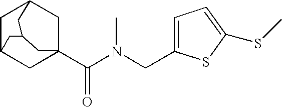 |
| 2124 | XDS04097 | 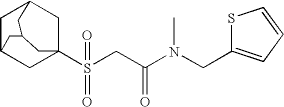 |
| 2125 | XDS04098 | 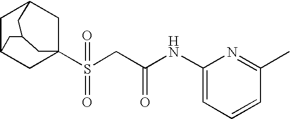 |
| 2126 | XDS04102P | 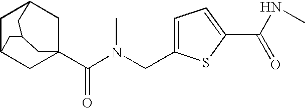 |
| 2128 | XDS04104P | 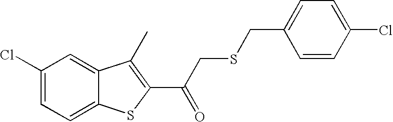 |
| 2130 | XDS04109 | 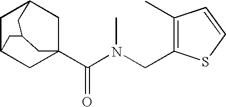 |
| 2131 | XDS04110 | 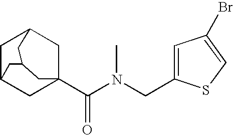 |
| 2132 | XDS04111 | 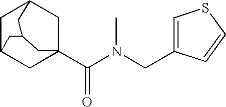 |
| 2133 | XDS04112 | 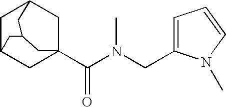 |
| 2134 | XDS04113 | 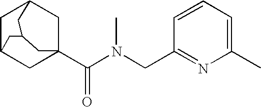 |
| 2135 | XDS04114 | 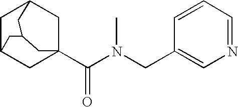 |
| 2136 | XDS04115 | 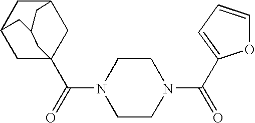 |
| 2137 | XDS04116 | 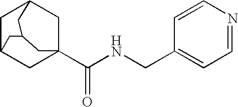 |
| 2153 | XDS04118 | 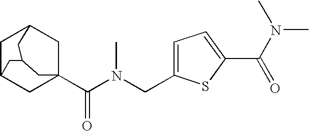 |
| 2154 | FPC01027 | 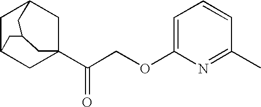 |
| 2155 | FPC01028B | 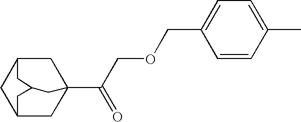 |
| 2163 | FPC01029A-031A | 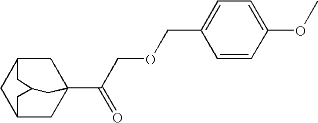 |
| 2164 | FPC01033 | 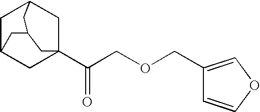 |
| 2238 | XDS04128 | 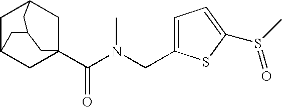 |
| 2239 | XDS04130 | 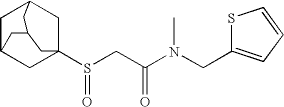 |
| 2241 | XDS04132 | 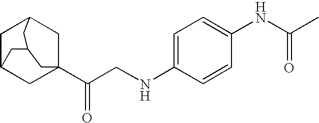 |
| 2242 | XDS04133 | 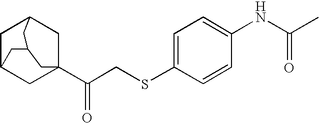 |
| 2243 | XDS04135 | 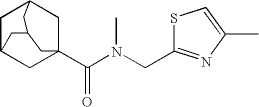 |
| 2244 | XDS04136 | 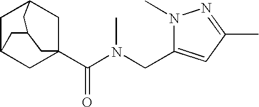 |
| 2245 | XDS04137 | 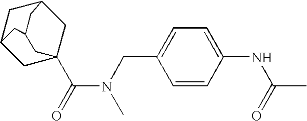 |
| 2246 | XDS04138 | 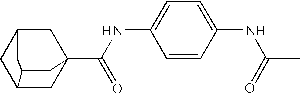 |
| 2247 | XDS04139 | 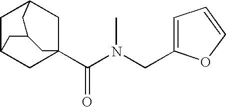 |
| 2248 | XDS04140 | 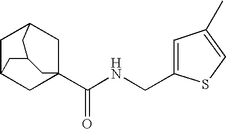 |
| 2249 | XDS04141 | 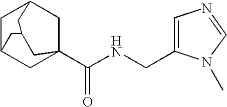 |
| 2250 | XDS04143 | 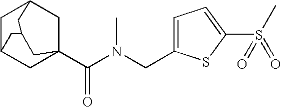 |
| 2251 | XDS04145 | 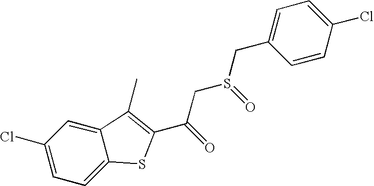 |
| 2253 | XDS04147 | 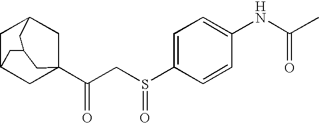 |
| 2254 | XDS04149 | 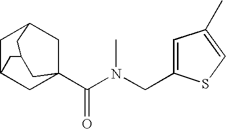 |
| 2286 | FPC01037B | 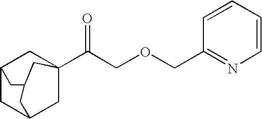 |
| 2287 | FPC01038-2 | 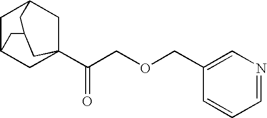 |
| 2288 | FPC01039 | 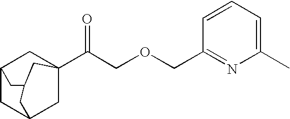 |
| 2289 | FPC01041A | 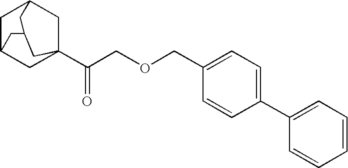 |
| 2290 | FPC01045 | 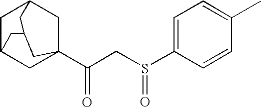 |
| 2291 | FPC01046 | 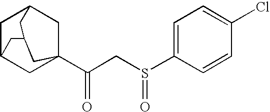 |
| 2292 | FPC01047 | 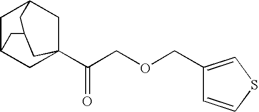 |
| 2293 | FPC01051 | 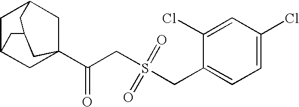 |
| 2294 | XDS04151 | 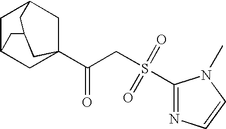 |
| 2295 | XDS04152 | 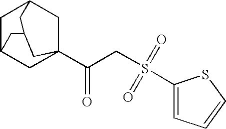 |
| 2296 | XDS04153 | 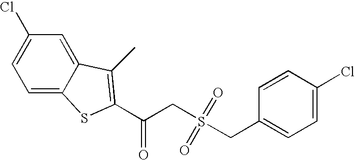 |
| 2298 | XDS04155 | 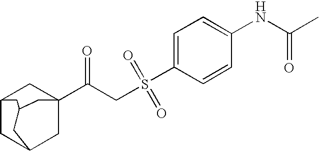 |
| 2299 | XDS04156 | 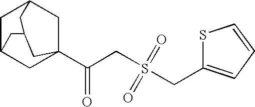 |
| 2303 | FPC01053 | 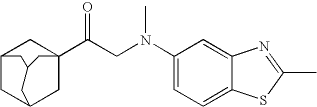 |
| 2304 | XDS04157 | 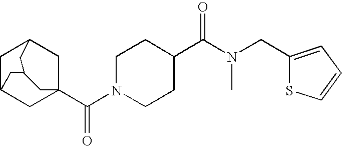 |
| 2305 | XDS04158 | 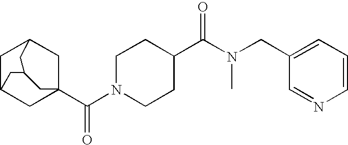 |
| 2306 | XDS04159 | 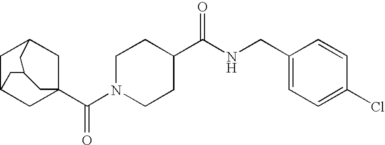 |
| 2307 | XDS04160 | 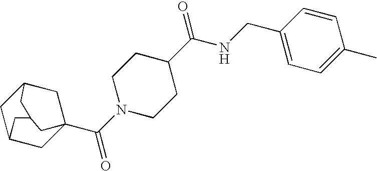 |
| 2314 | FPC01056A | 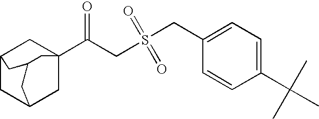 |
| 2315 | FPC01057A | 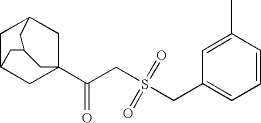 |
| 2316 | FPC01048 | 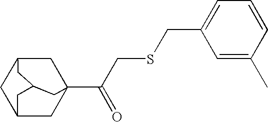 |
| 2317 | FPC01058B | 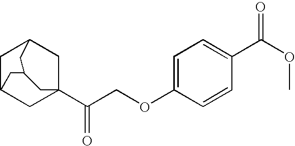 |
| 2318 | FPC01060 | 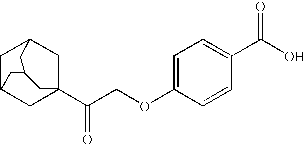 |
| 2319 | FPC01061 | 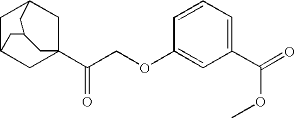 |
| 2320 | FPC01062 | 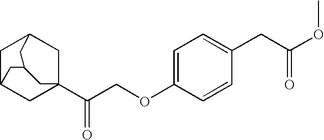 |
| 2361 | FPC01064-2 | 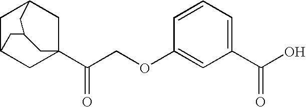 |
| 2362 | FPC01065A | 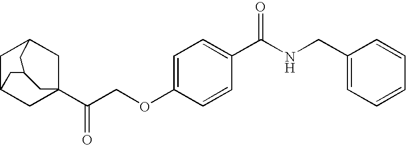 |
| 2363 | FPC01066A2 | 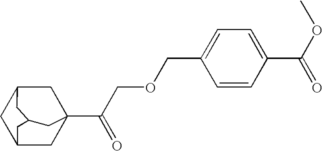 |
| 2364 | FPC01067cr | 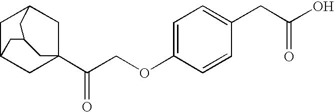 |
| 2365 | FPC01068A | 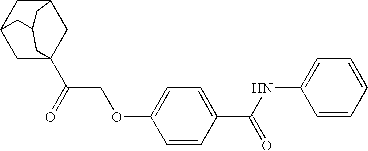 |
| 2366 | FPC01069B1 | 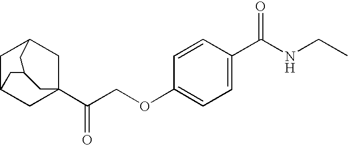 |
| 2367 | FPC01070 | 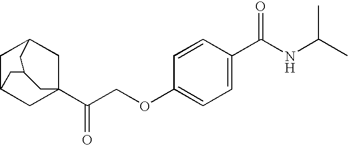 |
| 2368 | FPC01071A | 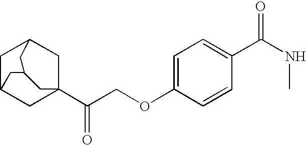 |
| 2375 | FPC01072 | 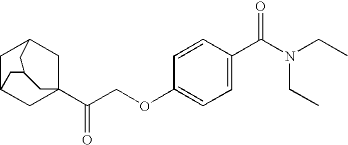 |
| 2376 | FPC01073 | 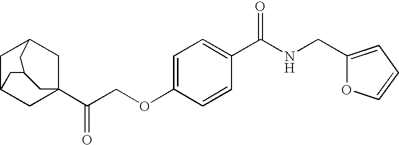 |
| 2377 | FPC01074 | 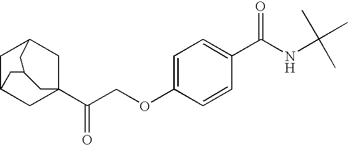 |
| 2393 | XDS04163 | 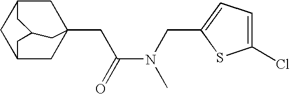 |
| 2394 | XDS04164 | 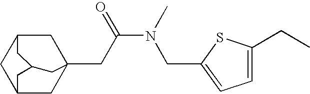 |
| 2395 | XDS04165 | 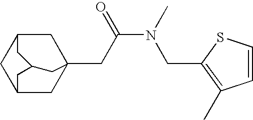 |
| 2396 | XDS04166 | 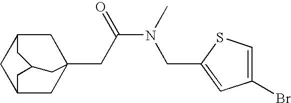 |
| 2397 | XDS04171 | 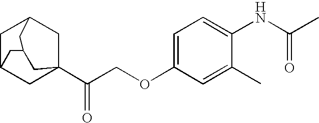 |
| 2398 | XDS04173 | 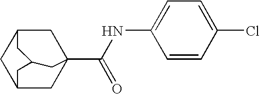 |
| 2399 | XDS04174 | 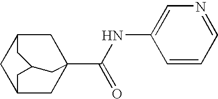 |
| 2400 | XDS04175 | 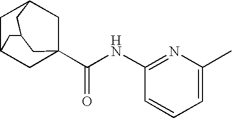 |
| 2401 | XDS04176 | 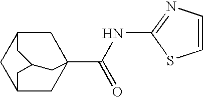 |
| 2402 | XDS04178 | 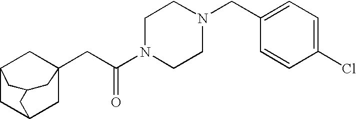 |
| 2403 | XDS04179 | 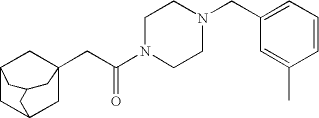 |
| 2404 | XDS04180 | 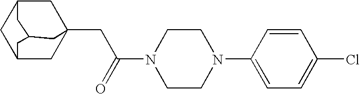 |
| 2405 | XDS04181 | 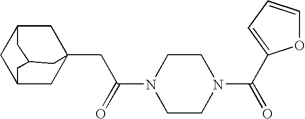 |
| 2406 | XDS04182 | 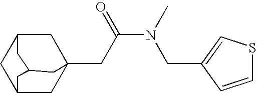 |
| 2407 | XDS04183 | 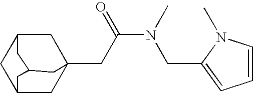 |
| 2408 | XDS04184 | 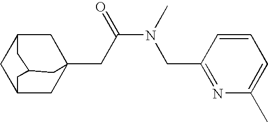 |
| 2409 | XDS04185 | 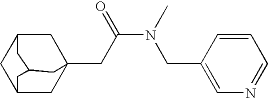 |
| 2410 | XDS04186 | 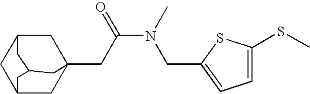 |
| 2411 | XDS04187 | 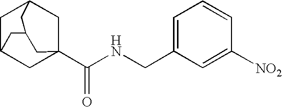 |
| 2412 | XDS04188 | 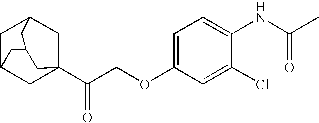 |
| 2413 | XDS04189 | 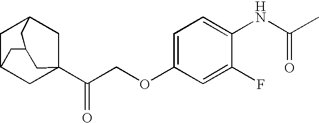 |
| 2415 | FPC01075 | 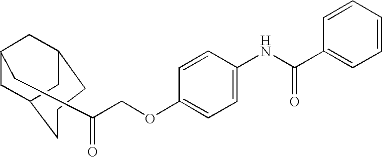 |
| 2416 | FPC01076C | 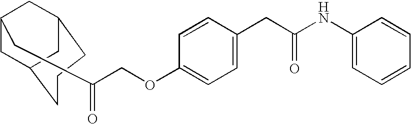 |

### Hydroxysteroid Dehydrogenase

11β Hydroxysteroid dehydrogenase may be referred to as “11β-HSD” or “HSD” for short

In some aspects of the invention 11β-HSD is preferably 11β-HSD Type 1 (EC1.1.1.146).

In some aspects of the invention 11β-HSD is preferably 11β-HSD Type 2 (EC1.1.1.146).

### Hydroxysteroid Dehydrogenase Inhibition

It is believed that some disease conditions associated with HSD activity are due to conversion of a inactive, cortisone to an active, cortisol. In disease conditions associated with HSD activity, it would be desirable to inhibit HSD activity.

Here, the term “inhibit” includes reduce and/or eliminate and/or mask and/or prevent the detrimental action of HSD.

### HSD Inhibitor

In accordance with the present invention, the compound of the present invention is capable of acting as an HSD inhibitor.

Here, the term “inhibitor” as used herein with respect to the compound of the present invention means a compound that can inhibit HSD activity—such as reduce and/or eliminate and/or mask and/or prevent the detrimental action of HSD. The HSD inhibitor may act as an antagonist.

The ability of compounds to inhibit hydroxysteroid dehydrogenase activity can be assessed using the suitable biological assay presented in the Examples section.

It is to be noted that the compound of the present invention may have other beneficial properties in addition to or in the alternative to its ability to inhibit HSD activity.

### Therapy

The compounds of the present invention may be used as therapeutic agents—i.e. in therapy applications.

The term “therapy” includes curative effects, alleviation effects, and prophylactic effects.

The therapy may be on humans or animals, preferably female animals or humans, such as female humans.

### Pharmaceutical Compositions

In one aspect, the present invention provides a pharmaceutical composition, which comprises a compound according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Combination Pharmaceutical

The compound of the present invention may be used in combination with one or more other active agents, such as one or more other pharmaceutically active agents.

By way of example, the compounds of the present invention may be used in combination with other 11β-HSD inhibitors and/or other inhibitors such as an aromatase inhibitor (such as for example, 4hydroxyandrostenedione (4-OHA)), and/or a steroid sulphatase inhibitors such as EMATE and/or steroids—such as the naturally occurring stemeurosteroids dehydroepiandrosterone sulfate (DHEAS) and pregnenolone sulfate (PS) and/or other structurally similar organic compounds.

In, addition, or in the alternative, the compound of the present invention may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)— such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight. By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestible solution; a parenteral route where delivery is by an injectable form, such as, for example, an intravenous, intramuscular or subcutaneous route.

Thus, for pharmaceutical administration, the compounds of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

### Cell Cycling

The compounds of the present invention may be useful in the method of treatment of a cell cycling disorder.

As discussed in “Molecular Cell Biology” 3rd Ed. Lodish et al. pages 177-181 different eukaryotic cells can grow and divide at quite different rates. Yeast cells, for example, can divide every 120 min., and the first divisions of fertilised eggs in the embryonic cells of sea urchins and insects take only 1530 min. because one large preexisting cell is subdivided. However, most growing plant and animal cells take 10-20 hours to double in number, and some duplicate at a much slower rate. Many cells in adults, such as nerve cells and striated muscle cells, do not divide at all; others, like the fibroblasts that assist in healing wounds, grow on demand but are otherwise quiescent.

Still, every eukaryotic cell that divides must be ready to donate equal genetic material to two daughter cells. DNA synthesis in eukaryotes does not occur throughout the cell division cycle but is restricted to a part of it before cell division.

The relationship between eukaryotic DNA synthesis and cell division has been thoroughly analysed in cultures of mammalian cells that were all capable of growth and division. In contrast to bacteria, it was found, eukaryotic cells spend only a part of their time in DNA synthesis, and it is completed hours before cell division (mitosis). Thus a gap of time occurs after DNA synthesis and before cell division; another gap was found to occur after division and before the next round of DNA synthesis. This analysis led to the conclusion that the eukaryotic cell cycle consists of an M (mitotic) phase, a G₁ phase (the first gap), the S (DNA synthesis) phase, a G₂ phase (the second gap), and back to M. The phases between mitoses (G₁, S, and G₂) are known collectively as the interphase.

Many nondividing cells in tissues (for example, all quiescent fibroblasts) suspend the cycle after mitosis and just prior to DNA synthesis; such “resting” cells are said to have exited from the cell cycle and to be in the G₀ state.

It is possible to identify cells when they are in one of the three interphase stages of the cell cycle, by using a fluorescence-activated cell sorter (FACS) to measure their relative DNA content: a cell that is in G₁ (before DNA synthesis) has a defined amount x of DNA; during S (DNA replication), it has between x and 2x; and when in G₂ (or M), it has 2x of DNA.

The stages of mitosis and cytokinesis in an animal cell are as follows

(a) Interphase. The G₂ stage of interphase immediately precedes the beginning of mitosis. Chromosomal DNA has been replicated and bound to protein during the S phase, but chromosomes are not yet seen as distinct structures. The nucleolus is the only nuclear substructure that is visible under light microscope. In a diploid cell before DNA replication there are two morphologic chromosomes of each type, and the cell is said to be 2n. In G₂, after DNA replication, the cell is 4n. There are four copies of each chromosomal DNA. Since the sister chromosomes have not yet separated from each other, they are called sister chromatids.
b) Early prophase. Centrioles, each with a newly formed daughter centriole, begin moving toward opposite poles of the cell; the chromosomes can be seen as long threads. The nuclear membrane begins to disaggregate into small vesicles.
(c) Middle and late-prophase. Chromosome condensation is completed; each visible chromosome structure is composed of two chromatids held together at their centromeres. Each chromatid contains one of the two newly replicated daughter DNA molecules. The microtubular spindle begins to radiate from the regions just adjacent to the centrioles, which are moving closer to their poles. Some spindle fibres reach from pole to pole; most go to chromatids and attach at kinetochores.
(d) Metaphase. The chromosomes move toward the equator of the cell, where they become aligned in the equatorial plane. The sister chromatids have not yet separated.
(e) Anaphase. The two sister chromatids separate into independent chromosomes. Each contains a centromere that is linked by a spindle fibre to one pole, to which it moves. Thus one copy of each chromosome is donated to each daughter cell. Simultaneously, the cell elongates, as do the pole-to-pole spindles. Cytokinesis begins as the cleavage furrow starts to form.
(f) Telophase. New membranes form around the daughter nuclei; the chromosomes uncoil and become less distinct, the nucleolus becomes visible again, and the nuclear membrane forms around each daughter nucleus. Cytokinesis is nearly complete, and the spindle disappears as the microtubules and other fibres depolymerise. Throughout mitosis the “daughter” centriole at each pole grows until it is full-length. At telophase the duplication of each of the original centrioles is completed, and new daughter centrioles will be generated during the next interphase.
(g) Interphase. Upon the completion of cytokinesis, the cell enters the G₁ phase of the cell cycle and proceeds again around the cycle.

It will be appreciated that cell cycling is an extremely important cell process. Deviations from normal cell cycling can result in a number of medical disorders. Increased and/or unrestricted cell cycling may result in cancer. Reduced cell cycling may result in degenerative conditions. Use of the compound of the present invention may provide a means to treat such disorders and conditions.

Thus, the compound of the present invention may be suitable for use in the treatment of cell cycling disorders such as cancers, including hormone dependent and hormone independent cancers.

In addition, the compound of the present invention may be suitable for the treatment of cancers such as breast cancer, ovarian cancer, endometrial cancer, sarcomas, melanomas, prostate cancer, pancreatic cancer etc. and other solid tumours.

For some applications, cell cycling is inhibited and/or prevented and/or arrested, preferably wherein cell cycling is prevented and/or arrested. In one aspect cell cycling may be inhibited and/or prevented and/or arrested in the G₂/M phase. In one aspect cell cycling may be irreversibly prevented and/or inhibited and/or arrested, preferably wherein cell cycling is irreversibly prevented and/or arrested.

By the term “irreversibly prevented and/or inhibited and/or arrested” it is meant after application of a compound of the present invention, on removal of the compound the effects of the compound, namely prevention and/or inhibition and/or arrest of cell cycling, are still observable. More particularly by the term “irreversibly prevented and/or inhibited and/or arrested” it is meant that when assayed in accordance with the cell cycling assay protocol presented herein, cells treated with a compound of interest show less growth after Stage 2 of the protocol I than control cells. Details on this protocol are presented below.

Thus, the present invention provides compounds which: cause inhibition of growth of oestrogen receptor positive (ER+) and ER negative (ER−) breast cancer cells in vitro by preventing and/or inhibiting and/or arresting cell cycling; and/or cause regression of nitroso-methyl urea (NMU)-induced mammary tumours in intact animals (i.e. not ovariectomised), and/or prevent and/or inhibit and/or arrest cell cycling in cancer cells; and/or act in vivo by preventing and/or inhibiting and/or arresting cell cycling and/or act as a cell cycling agonist.

### Cell Cycling Assay

### Protocol 2

### Procedure

### Stage 1

MCF-7 breast cancer cells are seeded into multi-well culture plates at a density of 105 cells/well. Cells were allowed to attach and grown until about 30% confluent when they are treated as follows:

Control—no treatment

### Compound of Interest (COI) 20 μM

Cells are grown for 6 days in growth medium containing the COI with changes of medium/COI every 3 days. At the end of this period cell numbers were counted using a Coulter cell counter.

### Stage 2

After treatment of cells for a 6-day period with the COI cells are re-seeded at a density of 10⁴ cells/well. No further treatments are added. Cells are allowed to continue to grow for a further 6 days in the presence of growth medium. At the end of this period cell numbers are again counted.

### Cancer

As indicated, the compounds of the present invention may be useful in the treatment of a cell cycling disorder. A particular cell cycling disorder is cancer.

Cancer remains a major cause of mortality in most Western countries. Cancer therapies developed so far have included blocking the action or synthesis of hormones to inhibit the growth of hormone-dependent tumours. However, more aggressive chemotherapy is currently employed for the treatment of hormone-independent tumours.

Hence, the development of a pharmaceutical for anti-cancer treatment of hormone dependent and/or hormone independent tumours, yet lacking some or all of the side-effects associated with chemotherapy, would represent a major therapeutic advance.

We believe that the compound of the present invention provides a means for the treatment of cancers and, especially, breast cancer.

In addition or in the alternative the compound of the present invention may be useful in the blocking the growth of cancers including leukaemias and solid tumours such as breast, endometrium, prostate, ovary and pancreatic tumours.

### Other Therapies

As previously mentioned, in one aspect the present invention provides use of a compound as described herein in the manufacture of a medicament for use in the therapy of a condition or disease associated with 11β-HSD.

Conditions and diseases associated with 11β-HSD have been reviewed in Walker, E. A.; Stewart, P. M.; Trends in Endocrinology and Metabolism, 2003, 14 (7), 334-339.

In a preferred aspect, the condition or disease is selected from the group consisting of:
metabolic disorders, such as diabetes and obesity
cardiovascular disorders, such as hypertension
glaucoma
inflammatory disorders, such as arthritis or asthma
immune disorders
bone disorders, such as osteoporosis
cancer
intra-uterine growth retardation
apparent mineralocorticoid excess syndrome (AME)
polycystic ovary syndrome (PCOS)
hirsutism
acne
oligo- or amenorrhea
adrenal cortical adenoma and carcinoma
Cushing's syndrome
pituitary tumours
invasive carcinomas
wound healing
CNS disorders
breast cancer; and
endometrial cancer.

It is also to be understood that the compound/composition of the present invention may have other important medical implications.

For example, the compound or composition of the present invention may be useful in the treatment of the disorders listed in WO-A-99/52890—viz:

In addition, or in the alternative, the compound or composition of the present invention may be useful in the treatment of the disorders listed in WO-A-98/05635. For ease of reference, part of that list is now provided: diabetes including Type II diabetes, obesity, cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimers disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### SUMMARY

In summation, the present invention provides compounds for use as hydroxysteroid dehydrogenase inhibitors, and pharmaceutical compositions for the same.

### BRIEF DESCRIPTION OF THE FIGURES

The present invention will be described in further detail by way of example only with reference to the accompanying figures in which: —

FIG. 1 is a scheme;

FIG. 2 is a graph;

FIG. 3 is a graph;

FIG. 4 is a graph;

FIG. 5 is a graph; and

FIG. 6 is a graph;

The present invention will now be described in further detail in the following examples.

### EXAMPLES

The present invention will now be described only by way of example.

### Synthesis of Adamantyl Ketone Derivatives

### 1-Adamantan-1-yl-2-(2-methyl-benzothiazol-5-ylamino)ethanone (XDS03133, STX1487)

To a solution of adamantan-1-yl bromomethyl ketone (129 mg, 0.50 mmol) and 2-methylbenzo[d]thiazol-5-amine (164 mg, 1.0 mmol) in acetonitrile (10 mL) was added potassium carbonate (150 mg). The mixture was stirred at ambient temperature for 18 h, then at 60° C. for 6 h, partitioned between ethyl acetate and brine. The organic phase was washed with brine, dried over sodium sulphate and concentrated in vacuo to give the crude product. Purification with flash column (DCM-ethyl acetate; gradient elution) yielded the title compound as off-white solid (90 mg, 53%). TLC single spot at R_{f}: 0.66 (10% EtOAc/DCM); ¹H NMR (270 MHz, CDCl₃) δ 1.69 (6H, m, 3×CH₂), 1.84 (6H, d, J=2.7 Hz, 3×CH₂), 2.02 (3H, broad, 3×CM), 2.71 (3H, s, CH₃), 4.06 (2H, s, CH₂), 4.75 (1H, broad, NH), 6.68 (1H, dd, J=8.6, 2.4 Hz, ArH), 7.03 (1H, d, J=2.2 Hz, ArH) and 7.48 (1H, d, J=8.8 Hz, ArH); LC/MS (APCI) m/z 341 (M⁺+H); HPLC tᵣ=3.53 min (>96%) in 20% water-acetonitrile.

### 1-Adamantan-1-yl-2-(4-chloro-phenylsulfanyl)-ethanone (XDS03139B, STX1535)

The solution of adamantan-1-yl bromomethyl ketone (128 mg, 0.50 mmol) and 4-chlorobenzenethiol (145 mg, 1.0 mmol) in acetonitrile/triethylamine (6 mL/0.6 mL) was stirred at ambient temperature for 18 h, partitioned between ethyl acetate and 5% sodium carbonate solution. The organic phase was washed with brine, dried over sodium sulphate and concentrated in vacuo to give the crude product. Purification with flash column (hexane-DCM; gradient elution) yielded the title compound as white crystalline solid (98 mg, 61%). TLC single spot at R_{f}: 0.59 (20% EtOAc/DCM); ¹H NMR (270 MHz, CDCl₃) δ 1.64-1.76 (6H, m, 3×CH₂), 1.83 (6H, d, J=2.8 Hz, 3×CH₂), 2.04 (3H, broad, 3×CH), 3.88 (2H, s, CH₂) and 7.20-7.26 (4H, m, ArH); LC/MS (APCI) m/z 319 (M⁺−H); HPLC tᵣ=4.23 min (>98%) in 20% water-acetonitrile.

### 1-Adamantan-1-yl-2-p-tolylsulfanyl-ethanone (XDS03141, STX1537)

The solution of adamantan-1-yl bromomethyl ketone (128 mg, 0.50 mmol) and 4-methylbenzenethiol (124 mg, 1.0 mmol) in acetonitrile/triethylamine (6 mL/0.6 mL) was stirred at ambient temperature for 3 h, partitioned between ethyl acetate and 5% sodium carbonate solution. The organic phase was washed with brine, dried over sodium sulphate and concentrated in vacuo to give the crude product. Purification with flash column (hexane-DCM; gradient elution) yielded the title compound as white crystalline solid (107 mg, 71%). TLC single spot at R_{f}: 0.66 (20% EtOAc/hexane); ¹H NMR (270 MHz, CDCl₃) δ 1.62 (6H, m, 3×CH₂), 1.77 (6H, d, J=3.0 Hz, 3×CH₂), 1.97 (3H, broad s, 3×CH), 2.23 (3H, s, CH₃), 3.80 (2H, s, CH₂), 7.02 (2H, d, J=7.9 Hz, ArH) and 7.21 (2H, d, J=7.9 Hz, ArH); LC/MS (APCI) m/z 299 (M⁺−H); HPLC tᵣ=4.44 min (>99%) in 20% water-acetonitrile.

### 1-Adamantan-1-yl-2-(2,5-dichloro-phenylsulfanyl)-ethanone (XDS03142, STX1538)

The solution of adamantan-1-yl bromomethyl ketone (128 mg, 0.50 mmol) and 2,5-dichlorobenzenethiol (179 mg, 1.0 mmol) in acetonitrile/triethylamine (6 mL/0.6 mL) was stirred at ambient temperature for 5 h, partitioned between ethyl acetate and 5% sodium carbonate solution. The organic phase was washed with brine, dried over sodium sulphate and concentrated in vacuo to give the crude product. Purification with flash column (hexane-DCM; gradient elution) yielded the title compound as white crystalline solid (139 mg, 79%). TLC single spot at R_{f}: 0.55 (40% DCM/hexane); ¹H NMR (270 MHz, CDCl₃) δ 1.74 (6H, ma, 3×CH₂), 1.88 (6H, d, J=3.0 Hz, 3×CH₂), 2.07 (3H, broad s, 3×CM), 3.96 (2H, s, CH₂), 7.08 (1H, dd, J=8.4, 2.3 Hz, ArH), 7.19 (1H, d, J=2.2 Hz, ArH) and 7.27 (1H, d, J=8.4 Hz, ArH); LC/MS (APCI) m/z 353 (M⁺−H); HPLC tᵣ=4.88 min (>99%) in 10% water-acetonitrile.

### 1-Adamantan-1-yl-2-(4-chloro-benzenesulfonyl)-ethanone (XDS03145, STX1539)

To a solution of 1-Adamantan-1-yl-2-(4-chloro-phenylsulfanyl)-ethanone (XDS03139B, 72 mg, 0.23 mmol) in DCM (6 mL) was added mCPBA (106 mg, purity 60-77%). The mixture was stirred at 0° C. for 3 h, partitioned between DCM and 5% sodium bicarbonate solution. The organic phase was washed with brine, dried over sodium sulphate and concentrated in vacuo to give the crude product as white solid. Purification with flash column (hexane-ethyl acetate; gradient elution) yielded the title compound as white crystalline solid (52 mg, 64%). TLC single spot at R_{f}: 0.39 (20% EtOAc/DCM); ¹H NMR (270 MHz, CDCl₃) δ 1.69 (6H, m, 3×CH₂), 1.75 (6H, d, J=2.8 Hz, 3×CH₂), 2.05 (3H, broad, 3×CH), 4.28 (2H, s, CH₂), 7.53 (2H, dt, J=7.6, 2.3 Hz, ArH) and 7.88 (2H, dt, J=7.5, 2.1 Hz, ArH); LC/MS (APCI) m/z 353 (M⁺+H; HPLC tᵣ=3.42 min (>99%) in 20% water-acetonitrile.

### 1-Adamantan-1-yl-2-(toluene-4-sulfonyl)-ethanone (XDS03152, STX1562)

To a solution of 1-adamantan-1-yl-2-p-tolylsulfanyl-ethanone (XDS03141, 66 mg, 0.22 mmol) in DCM (5 mL) was added mCPBA (110 mg, purity 60-77%). The mixture was stirred at 0° C. for 4 h, partitioned between ethyl acetate and 5% sodium carbonate solution. The organic phase was washed with brine, dried over sodium sulphate and concentrated in vacuo to give the crude product as white solid. Purification with flash column (hexane-ethyl acetate; gradient elution) yielded the title compound as white crystalline solid (60 mg, 82%). TLC single spot at R_{f}: 0.55 (30% EtOAc/DCM); ¹H NMR (270 MHz, CDCl₃) δ 1.53-1.74 (12H, m, 6×CH₂), 2.04 (3H, broad, 3×CH), 2.44 (3K, s, CH₃), 4.26 (2H, s, CH₂), 7.53 (2H, d, J=8.5 Hz, ArH) and 7.80 (2H, d, J=8.5 Hz, ArH); LC/MS (APCI) m/z 3311-H); HPLC tᵣ=2.99 min (>99%) in 10% water-acetonitrile.

### 1-Adamantan-1-yl-2-(2,5-dichloro-benzenesulfinyl)-ethanone (XDS03153, STX1563)

To a solution of 1-adamantan-1-yl-2-(2,5-dichloro-phenylsulfanyl)-ethanone (XDS03142, 98 mg, 0.25 mmol) in DCM (5 mL) was added mCPBA (121 mg, purity 60-77%). The mixture was stirred at 0° C. for 4 h, partitioned between ethyl acetate and 5% sodium carbonate solution. The organic phase was washed with brine, dried over sodium sulphate and concentrated in vacuo to give the crude product as white solid. Purification with flash column (hexane-ethyl acetate; gradient elution) yielded the title compound as white crystalline solid (71 mg, 77%). TLC single spot at R_{f}: 0.55 (30% EtOAc/DCM); ¹H NMR (270, CDCl₃) δ 1.63-1.88 (12H, m, 6×CH₂), 2.06 (3H, broad, 3×CH), 3.87 (1H, d, J=15 Hz, CH), 4.07 (1H, d, J=15 Hz, CM), 7.33 (1H, d, J=8.5 Hz, ArH), 7.41 (1H, dd, J=8.4, 2.5 Hz, ArH) and 7.93 (1H, d, J=2.5 Hz, ArH); LC/MS (APCI) m/z 371 (M⁺+H); HPLC tᵣ=3.78 min (>99%) in 10% water-acetonitrile.

### 1-Adamantan-1-yl-2-biphenyl-4-yloxy)-ethanone (XDS03159, STX1567)

To a solution of 1-adamantyl bromomethyl ketone (200 mg, 0.78 mmol) and biphenyl-4-ol (139 mg, 0.82 mmol) in acetone (8 mL) was added potassium carbonate (215 mg, 1.56 mmol). The mixture was stirred at ambient temperature for 48 h, partitioned between ethyl acetate and 5% sodium carbonate solution. The organic phase was washed with brine, dried over sodium sulphate and concentrated in vacuo to give the crude product as white solid. Purification with flash column (hexane-ethyl acetate; gradient elution) yielded the title compound as white solid (155 mg, 57%). TLC single spot at R_{f}: 0.70 (30% ethyl acetate/hexane); ¹H NMR (270 MHz, CDCl₃) δ 1.70-1.81 (6H, m, 3×CH₂), 1.94 (6H, d, J=2.8 Hz, 3×CH₂), 2.08 (3H, broad s, 3×CH), 4.89 (2H, s, CH₂), 6.92 (2H, dt, J=8.7, 3.2 Hz, ArH), 7.30 (1H, dt, J=7.2, 1.3 Hz, ArH), 7.37-7.42 (2H, m, ArH) and 7.47-7.54 (4H, m, ArH); LC/MS (APCI) m/z 347 (M⁺+H); HPLC tᵣ=5.25 min (>99%) in 10% water-acetonitrile.

### 1-Adamantan-1-yl-2-(3,4-dimethoxy-phenoxy)-ethanone (XDS03160, STX1568)

The title compound was synthesized as described for STX1567. White crystalline solid (215 mg, 84%) was obtained. TLC single spot at R_{f}: 0.43 (30% ethyl acetate/hexane); ¹H NMR (270 MHz, CDCl₃) δ 1.69-1.79 (6H, m, 3×CH₂), 1.90 (6H, d, J=2.8 Hz, 3×CH₂), 2.07 (3H, broad s, 3×CH), 3.81 (3H, s, CH₃), 3.83 (3H, s, CH₃), 4.79 (2H, s, CH₂), 6.27 (1H, dd, J=8.9, 3.0 Hz, ArH), 6.59 (1H, d, J=2.8 Hz, ArH) and 6.73 (1H, d, J=8.7 Hz, ArH); LC/MS (APCI) m/z 331 (M⁺+H); HPLC tᵣ=3.15 min (>99%) in 10% water-acetonitrile.

### N-[4-(2-Adamantan-1-yl-2-oxo-ethoxy)-phenyl]-acetamide (XDS03161, STX1569)

The title compound was synthesized as described for STX1567. White crystalline solid (220 mg, 86%) was obtained. TLC single spot at R_{f}: 0.33 (30% ethyl acetate/DCM); ¹H NMR (270 MHz, CDCl₃) δ 1.66-1.82 (6H, m, 3×CH₂), 1.90 (6H, d, J=2.7 Hz, 3×CH₂), 2.07 (3H, broad s, 3×CH), 2.13 (3H, s, CH₃), 4.82 (2H, s, CH₂), 6.80 (2H, dt, J=8.9, 2.2 Hz, ArH), 7.13 (1H, broad, NH) and 7.36 (2H, dt, J=9.0, 2.3 Hz, ArH); LC/MS (APCI) m/z 326 (M⁺−H); HPLC tᵣ=2.45 min (>99%) in 10% water-acetonitrile.

### 1-Adamantan-1-yl-2-(4-chloro-phenoxy)-ethanone (XDS03183, STX1610)

The title compound was synthesized as described for STX1567. White crystalline solid (190 mg, 80%) was obtained. TLC single spot at R_{f}: 0.67 (30% ethyl acetate/hexane); ¹H NMR (300 MHz, CDCl₃) δ 1.71-1.83 (6H, m, 3×CH₂), 1.93 (6H, d, J=2.7 Hz, 3×CH₂), 2.10 (3H, broad s, 3×CH), 4.84 (2H, s, CH₂), 6.80 (2H, dt, J=8.9, 2.2 Hz, ArH) and 7.23 (2H, dt, J=9.0, 2.2 Hz, ArH); LC/MS (APCI) m/z 305 (M⁺+H); HPLC T=2.41 min (>99%) in 4% water-acetonitrile.

### 1-Adamantan-1-yl-2-(2,5-dichloro-phenoxy)-ethanone (XDS03184, STX1618)

The title compound was synthesized as described for STX1567. White crystalline solid (180 mg, 680%) was obtained. TLC single spot at R_{f}: 0.69 (30% ethyl acetate/hexane); ¹H NMR (300 MHz, CDCl₃) δ 1.71-1.83 (6H, m, 3×CH₂), 1.93 (6H, d, J=2.7 Hz, 3×CH₂), 2.09 (3H, broad s, 3×CH), 4.92 (2H, s, CH₂), 6.67 (1H, d, J=2.3 Hz, ArH), 6.90 (1H, dd, J=8.5, 2.3 Hz, ArH) and 7.29 (1H, d, J=8.5 Hz, ArH); LC/MS (APCI) m/z 339 (M⁺+H); HPLC tᵣ=2.62 min (>99%) in 4% water-acetonitrile.

### 1-Adamantan-1-yl-2-p-tolyloxy-ethanone (XDS03185, STX1619)

The title compound was synthesized as described for STX1567. White crystalline solid (190 mg, 86%) was obtained. TLC single spot at R_{f}: 0.68 (30% ethyl acetate/hexane); ¹H NMR (300 MHz, CDCl₃) δ 1.71-1.83 (6H, m, 3×CH₂), 1.93 (6H, d, J=2.7 Hz, 3×CH₂), 2.09 (3H, broad s, 3×CH), 2.28 (3H, s, CH₃), 4.83 (2H, s, CH₂), 6.78 (2H, dt, J=8.9, 2.0 Hz, ArH) and 7.09 (2H, dt, J=8.8, 2.0 Hz, ArH); LC/MS (APCI) m/z 285 (M⁺+H); HPLC tᵣ=2.44 min (>99%) in 4% water-acetonitrile.

### 1-Adamantan-1-yl-2-(4-chloro-benzylsulfanyl)-ethanone (XDS03186, STX1621)

The solution of adamantan-1-yl bromomethyl ketone (257 mg, 1.0 mmol) and (4-Chloro-phenyl)-methanethiol (159 mg, 1.0 mmol) in acetonitrile/triethylamine (5 mL/0.5 mL) was stirred at ambient temperature for 18 h, partitioned between ethyl acetate and 5% sodium carbonate solution. The organic phase was washed with brine, dried over sodium sulphate and concentrated in vacuo to give the crude product. Purification with flash column (hexane-EtOAc; gradient elution) yielded the title compound as white crystalline solid (330 mg, 99%); TLC single spot at R_{f}: 0.52 (20% EtOAc/DCM); ¹H NMR (270 MHz, CDCl₃) δ 1.66-1.78 (6H, m, 3×CH₂), 1.83 (6H, d, J=2.8 Hz, 3×CH₂), 2.06 (3H, broad, 3×CH), 3.20 (2H, s, CH₂), 3.70 (2H, s, CH₂) and 7.26 (4H, s, ArH); LC/MS (APCI) m/z 333 (M⁺−H); HPLC tᵣ=3.55 min (>98%) in 4% water-acetonitrile.

### 1-Adamantan-1-yl-2-(4-chloro-benzyloxy)-ethanone (XDS03189, STX1626)

To a solution of 4-chlorobenzyl alcohol (117 mg, 0.82 mmol) in anhydrous THF (6 mL) was added sodium hydride (65% dispersion, 33 mg, 0.82 mmol), the mixture was stirred at ambient-temperature for 15 min. Adamantan-1-yl bromomethyl ketone (200 mg, 0.78 mmol) was added and the mixture was stirred at ambient temperature for 2 h, partitioned between ethyl acetate and brine. The organic phase was washed with brine, dried over sodium sulphate and concentrated in vacuo to give the crude product. Purification with flash column (hexane-EtOAc; gradient elution) yielded the title compound as white solid (52 mg, 21%). TLC single spot at R_{f}: 0.52 (30% EtOAc/DCM); ¹H NMR (300 MHz, CDCl₃) δ 1.58-1.72 (6H, m, 3×CH₂), 1.75 (6H, d, J=2.7 Hz, 3×CH₂), 2.00 (3H, broad, 3×CH), 4.20 (2H, s, CH₂), 4.48 (2H, s, CH₂) and 7.25 (4H, s, ArH); LC/MS (APCI) m/z 319 (M⁺+H); HPLC tᵣ=4.69 min (>98%) in 10% water-acetonitrile.

### 1-Adamantan-1-yl-2-(4-chloro-phenylmethanesulfinyl)-ethanone (XDS03192, STX1628)

To a solution of 1-adamantan-1-yl-2-(4-chloro-benzylsulfanyl)-ethanone (195 mg, 0.59 mmol) in DCM (15 mL) was added mCPBA (189 mg, purity 60-77%). The mixture was stirred at 0° C. for 2 h, partitioned between ethyl acetate and 5% sodium carbonate solution. The organic phase was washed with brine, dried over sodium sulphate and concentrated in vacuo to give the crude product as white solid. Purification with flash column (DCM-ethyl acetate; gradient elution) yielded the title compound as white crystalline solid (95 mg, 46%). TLC single spot at R_{f}: 0.48 (20% EtOAc/DCM); ¹H NMR (300 MHz, CDCl₃) δ 1.65-1.75 (6H, m, 3×CH₂), 1.86 (6H, broad, 3×CH₂), 2.10 (3H, broad, 3×CH), 3.57 (1H, d, J=15.6 Hz, CH), 3.90 (1H, d, J=15.7 Hz, CM), 4.00 (1H, d, J=13.2 Hz, CH), 4.11 (1H, d, J=13.2 Hz, CH), 7.25 (2H, dt, J=8.3, 2.0 Hz, ArH) and 7.37 (2H, dt, J=8.2, 2.0 Hz, ArH); LC/MS (APCI) m/z 351 (M⁺+H); HPLC tᵣ=3.09 min (>99%) in 10% water-acetonitrile.

### 1-Adamantan-1-yl-2-(4-chloro-phenylmethanesulfonyl)-ethanone XDS04003, STX1632)

To a solution of 1-adamantan-1-yl-2-(4-chloro-phenylmethanesulfinyl)-ethanone (55 mg, 0.16 mmol) in DCM (5 mL) was added mCPBA (42 mg, purity 60-77%). The mixture was stirred at ambient temperature for 4 h, partitioned between ethyl acetate and 5% sodium carbonate solution. The organic phase was washed with brine, dried over sodium sulphate and concentrated in vacuo to give the title compound as white solid (40 mg, 68%). TLC single spot at R_{f}: 0.33 (20% EtOAc/DCM); ¹H NMR (300 MHz, CDCl₃) δ 1.58-1.68 (6H, m, 3×CH₂), 1.73 (6H, d, J=2.8 Hz, 3×CH₂), 2.02 (3H, broad, 3×CH), 3.80 (2H, s, CM), 4.40 (2H, s, CH), 7.30 (2H, dt, J=8.2, 2.0 Hz, ArH) and 7.36 (2H, dt, J=8.2, 2.0 Hz, ArH); LC/MS (APCI) m/z 367 (M⁺+H); HPLC tᵣ=3.14 min (>98%) in 10% water-acetonitrile.

### 1-Adamantan-1-yl-2-(thiophen-2-ylmethoxy)-ethanone (XDS04019A, STX1692)

To a cold suspension of NaH (60 mg, 1.5 mmol, 60% dispersion) in THF (3 mL) was added 2-thiophene methanol (114 mg, 1.0 mmol), followed by 1-adamantyl bromomethyl ketone (257 mg, 1.0 mmol). The mixture was stirred at 0° C. for 20 min, partitioned between ethyl acetate and saturated ammonium chloride solution. The organic phase was washed with brine, dried over sodium sulfate and concentrated in vacuo to give the crude product. Purification with flash column (hexane-ethyl acetate; gradient elution) yielded the title compound as off-white solid (49 mg, 17%). mp 57.2-58.3° C.; TLC single spot at R_{f}: 0.37 (30% ethyl acetate/hexane); ¹H NMR (270 MHz, CDCl₃) δ 1.56-1.79 (12H, m, 6×CH₂), 2.21 (3H, broad s, 3×CH), 4.30 (2H, s, CH₂), 4.75 (2H, s, CH₂), 6.98 (2H, m, ArH) and 7.30 (1H, m, ArH); LC/MS (APCI) m/z 291 (M⁺+H); HPLC tᵣ=2.98 min (>99%) in 10% water-acetonitrile.

### Synthesis of Adamantyl Amide Derivatives

### General Method a for Synthesis of Adamantyl Amide Derivatives:

To a solution of 1-adamantanecarbonyl chloride (52 mg, 0.26 mmol) in DCM (5 mL) was added triethylamine (0.1 mL), followed by the corresponding amine (0.25 mmol). The reaction mixture was stirred at ambient temperature under nitrogen overnight. PS-PS-trisamine (4.1 mmol/g, 100 mg) was added, the mixture was kept stirring for another 2 h, filtered and concentrated in vacuo to give the crude product that was purified by flash chromatography (Ethyl acetate-hexane gradient elution) to give desired adamantyl amide as crystalline solid.

### Adamantane-1-carboxylic acid (2,5-dimethyl-benzothiazol-4-yl)-amide XDS03084, STX1372)

The compound was prepared with general method A. White crystals (45 mg, 53%) were obtained. mp 183.5-185° C.; TLC single spot at R_{f}: 0.25 (20% ethyl acetate/hexane); ¹H NMR (270 MHz, CDCl₃) δ 1.71-1.90 (6H, m, 3×CH₂), 2.09 (9H, s, 3×CH and 3×CH₂), 2.30 (3H, s, CH₃), 2.78 (3H, s, CH₃), 7.21 (1H, d, J=8.2 Hz, ArH), 7.54 (1H, d, J=8.5 Hz, ArH) and 7.93 (1H, s, NH)); HRMS (FAB+) calcd. for C₂₀H₂₅N₂OS (M⁺+H) 341.1688, found 341.1688; LC/MS (APCI) m/z 341 (M⁺+H); HPLC tᵣ=3.11 min (>98%) in 10% water-acetonitrile.

### Adamantane-1-carboxylic acid (4-chloro-2-methyl-benzothiazol-5-yl)-amide (XDS03085, STX1373)

The compound was prepared with general method A. White crystals (48 mg, 53%) were obtained. mp 183.5-185.5° C.; TLC single spot at R_{f}: 0.31 (20% ethyl acetate/hexane); ¹H NMR (270 MHz, CDCl₃) δ 1.78 (6H, m, 3×CH₂), 2.03 (6H, d, J=3.0 Hz, 3×CH₂), 2.12 (3H, s, 3×CM), 2.86 (3H, s, CH₃), 7.68 (1H, d, J=8.9 Hz, ArH), 8.12 (1H, s, NH) and 8.50 (1H, d, J=8.9 Hz, ArH); HRMS (FAB+) calcd. for C₁₉H₂₂ClN₂OS (M⁺+H) 361.1141, found 361.1147; LC/MS (APCI) tᵣ=5.47 min, m/z 361 (1+H); HPLC tᵣ=3.90 min (>99%) in 10% water-acetonitrile.

### Adamantane-1-carboxylic acid (thiophen-2-ylmethyl)-amide (XDS03086, STX1374)

The compound was prepared with general method A. White crystals (60 mg, 87%) were obtained. mp 155-155.5° C.; TLC single spot at R_{f}: 0.22 (20% ethyl acetate/hexane); ¹H NMR (270 MHz, CDCl₃) δ 1.70 (6H, m, 3×CH₂), 1.86 (6H, d, J=3.0 Hz, 3×CH₂), 2.03 (3H, s, 3×CH), 4.60 (2H, d, J=5.6 Hz, NCH₂), 5.88 (1H, broad s, NH), 6.93 (2H, m, ArH) and 7.21 (1H, dd, J=3.0, 1.0 Hz, ArH); HRMS (FAB+) calcd. for C₁₆H₂₂NOS (M⁺+H) 276.1422, found 276.1422; LC/MS m/z 276 (M⁺+H); HPLC tᵣ=2.52 min (>99%) in 10% water-acetonitrile.

### Adamantane-1-carboxylic acid (2-thiophen-2-yl-ethyl)-amide (XDS03087, STX1375)

The compound was prepared with general method A. White crystals (56 mg, 78%) were obtained. mp 123-126° C.; TLC single spot at R_{f}: 0.26 (20% ethyl acetate/hexane); ¹H NMR (270 MHz, CDCl₃) δ 1.69 (6H, m, 3×CH₂), 1.80 (6H, d, J=2.8 Hz, 3×CH₂), 2.01 (3H, s, 3×CH), 3.01 (2H, t, J=5.9 Hz, CH₂), 3.49 (2H, q, J=6.1 Hz, NCH₂), 5.73 (1H, broad s, NH), 6.81 (1H, m, ArH), 6.95 (1H, dd, J=5.1, 1.0 Hz, ArH) and 7.16 (1H, dd, J=5.2, 1.2 Hz, ArH), HRMS (FAB+) calcd. for C₁₇H₂₄NOS (M⁺+H) 290.1579, found 290.1576; LC/MS (APCI) m/z 290 (M⁺+H); HPLC tᵣ=2.65 min (>99%) in 10% water-acetonitrile.

### Adamantane-1-carboxylic acid (1-methyl-3-oxo-butylidene)-hydrazide (XDS03123, STX1480)

The solution of adamantane-1-carboxylic acid hydrazide (97 mg, 0.5 mmol) and pentane-2,4-dione (100 mg, 1.0 mmol) in ethanol (6 mL) was refluxed for 6 h, concentrated in vacuo to give a white solid which was subjected to flash column chromatography (hexane-ethyl acetate; gradient elution) to yield the title product as white solid (90 mg, 65%). TLC single spot at R_{f}: 0.46 (30% ethyl acetate/hexane); ¹H NMR (270 MHz, CDCl₃) δ 1.65 (61, t, J=2.6 Hz, 3×CH₂), 1.69 (3H, s, CH₃), 1.95 (6H, t, J=2.0 Hz, 3×CH₂), 2.01 (3H, s, CH₃), 2.02 (3H, broad s, 3×CH), 2.56 (1H, d, J=18 Hz, CH), 2.84 (1H, d, J=18 Hz, CH) and 5.06 (1H, s, NH); LC/MS (APCI) m/z 275 (M⁺−H); HPLC tᵣ=3.57 min (>99%) in 20% water-acetonitrile.

### General Method B for Synthesis of Adamantyl Amide Derivatives:

To a solution of 1-adamantanecarbonyl chloride (100 mg, 0.50 mmol, 1.06 eq.) in DCM (5 mL) was added triethylamine (0.15 mL), followed by the corresponding amine (0.47 mmol, 1 eq.). The reaction mixture was stirred at ambient temperature under nitrogen overnight. PS-PS-trisamine (10-20 mg, 4.1 mmol/g) was added. After stirred at ambient temperature for another 2 h, the mixture was filtered and evaporation of the solvent gave a residue that was purified by flash chromatography (Ethyl acetate-DCM gradient elution) to give desired adamantyl amide as crystalline solid or amorphous solid.

### Adamantane-1-carboxylic acid (pyridin-2-ylmethyl)-amide (XDS03146, STX1540)

The title compound was synthesized with general method B. White crystalline solid (89 mg, 70%) was obtained. TLC single spot at R_{f}: 0.30 (50% ethyl acetate/DCM); ¹H NMR (270 MHz, CDCl₃) δ 1.70 (6H, m, 3×CH₂), 1.91 (6H, d, J=3.0 Hz, 3×CH₂), 2.05 (3H, broad s, 3×CH), 4.52 (2H, d, J=4.7 Hz, CH₂), 6.95 (1H, broad, NH), 7.15-7.24 (2H, m, ArH), 7.64 (1H, td, J=7.9, 2.0 Hz, ArH) and 8.53 (1H, d, J=4.5 Hz, ArH); LC/MS (APCI) m/z 269 (M⁺−H); HPLC tᵣ=2.49 min (>99%) in 10% water-acetonitrile.

### Adamantane-1-carboxylic acid (2-pyridin-2-yl-ethyl)-amide (XDS03147, STX1541)

The title compound was synthesized with general method B. White crystalline solid (117 mg, 88%) was obtained. TLC single spot at R_{f}: 0.68 (8% methanol/DCM); ¹H NMR (270 MHz, CDCl₃) δ 1.62-1.72 (6H, m, 3×CH₂), 1.78 (6H, d, J=2.6 Hz, 3×CH₂), 2.00 (3H, broad s, 3×CH), 2.98 (2H, t, J=6.4 Hz, CH₂), 3.60 (2H, q, J=6.4 Hz, CH₂), 6.70 (1H, broad, NH), 7.10-7.18 (2H, m, ArH), 7.62 (1H, td, J=7.9, 1.7 Hz, ArH) and 8.52 (1H, d, J=4.4 Hz, ArH); LC/MS (APCI) m/z 285 (M⁺+H); HPLC tᵣ=2.49 min (>99%) in 10% water-acetonitrile.

### Adamantane-1-carboxylic acid (pyridin-3-ylmethyl)-amide (XDS03148, STX1542)

The title compound was synthesized with general method B. White crystalline solid (97 mg, 76%) was obtained. TLC single spot at R_{f}: 0.62 (8% methanol/DCM); ¹H NMR (270 MHz, CDCl₃) δ 1.64-1.78 (6H, m, 3×CH₂), 1.86 (6H, d, J=2.8 Hz, 3×CH₂), 2.04 (3H, broad s, 3×CH), 4.44 (2H, d, J=6.0 Hz, CH₂), 5.94 (1H, broad, NH), 7.24 (1H, m, ArH), 7.58 (1H, dt, J=8.0, 1.7 Hz, ArH) and 8.52 (2H, m, ArH); LC/MS (APCI) m/z 269 (M⁺−H); HPLC tᵣ=2.38 min (>99%) in 10% water-acetonitrile.

### Adamantane-1-carboxylic acid (2-pyridin-3-yl-ethyl)-amide (XDS03149, STX1543)

The title compound was synthesized with general method B. White crystalline solid (96 mg, 72%) was obtained. TLC single spot at R_{f}: 0.60 (8% methanol/DCM); ¹H NMR (270 MHz, CDCl₃) δ 1.02-1.12 (12H, m, 6×CH₂), 1.34 (3H, broad s, 3×CH), 2.16 (2H, t, J=6.2 Hz, CH₂), 2.82 (2H, q, J=6.2 Hz, CH₂), 4.95 (1H, broad, NH), 6.58 (1H, m, ArH), 6.85 (1H, m, ArH) and 7.82 (2H, m, ArH); LC/MS (APCI) m/z 283-H); HPLC tᵣ=2.41 min (>99%) in 10% water-acetonitrile.

### Adamantane-1-carboxylic acid (6-methoxy-pyridin-3-yl)-amide (XDS03150, STX1544)

The title compound was synthesized with general method B. Off-white solid (112 mg, 83%) was obtained. TLC single spot at R_{f}: 0.79 (8% methanol/DCM); ¹H NMR (270 MHz, CDCl₃) δ 1.68-2.00 (12H, m, 6×CH₂), 2.10 (3H, broad s, 3×CH), 3.89 (3H, s, OCH₃), 6.71 (1H, d, J=8.9 Hz, ArH), 7.17 (1H, broad, NH), 7.93 (1H, dd, J=8.9, 2.8 Hz, ArH) and 8.11 (1H, d, J=2.5 Hz, ArH); LC/MS (APCI) m/z 285 (M⁺−H); HPLC tᵣ=2.78 min (>99%) in 10% water-acetonitrile.

### Adamantane-1-carboxylic acid (5-methyl-pyrazin-2-ylmethyl)-amide (XDS03151, STX1545)

The title compound was synthesized with general method B. White crystalline solid (102 mg, 76%) was obtained. TLC single spot at R_{f}: 0.67 (8% methanol/DCM); ¹H NMR (270 MHz, CDCl₃) δ 1.71 (6H, m, 3×CH₂), 1.89 (6H, m, 3×CH₂), 2.03 (3H, broad s, 3×CH), 2.55 (3H, s, CH₃), 4.53 (2H, d, J=5.0 Hz, CH₂), 6.63 (1H, broad, NH), 8.37 (1H, s, ArH) and 8.45 (1H, s, ArH); LC/MS (APCI) m/z 284 (M⁺−H); HPLC tᵣ=2.69 min (>99%) in 20% water-acetonitrile.

### 2-Adamantan-1-yl-N-thiophen-2-ylmethyl-acetamide (XDS03154, STX1564)

To a solution of 1-adamantyl acetic acid (155 mg, 0.80 mmol) in DCM (8 mL) were added EDCI (172 mg, 0.90 mmol), DMAP (55 mg, 0.40 mmol) and triethylamine (0.13 mL, 1.0 mmol). The mixture was stirred at ambient temperature for 20 min, 2-thiophene methylamine (88 mg, 0.78 mmol) was added. After stirred at ambient temperature for 24 h, the mixture was partitioned between ethyl acetate and 1% HCl solution. The organic phase was washed with 5% sodium bicarbonate and brine, dried over sodium sulphate and concentrated in vacuo to give the crude product. Purification with flash column (DCM-ethyl acetate; gradient elution) yielded the title compound as white solid (140 mg, 62%). TLC single spot at R_{f}: 0.66 (10% ethyl acetate/DCM); ¹H NMR (270 MHz, CDCl₃) δ 1.56-1.71 (12H, m, 6×CH₂), 1.93 (2H, s, CH₂), 1.95 (3H broad s, 3×CH), 4.60 (2H, d, J=5.2 Hz, CH₂), 5.63 (1H, broad, NH), 6.90-6.95 (2H, m, ArH) and 7.21 (1H, dd, J=4.7, 1.5 Hz, ArH); LC/MS (APCI) m/z 288 (M⁺−H); HPLC tᵣ=2.84 min (>99%) in 10% water-acetonitrile.

### 2-Adamantan-1-yl-N-pyridin-2-ylmethyl-acetamide (XDS03155, STX1565)

The title compound was synthesized according to procedure described for XDS03154. White crystalline solid (190 mg, 86%) was obtained. TLC single spot at R_{f}: 0.55 (10% methanol/DCM); ¹H NMR (270 MHz, CDCl₃) δ 1.58-1.70 (12H, m, 6×CH₂), 1.95 (3H, broad s, 3×CH), 2.02 (2H, s, CH₂), 4.54 (2H, d, J=4.9 Hz, CH₂), 6.60 (1H, broad, NH), 7.16-7.29 (2H, m, ArH), 7.65 (1H, td, J=7.7, 2.0 Hz, ArH) and 8.52 (1H, d, J=5.0 Hz, ArH); LC/MS (APCI) m/z 285 (M⁺+H); HPLC tᵣ=2.53 min (>99%) in 10% water-acetonitrile.

### 2-Adamantan-1-yl-N-(2-methyl-benzothiazol-5-yl)-acetamide (XDS03156, STX1566)

The title compound was synthesized according to procedure described for XDS03154. Off-white solid (121 mg, 46%) was obtained. TLC single spot at R_{f}: 0.47 (40% ethyl acetate/DCM); ¹H NMR (270 MHz, CDCl₃) δ 1.61-1.71 (12H, m, 6×CH₂), 1.98 (3H, broad s, 3×CH), 2.12 (2H, s, CH₂), 2.82 (3H, s, CH₃), 7.17 (1H, broad, NH), 7.65 (1H, dd, J=8.8, 2.0 Hz, ArH), 7.73 (1H, d, J=8.7 Hz, ArH) and 7.96 (1H, d, J=2.0 Hz, ArH); LC/MS (APCI) m/z 341 (W+H); HPLC tᵣ=3.26 min (>99%) in 10% water-acetonitrile.

### Adamantan-1-yl-acetyl chloride (XDS03162)

To a cold (0° C.) solution of 1-adamantyl acetic acid (477 mg, 2.46 mmol) in ethyl acetate (12 mL) was added DMF (20 μL), followed by oxalyl chloride (328 mg, 2.58 mmol). The mixture was stirred at ambient temperature for 3 h, concentrated in vacuo to give the title compound as oil (520 mg, 99%). The compound was used in the next step without further purification.

### 2-Adamantan-1-yl-N-(2-thiophen-2-yl-ethyl)-acetamide (XDS03163, STX1570)

To a solution of adamantan-1-yl-acetyl chloride (XDS03162, 174 mg, 0.82 mmol) in DCM (8 mL) was added triethylamine (0.20 mL), followed by the 2-thiopheneethylamine (99 mg, 0.78 mmol). The reaction mixture was stirred at ambient temperature under nitrogen overnight. PS-PS-trisamine (30 mg, 4.1 mmol/g) was added. After stirred at ambient temperature for another 2 h, the mixture was filtered and evaporation of the solvent gave a residue that was purified by flash chromatography (Ethyl acetate-DCM gradient elution) to give the title compound as crystalline solid (218 mg, 92%). TLC single spot at R_{f}: 0.36 (40% EtOAc/hexane); ¹H NMR (270 MHz, CDCl₃) δ 1.55-1.70 (12H, m, 6×CH₂), 1.87 (2H, s, CH₂), 1.93 (3H, broad s, 3×CH), 3.03 (211, t, J=6.3 Hz, CH₂), 3.53 (2H, q, J=6.2 Hz, CH₂), 5.42 (1H, broad s, NH), 6.83 (1H, d, J=2.4 Hz, ArH), 6.93 (1H, dd, J=5.3, 3.5 Hz, ArH) and 7.15 (1H, dd, J=5.2, 1.3 Hz, ArH); LC/MS (APCI) m/z 302 (M⁺−H); HPLC tᵣ=3.14 min (>98%) in 10% water-acetonitrile.

### 2-Adamantan-1-yl-N-(3,4-dichloro-phenyl)-acetamide (XDS03164, STX1571)

The title compound was synthesized according to the procedure described for XDS03163. White crystalline solid (206 mg, 78%) was obtained. TLC single spot at R_{f}: 0.70 (40% EtOAc/hexane); ¹H NMR (270 MHz, CDCl₃) δ 1.57-1.73 (12H, m, 6×CH₂), 1.98 (3H, broad s, 3×CH), 2.07 (2H, s, CH₂), 7.06 (1H, broad, NM, 7.29 (1H, dd, J=8.8, 2.0 Hz, ArH), 7.35 (1H, d, J=8.7 Hz, ArH) and 7.77 (1H, d, J=2.0 Hz, ArH); LC/MS (APCI) m/z 336 (M⁺−H); HPLC tᵣ=5.87 min (>99%) in 10% water-acetonitrile.

### 2-Adamantan-1-yl-N-(2,5-dimethyl-benzothiazol-4-yl)-acetamide (XDS03165, STX1572)

The title compound was synthesized according to the procedure described for XDS03163. Off-white solid (95 mg, 34%) was obtained. TLC single spot at R_{f}: 0.22 (40% EtOAc/hexane); ¹H NMR (270 MHz, CDCl₃) δ 1.65-1.80 (12H, m, 6×CH₂), 2.00 (3H, broad s, 3×CH), 2.25 (2H, s, CH₂), 2.39 (3H, s, CH₃), 2.76 (3H, s, CH₃), 7.22 (1H, d, J=8.1 Hz, ArH), 7.55 (1H, d, J=8.1 Hz, ArH) and 7.63 (1H, s, NM; LC/MS (APCI) m/z 353 (M⁺−H); HPLC tᵣ=3.72 min (>99%) in 10% water-acetonitrile.

### Adamantane-1-carboxylic acid methyl-thiophen-2-ylmethyl-amide (XDS03171, STX1576)

The title compound was synthesized with general method B. Colourless thick oil (110 mg, 81%) was obtained. TLC single spot at R_{f}: 0.80 (25% ethyl acetate/DCM); ¹H NMR (270 MHz, CDCl₃) δ 1.71 (6H, m, 3×CH₂), 2.03 (9H, s, 3×CH₂ and 3×CH), 3.07 (3H, s, CH₃), 4.73 (2H, s, CH₂), 6.90-6.95 (2H, m, ArH) and 7.21 (1H, dd, J=4.4, 1.7 Hz, ArH), LC/MS (APCI) m/z 290 (M⁺+H); HPLC tᵣ=3.89 min (>97%) in 10% water-acetonitrile.

### Adamantane-1-carboxylic acid (1-methyl-1H-imidazol-4-ylmethyl)-amide (XDS03172, STX1577)

The title compound was synthesized with general method B. Off-white solid (95 mg, 74%) was obtained. TLC single spot at R_{f}: 0.79 (12% methanol/DCM); ¹H NMR (270 MHz, CDCl₃) δ 1.63-1.73 (6H, m, 3×CH₂), 1.84 (6H, d, J=2.7 Hz, 3×CH₂), 2.01 (3H, broad s, 3×CH), 3.63 (3H, s, CH₃), 4.30 (2H, d, J=4.7 Hz, CH₂), 6.21 (1H, broad, NH), 6.78 (1H, s, ArH) and 7.35 (1H, s, ArH); LC/MS (APCI) tᵣ=1.3 min (>98%) in 5% water-methanol, m/z 274 (M⁺+H).

### Adamantane-1-carboxylic acid (1-methyl-11H-pyrrol-2-ylmethyl)-amide (XDS03173, STX1578)

The title compound was synthesized with general method B. White solid (97 mg, 76%) was obtained. TLC single spot at R_{f}: 0.69 (15% methanol/DCM); ¹H NMR (270 MHz, CDCl₃) δ 1.58-1.75 (6H, m, 3×CH₂), 1.83 (6H, d, J=2.8 Hz, 3×CH₂), 2.02 (311, broad s, 3×CH), 3.53 (3H, s, CH₃), 4.42 (2H, d, J=5.2 Hz, CH₂), 5.60 (1H, broad, NH), 6.05 (2H, d, J=2.2 Hz, ArH) and 6.65 (1H, t, J=2.2 Hz, ArH); LC/MS (APCI) m/z 271 (M⁺−H); HPLC tᵣ=2.67 min (>99%) in 10% water-acetonitrile.

### Adamantane-1-carboxylic acid (furan-2-ylmethyl)-amide (XDS03174, STX1579)

The title compound was synthesized with general method B. White crystalline solid (95 mg, 78%) was obtained. TLC single spot at R_{f}: 0.50 (10% EtOAc/DCM); ¹H NMR (270 MHz, CDCl₃) δ 1.65-1.75 (6H, m, 3×CH₂), 1.85 (6H, d, J=2.8 Hz, 3×CH₂), 2.03 (3H, broad s, 3×CH), 4.41 (2H, d, J=5.8 Hz, CH₂), 5.85 (1H, broad, NH), 6.19 (1H, d, J=2.8 Hz, ArH), 6.30 (1H, dd, J=3.3, 2.0 Hz, ArH) and 7.34 (1H, d, J=1.9 Hz, ArH); LC/MS (APCI) m/z 258 (M⁺−H); HPLC tᵣ=2.61 min (>99%) in 10% water-acetonitrile.

### Adamantane-1-carboxylic acid (3-imidazol-1-yl-propyl)-amide (XDS03175, STX1580)

The title compound was synthesized with general method B. White solid (87 mg, 64%) was obtained. TLC single spot at R_{f}: 0.69 (15% methanol/DCM); ¹H NMR (270 MHz, CDCl₃) δ 1.62-1.75 (6H, m, 3×CH₂), 1.80 (6H, d, J=2.5 Hz, 3×CH₂), 1.98 (2H, p, CH₂), 2.03 (3H, broad, 3×CH), 3.26 (2H, q, J=6.7 Hz, CH₂), 3.96 (2H, t, J=6.9 Hz, CH₂), 5.59 (1H, broad, NH), 6.94 (1H, s, ArH), 7.06 (1H, s, ArH) and 7.48 (1H, s, ArH); LC/MS (APCI) tᵣ=1.1 min (>98%) in 5% water-methanol, m/z 286 (M⁺−H).

### Adamantane-1-carboxylic acid [2-(1H-indol-3-yl)-ethyl]-amide (XDS03178, STX1581)

The title compound was synthesized with general method B. White solid (110 mg, 73%) was obtained. TLC single spot at R_{f}: 0.52 (25% EtOAc/DCM); ¹H NMR (270 MHz, CDCl₃) δ 1.60-1.73 (6H, m, 3×CH₂), 1.76 (6H, d, J=2.7 Hz, 3×CH₂), 1.98 (3H, broad, 3×CH), 2.96 (2H, t, J=6.7 Hz, CH₂), 3.56 (2H, q, J=6.8 Hz, CH₂), 5.67 (1H, broad, NH), 7.02 (1H, d, J=2.2 Hz, ArH), 7.10-7.23 (2H, m, ArH), 7.38 (1H, d, J=8.2 Hz, ArH), 7.62 (1H, d, J=7.7 Hz, ArH) and 8.06 (1H, s, NH); LC/MS (APCI) m/z 321 (M−H); HPLC tᵣ=2.67 min (>99%) in 10% water-acetonitrile.

### Adamantane-1-carboxylic acid [2-(1-methyl-pyrrolidin-2-yl)-ethyl]-amide (XDS03176, STX1597)

The title compound was synthesized with general method B. White solid (65 mg, 48%) was obtained. TLC single spot at R_{f}: 0.32 (12% methanol/DC); ¹H NMR (270 CDCl₃) δ 1.57-1.78 (18H, m, 9×CH₂), 1.85 (1H, m, CH), 2.11 (3H, broad, 3×CH), 2.17 (1H, m, CH), 2.28 (1H, m, CH), 2.31 (3H, s, CH₃), 3.20 (1H, m, CH), 3.44 (1H, m, CH), and 7.31 (1H, broad, NH); LC/MS (APCI) t=1.8 min (>96%) in 5% water-methanol, m/z 291 (M⁺+H).

### Adamantane-1-carboxylic acid (2-piperidin-1-yl-ethyl)-amide (XDS03177, STX1598)

The title compound was synthesized with general method B. White solid (82 mg, 60%) was obtained. TLC single spot at R_{f}: 0.69 (12% methanol/DCM); ¹H NMR (270 MHz, CDCl₃) δ 1.42 (2H, m, CH₂), 1.56 (4H, m, 2×CH₂), 1.65-1.75 (6H, m, 3×CH₂), 1.85 (6H, d, J=2.8 Hz, 3×CH₂), 2.02 (3H, broad, 3×CH), 2.38 (4H, t, J=5.8 Hz, 2×CH₂), 2.43 (2H, t, J=6.9 Hz, CH₂), 3.28 (2H, q, J=5.9 Hz, CH₂) and 6.37 (1H, broad, NH); LC/MS (APCI) tᵣ=1.7 min (>98%) in 5% water-methanol, m/z 291 (M⁺+H).

### 2-Adamantan-1-yl-N-methyl-N-thiophen-2-ylmethyl-acetamide (XDS04004, STX1661)

To a solution of 1-adamantyl acetic acid (155 mg, 0.80 mmol) in DCM (8 mL) were added EDCI (172 mg, 0.90 mmol), DMAP (55 mg, 0.40 mmol) and triethylamine (0.13 mL, 1.0 mmol). The mixture was stirred at ambient temperature for 20 min, N-methyl-thiophen-2-ylmethyl-amine (99 mg, 0.78 mmol) was added. After stirred at ambient temperature for 18 h, the mixture was partitioned between ethyl acetate and 2% HCl solution. The organic phase was washed with 5% sodium bicarbonate and brine, dried over sodium sulphate and concentrated in vacuo to give the crude product. Purification with flash column (hexane-ethyl acetate; gradient elution) yielded the title compound as white solid (160 mg, 68%). TLC single spot at R_{f}: 0.46 (30% ethyl acetate/hexane); ¹H NMR (300 MHz, CDCl₃) δ 1.62-1.77 (12H, m, 6×CH₂), 2.00 (3H, broad s, 3×CH), 2.19 (2H, s, CH₂), 3.00 (3H, s, CH₃), 4.52 (2H, s, CH₂), 6.95 (2H, m, ArH) and 7.23 (1H, dd, J=5.0, 1.3 Hz, ArH); LC/MS (APCI) m/z 304 (M⁺+H); HPLC tᵣ=4.04 min (>99%) in 10% water-acetonitrile.

### 2-Adamantan-1-yl-N-furan-2-ylmethyl-acetamide (XDS04005, STX1662)

The title compound was synthesized according to procedure described for STX1654. White crystalline solid (170 mg, 80%) was obtained. TLC single spot at R_{f}: 0.37 (30% ethyl acetate/hexane); ¹H NMR (300 MHz, CDCl₃) δ 1.53 (6H, d, J=2.8 Hz, 3×CH₂), 1.55-1.66 (6H, m, 3×CH₂), 1.87 (2H, s, CH₂), 1.90 (3H, broad, 3×CH), 4.37 (2H, d, J=5.5 Hz, CH₂), 5.15 (1H, broad, NH), 6.16 (1H, dd, J=3.2, 0.8 Hz, ArH), 6.25 (1H, dd, J=3.2, 1.9 Hz, ArH) and 7.28 (1H, dd, J=1.8, 0.8 Hz, ArH); LC/MS (APCI) m/z 274 (M⁺+H); HPLC tᵣ=2.71 min (>99%) in 10% water-acetonitrile.

### 2-Adamantan-1-yl-N-(1-methyl-1H-pyrrol-2-ylmethyl)acetamide (XDS04006, STX1663)

The title compound was synthesized according to procedure described for STX1654. White crystalline solid (170 mg, 76%) was obtained. TLC single spot at R_{f}: 0.37 (40% ethyl acetate/hexane); ¹H NMR (300 MHz, CDCl₃) δ 1.55 (6H, d, J=2.8 Hz, 3×CH₂), 1.57-1.62 (6H, m, 3×CH₂), 1.85 (2H, s, CH₂), 1.91 (3H, broad, 3×CH), 3.51 (3H, s, CH₃), 4.35 (2H, d, J=5.2 Hz, CH₂), 5.30 (1H, broad, NH), 6.00 (2H, d, J=2.3 Hz, ArH) and 6.55 (1H, t, J=2.2 Hz, ArH); LC/MS (APCI) m/z 285 (M⁺−H); HPLC tᵣ=2.87 min (>99%) in 10% water-acetonitrile.

### Adamantane-1-carboxylic acid (benzo[b]thiophen-2-ylmethyl)-amide (XDS04013, STX1688)

The title compound was synthesized with general method B. White crystalline solid (145 mg, 95%) was obtained. TLC single spot at R_{f}: 0.66 (10% ethyl acetate/DCM); ¹H NMR (270 MHz, CDCl₃) δ 1.67-1.76 (6H, m, 3×CH₂), 1.87 (6H, d, J=2.8 Hz, 3×CH₂), 2.04 (3H, broad s, 3×CH), 4.67 (2H, d, J=5.7 Hz, CH₂), 6.00 (1H, broad, NM, 7.22-7.34 (3H, m, ArH) and 7.64-7.78 (3H, m, ArH), LC/MS (APCI) m/z 326 (M⁺+H); HPLC tᵣ=2.80 min (>99%) in 10% water-acetonitrile.

### Adamantane-1-carboxylic acid (benzo[b]thiophen-3-ylmethyl)-amide (XDS04014, STX1689)

The title compound was synthesized with general method B. White crystalline solid (112 mg, 73%) was obtained. TLC single spot at R_{f}: 0.61 (10% ethyl acetate/DCM); ¹H NMR (270 MHz, CDCl₃) δ 1.62-1.74 (6H, m, 3×CH₂), 1.85 (6H, d, J=2.8 Hz, 3×CH₂), 2.02 (3H, broad s, 3×CH), 4.68 (2H, d, J=5.2 Hz, CH₂), 5.80 (1H, broad, NH), 7.29-7.43 (3H, m, ArH) and 7.73-7.87 (3H, m, ArH); LC/MS (APCI) m/z 326 (M⁺+H); HPLC tᵣ=2.83 min (>99%) in 10% water-acetonitrile.

### 2-Adamantan-1-yl-N-benzo[b]thiophen-2-ylmethyl-acetamide (XDS04015, STX1690)

To a solution of 1-adamantyl acetic acid (97 mg, 0.50 mmol) in DCM (5 mL) were added EDCI (105 mg, 0.55 mmol), DMAP (16 mg, 0.25 mmol) and triethylamine (0.13 mL, 1.0 mmol). The mixture was stirred at ambient temperature for 20 min. Benzo[b]thiophen-2-yl-methylamine (81 mg, 0.50 mmol) was added. After stirring at ambient temperature overnight, the mixture was partitioned between ethyl acetate and 2% HCl solution. The organic phase was washed with 5% sodium bicarbonate and brine, dried over sodium sulphate and concentrated in vacuo to give the crude product. Purification with flash column (DCM-ethyl acetate; gradient elution) yielded the title compound as white solid (126 mg, 74%), TLC single spot at R_{f}: 0.39 (10% ethyl acetate/DCM); ¹H NMR (270 MHz, CDCl₃) δ 1.60-1.71 (12H, m, 6×CH₂), 1.95 (3H, broad s, 3×CH), 1.97 (2H, s, CH₂), 4.69 (2H, d, J=5.3 Hz, CH₂), 5.73 (1H, broad, NH), 7.17-7.33 (3H, m, ArH) and 7.67-7.79 (3H, m, ArH); LC/MS (APCI) m/z 340 (M⁺+H); HPLC tᵣ=3.00 min (>99%) in 10% water-acetonitrile.

### 2-Adamantan-1-yl-N-benzo[b]thiophen-3-ylmethyl-acetamide (XDS04016, STX1691)

The title compound was synthesized according to method described for XDS04015. Purification with flash column (DCM-ethyl acetate; gradient elution) yielded the title compound as white solid (80 mg, 47%). TLC single spot at R_{f}: 0.31 (10% ethyl acetate/DCM); ¹H NMR (270 MHz, CDCl₃) δ 1.55-1.70 (12H, m, 6×CH₂), 1.94 (5H, broad s, CH₂ and 3×CH), 4.68 (2H, d, J=5.5 Hz, CH₂), 5.52 (1H, broad, NH), 7.30-7.41 (3H, m, ArH) and 7.80-7.87 (3H, m, ArH); LC/MS (APCI) m/z 340 (M⁺+H); HPLC tᵣ=2.97 min (>99%) in 10% water-acetonitrile.

### Synthesis of Adamantane Arylsulphonamide Derivatives

### General Method for Synthesis of Adamantane Arylsulphonamide Derivatives:

To a solution of arylsulphonyl chloride (0.53 mmol, 1.06 eq.) in DCM (8 mL) was added pyridine (0.2 mL), followed by the corresponding amine (0.50 mmol, 1 eq.). The reaction mixture was stirred at ambient temperature under nitrogen for 3-5 h. After TLC showed completion of the reaction, the mixture was partitioned between ethyl acetate and brine. The organic layer was washed with 5% HCl, 5% sodium carbonate and brine, dried over sodium sulphate, and concentrated in vacuo to give the crude product that was purified by lash chromatography (Ethyl acetate-hexane gradient elution) to give desired arylsulphonamide as crystalline solid or amorphous solid.

### N-Adamantan-1-yl-4-propyl-benzenesulfonamide (XDS03072, STX1341)

The compound was prepared with general method. White crystals (68 mg, 41%) were obtained. mp 178-178.5° C.; TLC single spot at R_{f}: 0.20 (15% ethyl acetate/hexane); ¹H NMR (270 MHz, CDCl₃) δ 0.93 (3H, t, J=7.2 Hz, CH₃), 1.56 (6H, m, 3×CH₂), 1.66 (2H, sextet, J=7.3 Hz, CH₂), 1.77 (6H, d, J=3.0 Hz, 3×CH₂), 1.99 (3H, broad s, 3×CH), 2.64 (2H, t, J=7.2 Hz, CH₂), 4.40 (1H, s, NM, 7.26 (2H, m, ArH), and 7.78 (2H, m, ArH); HRMS (FAB+) calcd. for C₁₉H₂₈NO₂S (M⁺+H) 334.1841, found 334.1836; LC/MS (APCI) tᵣ=5.47 min, m/z 332 (M+f-H); HPLC tᵣ=2.93 min (>99%) in 10% water-acetonitrile.

### N-Adamantan-1-yl-2,5-dichloro-benzenesulfonamide (XDS03073, STX1342)

The compound was prepared with general method. White crystals (60 mg, 33%) were obtained. mp 189-190° C.; TLC single spot at R_{f}: 0.23 (15% ethyl acetate/hexane); ¹H NMR (270 MHz, CDCl₃) δ 1.58 (6H, m, 3×CH₂), 1.77 (6H, d, J=3.2 Hz, 3×CH₂), 2.01 (3H, broad s, 3×CH), 4.91 (1H, s, NH), 7.43 (2H, d, J=1.1 Hz, ArH) and 8.10 (1H, t, J=1.5 Hz, ArH); HRMS (FAB+) calcd. for C₁₆H₂₀Cl₂NO₂S (M⁺+H) 360.0592, found 360.0539; LC/MS (APCI) tᵣ=5.45 min (>99%), m/z 357.8 (M⁺−H); HPLC tᵣ=3.10 min (>97%) in 10% water-acetonitrile.

### N-Adamantan-1-yl-3-chloro-2-methyl-benzenesulfonamide (XDS03074, STX1343)

The compound was prepared with general method. White crystals (60 mg, 35%) were obtained. mp 197.5-198° C.; TLC single spot at R_{f}: 0.27 (12% ethyl acetate/hexane); ¹H NMR (270 MHz, CDCl₃) δ 1.57 (6H, m, 3×CH₂), 1.76 (6H, d, J=2.8 Hz, 3×CH₂), 2.00 (3H, broad s, 3×CH), 2.70 (3H, s, CH₃), 4.39 (1H, s, NH), 7.23 (1H, t, J=8.0 Hz, ArH), 7.54 (1H, dd, J=7.8, 1.2 Hz, ArH) and 7.99 (1H, dd, J=7.9, 1.2 Hz, ArH); HRMS (FAB+) calcd. for C₁₇H₂₃ClNO₂S (M⁺+H) 340.1138, found 340.1116; LC/MS (APCI) tᵣ=5.51 min, m/z 338 (M⁺−H); HPLC tᵣ=3.04 min (>99%) in 10% water-acetonitrile.

### Biphenyl-4-sulfonic acid adamantan-1-ylamide (XDS03075, STX1344)

The compound was prepared with general method. White crystals (78 mg, 43%) were obtained. mp 205-205.5° C.; TLC single spot at R_{f}: 0.30 (30% ethyl acetate/hexane); ¹H NMR (270 MHz, CDCl₃) δ 1.56 (6H, m, 3×CH₂), 1.82 (6H, d, J=2.7 Hz, 3×CH₂), 2.01 (3H, broad s, 3×CH), 4.46 (1H, s, NH), 7.39-7.50 (3H, m, ArH), 7.61 (2H, m, ArH), 7.68 (2H, m, ArH) and 7.94 (2H, m, ArH); HRMS (FAB+) calcd. for C₂₂H₂₆NO₂S (M⁺+H) 368.1684, found 368.1680; LC/MS (APCI) tᵣ=5.58 min (>99%), m/z 366 (M⁺−H); HPLC tᵣ=2.92 min (>99%) in 10% water-acetonitrile.

### General Method for Synthesis of Adamantylmethyl Arylsulphonamide Derivatives:

To a solution of arylsulphonyl chloride (0.53 mmol, 1.06 eq.) in DCM (6 mL) was added pyridine (0.2 mL), followed by the corresponding amine (0.50 mmol, 1 eq.). The reaction mixture was stirred at ambient temperature under nitrogen overnight. PS-PS-trisamine (4.1 mmol/g, 100 mg) was added, the mixture was kept stirring for another 2 h, filtered and concentrated in vacuo to give the crude product that was purified by flash chromatography (Ethyl acetate-hexane gradient elution) to give desired arylsulphonamide as crystalline solid or amorphous solid.

### N-Adamantan-1-ylmethyl-4-propyl-benzenesulfonamide (XDS03080, STX1368)

The compound was prepared with general method. White crystals (102 mg, 59%) were obtained. mp 148-149° C.; TLC single spot at R_{f}: 0.50 (20% ethyl acetate/hexane); ¹H NMR (270 MHz, CDCl₃) δ 0.93 (3H, t, J=7.2 Hz, CH₃), 1.42 (6H, d, J=2.6 Hz, 3×CH₂), 1.54-1.70 (8H, m, 4×CH₂), 1.95 (3H, broad s, 3×CH), 2.57 (2H, d, J=6.7 Hz, NCH₂), 2.64 (2H, t, J=7.2 Hz, CH₂), 4.60 (1H, t, J=6.6 Hz, ArH), 7.29 (2H, m, ArH), and 7.73 (2H, m, ArH); HRMS (FAB+) calcd. for C₂₀H₃₀NO₂S (M⁺+H) 348.1997, found 348.2007; LC/MS (APCI) m/z 348 (M⁺+H); HPLC t=3.29 min (>99%) in 10% water-acetonitrile.

### N-Adamantan-1-ylmethyl-2,5-dichloro-benzenesulfonamide (XDS03081, STX1369)

The compound was prepared with general method. White crystals (100 mg, 53%) were obtained. mp 197-198° C.; TLC single spot at R_{f}: 0.49 (20% ethyl acetate/hexane); ¹H NMR (270 MHz, CDCl₃) δ 1.45 (6H, d, J=2.5 Hz, 3×CH₂), 1.54-1.68 (6H, m, 3×CH₂), 1.97 (3H, broad s, 3×CH), 2.53 (2H, d, J=6.7 Hz, NCH₂), 4.92 (1H, t, J=6.6 Hz, NH), 7.46 (2H, m, ArH) and 8.07 (1H, dd, J=2.0, 1.0 Hz, ArH); HRMS (FAB+) calcd. for C₁₇H₂₂Cl₂NO₂S (M⁺+H) 374.0748, found 374.0702; LC/MS (APCI) m/z 374 (M⁺+H); HPLC tᵣ=3.50 min (>99%) in 20% water-acetonitrile.

### N-Adamantan-1-ylmethyl-3-chloro-2-methyl-benzenesulfonamide (XDS03082, STX1370)

The compound was prepared with general method. White crystals (97 mg, 55%) were obtained. mp 183-184° C.; TLC single spot at R_{f}: 0.50 (20% ethyl acetate/hexane); ¹H NMR (270 MHz, CDCl₃) δ 1.41 (6H, d, J=3.2 Hz, 3×CH₂), 1.55-1.72 (6H, m, 3×CH₂), 1.96 (3H, broad s, 3×CH), 2.57 (2H, d, J=6.6 Hz, NCH₂), 2.69 (3H, s, CH₃), 4.41 (1H, t, J=6.5 Hz, NH), 7.25 (1H, t, J=7.6 Hz, ArH), 7.57 (1H, dd, J=7.6, 1.2 Hz, ArH) and 7.89 (1H, dd, J=7.7, 1.2 Hz, ArH); HRMS (FAB+) calcd. for C₁₈H₂₅ClNO₂S (M⁺+H) 354.1294, found 354.1283; LC/MS (APCI) m/z 354 (M⁺+H); HPLC tᵣ=3.29 min (>99%) in 20% water-acetonitrile.

### Biphenyl-4-sulfonic acid (adamantan-1-ylmethyl)-amide (XDS03083, STX1371)

The compound was prepared with general method. White crystals (110 mg, 58%) were obtained. mp 188-189.5° C.; TLC single spot at R_{f}: 0.31 (20% ethyl acetate/hexane); ¹H NMR (270 MHz, CDCl₃) δ 1.45 (6H, d, J=2.8 Hz, 3×CH₂), 1.54-1.72 (6H, m, 3×CH₂), 1.96 (3H, broad s, 3×CH), 2.62 (2H, d, J=6.6 Hz, NCH₂), 4.40 (1H, t, J=6.5 Hz, NH), 7.40-7.50 (3H, m, ArH), 7.59-7.62 (2H, m, ArH), 7.71 (2H, m, ArH) and 7.90 (2H, m, ArH); HRMS (FAB+) calcd. for C₂₃H₂₈NO₂S (M⁺+H) 382.1841, found 382.1833; LC/MS (APCI) m/z 382 (M⁺+H); HPLC tᵣ=3.15 min (>98%) in 10% water-acetonitrile.

### N-Adamantan-1-yl-C-(4-chloro-phenyl)-methanesulfonamide (XDS03137, STX1499)

To a solution of 4-chlorophenyl methanesulfonyl chloride (91 mg, 0.41 mmol) in DCM (5 mL) was added pyridine (0.1 mL), followed by the 1-adamantamine (59 mg, 0.39 mmol). The reaction mixture was stirred at ambient temperature under nitrogen for 18 h, partitioned between ethyl acetate and brine. The organic layer was washed with 5% HCl, 5% sodium carbonate and brine, dried over sodium sulphate, and concentrated in vacuo to give the crude product that was purified by flash chromatography (Ethyl acetate-hexane gradient elution) to give the title compound as crystalline solid (27 mg, 20%). TLC single spot at R_{f}: 0.67 (30% ethyl acetate/hexane); ¹H NMR (270 MHz, CDCl₃) δ 1.65 (6H, m, 3×CH₂), 1.93 (6H, d, J=3.0 Hz, 3×CH₂), 2.10 (3H, broad s, 3×CH), 3.82 (1H, s, NH), 4.20 (2H, s, CH₂) and 7.34 (4H, s, ArH); LC/MS (APCI) m/z 338 (M⁺−H); HPLC tᵣ=2.80 min (>98%) in 20% water-acetonitrile.

### N-Adamantan-1-yl-C-(3-chloro-phenyl)-methanesulfonamide (XDS03138, STX1500)

The title compound was synthesized according to the method described for XDS03137. Crystalline solid (31 mg, 23%) was obtained. TLC single spot at R_{f}: 0.67 (30% ethyl acetate/hexane); ¹H NMR (270 MHz, CDCl₃) δ 1.65 (6H, m, 3×CH₂), 1.93 (6H, d, J=2.7 Hz, 3×CH₂), 2.10 (3H, broad s, 3×CH), 3.85 (1H, s, NH), 4.20 (2H, s, CH₂), 7.27-7.34 (3H, m, ArH) and 7.40 (1H, d, J=1.3 Hz, ArH); LC/MS (APCI) m/z 338 (M⁺−H); HPLC tᵣ=2.78 min (>99%) in 10% water-acetonitrile.

### Thiophene-2-sulfonic acid adamantan-1-ylamide (XDS03168, STX1573)

To a solution of thiophene-2-sulfonyl chloride (97 mg, 0.53 mmol, 1.06 eq.) in DCM (3 mL) was added pyridine (0.2 mL), followed by 1-adamantamine (0.50 mmol, 1 eq.). The reaction mixture was stirred at ambient temperature under nitrogen for 5 h. PS-PS-trisamine (4.1 mmol/g, 50 mg) was added. The mixture was kept stirring for another 1 h, filtered and concentrated in vacuo to give the crude product that was purified by flash chromatography (Ethyl acetate-hexane gradient elution) to give the title compound as crystalline solid (89 mg, 60%). mp 129-131° C.; TLC single spot at R_{f}: 0.51 (35% ethyl acetate/hexane); ¹H NMR (270 MHz, CDCl₃) δ 1.54-1.65 (6H, d, J=2.5 Hz, 3×CH₂), 1.85 (6H, d, J=3.0 Hz, 3×CH₂), 2.03 (3H, broad s, 3×CH), 4.47 (1H, s, NH), 7.02 (2H, dd, J=5.2, 3.6 Hz, ArH), 7.53 (2H, dd, J=5.2, 1.3 Hz, ArH) and 7.59 (1H, dd, J=3.7, 1.2 Hz, ArH); LC/MS (APCI) m/z 296 (M⁺−H); HPLC tᵣ=2.88 min (>99%) in 10% water-acetonitrile.

### Thiophene-2-sulfonic acid adamantan-2-ylamide (XDS03169, STX1574)

The title compound was synthesized according to the procedure described for XDS03168. White crystalline solid (107 mg, 72% a) was obtained. TLC single spot at R_{f}: 0.50 (35% ethyl acetate/hexane); ¹H NMR (270 MHz, CDCl₃) δ 1.54-1.79 (14H, m, 6×CH₂ and 2×CH), 3.50 (1H, m, CH), 4.86 (1H, d, J=7.5 Hz, NH), 7.05 (2H, dd, J=4.9, 3.9 Hz, ArH), 7.55 (2H, dd, J=5.0, 1.4 Hz, ArH) and 7.60 (1H, dd, J=3.7, 1.2 Hz, ArH); LC/MS (APCI) m/z 296 (M⁺−H); HPLC tᵣ=2.89 min (>93%) in 10% water-acetonitrile.

### Thiophene-2-sulfonic acid (adamantan-1-ylmethyl)-amide (XDS03170, STX1575)

The title compound was synthesized according to the procedure described for XDS03168. White crystalline solid (109 mg, 70%) was obtained. TLC single spot at R_{f}: 0.50 (35% ethyl acetate/hexane); ¹H NMR (270 MHz, CDCl₃) δ 1.44 (6H, d, J=2.7 Hz, 3×CH₂), 1.54-1.72 (6H, m, 3×CH₂), 1.96 (3H, broad s, 3×CH), 2.66 (2H, d, J=6.8 Hz, CH₂), 4.42 (1H, broad, NH), 7.07 (1H, dd, J=4.9, 3.7 Hz, ArH) and 7.56-7.58 (2H, m, ArH). LC/MS (APCI) m/z 310 (M⁺−H); HPLC tᵣ=3.24 min (>99%) in 10% water-acetonitrile.

### Synthesis of Adamantyl Oxadiazole Derivatives

### 5-Adamantan-1-yl-3-p-tolyl-[1,2,4]oxadiazole (XDS03180, STX1599)

To a solution of adamantan-1-yl-carbonyl chloride (99 mg, 0.50 mmol) in toluene (5 mL) was added pyridine (1 mL), followed by 4-methylbenzamide oxime (75 mg, 0.5 mmol). The reaction mixture was stirred at ambient temperature under nitrogen for 3 h, at 105° C. for 6 h and then partitioned between ethyl acetate and brine. The organic phase was washed with brine, dried over sodium sulphate and evaporated to give a yellow solid that was purified by flash chromatography (Ethyl acetate-hexane gradient elution) to give the title compound as crystalline solid (98 mg, 67%). TLC single spot at R_{f}: 0.77 (30% EtOAc/hexane); ¹H NMR (270 MHz, CDCl₃) δ 1.80 (6H, broad, 3×CH₂), 2.13 (9H, s, 3×CH₂ and 3×CH), 2.39 (3H, s, CH₃), 7.25 (2H, dt, J=8.1, 1.2 Hz, ArH) and 7.95 (2H, dt, J=8.2, 1.2 Hz, ArH); LC/MS (APCI) m/z 295 (M⁺+H); HPLC tᵣ=5.9 min (>98%) in 6% water-acetonitrile.

### 5-Adamantan-1-ylmethyl-3-p-tolyl-[1,2,4]oxadiazole (XDS03187, STX1620)

To a solution of adamantan-1-yl-acetic acid (87 mg, 0.50 mmol) in DMF (3 mL) was added diisopropylethylamine (0.22 mL, 1.25 mmol), followed by HBTU and 4-methylbenzamide oxime (75 mg, 0.5 mmol). The reaction mixture was irradiated with microwave at 192° C. for 3 minutes and evaporated in vacuo to give a residue which was partitioned between ethyl acetate and brine. The organic phase was washed with brine, dried over sodium sulphate and evaporated to give a yellow solid that was purified by flash chromatography (Ethyl acetate-hexane gradient elution) to give the title compound as yellow solid (59 mg, 38%). TLC single spot at R_{f}: 0.55 (20% EtOAc/hexane); ¹H NMR (270 MHz, CDCl₃) δ 1.52-1.67 (12H, m, 6×CH₂), 1.93 (3H, broad, 3×CH), 2.33 (3H, s, CH₃), 2.65 (2H, s, CH₂), 7.25 (2H, d, J=8.8 Hz, ArH) and 7.90 (2H, d, J=8.8 Hz, ArH); LC/MS (APCI) m/z 309 (M⁺+H); HPLC tᵣ=5.24 min (>96%) in 10% water-acetonitrile.

### 5-Adamantan-1-yl-3-(4-chloro-phenyl)-[1,2,4]oxadiazole (XDS03191, STX1627)

To a solution of adamantan-1-yl-carbonyl chloride (99 mg, 0.50 mmol) in toluene (5 mL) was added pyridine (1 mL), followed by 4-chlorobenzamide oxime (85 mg, 0.5 mmol). The reaction mixture was stirred at ambient temperature under nitrogen for 3 h, at 105° C. for 6 h and then partitioned between ethyl acetate and brine. The organic phase was washed with brine, dried over sodium sulphate and evaporated to give a yellow solid that was purified by flash chromatography (Ethyl acetate-hexane gradient elution) to give the title compound as off-white solid (59 mg, 38%). TLC single spot at R_{f}: 0.67 (20% EtOAc/hexane); ¹H NMR (300 MHz, CDCl₃) δ 1.85 (6H, broad, 3×CH₂), 2.10 (9H, s, 3×CH₂ and 3×CH), 7.38 (2H, dt, J=8.3, 2.0 Hz, ArH) and 7.96 (2H, dt, J=8.2, 2.0 Hz, ArH); LC/MS (APCI) m/z 315 (M⁺+H); HPLC tᵣ=11.7 min (>99%) in 10% water-acetonitrile.

### 5-Adamantan-1-ylmethyl-3-(4-chloro-phenyl)-[1,2,4]oxadiazole (XDS04001P, STX1659)

To a solution of adamantan-1-yl-acetic acid (97 mg, 0.50 mmol) in DMF (3 mL) was added diisopropylethylamine (0.22 mL, 1.25 mmol), followed by HBTU (190 mg, 0.50 mmol) and 4-chlorobenzamide oxime (85 mg, 0.5 mmol). The reaction mixture was stirred at ambient temperature for 30 min, then irradiated with microwave at 195° C. for 10 minutes and evaporated in vacuo to give a residue which was partitioned between ethyl acetate and brine. The organic phase was washed with brine, dried over sodium sulphate and evaporated to give a yellow solid that was purified by flash chromatography (Ethyl acetate-hexane gradient elution) to give the title compound as white solid (41 mg, 25%). TLC single spot at R_{f}: 0.69 (30% EtOAc/hexane); ¹H NMR (300 MHz, CDCl₃) δ 1.52-1.68 (12H, m, 6×CH₂), 1.93 (3H, broad, 3×CH), 2.65 (2H, s, CH₂), 7.38 (2H, dt, J=8.5, 2.3 Hz, ArH) and 7.96 (2H, dt, J=8.6, 2.3 Hz, ArH); LC/MS (APCI) m/z 329 (M⁺+H).

### 5-Adamantan-1-yl-3-(4-methyl-benzyl)-[1,2,4]oxadiazole (XDS04002, STX1660)

To a solution of adamantan-1-yl-carbonyl chloride (99 mg, 0.50 mmol) in toluene (3 mL) was added pyridine (1 mL), followed by N-hydroxy-2-p-tolyl-acetamidine (82 mg, 0.5 mmol). The reaction mixture was stirred at ambient temperature under nitrogen for 2 h, then at 110° C. for 5 h and partitioned between ethyl acetate and brine. The organic phase was washed with brine, dried over sodium sulphate and evaporated to give a yellow solid that was purified by flash chromatography Ethyl acetate-hexane gradient elution) to give the title compound as off-white solid (60 mg, 39%). TLC single spot at R_{f}: 0.75 (20% EtOAc/hexane); ¹H NMR (300 MHz, CDCl₃) δ 1.65-1.75 (6H, m, 3×CH₂), 1.98 (6H, broad, 3×CH₂), 2.02 (3H, s, 3×CH), 2.25 (3H, s, CH₃), 3.92 (2H, s, CH₂), 7.05 (2H, d, J=8.0 Hz, ArH) and 7.15 (2H, d, J=8.0 Hz, ArH); LC/MS (APCI) m/z 309 (M⁺+H); HPLC tᵣ=5.4 min (>99%) in 4% water-acetonitrile.

### 5-Adamantan-1-ylmethyl-3-(4-methyl-benzyl)-[1,2,4]oxadiazole (XDS04008, STX1664)

To a solution of adamantan-1-yl-acetic acid (97 mg, 0.50 mmol) in DMF (5 mL) was added diisopropylethylamine (0.43 mL, 2.50 mmol), followed by TBTU (161 mg, 0.50 mmol), HOBt (14 mg, 0.10 mmol) and N-hydroxy-2-p-tolyl-acetamidine (82 mg, 0.5 mmol). The reaction mixture was stirred at ambient temperature for 2 h, then at 110° C. for 3 h and evaporated in vacuo to give a residue which was partitioned between ethyl acetate and brine. The organic phase was washed with brine, dried over sodium sulphate and evaporated to give a yellow solid that was purified by flash chromatography (Ethyl acetate-hexane gradient elution) to give the title compound as white solid (30 mg, 19%). TLC single spot at R_{f}: 0.71 (20% EtOAc/hexane); ¹H NMR (300 MHz, CDCl₃) δ 1.51 (6H, d, J=2.7 Hz, 3×CH₂), 1.52-1.65 (6H, m, 3×CH₂), 1.91 (3H, broad, 3×CH), 2.26 (3H, s, CH₃), 2.56 (2H, s, CH₂), 3.95 (2H, s, CH₂), 7.07 (2H, d, J=8.2 Hz, ArH) and 7.15 (2H, d, J=8.2 Hz, ArH); LC/MS (APCI) m/z 323 (M⁺+H); HPLC tᵣ=5.24 min (>99%) in 10% water-acetonitrile.

### 5-Adamantan-1-yl-3-thiophen-2-ylmethyl-[1,2,4]oxadiazole (XDS04011, STX1687)

To a solution of adamantan-1-yl-carbonyl chloride (198 mg, 1.0 mmol) in toluene (8 mL) was added pyridine (2 mL), followed by N-hydroxy-2-thiophen-2-yl-acetamidine (XDS04007, 157 mg, 1.0 mmol). The reaction mixture was stirred at ambient temperature under nitrogen for 8 h, then at 110° C. for 3 h and partitioned between ethyl acetate and brine after cooling to room temperature. The organic phase was washed with brine, dried over sodium sulphate and evaporated to give a brown residue that was purified by flash chromatography (Ethyl acetate-hexane gradient elution) to give the title compound as off-white solid (49 mg, 16%). TLC single spot at R_{f}: 0.39 (20% EtOAc/hexane); ¹H NMR (300 MHz, CDCl₃) δ 1.68-1.76 (6H, m, 3×CH₂), 2.01 (9H, broad, 3×CH and 3×CH₂), 4.20 (2H, s, CH₂), 6.86-6.92 (2H, m, ArH) and 7.13 (2H, dd, J=4.9, 1.5 Hz, ArH); LC/MS (APCI) m/z 301+H); HPLC tᵣ=4.2 min (>99%) in 10% water-acetonitrile.

### Synthesis of 2-Thiophenylmethyl Amide Derivatives

### Cyclohexanecarboxylic acid (thiophen-2-ylmethyl)-amide (CCM01117, STX1638)

To a stirred solution of cyclohexylcarbonyl chloride (134 μL, 1.0 mmol) in DCM (1 mL) is added 2-thiophenemethylamine (114 μL, 1.1 mmol) at room temperature. After completion, silica is added. After filtration and evaporation under reduce pressure, the crude product is then crystallised in Ethyl acetate and hexane. The resulting white powder is then washed by a saturated solution of sodium bicarbonate and by water to give cyclohexanecarboxylic acid (thiophen-2-ylmethyl)-amide as a white powder. M.p.: 105° C. ¹H NMR (300 MHz, CDCl₃) δ_{H} 1.18-1.34 (3H, m, CH₂), 1.40-1.53 (2H, m, CH₂), 1.62-1.69 (1H, m, CH₂), 1.78-1.82 (m, 2H, CH₂), 1.86-1.91 (2H, m, CH₂), 2.10 (1H, dddd, J=3.3, 3.3, 11.5, 11.5 Hz, CHCO), 4.62 (2H, d, J=5.6 Hz, CH₂NH), 5.72 (bs, 1H, NH), 6.92-6.96 (m, 2H, H_{Ar}thiophene), 7.23 (dd, 1H, J=2.0, 4.3. Hz, H_{Ar}thiophene). LC/MS (AP−) m/z 220.2 (M−H). HPLC t_{R}=2.2 min (99.1%).

### 3,3-Dimethyl-N-thiophen-2-ylmethyl-butyramide (CCM01121, STX1639)

To a solution of 2-thiophenemethylamine (104 μL, 1.0 mmol) in DCM (5 mL) at room temperature is added tertbutylacetyl chloride (210 mL, 1.5 mmol). The reaction is stirred for 1 h and PS-trisamine (250 mg) is added. After 16 h, the reaction mixture is filtered and evaporated under reduce pressure. The crude product is purified by flash chromatography (EtOAc/DCM 1/9 to 3/7) to give 3,3-dimethyl-N-thiophen-2-ylmethyl-butyramide (80 mg, 0.38 mmol, 38% yield) as a white powder. M.p.: 83-84° C. (Hexane). ¹H NMR (300 MHz, CDCl₃) δ_{H} 1.05 (9H, s, tBu), 2.08 (2H, s, CH₂CO), 4.62 (2H, d, J=5.6 Hz, CH₂NH), 5.65 (bs, 1H, NH), 6.94-6.99 (m, 2H, H_{Ar}thiophene), 7.23 (dd, 1H, J=1.4, 4.8. Hz, H_{Ar}thiophene). LC/MS (AP−) m/z 210.2 (M−H). HPLC t_{R}=2.4 min (99.1%).

### General Procedures for the Synthesis of Amide:

To a solution of the acid (n mmol) in DCM (10n mL) are added EDCI (1.2n mmol), DMAP (1.2n mmol) and TEA (1.2n mmol) at room temperature. After 30 minutes, the amine (1.2n mmol) is added to the reaction mixture. After completion, the organic layer is washed with a solution of ammonium chloride and a solution of sodium bicarbonate, dried (MgSO₄), filtered and evaporated under reduce pressure. The crude product is purified to give the amide.

### 2,2-Diphenyl-N-thiophen-2-ylmethyl-propionamide (CCM01122, STX1640)

Reaction of 2,2-diphenylpropionic acid (226 mg, 1.0 mmol) in DCM (10 mL) with 2-thiophenemethylamine (124 μL, 1.2 mmol) in presence of triethylamine (170 μL, 1.2 mmol), EDCI (230 mg, 1.2 mmol) and DMAP (147 mg, 1.2 mmol) according to the general procedure gave 2,2-diphenyl-N-thiophen-2-ylmethyl-propionamide (125 mg, 0.39 mmol, 39% yield) as a white powder after purification by flash chromatography on silica gel (DCM). M.p.: 90-91° C. ¹H NMR (300 MHz, CDCl₃) δ_{H} 2.03 (3H, s, CH₃), 4.63 (2H, dd, J=0.6, 5.8 Hz, CH₂NH), 5.81 (bs, 1H, NH), 6.85-6.87 (m, 1H, H_{Ar}thiophene), 6.91 (dd, 1H, J=3.5, 5.0. Hz, H_{Ar}thiophene), 7.20 (dd, 1H, J=1.2, 4.0 Hz, H_{Ar}thiophene), 7.22-7.35 (10H, m, H_{Ar}). LC/MS (AP+) m/z 322.5 (M⁺+H). HPLC t_{R}=2.6 min (99.2%).

### 1-Methyl-cyclopropanecarboxylic acid (thiophen-2-ylmethyl)-amide (CCM01123, STX1641)

Reaction of 1-methylcyclopropanecarboxylic acid (100 mg, 1.0 mmol) in DCM (10 mL) with 2-thiophenemethylamine (124 mL, 1.2 mmol) in presence of triethylamine (170 μL, 1.2 mmol), EDCI (230 mg, 1.2 mmol) and DMAP (147 mg, 1.2 mmol) according to the general procedure gave 1-methyl-cyclopropanecarboxylic acid (thiophen-2-ylmethyl)-amide (125 mg, 0.39 mmol, 39% yield) as a white powder after purification by flash chromatography on silica gel (DCM). M.p.: 70-71° C. ¹H NMR (300 MHz, CDCl₃) δ_{H} 0.52 (2H, dd, J=3.8, 6.6 Hz, CH₂), 1.17 (2H, dd, J=3.8, 6.6 Hz, CH₂), 1.25 (3H, s, CH₃), 4.56 (2H, dd, J=0.5, 5.5 Hz, CH₂NH, 5.96 (bs, 1H, NH), 6.88-6.92 (m, 2H, H_{Ar}thiophene), 7.16 (dd, 1H, J=1.5, 4.8 Hz, H_{Ar}thiophene). LC/MS (AP−) m/z 194.2 (M−H). HPLC t_{R}=2.1 min (99.9%).

### 1-Methyl-cyclohexanecarboxylic acid (thiophen-2-ylmethyl)-amide (CCM01124, STX1642)

Reaction of 1-methyl-1-cyclohexanecarboxylic acid (153 mg, 1.1 mmol) in DCM (10 mL) with 2-thiophenemethylamine (124 μL, 1.2 mmol) in presence of triethylamine (170 μL, 1.2 mmol), EDCI (230 mg, 1.2 mmol) and DMAP (147 mg, 1.2 mmol) according to the general procedure gave 1-methyl-cyclohexanecarboxylic acid (thiophen-2-ylmethyl)-amide (195 mg, 0.82 mmol, 82% yield) as a white powder after purification by flash chromatography on silica gel (DCM). M.p.: 72-73° C. ¹H NMR (300 MHz, CDCl₃) δ_{H} 1.17 (3H, s, CH₃), 1.31-1.40 (4H, m, CH₂cyclohexane), 1.44-1.53 (4H, m, CH₂cyclohexane), 1.89-1.95 (2H, m, CH₂cyclohexane), 4.65 (2H, d, J=5.5 Hz, CH₂NH), 5.95 (bs, 1H, NH), 6.94-6.97 (m, 2H, H_{Ar}thiophene), 7.23 (dd, 1H, J=2.0, 4.3 Hz, H_{Ar}thiophene). LC/MS (AP−) m/z 236.3 (M−H). HPLC t_{R}=2.5 min (98.9%)

### Hexahydro-2,5-methano-pentalene-3a-carboxylic acid (thiophen-2-ylmethyl)amide (CCM01126, STX1648)

Reaction of 3-noradamantanecarboxylic acid (166 mg, 1.0 mmol) in DCM (10 mL) with 2-thiophenemethylamine (124 μL, 1.2 mmol) in presence of triethylamine (170 mL, 1.2 mmol), EDCI (230 mg, 1.2 mmol) and DMAP (147 mg, 1.2 mmol) according to the general procedure gave hexahydro-2,5-methano-pentalene-3a-carboxylic acid (thiophen-2-ylmethyl)-amide (216 mg, 0.83 mmol, 83% yield) as a white powder after purification by flash chromatography on silica gel (DCM). M.p.: 143-145° C. ¹H NMR (300 MHz, CDCl₃) δ_{H} 1.59-1.68 (4H, m, CH₂adamantane), 1.77-1.86 (4H, m, CH₂adamantane), 1.99-2.03 (2H, m, Hadamantane), 2.33 (2H, bs, Hadamantane), 2.70 (1H, t, J=6.8 Hz, Hadamantane), 4.65-4.67 (2H, m, CH₂NH), 5.84 (bs, 1H, NH), 6.95-6.97 (m, 2H, H_{Ar}thiophene), 7.23 (dd, 1H, J=1.9, 4.5 Hz, H_{Ar}thiophene). LC/MS (AP−) m/z 262.5 (M−H). HPLC t_{R}=2.6 min (97.7%).

### 1-(4-Chloro-phenyl)-cyclopentanecarboxylic acid (thiophen-2-ylmethyl)-amide (STX1649, CCM01128)

To a solution of 2-thiophenemethylamine (104 μL, 1.0 mmol) in DCM (10 mL) at room temperature is added 1-(4-chlorophenyl)-1-cyclopentanecarbonyl chloride (361 mg, 1.5 mmol). The reaction is stirred overnight and PS-trisamine (275 mg) is added. After 1 h 30, the reaction mixture is filtered and evaporated under reduce pressure. The crude product is purified by flash chromatography (EtOAc/DCM 1/9 to 3/7) to give 1-(4-chloro-phenyl)-cyclopentanecarboxylic acid (thiophen-2-ylmethyl)-amide (114 mg, 0.36 mmol, 36% yield) as a white powder. M.p.: 119-120° C. (Hexane). ¹H NMR (300 MHz, CDCl₃) δ_{H} 1.62-1.75 (2H, m, CH₂cyclohexane), 1.81-1.88 (2H, m, CH₂cyclohexane), 1.94-2.02 (2H, m, CH₂cyclohexane), 2.44-2.55 (2H, m, CH₂cyclohexane), 4.53 (2H, dd, J=0.6, 6.1 Hz, CH₂NH), 5.50 (1H, bs, NH), 6.81-6.82 (1H, m, H_{Ar}thiophene), 6.90 (1H, dd, J=3.5, 5.4 Hz, H_{Ar}thiophene), 7.23 (1H, dd, J=1.4, 4.8. Hz, H_{Ar}thiophene). LC/MS (AP−) m/z 318.3 (M−H). HPLC t_{R}=3.0 min (99.9%).

### 3,5-Dimethyl-adamantane-1-carboxylic acid (thiophen-2-ylmethyl)-amide (CCM01139, STX1671)

Reaction of 3,5-dimethyladamantanecarboxylic acid (80 mg, 0.38 mmol) in DCM (4 mL) with 2-thiophenemethylamine (46 μL, 0.45 mmol) in presence of triethylamine (63 μL, 0.45 mmol), EDCI (87 mg, 0.45 mmol) and DMAP (55 mg, 0.45 mmol) according to the general procedure gave 3,5-dimethyl-adamantane-1-carboxylic acid (thiophen-2-ylmethyl)-amide (67 mg, 0.22 mmol, 58% yield) as a white powder after purification by flash chromatography on silica gel (EtOAc/DCM 0/10 to 1/9). M.p.: 101-103° C.; ¹H NMR (270 MHz, CDCl₃) δ_{H} 0.83 (6H, s, 2*CH₃), 1.14-1.15 (2H, m, CH₂ adamantane), 1.34 (4H, bs, CH₂ adamantane), 1.47 (4H, bd, J=5.7 Hz, CH₂ adamantane), 1.68 (2H, bd, J=3.2 Hz, CH₂ adamantane), 2.10-2.14 (1H, m, CH adamantane), 4.59 (2H, d, J=5.5 Hz, CH₂—NH), 5.87 (1H, bs, NH), 6.94 (2H, d, J=3.5 Hz, H_{Ar}thiophene), 7.20-7.22 (1H, m, H_{Ar}thiophene); LC/MS (AP+) m/z 304.4 (M+H); HPLC t_{R}=3.8 min (99.9%).

### 2,2-Dimethyl-N-thiophen-2-ylmethyl-propionamide (CCM01145, STX1673)

To a solution of 2-thiophenemethylamine (104 μL, 1.0 mmol) in DCM (10 mL) at room temperature is added trimethylacetyl chloride (184 μL, 1.5 mmol) and TEA (120 μL, 1.2 mmol). The reaction is stirred overnight and PS-trisamine (125 mg) is added. After 3 h, the reaction mixture is filtered and evaporated under reduce pressure. The crude product is purified by flash chromatography (EtOAc/DCM 0/10 to 1/9) to give 2,2-dimethyl-N-thiophen-2-ylmethyl-propionamide (138 mg, 0.70 mmol, 70% yield) as a white powder. M.p.: 64-65° C.; ¹H NMR (270 MHz, CDCl₃) δ_{H} 1.20 (9H, s, tBu), 4.59 (2H, d, J=5.7 Hz, CH₂NH), 5.94 (1H, bs, NH), 6.92-6.95 (2H, m, H_{Ar}thiophene), 7.20-7.22 (1H, m, H_{Ar}thiophene); LC/MS (AP+) m/z 198.3 (M+H); HPLC t_{R}=2.6 min (99.9%).

### 3-Hydroxy-adamantane-1-carboxylic acid (thiophen-2-ylmethyl)-amide (CCM01149, STX1675)

Reaction of 3-hydroxyadamantane-1-carboxylic acid (196 mg, 1.0 mmol) in DCM (10 mL) with 2-thiophenemethylamine (124 μL, 1.2 mmol) in presence of triethylamine (170 μL, 1.2 mmol), EDCI (230 mg, 1.2 mmol) and DMAP (147 mg, 1.2 mmol) according to the general procedure gave 3-hydroxy-adamantane-1-carboxylic acid (thiophen-2-ylmethyl)-amide (165 mg, 0.56 mmol, 56% yield) as a white powder after purification by flash chromatography on silica gel (MeOH/DCM 0/10 to 1.5/8.5). M.p.: 140-141° C.; ¹H NMR (270 MHz, CDCl₃) δ_{H} 1.57-1.59 (2H, m, CH/CH₂ adamantane), 1.70 (4H, bs, CH/CH₂ adamantane), 1.76 (4H, bd, J=2.5 Hz, CH/CH₂ adamantane), 1.80 (2H, bs, CH/CH₂ adamantane), 2.26-2.30 (2H, m, CH/CH₂ adamantane), 4.59 (2H, d, J=5.7 Hz, CH₂NH), 5.89 (1H, bs, NH), 6.94 (2H, d, J=3.9 Hz, H_{Ar}thiophene), 7.20-7.22 (1H, m, H_{Ar}thiophene); LC/MS (AP+) m/z 292.4 (M+H); HPLC t_{R}=2.1 min (99.9%).

### Cyclohexanecarboxylic acid methyl-thiophen-2-ylmethyl-amide (CCM01151, STX1693)

To a stirred solution of methyl-thiophen-2-ylmethyl-amine (127 mg, 1.0 mmol) in DCM (10 mL) is added cyclohexylcarbonyl chloride (200 μL, 1.5 mmol) and TEA (170 μL; 1.2 mmol) at room temperature. After overnight, PS-trisamine (125 mg) is added. After filtration and evaporation under reduce pressure, the crude product is purified by flash chromatography on silica gel (EtOAc/DCM 0/10 to 0.5/9.5) to give cyclohexanecarboxylic acid methyl-thiophen-2-ylmethyl-amide (125 mg, 0.53 mmol, 53%) as a colourless oil. ¹H NMR (270 MHz, CDCl₃) δ_{H} 1.15-1.24 (3H, m, CH₂ cyclohexane), 1.46-1.56 (2H, m, CH₂ cyclohexane), 1.59-1.80 (4H, m, CH₂ cyclohexane), 2.47 and 2.58 (1H, 2*dddd, J=3.4, 3.4, 11.6, 11.6 Hz, CHCO, rotamers), 2.93 and 2.99 (3H, 2*s, CH3, rotamers), 4.67 and 4.69 (2H, 2 s, CH₂NH, rotamers), 6.90-6.98 (2H, m, H_{Ar}thiophene), 7.19-7.24 (1H, m, H_{Ar}thiophene); LC/MS (AP+) m/z 238.3 (M+H); HPLC t_{R}=2.5 min (99.9%).

### 3,3,N-Trimethyl-N-thiophen-2-ylmethyl-butyramide (CCM01152, STX1694)

To a solution of methyl-thiophen-2-ylmethyl-amine (127 mg, 1.0 mmol) in DCM (10 mL) at room temperature is added tertbutylacetyl chloride (210 μL, 1.5 mmol) and TEA (170 μL, 1.2 mmol). The reaction is stirred overnight and PS-trisamine (125 mg) is added. After 2 h, the reaction mixture is filtered and evaporated under reduce pressure. The crude product is purified by flash chromatography (EtOAc/DCM 0/10 to 0.5/9.5) to give 3,3,1-trimethyl-N-thiophen-2-ylmethyl-butyramide (175 mg, 0.78 mmol, 78% yield) as a colourless oil. ¹H NMR (270 MHz, CDCl₃) δ_{H} 1.05 and 1.06 (9H, 2*s, tBu, rotamers), 2.26 and 2.34 (2H, 2*s, CH₂CO, rotamers), 2.94 and 2.99 (3H, 2*s, N—CH₃, rotamers), 4.67 and 4.71 (2H, 2*s, CH₂NH, rotamers), 6.88-6.98 (2H, m, H_{Ar}thiophene), 7.20-7.24 (1H, m, H_{Ar}thiophene); LC/MS (AP+) m/z 226.3 (M+H); HPLC t_{R}=2.4 min (99.9%).

### 2,2,N-Trimethyl-N-thiophen-2-ylmethyl-propionamide (CCM01153, STX1695)

To a solution of methyl-thiophen-2-ylmethyl-amine (127 mg, 1.0 mmol) in DCM (10 mL) at room temperature is added trimethylacetyl chloride (184 μL, 1.5 mmol) and TEA (170 μL, 1.2 mmol). The reaction is stirred overnight and PS-trisamine (125 mg) is added. After 3 h, the reaction mixture is filtered and evaporated under reduce pressure. The crude product is purified by flash chromatography (EtOAc/DCM 0/10 to 1/9) to give 2,2,N-trimethyl-N-thiophen-2-ylmethyl-propionamide (170 mg, 0.80 mmol, 80% yield) as a colourless oil. ¹H NMR (270 MHz, CDCl₃) δ_{H} 1.30 (9H, s, tBu), 3.04 (3H, s, N—CH₃), 4.71 (2H, s, CH₂NH), 6.93 (2H, d, J=3.5 Hz, H_{Ar}thiophene), 7.20-7.22 (1H, m, H_{Ar}thiophene); LC/MS (AP+) m/z 212.3 (M+H); HPLC t_{R}=2.4 min (99.9%).

### 1-(4-Chloro-phenyl)-cyclopentanecarboxylic acid methyl-thiophen-2-ylmethyl-amide (CCM01154, STX1696)

To a solution of methyl-thiophen-2-ylmethyl-amine (127 mg, 1.0 mmol) in DCM (10 mL) at room temperature is added 1-(4-chlorophenyl)-1-cyclopentanecarbonyl chloride (361 mg, 1.5 mmol) and TEA (170 μL, 1.2 mmol). The reaction is stirred overnight and PS-PS-PS-trisamine (125 mg) is added. After 1.5 h the reaction mixture is filtered and evaporated under reduce pressure. The crude product is purified by flash chromatography EtOAc/DCM 0/10 to 1/9) to give 1-(4-chloro-phenyl)-cyclopentanecarboxylic acid methyl-thiophen-2-ylmethyl-amide (305 mg, 0.91 mmol, 91% yield) as a colourless oil. ¹H NMR (270 MHz, CDCl₃) δ_{H} 1.62-1.82 (4H, m, CH₂ cyclopentane), 1.93-2.10 (3H, m, CH₂ cyclopentane), 2.43-2.52 (1H, m, CH₂ cyclopentane), 2.53 (311, bs, N—CH₃), 4.69 (2H, bs, CH₂N), 6.92 (2H, d, J=3.0 Hz, H_{Ar}thiophene), 7.12-7.24 (5H, m, H_{Ar}+H_{Ar}thiophene); LC/MS (AP+) m/z 334.2 (M+H); HPLC t_{R}=3.1 min (99.9%)

### 1-Methyl-cyclopropanecarboxylic acid methyl-thiophen-2-ylmethyl-amide (CCM01155, STX1697)

Reaction of 1-methylcyclopropanecarboxylic acid (100 mg, 1.0 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gave 1-methyl-cyclopropanecarboxylic acid methyl-thiophen-2-ylmethyl-amide (195 mg, 0.93 mmol, 93% yield) as a colourless oil after purification by flash chromatography on silica gel (EtOAc/DCM 0/10 to 1/9). ¹H NMR (270 MHz, CDCl₃) δ_{H} 0.59 (2H, dd, J=4.4, 6.2 Hz, 2×CH cyclopropane), 0.98 (2H, dd, J=4.4, 6.2 Hz, 2*CH cyclopropane), 1.32 (3H, s, CH₃), 3.03 (3H, s, N—CH₃), 4.75 (2H, s, CH₂—NH), 6.92-6.95 (2H, m, H_{Ar}thiophene), 7.22 (1H, dd, J=1.7, 4.4 Hz, H_{Ar}thiophene); LC/MS (AP+) m/z 210.1 (M−H); HPLC t_{R}=2.2 min (99.9%).

### N-Methyl-2,2-diphenyl-N-thiophen-2-ylmethyl-propionamide (CCM01156, STX1698)

Reaction of 2,2-diphenylpropionic acid (226 mg, 1.0 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gave N-Methyl-2,2-diphenyl-N-thiophen-2-ylmethyl-propionamide (180 mg, 0.54 mmol, 54% yield) as a colourless oil after purification by flash chromatography on silica gel (EtOAc/DCM 0/10 to 1/9). ¹H NMR (270 MHz, CDCl₃) δ_{H} 1.93 (3H, s, C(Ph)₂-CH₃), 2.37 (3H, bs, N—CH₃), 4.76 (2H, bs, CH₂—NH), 6.95 (2H, bs, H_{Ar}thiophene), 7.22-7.37 (11H, m, H_{Ar}thiophene+H_{Ar}); LC/MS (AP+) m/z 336.3 (M+H); HPLC t_{R}=2.6 min (99.9%).

### 1-Methyl-cyclohexanecarboxylic acid methyl-thiophen-2-ylmethyl-amide (CCM01157, STX1699)

Reaction of 1-methyl-1-cyclohexanecarboxylic acid (153 mg, 1.1 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gave 1-methyl-cyclohexanecarboxylic acid methyl-thiophen-2-ylmethyl-amide (210 mg, 0.83 mmol, 83% yield) as a colourless oil after purification by flash chromatography on silica gel (DCM). ¹H NMR (270 MHz, CDCl₃) δ_{H} 1.25 (3H, s, CH₃), 1.31-1.41 (3H, m, CH₂ cyclohexane), 1.47-1.58 (5H, m, CH₂ cyclohexane), 2.10-2.16 (2H, m, CH₂ cyclohexane), 3.06 (3H, s, N—CH₃), 4.73 (2H, s, CH₂—NH), 6.94-6.96 (2H, m, H_{Ar}thiophene), 7.22-7.24 (1H, m, H_{Ar}thiophene); LC/MS (AP+) m/z 252.4 (M+H); HPLC t_{R}=2.7 min (99.2%).

### Hexahydro-2,5-methano-pentalene-3a-carboxylic acid methyl-thiophen-2-ylmethyl-amide (CCM01159, STX1700)

Reaction of 3-noradamantanecarboxylic acid (166 mg, 1.0 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gave hexahydro-2,5-methano-pentalene-3a-carboxylic acid methyl-thiophen-2-ylmethyl-amide (230 mg, 0.83 mmol, 83% yield) as a colourless oil after purification by flash chromatography on silica gel (EtOAc/DCM 0/10 to 0.5/9.5). ¹H NMR (270 MHz, CDCl₃) δ_{H} 1.59-1.68 (4H, m, CH/CH₂ noradamantane), 1.87-1.95 (4H, m, CH/CH₂ noradamantane), 2.10-2.15 (2H, m, CH/CH₂ noradamantane), 2.32 (2H, bs, CH/CH₂ noradamantane), 2.91 (1H, bt, J=6.6 Hz, CH-Cq-CO), 3.02 (3H, bs, N—CH₃), 4.74 (2H, s, CH₂—NH), 6.95 (2H, m, J=3.4 Hz, H_{Ar}thiophene), 7.21-7.25 (1H, m, H_{Ar}thiophene); LC/MS (AP+) m/z 276.4 (M+H).

### Synthesis of 2-Thiophenylethyl Amide Derivatives

### 2,2-Diphenyl-N-(2-thiophen-2-yl-ethyl)propionamide (CCM01129, STX1650)

Reaction of 2,2-diphenylpropionic acid (226 mg, 1.0 mmol) in DCM (10 mL) with 2-thiophenethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gave 2,2-diphenyl-N-(2-thiophen-2-yl-ethyl)-propionamide (137 mg, 0.40 mmol, 40% yield) as a colourless oil after purification by flash chromatography on silica gel (DCM/EtOAc 0110 to 19). ¹H NMR (300 MHz, CDCl₃) δ_{H} 1.91 (3H, s, CH₃), 2.91 (2H, t, J=6.4 Hz, CH₂-thiophene), 3.46 (2H, q, J=6.4 Hz, CH₂NH), 5.50 (1H, bs, NH), 6.52 (1H, dd, J=1.0, 3.4 Hz, H_{Ar}thiophene), 6.78 (1H, dd, J=3.4, 5.1 Hz, H_{Ar}thiophene), 7.02 (1H, dd, J=1.0, 5.1 Hz, H_{Ar}thiophene), 7.07-7.09 (2H, m, H_{Ar}), 7.09-7.11 (2H, m, H_{Ar}), 7.17-7.20 (4H, m, H_{Ar}) 7.21-7.25 (2H, m, H_{Ar}). LC/MS (AP+) m/z 336.4 (M+H). HPLC t_{R}=2.9 min (99.9%).

### 1-(4-Chloro-phenyl)-cyclopentanecarboxylic acid (2-thiophen-2-yl-ethyl)-amide (CCM01130, STX1651)

To a solution of 2-thiophenethylamine (116 μL, 1.0 mmol) in DCM (10 mL) at room temperature is added 1-(4-chlorophenyl)-1-cyclopentanecarbonyl chloride (361 mg, 1.5 mmol) and TEA (210 μL, 1.5 mmol). The reaction is stirred for 3 h and PS-trisamine (125 mg) is added. After 1 h 30, the reaction mixture is filtered and evaporated under reduce pressure. The crude product is purified by flash chromatography (EtOAc/DCM 1/9 to 3/7) to give 1-(4-chloro-phenyl)-cyclopentanecarboxylic acid (2-thiophen-2-yl-ethyl)-amide (142 mg, 0.43 mmol, 43% yield) as a white powder. LC/MS (AP+) m/z 334.4 (M+H). HPLC t_{R}=3.2 min (99.9%).

### 3,3-Dimethyl-N-(2-thiophen-2-yl-ethyl)-butyramide (CCM0131, STX1652)

To a solution of 2-thiophenethylamine (116 μL, 1.0 mmol) in DCM (10 mL) at room temperature is added tert-butylacetyl chloride (210 μL, 1.5 mmol) and TEA (210 μL, 1.5 mmol). The reaction is stirred for 3 h and PS-trisamine (125 mg) is added. After 1 h, the reaction mixture is filtered and evaporated under reduce pressure. The crude product is purified by flash chromatography (EtOAc/DCM 1/9 to 3/7) to give 3,3-dimethyl-N-(2-thiophen-2-yl-ethyl)-butylamide (180 mg, 0.80 mmol, 80% yield) as a yellow oil. ¹H NMR (300 MHz, CDCl₃) δ_{H} 0.94 (9H, s, tBu), 1.95 (2H, s, CH₂CO), 2.97 (2H, t, J=0.5 Hz, CH₂-thiophene), 3.47 (2H, pseudo q, J=6.5 Hz, CH₂—NH), 5.35 (1H, bs, NH), 6.76-6.77 (1H, m, H_{Ar}thiophene), 6.88 (1H, dd, J=3.4, 5.1 Hz, H_{Ar}thiophene), 7.09 (1H, dd, J=1.1, 5.1 Hz, H_{Ar}thiophene); LC/MS (AP−) m/z 224.0 (M−H); HPLC t_{R}=2.4 min.

### Cyclohexanecarboxylic acid (2-thiophen-2-yl-ethyl)-amide (CCM01132, STX1653)

To a stirred solution of 2-thiophenethylamine (116 μL, 1.0 mmol) in DCM (10 mL) is added cyclohexylcarbonyl chloride (200 μL, 1.5 mmol) and TEA (210 μL, 1.5 mmol) at room temperature. The reaction is stirred for 3 h and PS-trisamine (125 mg) is added. After 1 h, the reaction mixture is filtered and evaporated under reduce pressure. The crude product is purified by flash chromatography (EtOAc/DCM 1/9 to 3/7) to give cyclohexanecarboxylic acid (2-thiophen-2-yl-ethyl)-amide (180 mg, 0.76 mmol, 76% yield) as a white powder. M.p.: 94-95° C.; ¹H NMR (300 MHz, CDCl₃) δ_{H} 1.10-1.20 (3H, m, CH₂ cyclohexane), 1.24-1.39 (2H, m, CH₂ cyclohexane), 1.58-1.61 (1H, m, CH₂ cyclohexane), 1.69-1.78 (4H, m, CH₂ cyclohexane), 1.96 (1H, dddd, J=3.2, 3.2, 11.3, 11.3 Hz, CHCO), 2.96 (2H, t, J=6.4 Hz, CH₂-thiophene), 3.45 (2H, pseudo q, J=6.4 Hz, CH₂NH), 5.48 (1H, bs, NH), 6.75-6.76 (1H, m, H_{Ar}thiophene), 6.88 (1H, dd, J=3.4, 5.1 Hz, H_{Ar}thiophene), 7.10 (1H, dd, J=1.1, 5.1 Hz, H_{Ar}thiophene); LC/MS (AP+) m/z 238.3 (M+H); HPLC t_{R}=2.4 min (99.9%).

### 1-Methyl-cyclopropanecarboylic acid (2-thiophen-2-yl-ethyl)-amide (CCM01134, STX1654)

Reaction of 1-methylcyclopropanecarboxylic acid (100 mg, 1.0 mmol) in DCM (10 mL) with 2-thiophenethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gave 1-methyl-cyclopropanecarboxylic acid (2-thiophen-2-yl-ethyl)-amide (178 mg, 0.85 mmol, 85% yield) as a white powder after purification by flash chromatography on silica gel (EtOAc/DCM 0/10 to 1/9). M.p.: 86-88° C. LC/MS (AP+) m/z 210.2 (M−H). HPLC t_{R}=2.3 min (99.9%).

### 1-Methyl-cyclohexanecarboxylic acid (2-thiophen-2-yl-ethyl)-amide (CCM01135, STX1655)

Reaction of 1-methyl-1-cyclohexanecarboxylic acid (142 mg, 1.0 mmol) in DCM (10 mL) with 2-thiophenemethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gave 1-methyl-cyclohexanecarboxylic acid (2-thiophen-2-yl-ethyl)-amide (203 mg, 0.80 mmol, 80% yield) as a white powder after purification by flash chromatography on silica gel (EtOAc/DCM 0/10 to 1/9). M.p.: 79-80° C. LC/MS (AP−) m/z 236.3 (M−H). HPLC t_{R}=2.5 min (98.9%)

### Hexahydro-2,5-methano-pentalene-3a-carboxylic acid (2-thiophen-2-yl-ethyl)-amide (CCM01136, STX1656)

Reaction of 3-noradamantanecarboxylic acid (166 mg, 1.0 mmol) in DCM (10 mL) with 2-thiophenemethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gave hexahydro-2,5-methano-pentalene-3a-carboxylic acid (2-thiophen-2-yl-ethyl)-amide (240 mg, 0.87 mmol, 87% yield) as a white powder after purification by flash chromatography on silica gel (DCM). M.p.: 113-114° C. LC/MS (AP+) m/z 276.3 (M+H). HPLC t_{R}=2.7 min (97.5%).

### 3,5-Dimethyl-adamantane-1-carboxylic acid (2-thiophen-2-yl-ethyl)amide (CCM01140, STX1672)

Reaction of 3,5-dimethyladamantanecarboxylic acid (80 mg, 0.38 mmol) in DCM (4 mL) with 2-thiophenethylamine (54 mL, 0.45 mmol) in presence of triethylamine (63 μL, 0.45 mmol), EDCI (87 mg, 0.45 mmol) and DMAP (55 mg, 0.45 mmol) according to the general procedure gave 3,5-dimethyl-adamantane-1-carboxylic acid (2-thiophen-2-yl-ethyl)-amide (77 mg, 0.24 mmol, 63% yield) as a white powder after purification by flash chromatography on silica gel (EtOAc/DCM 0/10 to 1/9). M.p.: 131-132° C.; ¹H NMR (270 MHz, CDCl₃) δ_{H} 0.82 (6H, s, 2×CH₃), 1.14-1.15 (2H, m, CH₂ adamantane), 1.32 (4H, d, J=2.7 Hz, CH₂ adamantane), 1.42 (4H, s, CH₂ adamantane), 1.62 (2H, bd, J=3.2 Hz, CH₂ adamantane), 2.08-2.13 (1H, m, CH adamantane), 3.01 (2H, t, J=6.4 Hz, CH₂-thiophene), 3.48 (2H, pseudo q, J=6.4 Hz, CH₂—NH), 5.72 (1H, bs, NH), 6.80-6.82 (1H, m, H_{Ar}thiophene), 6.94 (1H, dd, J=3.2, 5.1 Hz, H_{Ar}thiophene), 7.16 (1H, dd, J=1.3, 5.1 Hz, H_{Ar}thiophene); LC/MS (AP+) m/z 318.4 (M+H); HPLC t_{R}=4.2 min (99.1%).

### 2,2-Dimethyl-N-(2-thiophen-2-yl-ethyl)-propionamide (CCM01146, STX1674)

To a solution of 2-thiophenethylamine (116 μL, 1.0 mmol) in DCM (10 mL) at room temperature is added trimethylacetyl chloride (184 μL, 1.5 mmol) and TEA (120 μL, 1.2 mmol). The reaction is stirred overnight and PS-trisamine (125 mg) is added. After 3 h, the reaction mixture is filtered and evaporated under reduce pressure. The crude product is purified by flash chromatography (EtOAc/DCM 0/10 to 1/9) to give 2,2-dimethyl-N-(2-thiophen-2-yl-ethyl)-propionamide (180 mg, 0.85 mmol, 85% yield) as a white solid. M.p.: 79-80° C.; ¹H NMR (270 MHz, CDCl₃) δ_{H} 1.15 (9H, s, tBu), 3.02 (2H, t, J=6.5 Hz, CH₂-thiophene), 3.49 (2H, pseudo q, J=6.5 Hz, CH₂—NH, 5.76 (1H, bs, NH), 6.80-6.82 (1H, m, H_{Ar}thiophene), 6.94 (1H, dd, J=3.4, 5.2 Hz, H_{Ar}thiophene), 7.15 (dd, 1H, J=1.2, 5.2 Hz, H_{Ar}thiophene); LC/MS (AP−) m/z 210.2 (M−H); HPLC t_{R}=2.7 min (98.5%).

### 3-Hydroxy-adamantane-1-carboxylic acid (2-thiophen-2-yl-ethyl)-amide (CCM01150, STX1676)

Reaction of 3-hydroxyadamantane-1-carboxylic acid (196 mg, 1.0 mmol) in DCM (10 mL) with 2-thiophenemethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gave 3-hydroxy-adamantane-1-carboxylic acid (2-thiophen-2-yl-ethyl)-amide (265 mg, 0.87 mmol, 87% yield) as a white powder after purification by flash chromatography on silica gel (DCM). M.p.: 118-119° C.; ¹H NMR (270 MHz, CDCl₃) δ_{H} 1.55-1.58 (2H, m, CH₂/CH adamantane), 1.69-1.71 (8H, m, CH₂/CH adamantane), 1.75 (2H, s, CH₂/CH adamantane), 2.21-2.24 (2H, m, CH₂/CH adamantane), 3.02 (2H, t, J=6.2 Hz, CH₂-thiophene), 3.50 (2H, pseudo q, J=6.2 Hz, CH₂—NH), 5.71 (1H, bs, NM, 6.79-6.82 (1H, m, H_{Ar}thiophene), 6.94 (1H, dd, J=3.5, 5.0 Hz, H_{Ar}thiophene), 7.16 (1H, dd, J=1.2, 5.0 Hz, H_{Ar}thiophene); LC/MS (AP+) m/z 306.3 (+H); HPLC t_{R}=2.2 min (99.9%).

### Synthesis of Other Compounds

### General Method for Synthesis of Amide Derivatives

Method 1: To a solution of the amine (1 eq) in DCM (10 mL) was added TEA (1.2 eq), followed by the acyl chloride (1.2 eq). The mixture was stirred for at ambient temperature until completion. PS-trisamine (0.5 eq) was added to the reaction mixture. After stirred at ambient temperature for another 2 h, the mixture was filtered and evaporation of the solvent gave a residue that was purified by flash chromatography to give the desired amide.

Method 2: To a solution of the acid (1 eq) in DCM (10 mL) were added EDCI (1.2 eq), DMAP (0.25 eq) and TEA (1.2 eq) at room temperature. After stirring for 30 minutes, the amine (1.2 eq) was added to the reaction mixture. The mixture was stirred at ambient temperature until completion, partitioned between DCM and a solution of sodium bicarbonate. The organic layer was washed with brine, dried (MgSO₄) and evaporated under reduce pressure. The crude product was purified by flash chromatography to give the amide.

### Bicyclo[2.2.1]hept-5-ene-2-carboxylic acid (thiophen-2-ylmethyl)-amide (exo) (STX1706, CCM01127)

Reaction of 5-norbornene-2-carboxylic acid (mixture of endo and exo) (122 μL, 1.0 mmol) in DCM (10 mL) with 2-thiophenemethylamine (124 μL, 1.2 mmol) in presence of triethylamine (170 μL, 1.2 mmol), EDCI (230 mg, 1.2 mmol) and DMAP (147 mg, 1.2 mmol) according to the general procedure gives bicyclo[2.2.1]hept-5-ene-2-carboxylic acid (thiophen-2-ylmethyl)-amide (exo) (150 mg, 0.64 mmol, 64% yield) as a white powder after purification by crystallisation in EtOAc/hexane. mp 123-124° C.; ¹H NMR (400 MHz, CDCl₃) δ_{H} 1.32 (1H, d, J=8.2 Hz, CO—CH—CH—CH₂), 1.40 (1H, ddd, J=2.7, 4.7, 12.1 Hz, CO—CH—CH₂), 1.47 (1H, ddd, J=2.0, 4.0, 8.2 Hz, CO—CH—CH—CH₂), 1.96 (1H, ddd, J=3.5, 9.4, 12.1 Hz, CO—CH—CH₂), 2.90-2.93 (1H, m, CO—CH), 2.94 (1H, bs, CH₂—CH—CH₂), 3.17 (1H, bs, CO—CH—CH), 4.54-4.65 (2H, m, CH₂—NH), 5.83 (1H, Bs, NH), 6.00 (1H, dd, J=2.7, 5.8 Hz, CO—CH—CH═CH), 6.26 (1H, dd, J=3.1, 5.8 Hz, CO—CH—CH═CH), 6.96-6.99 (2H, m, H_{Ar}thiophene), 7.24 (1H, dd, J=2.3, 4.3 Hz, H_{Ar}thiophene); ¹³C NMR (100 MHz, CDCl₃) δ_{C} 29.9 (CO—CH—CH—CH₂), 38.4 (CH₂), 42.8 (CH₂—CH—CH₂), 44.9 (CH—CO), 46.3 (CO—CH—CH), 50.1 (CO—CH—CH₂), 125.2 (CH), 125.9 (CH), 126.9 (CH), 132.4 (CO—CH—CH═CH), 138.0 (CO—CH—CH═CH), 141.5 (Cq_{Ar}), 174.0 (CO); LC/MS (AP+) m/z 234.4 (M−H); HPLC t_{R}=2.2 min (99.9%).

### Bicyclo[2.2.1]hept-5-ene-2-carboxylic acid (2-thiophen-2-yl-ethyl)-amide (exo) (STX1707, CCM01137A)

Reaction of 5-norbornene-2-carboxylic acid (122 μL, 1.0 mmol) in DCM (10 mL) with 2-thiophenemethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gives bicyclo[2.2.1]hept-5-ene-2-carboxylic acid (2-thiophen-2-yl-ethyl)-amide (exo) (170 mg, 0.68 mmol, 68% yield) as a white powder after purification by flash chromatography on silica gel (EtOAc/DCM 0/10 to 1/9). mp 71-72° C.; ¹H NMR (400 MHz, CDCl₃) δ_{H} 1.26 (1H, dd, J=2.0, 8.0 Hz, CH—CH—CH₂—CH), 1.29 (1H, dd, J=2.7, 4.2 Hz, CH—CH₂—CH), 1.41 (1H, ddd, J=2.0, 4.4, 8.0 Hz, CH—CH—CH₂—CH), 1.89 (1H, ddd, J=3.5, 9.4, 12.9 Hz, CH—CH₂—CH), 2.82 (1H, ddd, J=4.2, 4.2, 9.4 Hz, CH—CO), 2.88 (1H, bs, CO—CH—CH₂—CH), 2.99 (2H, t, J=6.6 Hz, CH₂-thiophene), 3.06 (1H, bs, CO—CH—CH), 3.40-3.52 (2H, m, CH₂—NH), 5.53 (1H, bs, NH), 5.86 (1H, dd, J=3.1, 5.8 Hz, CO—CH—CH—CH═CH) 6.17 (1H, dd, J=3.1, 5.5 Hz, CO—CH—CH—CH═CH) 6.80-6.82 (1H, m, H_{Ar}thiophene), 6.94 (1H, dd, J=3.5, 5.1 Hz, H_{Ar}thiophene), 7.15 (1H, dd, J=1.2, 5.1 Hz, H_{Ar}thiophene); ³C NMR (100 MHz, CDCl₃) δ_{C} 29.1 (CO—CH—CH₂—CH and CH₂-thiophene), 40.7 (CH₂—NH), 42.7 (CO—CH—CH₂—CH), 44.8 (CO—CH), 46.1 (CO—CH—CH), 50.0 (CO—CH—CH—CH₂), 123.9 (CH_{Ar} thiophene), 125.3 (CH_{Ar} thiophene), 127.0 (CH_{Ar} thiophene), 132.1 (CO—CH—CH—CH═CCH), 137.8 (CO—CH—CH—CH═CH) 141.5 (Cq_{Ar}), 174.3 (CO); LC/MS (AP−) m/z 246.0 (M−H); HPLC t_{R}=2.3 min (99.9%).

### Bicyclo[2.2.1]hept-5-ene-2-carboxylic acid (2-thiophen-2-yl-ethyl)-amide (endo) (STX1708, CCM01137B)

Reaction of 5-norbornene-2-carboxylic acid (122 μL, 1.0 mmol) in DCM (10 mL) with 2-thiophenemethylamine (140 μL, 1.2 mmol) in presence of triethylamine (170 μL, 1.2 mmol), EDCI (230 mg, 1.2 mmol) and DMAP (147 mg, 1.2 mmol) according to the general procedure gives bicyclo[2.2.1]hept-5-ene-2-carboxylic acid (2-thiophen-2-yl-ethyl)-amide (endo) (16 mg, 0.06 mmol, 6% yield) as a white powder after purification by flash chromatography on silica gel (EtOAc/DCM 0/10 to 1/9). mp 122-124° C.; H NMR (270 MHz, CDCl₃) δ_{H} 1.28-1.35 (2H, m, H norbornene), 1.66-1.70 (1H, m, H norbornene), 1.86-1.95 (2H, m, H norbornene), 2.88-2.92 (2H, m, H norbornene), 3.04 (2H, t, J=7.0 Hz, CH₂-thiophene), 3.53 (2H, aq, J=6.2 Hz, CH₂—NH), 5.56 (1H, bs, NH), 6.07-6.14 (2H, m, CH═CH), 6.82-6.84 (1H, m, H_{Ar} thiophene), 6.94 (1H, dd, J=3.2, 5.0 Hz, H_{Ar} thiophene), 7.16 (1H, dd, J=1.3, 5.0 Hz, H_{Ar} thiophene); LC/MS (AP−) m/z 246.0 (M−H); HPLC t_{R}=2.2 min (97.5%).

### Bicyclo[2.2.1]hept-5-ene-2-carboxylic acid methyl-thiophen-2-ylmethyl-amide (exo) (STX1709, CCM01158A)

Reaction of 5-norbornene-2-carboxylic acid (mixture of endo and exo) (145 mg, 1.0 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gives bicyclo[2.2.1]hept-5-ene-2-carboxylic acid methyl-thiophen-2-ylmethyl-amide (exo) (95 mg, 0.38 mmol, 38% yield) as a white solid after purification by flash chromatography on silica gel (EtOAc/DCM 0/10 to 0.5/9.5). mp 74-75° C.; ¹H NMR (400 MHz, CDCl₃) δ_{H} 1.28 (1H, bd, J=8.2 Hz, CO—CH—CH—CH₂), 1.40-1.43 (H, m, CO—CH—CH—CH₂), 1.45-1.52 (1H, m, CO—CH—CH₂), 1.89-1.98 (1H, m, CO—CH—CH₂), 2.86 and 3.05 (3H, 2 s, N—CH₃, rotamers), 2.89 (1H, bs, CO—CH—CH₂—CH), 3.03-3.07 and 3.14-3.16 (1H, 2*m, CO—CH—CH, rotamers), 3.12 (1H, bs, CO—CH), 4.45 (⅓ of 2H, d, J=15.0 Hz, CH₂-thiophene, rotamers), 4.78 (⅓ of 2H, s, CH₂-thiophene, rotamers), 4.85 (⅓ of 2H, d, J=15.0 Hz, CH₂-thiophene, rotamers), 6.02 and 6.07 (1H, dd, J=2.8, 5.5 Hz, CO—CH—CH—CH═CH, rotamers), 6.19 (1H, dd, J=3.2, 5.5 Hz, CO—CH—CH—CH═CH), 6.90-6.93 and 6.96-6.98 (2H, 2*m, CH_{Ar}, rotamers), 7.19 and 7.23 (1H, dd and m, J=2.0, 4.3 Hz, CH_{Ar}, rotamers); ¹³C NMR (100 MHz, CDCl₃) δ_{C} 30.7 and 31.2 (CO—CH—CH—CH₂, rotamers), 33.5 and 34.5 (N—CH₃, rotamers), 41.8 and 42.1 (CH—CO, rotamers), 42.7 (CH₂—CH—CH₂), 45.5 and 46.3 (CO—CH—CH, rotamers), 46.1 and 48.3 (N—CH₂, rotamers), 49.6 and 49.7 (CO—CH—CH₂), 125.1, 125.3, 125.4, 126.4, 126.5 and 127.0 (3*CH thiophene, rotamers), 132.8 (CO—CH—CH═CH), 136.8 (CO—CH—CH═CH), 140.4 (Cq_{Ar}), 174.0 (CO); LC/MS (AP+) m/z 248.4 (M+H); HPLC t_{R}=2.3 min (99.5%).

### Bicyclo[2.2.1]hept-5-ene-2-carboxylic acid methyl-thiophen-2-ylmethyl-amide (endo) (STX1710, CCM01158B)

Reaction of 5-norbornene-2-carboxylic acid (endo and exo) (145 mg, 1.0 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gives bicyclo[2.2.1]hept-5-ene-2-carboxylic acid methyl-thiophen-2-ylmethyl-amide (endo) (18 mg, 0.07 mmol, 7% yield) as a colourless oil after purification by flash chromatography on silica gel (EtOAc/DCM 0/10 to 0.5/9.5). ¹H NMR (270 MHz, CDCl₃) δ_{H} 1.34-1.44 (2H, m, CH₂ norbornene), 1.64-1.75 (1H, m, H norbornene), 1.80-1.88 and 1.89-1.94 (1H, 2*m, H norbornene, rotamers), 2.29-2.34 and 2.40-2.45 (1H, 2*m, H norbornene, rotamers), 2.87-2.92 (1H, bm, H norbornene, rotamers), 2.95 and 2.98 (3H, 2*s, N—CH₃, rotamers), 2.95-3.00 (1H, m, H norbornene), 4.69 and 4.71 (2H, 2*s, N—CH₂, rotamers), 6.09-6.21 (2H, m, CH═CH), 6.86-7.00 (2H, m, H_{Ar}), 7.15-7.23 (1H, m, H_{Ar}); LC/MS (AP+) m/z 248.4 (M+H); HPLC t_{R}=2.4 min (99.4%).

### Adamantan-1-yl-carbamic acid thiophen-2-ylmethyl ester (STX1711, XDS04021)

To a solution of 1-adamantyl isocyanate (195 mg, 1.1 mmol) in pyridine-toluene (1:2 mL) was added CuCl (10 mg, 0.1 mmol), followed by 2-thiophenemethanol (95 μL, 1.0 mmol). The mixture was stirred at 80° C. for 24 h, partitioned between ethyl acetate and saturated sodium chloride solution after cooling to room temperature. The organic phase was washed with brine, dried over sodium sulphate and concentrated in vacuo to give the crude product. Purification with flash column (hexane-ethyl acetate; gradient elution) yielded the title compound as white solid (230 mg, 79%). mp 66-68° C.; TLC single spot at R_{f}: 0.80 (30% ethyl acetate/hexane); ¹H NMR (270 MHz, CDCl₃) δ 1.64-1.91 (12H, m, 6×CH₂), 2.05 (3H, broad s, 3×CH), 4.58 (1H, s, NH), 5.17 (2H, s, CH₂), 6.96 (1H, dd, J=5.0, 3.5 Hz, ArH), 7.06 (1H, broad d, J=3.4 Hz, ArH) and 7.30 (1H, dd, J=5.0, 1.2 Hz, ArH); LC/MS (APCI) m/z 292 (M⁺+H); HPLC tᵣ=2.89 min (>99%) in 10% water-acetonitrile.

### Adamantan-1-yl-carbamic acid 2-thiophen-2-yl-ethyl ester (STX1712, XDS04022)

To a solution of 1-adamantyl isocyanate (195 mg, 1.1 mmol) in pyridine-toluene (1:2 mL) was added CuCl (10 mg, 0.1 mmol), followed by 2-thiopheneethanol (96 μL, 1.0 mmol). The mixture was stirred at 80° C. for 24 h, partitioned between ethyl acetate and saturated sodium chloride solution after cooling to room temperature. The organic phase was washed with brine, dried over sodium sulphate and concentrated in vacuo to give the crude product. Purification with flash column (hexane-ethyl acetate; gradient elution) yielded the title compound as white solid (180 mg, 59%). mp 67-69° C.; TLC single spot at R_{f}: 0.51 (30% ethyl acetate/hexane); ¹H NMR (270 MHz, CDCl₃) δ 1.64 (6H, s, 6×CH₂), 1.90 (6H, s, 3×CH₂), 2.05 (3H, broad s, 3×CH), 3.11 (2H, t, J=6.7 Hz, CH₂), 4.21 (2H, t, J=6.5 Hz, CH₂), 4.57 (1H, s, NH), 6.84 (1H, dd, J=3.3, 1.2 Hz, ArH), 6.92 (1H, dd, J=5.1, 3.4 Hz, ArH) and 7.14 (1H, dd, J=5.2, 1.2 Hz, ArH); LC/MS (APCI) m/z 306 (M⁺+H); HPLC tᵣ=3.05 min (>96%) in 10% water-acetonitrile.

### 1-Adamantan-1-yl-3-thiophen-2-ylmethyl-urea (STX1713, XDS04023)

To a solution of 1-adamantyl isocyanate (177 mg, 1.0 mmol) in DCM (5 mL) was added 2-thiophenemethylamine (102 μL, 1.0 mmol). The mixture was stirred at ambient temperature overnight, concentrated in vacuo to give the crude product as white solid, which was purified through recrystallization from methanol to yield the title compound as white crystalline solid (130 mg, 45%). mp 199-200° C.; TLC single spot at R_{f}: 0.51 (30% ethyl acetate/hexane); ¹H NMR (270 MHz, CDCl₃) δ 1.63-1.66 (6H, m, 3×CH₂), 1.94 (6H, d, J=2.9 Hz, 3×CH₂), 2.05 (3H, broad s, 3×CH), 4.09 (1H, s, NH), 4.47 (2H, s, CH₂), 4.49 (1H, s, NH), 6.90-6.93 (2H, m, ArH) and 7.19 (1H, dd, J=4.8, 1.9 Hz, ArH); LC/MS (APCI) m/z 3291 (M⁺+H); HPLC tᵣ=2.96 min (>96%) in 10% water-acetonitrile.

### 1-Adamantan-1-yl-3-(2-thiophen-2-yl-ethyl)urea (STX1714, XDS04024)

To a solution of 1-adamantyl isocyanate (177 mg, 1.0 mmol) in DCM (5 mL) was added 2-thiophenemethylamine (116 μL, 1.0 mmol). The mixture was stirred at ambient temperature overnight, concentrated in vacuo to give the crude product as white solid, which was purified through recrystallization from methanol to yield the title compound as white crystalline solid (190 mg, 63%). mp 138-142° C.; TLC single spot at R_{f}: 0.50 (30% ethyl acetate/hexane); ¹H NMR (270 MHz, CDCl₃) δ 1.62-1.65 (6H, m, 3×CH₂), 1.91 (6H, d, J=2.9 Hz, 3×CH₂), 2.04 (3H, broad s, 3×CH), 3.00 (2H, t, J=6.1 Hz, CH₂), 3.40 (2H, q, J=5.9 Hz, CH₂), 4.01 (1H, s, NH), 4.23 (1H, t, J=5.5 Hz, NH), 6.82 (1H, dd, J=3.3, 1.3 Hz, ArH), 6.93 (1H, dd, J=5.1, 3.2 Hz, ArH) and 7.15 (1H, dd, J=5.2, 1.3 Hz, ArH); LC/MS (APCI) m/z 305 (M⁺+H); HPLC tᵣ=2.51 min (>99%) in 10% water-acetonitrile.

### 4,5,6,7-Tetrahydrothieno[3,2-c]pyridine (XDS04020)

To a mixture of 2-thiophenyl ethylamine (1.07 g, 8.4 mmol) and 1,3-dioxolane (5 mL) was added 37% HCl solution (0.76 mL). The mixture was stirred at 75° C. for 5 h, partitioned between ethyl acetate and water after cooling to room temperature. The aqueous phase was washed ethyl acetate, neutralized to pH 7.5 with sodium hydroxide with brine and extracted with DCM. The organic phase was washed with water, dried over sodium sulphate and concentrated in vacuo to give the crude product as yellow oil (650 mg). The product was used in the next step without further purification.

### Adamantan-1-yl-(6,7-dihydro-4H-thieno[3,2-c]pyridin-5-yl)-methanone (STX1721, XDS04025)

To a solution of 1-adamantanecarbonyl chloride (100 mg, 0.50 mmol, 1.06 eq.) in DCM (5 mL) was added triethylamine (0.15 mL), followed by the 4,5,6,7-Tetrahydrothieno[3,2-c]pyridine (155 mg, purity unknown). The reaction mixture was stirred at ambient temperature under nitrogen overnight. PS-Trisamine (10-20 mg) was added. After stirred at ambient temperature for another 2 h, the mixture was filtered and evaporation of the solvent gave a residue that was purified by flash chromatography (Hexane-Ethyl acetate/hexane gradient elution) to give crystalline solid (49 mg, 33%).

mp 141-143° C.; TLC single spot at R_{f}: 0.49 (20% ethyl acetate/hexane); ¹H NMR (270 MHz, CDCl₃) δ 1.72 (6H, s, 3×CH₂), 2.02 (9H, s, 3×CH and 3×CH₂), 2.88 (2H, t, J=4.6 Hz, CH₂), 3.94 (2H, t, J=4.5 Hz, CH₂), 4.69 (2H, s, CH₂), 6.79 (1H, d, J=5.2 Hz, ArH) and 7.11 (1H, d, J=5.2 Hz, ArH); LC/MS (APCI) m/z 302 (M⁺+H); HPLC tᵣ=3.24 min (>99%) in 10% water-acetonitrile.

### 2-Adamantan-1-yl-1-(6,7-dihydro-4H-thieno[3,2-c]pyridin-5-yl)-ethanone (STX1722, XDS04026)

To a solution of 1-adamantyl acetic acid (97 mg, 0.50 mmol) in DCM (5 mL) were added EDCI (105 mg, 0.55 mmol), DMAP (16 mg, 0.25 mmol) and triethylamine (0.13 mL, 1.0 mmol), followed by 4,5,6,7-Tetrahydrothieno[3,2-c]pyridine (155 mg. purity unknown). The mixture was stirred at ambient temperature for 24 h, partitioned between ethyl acetate and 2% HCl solution. The organic phase was washed with 5% sodium bicarbonate and brine, dried over sodium sulphate and concentrated in vacuo to give the crude product. Purification with flash column (ethyl acetate/hexane; gradient elution) yielded the title compound as white solid (49 mg, 31%). mp 121-125° C.; TLC single spot at R_{f}: 0.28 (20% ethyl acetate/hexane); ¹H NMR data indicated signals of rotamers in ˜1:1 ratio (270 MHz, CDCl₃) δ 1.63 (12H, m, 6×CH₂), 1.68 (12H, m, 6×CH₂), 1.95 (6H, broad s, 3×CH and 3×CH), 2.20 (2H, s, CH₂), 2.23 (2H, s, CH₂), 2.83 (2H, t, J=4.5 Hz, CH₂), 2.89 (2H, t, J=4.5 Hz, CH₂), 3.79 (2H, t, J=4.7 Hz, CH₂), 3.92 (2H, t, J=4.7 Hz, CH₂), 4.58 (2H, s, CH₂), 4.67 (2H, s, CH₂), 6.77 (1H, d, J=5.1 Hz, ArH), 6.80 (1H, d, J=5.2 Hz, ArH), 7.11 (1H, d, J=5.1 Hz, ArH) and 7.13 (1H, d, J=5.1 Hz, ArH); LC/MS (APCI) m/z 316 (+19; HPLC tᵣ=3.52 min (>98%) in 10% water-acetonitrile.

### 3-Hydroxy-adamantane-1-carboxylic acid methyl-thiophen-2-ylmethyl-amide (STX1737, CCM01162)

Reaction of 3-hydroxy-adamantane-1-carboxylic acid (196 mg, 1.0 mmol) in DCM (10 mL) with methyl-thiophen-2-ylmethyl-amine (152 mg, 1.2 mmol) in presence of triethylamine (170 μL, 1.2 mmol), EDCI (230 mg, 1.2 mmol) and DMAP (147 mg, 1.2 mmol) according to the general procedure gives 3-hydroxy-adamantane-1-carboxylic acid methyl-thiophen-2-ylmethyl-amide (112 mg, 0.37 mmol, 37% yield) as a beige solid after purification by flash chromatography on silica gel (EtOAc/DCM 0/10 to 0.5/9.5). mp 88-91° C.; ¹H NMR (270 MHz, CDCl₃) δ_{H} 1.57-1.58 (3H, m, CH/CH₂ adamantane), 1.65-1.70 (4H, m, CV/CH₂ adamantane), 1.87-1.97 (6H, m, CH/CH₂ adamantane), 2.24-2.28 (2H, bs, CH/CH₂ adamantane), 3.07 (31, bs, N—CH₃), 4.71 (2H, s, CH₂—NMe), 6.94-6.97 (2H, m, H_{Ar}), 7.21-7.23 (1H, dd, J=2.0, 3.4 Hz, H_{Ar}thiophene); ¹³C NMR (67.5 MHz, CDCl₃) δ_{C} 30.6 (2*CH adamantane), 35.2 (CH₂ adamantane), 36.2 (N—CH₃), 37.7 (CH₂ adamantane), 44.4 (CH₂), 45.3 (Cq-OH), 46.7 (CH₂), 48.9 (CH₂), 68.8 (Cq-CO), 125.4 (CH_{Ar}), 126.2 (CH_{Ar}), 126.6 (CH_{Ar}), 140.4 (Cq_{Ar}), 175.4 (CO); LC/MS (AP+) m/z 306.4 (M+H); HPLC t_{R}=1.6 min (96.4%).

### 3,3-Dimethyl-1-(2-methyl-benzothiazol-5-ylamino)-butan-2-one (STX1740, CCM01174)

To a solution of 2-methylbenzothiazol-5-amine (180 mg, 1.1 mmol) and 1-bromopinacolone (100 μL, 0.74 mmol) in acetonitrile (10 mL) is added TEA (100 μL, 0.74 mmol). The resulting suspension is heated at reflux and at room temperature (heated during the day and at room temperature overnight). After 6 days, the solvent is removed under reduced pressure and water and DCM are added. The aqueous layer is extracted with DCM, the combined organic layers are washed with brine and dried over MgSO₄. After evaporation, the crude product is purified by flash chromatography (MeOH/DCM 0/100 to 3/97) to give 1-(2-methylbenzo[d]thiazol-5-ylamino)-3,3-dimethylbutan-2-one (57 mg, 0.22 mmol, 30% yield) as an yellow powder. mp 108-111° C.; ¹H NMR (270 MHz, CDCl₃) δ_{H} 1.24 (9H, s, tBu), 2.78 (3H, s, CH₃), 4.15 (2H, d, J=4.5 Hz, CH₂), 4.78 (1H, bs, NH), 6.75 (1H, dd, J=2.5, 8.7 Hz, H_{Ar}), 7.09 (1H, d, J=2.5 Hz, H_{Ar}), 7.55 (1H, d, J=8.7 Hz, H_{Ar}); ¹³C NMR (67.5 MHz, CDCl₃) δ_{C} 20.2 (CH₃), 26.8 (3*CH₃), 43.3 (C_{q}—CO), 49.0 (CH₂), 104.1 (CH_{Ar}), 113.8 (CH_{Ar}), 121.7 (CH_{Ar}), 124.4 (C_{q}), 146.4 (C_{q}), 155.0 (C_{q}), 167.8 (C_{q}), 211.4 (C═O); LC/MS (AP+) m/z 263.3 (M+H); HPLC t_{R}=1.9 min (96.5%).

### N-Methyl-N-(2-(thiophen-2-yl)ethyl)pivalamide (STX1780, CCM01178)

A solution of 2,2-dimethyl-N-(2-thiophen-2-yl-ethyl)-propionamide (50 mg, 0.24 mmol), sodium hydride (40 mg, 1.0 mmol) and iodomethane (75 μL, 1.2 mmol) in DMF (2.5 mL) is stirred at room temperature for 9 days. The reaction mixture is then poured in water. The aqueous layer is extracted with ethyl acetate; the combined organic layers are washed with brine and dried over MgSO₄. After evaporation, the crude product is purified by flash chromatography (EtOAc/hexane 0/10 to 2/8) to give N-methyl-N-(2-(thiophen-2-yl)ethyl)pivalamide as a colourless oil (24 mg, 0.11 mmol, 450%). ¹H NMR (270 MHz, CDCl₃) δ_{H} 1.26 and 1.27 (9H, 2*s, tBu, rotamers), 2.99 and 3.00 (3H, 2*s, N—CH₃, rotamers), 3.07 and 3.08 (2H, t, J=6.1 Hz, CH₂), 3.59 (2H, bt, CH₂), 6.81-6.83 (1H, m, H_{Ar}), 6.92 (1H, dd, J=3.9, 5.7 Hz, H_{Ar}), 7.13 (1H, dd, J=1.4, 5.7 Hz, H_{Ar}); ¹³C NMR (67.5 MHz, CDCl₃) δ_{C} 28.0 and 29.8 (CH₂, rotamers), 28.3 (3*CH₃), 37.3 (N—CH₃), 38.9 (CH₃-Cq-CO), 52.5 (CH₂), 123.8 (CH_{Ar}), 125.3 (CH_{Ar}), 127.0 (CH_{Ar}), 135.1 (Cq_{Ar}), 177.5 (CO); LC/MS (AP+) m/z 226.2 (M+H); HPLC t_{R}=2.5 min (99.9%).

### N,1-Dimethyl-N-(2-thiophen-2-yl)ethyl)cyclohexanecarboxamide (STX1781, CCM01181)

A solution of 1-methyl-cyclohexanecarboxylic acid (2-thiophen-2-yl-ethyl)-amide (50 mg, 0.20 mmol), sodium hydride (24 mg, 0.60 mmol) and iodomethane (38 μL, 0.60 mmol) in DMF (2.5 mL) is stirred at room temperature for 6 days. The reaction mixture is then poured in water. The aqueous layer is extracted with ethyl acetate; the combined organic layers are washed with brine and dried over MgSO₄. After evaporation, the crude product is purified by flash chromatography (EtOAc/hexane 0/10 to 2/8) to give N,1-dimethyl-N-(2-(thiophen-2-yl)ethyl)cyclohexanecarboxamide as a colourless oil (24 mg, 0.09 mmol, 45%). ¹H NMR (270 MHz, CDCl₃) δ_{H} 1.21 (3H, s, CH₃), 1.28-1.37 (3H, m, cyclohexane), 1.41-1.51 (5H, m, cyclohexane), 2.02-2.09 (2H, m, cyclohexane), 3.00 (3H, s, N—CH₃), 3.07 (2H, t, J=7.9 Hz, CH₂-thiophene), 3.61 (2H, t, J=7.9 Hz, N—CH₂), 6.82-6.83 (1H, m, H_{Ar}), 6.92 (1H, dd, J=3.8, 5.8 Hz, H_{Ar}), 7.13 (1H, dd, J=1.4, 5.8 Hz, H_{Ar}); LC/MS (AP+) m/z 266.3 (M+H); HPLC t_{R}=3.3 min (99.9%).

### N-Methyl-2,2-diphenyl-N-(2-(thiophen-2-yl)ethyl)propanamide (STX1782, CCM01182)

A solution of 2,2-diphenyl-N-(2-thiophen-2-yl-ethyl)-propionamide (50 mg, 0.15 mmol), sodium hydride (18 mg, 0.45 mmol) and iodomethane (28 μL, 0.45 mmol) in DMF (2.5 mL) is stirred at room temperature for 3 days. The reaction mixture is then poured in water. The aqueous layer is extracted with ethyl acetate; the combined organic layers are washed with brine and dried over MgSO₄. After evaporation, the crude product is purified by flash chromatography (EtOAc/hexane 0/10 to 2/8) to give N-methyl-2,2-diphenyl-N-(2-(thiophen-2-yl)ethyl)propanamide as a colourless oil (46 mg, 0.13 mmol, 86%); LC/MS (AP+) m/z 350.4 (M+H); HPLC t_{R}=3.2 min (99.9%).

### Adamantane-1-carboxylic acid methyl-(2-pyridin-2-yl-ethyl)-amide (STX1887, XDS04027)

The title compound was synthesized with general method from 1-adamantanecarbonyl chloride (100 mg, 0.50 mmol). White crystalline solid (123 mg, 83%) was obtained. mp 64-65° C.; TLC single spot at R_{f}: 0.40 (10% methanol/DCM); ¹H NMR (270 MHz, CDCl₃) δ 1.69 (6H, s, 3×CH₂), 1.91-2.01 (9H, m, 3×CH and 3×CH₂), 3.01 (2H, t, J=7.3 Hz, CH₂), 3.04 (3H, s, NCH₃), 3.73 (2H, t, J=7.1 Hz, CH₂), 7.10-7.25 (2H, m, ArH), 7.59 (1H, td, J=7.7, 2.0 Hz, CH₂), and 8.53 (1H, dq, J=4.7, 0.8 Hz, ArH); LC/MS (APCI) m/z 299 (M⁺+H); HPLC tᵣ=3.64 min (>99%) in 30% water-acetonitrile.

### 2-Adamantan-1-yl-N-methyl-N-(2-pyridin-2-yl-ethyl)-acetamide (STX1888, XDS04028)

The title compound was synthesized with general method from 1-adamantanecarbonyl acetic acid (97 mg, 0.50 mmol). White crystalline solid (112 mg, 72%) was obtained. mp 59-60° C.; TLC single spot at R_{f}: 0.39 (10% methanol/DCM); ¹H NMR data indicated signals of rotamers in ˜1:1 ratio (270 MHz, CDCl₃) δ 1.59-1.69 (24H, m, 6×CH₂ and 6×CH₂), 1.90 (2H, s, CH₂), 1.91 (611, broad s, 3×CH and 3×CH), 2.08 (2H, s, CH₂), 2.92 (3H, s, CH₃), 2.94 (3H, s, CH₃), 2.96-3.06 (4H, m, 2×CH₂), 3.74 (4H, t, J=6.6 Hz, 2×NCH₂), 7.08-7.13 (4H, m, ArH), 7.55-7.60 (2H, m, ArH), 8.51 (1H, dq, J=5.0, 1.0 Hz, ArH) and 8.55 (1H, dq, J=5.0, 1.0 Hz, ArH); LC/MS (APCI) m/z 313 (M⁺+H); HPLC tᵣ=3.83 min (>99%) in 30% water-acetonitrile.

### 1,4-Dichloro-2-prop-2-ynylsulfanyl-benzene (XDS04029)

To a solution of 2,5-dichlorobenzenethiol (887 mg, 4.96 mmol) in acetone (80 mL) were added propargyl chloride (369 mg, 4.96 mmol) and potassium carbonate (1.2 g). The mixture was refluxed for 3 h, cooled to room temperature and filtered. The filtrate was concentrated in vacuo to give the crude product. Purification with flash column (ethyl acetate/hexane; gradient elution) yielded the title compound as yellow oil (780 mg, 72%). TLC single spot at R_{f}: 0.71 (10% ethyl acetate/hexane); ¹H NMR (270 MHz, CDCl₃) δ 2.27 (1H, t, J=2.5 Hz, CH), 3.66 (211, d, J=2.5 Hz, CH₂), 7.12 (1H, dd, J=8.5, 2.5 Hz, ArH), 7.27 (1H, d, J=8.6 Hz, ArH) and 7.39 (1H, d, J=2.5 Hz, ArH); LC/MS (APCI) m/z 177 (M⁺−C₃H₃); HPLC tᵣ=3.32 min (>98%) in 30% water-acetonitrile.

### 1-Adamantan-1-yl-4-(2,5-dichloro-phenylsulfanylmethyl)-1H-[1,2,3]triazole (STX1889, XDS04030)

To a suspension of 1-azidoadamantane (355 mg, 2.0 mmol) and 1,4-dichloro-2-prop-2-ynylsulfanyl-benzene (430 mg, 1.98 mmol) in t-butanol/water (5 mL/5 mL) were added sodium ascorbate (40 mg in 0.2 mL water), followed by CuSO₄ (5 mg in 0.2 mL water). The mixture was stirred at ambient temperature for 24 h, partitioned between DCM and water. The organic phase was washed with brine, dried over sodium sulphate and concentrated in vacuo to give the crude product. Purification with flash column (ethyl acetate/hexane; gradient elution) yielded the title compound as white crystalline solid (380 mg, 49%). mp 161-162° C.; TLC single spot at R_{f}: 0.78 (25% ethyl acetate/hexane); ¹H NMR (400 MHz, CDCl₃) δ 1.70-1.78 (6H, m, 3×CH₂), 2.16 (6H, d, J=2.2 Hz, 3×CH₂), 2.21 (3H, broad s, 3×CH), 4.23 (2H, s CH₂), 7.05 (1H, dd, J=8.2, 2.2 Hz, ArH), 7.25 (1H, d, J=8.3 Hz, ArH), 7.27 (1H, d, J=2.3 Hz, ArH) and 7.45 (1H, s, ArH); LC/MS (APCI) m/z 394 (M⁺+H); HPLC tᵣ=4.26 min (>99%) in 10% water-acetonitrile.

### Adamantane-1-carboxylic acid (5-pyridin-2-yl-thiophen-2-ylmethyl)-amide (STX1890, XDS04033)

The title compound was synthesized with general method from 1-adamantanecarbonyl chloride (10 mg, 0.50 mmol). White crystalline solid (120 mg, 68%) was obtained. mp 147-149° C.; TLC single spot at R_{f}: 0.62 (20% ethyl acetate/DCM); ¹H NMR (270 MHz, CDCl₃) δ δ 1.69 (6H, m, 3×CH₂), 1.86 (6H, d, J=2.2 Hz, 3×CH₂), 2.03 (3H, broad s, 3×CH), 4.61 (2H, d, J=5.7 Hz, CH₂), 5.94 (1H, broad, NH), 6.94 (1H, dt, J=3.8, 0.8 Hz, ArH), 7.10-7.15 (1H, m, ArH), 7.40 (1H, d, J=3.5 Hz, ArH), 7.59-7.67 (2H, m, ArH) and 8.53 (1H, dq, J=5.0, 0.7 Hz, ArH); LC/MS (APCI) m/z 353 (M⁺+H); HPLC tᵣ=2.79 min (>99%) in 10% water-acetonitrile.

### 2-Adamantan-1-yl-N-(5-pyridin-2-yl-thiophen-2-ylmethyl)acetamide (STX1891, XDS04034)

The title compound was synthesized with general method from 1-adamantyl acetic acid (97 mg, 0.50 mmol). White crystalline solid (99 mg, 54%) was obtained. mp 187-188° C.; TLC single spot at R_{f}: 0.49 (20% ethyl acetate/DCM); ¹H NMR (270 MHz, CDCl₃) δ δ 1.62 (12H, m, 6×CH₂), 2.27 (5H, broad s, CH₂ and 3×CH), 4.60 (2H, d, J=5.8 Hz, CH₂), 5.68 (1H, broad, NH), 6.96 (1H, dt, J=3.7, 0.8 Hz, ArH), 7.12 (1H, ddd, J=7.2, 4.7, 1.2 Hz, ArH), 7.41 (1H, d, J=3.7 Hz, ArH), 7.58-7.67 (2H, m, ArH) and 8.53 (1H, dq, J=4.9, 0.8 Hz, ArH); LC/MS (APCI) m/z 367 (M⁺+H); HPLC tᵣ=2.92 min (>99%) in 10% water-acetonitrile.

### 1-Adamantan-1-yl-4-(2,5-dichloro-benzenesulfinylmethyl)-1H-[1,2,3]triazole (STX1892, XDS04036)

To a solution of 1-Adamantan-1-yl-4-(2,5-dichloro-phenylsulfanylmethyl)-1H-[1,2,3]triazole (236 mg, 0.60 mmol) in DCM (25 mL) was added m-CPBA (180 mg, purity 60-77%). The mixture was stirred at 0° C. for 40 min, partitioned between ethyl acetate and 5% sodium carbonate solution. The organic phase was washed with brine, dried over sodium sulphate and concentrated in vacuo to give the crude product as white solid. Purification with flash column (hexane-ethyl acetate; gradient elution) yielded the title compound as white solid (235 mg, 96%). mp 125-128° C.; TLC single spot at R_{f}: 0.22 (30% EtOAc/hexane); ¹H NMR (270 MHz, CDCl₃) δ 1.72-1.82 (6H, m, 3×CH₂), 2.21 (6H, broad, 3×CH₂), 2.25 (3H, broad s, 3×CH), 4.36 (2H, s, CH₂), 7.23 (1H, dd, J=2.0, 0.7 Hz, ArH), 7.27-7.35 (2K, m, ArH) and 7.69 (1H, s ArH); -LC/MS (APCI) m/z 410 (M⁺+H); HPLC tᵣ=3.72 min (>99%) in 10% water-acetonitrile.

### 1,2-Dimethoxy-4-prop-2-ynyloxy-benzene (XDS04032)

To a solution of 3,4-dimethoxyphenol (616 mg, 4.0 mmol) in ethanol (50 mL) were added propargyl chloride (398 mg, 5.35 mmol) and potassium carbonate (1.2 g). The mixture was refluxed for 22 h, cooled to room temperature and filtered. The filtrate was concentrated in vacuo to give the crude product. Purification with flash column (ethyl acetate/hexane; gradient elution) yielded the title compound as yellow oil (250 mg, 33%). TLC single spot at R_{f}: 0.51 (20% ethyl acetate/hexane); ¹H NMR (270 MHz, CDCl₃) δ 2.50 (1H, t, J=2.5 Hz, CH), 3.79 (3H, s, OCH₃), 3.81 (3H, s, OCH₃), 4.60 (2H, d, J=2.5 Hz, CH₂), 6.45 (1H, dd, J=8.5, 2.5 Hz, ArH), 6.55 (1H, d, J=2.7 Hz, ArH) and 6.75 (1H, d, J=8.7 Hz, ArH); LC/MS (APCI) m/z 193 (M⁺+H); HPLC tᵣ=2.98 min (76%) in 30% water-acetonitrile.

### 1-Adamantan-1-yl-4-(3,4-dimethoxy-phenoxymethyl)-1H-[1,2,3]triazole (STX1893, XDS04037B)

To a suspension of 1-azidoadamantane (165 mg, 0.93 mmol) and 1,2-dimethoxy-4-prop-2-ynyloxy-benzene (178 mg, 0.93 mmol) in t-butanol/water (3 mL/3 mL) was added sodium ascorbate (20 mg in 0.1 mL water), followed by CuSO₄ (2.5 mg in 0.2 mL water). The mixture was stirred at ambient temperature for 20 h, partitioned between DCM and water. The organic phase was washed with brine, dried over sodium sulphate and concentrated in vacuo to give the crude product as yellow oil. Purification with flash column (ethyl acetate/hexane; gradient elution) yielded the title compound as white solid (160 mg, 85%). 80 mg of 1,2-dimethoxy-prop-2-ynyloxy-benzene was recovered. mp 108-112° C.; TLC single spot at R_{f}: 0.39 (40% ethyl acetate/hexane); ¹H NMR (400 MHz, CDCl₃) δ 1.77 (6H, m, 3×CH₂), 2.23 (9H, s, 3×CH₂ and 3×CH), 3.82 (3H, s, OCH₃), 3.83 (3H, s, OCH₃), 5.14 (2H, s, CH₂), 6.51 (1H, dd, J=8.7, 2.8 Hz, ArH), 6.60 (1H, d, J=2.9 Hz, ArH), 6.78 (1H, d, J=8.6 Hz, ArH) and 7.66 (1H, s, ArH); LC/MS (APCI) m/z 370 (M⁺+H); HPLC tᵣ=15.4 min (99%) in 30% water-acetonitrile.

### N-methyl-N-(thiophen-2-ylmethyl)prop-2-yn-1-amine XDS04035)

To a solution of N-methyl(thiophen-2-yl)methanamine (597 mg, 4.70 mmol) in DCM (15 mL) was added propargyl chloride (348 mg, 4.70 mmol), followed by diisopropylethylamine (0.5 mL). The mixture was stirred at ambient temperature for 20 h, partitioned between DCM and water. The organic phase was washed with brine, dried over sodium sulphate and concentrated in vacuo to give the crude product. Purification with flash column (ethyl acetate/hexane; gradient elution) yielded the title compound as clear oil (505 mg, 65%). TLC single spot at R_{f}: 0.37 (20% ethyl acetate/hexane); ¹H NMR (270 MHz, CDCl₃) δ 2.26 (1H, t, J=2.2 Hz, CH), 2.36 (3H, s, CH₃), 3.35 (2H, d, J=2.5 Hz, CH₂), 3.78 (2H, s, CH₂), 6.91-6.96 (2H, m, ArH) and 7.23 (1H, d, J=1.8 Hz, ArH); LC/MS (APCI) m/z 166 (+H); HPLC tᵣ=3.11 min (>98%) in 10% water-acetonitrile.

### (1-Adamantan-1-yl-1H-[1,2,3]triazol-4-ylmethyl)-methyl-thiophen-1-ylmethyl-amine (STX1894, XDS04038)

To a suspension of 1-azidoadamantane (266 mg, 1.5 mmol) and N-methyl-N-(thiophen-2-ylmethyl)prop-2-yn-1-amine (248 mg, 1.5 mmol) in t-butanol/water (5 mL/5 mL) was added sodium ascorbate (30 mg in 0.2 mL water), followed by CuSO₄ (5 mg in 0.1 mL water). The mixture was stirred at ambient temperature for 24 h, partitioned between DCM and water. The organic phase was washed with brine, dried over sodium sulphate and concentrated in vacuo to give the crude product. Purification with flash column (ethyl acetate/hexane; gradient elution) yielded the title compound as white solid (251 mg, 49%). mp 66-68° C.; TLC single spot at R_{f}: 0.29 (40% ethyl acetate/hexane); ¹H NMR (270 MHz, CDCl₃) δ 1.75 (6H, m, 3×CH₂), 2.23 (9H, s, 3×CH₂ and 3×CH), 2.30 (3H, s, NCH₃), 3.71 (2H, s, CH₂), 3.76 (2H, s, CH₂), 6.93-6.96 (2H, m, ArH), 7.22 (1H, dd, J=4.0, 2.2 Hz, ArH) and 7.55 (1H, s, ArH); LC/MS (APCI) m/z 343 (M⁺+H); HPLC tᵣ=3.76 min (>99%) in 10% water-acetonitrile.

### 1-Adamantan-1-yl-4-(2,5-dichloro-benzenesulfonylmethyl)-1H-[1,2,3]triazole (STX1895, XDS04039)

To a solution of 1-adamantan-1-yl-4-(2,5-dichloro-benzenesulfinylmethyl)-1H-[1,2,3]triazole (121 mg, 0.30 mmol) in DCM (5 mL) was added m-CPBA (100 mg, purity 60-77%). The mixture was stirred at ambient temperature for 20 h, partitioned between ethyl acetate and 5% sodium carbonate solution. The organic phase was washed with brine, dried over sodium sulphate and concentrated in vacuo to give the crude product as white solid. Purification with flash column (hexane-ethyl acetate; gradient elution) yielded the title compound as white solid (75 mg, 59%). mp 198-199° C.; TLC single spot at R_{f}: 0.56 (50% EtOAc/hexane); ¹H NMR (270 MHz, CDCl₃) δ 1.76 (6H, m, 3×CH₂), 2.16 (6H, d, J=2.2 Hz, 3×CH₂), 2.25 (3H, broad s, 3×CH), 4.83 (2H, s, CH₂), 7.46 (2H, m, ArH), 7.73 (1H, s, ArH) and 7.75 (1H, t, J=1.5 Hz, ArH); LC/MS (APCI) m/z 426 (M⁺+H); HPLC tᵣ=3.27 min (>99%) in 10% water-acetonitrile.

### 1-Adamantan-1-yl-2-methyl-thiophen-2-ylmethyl-amino)ethanone hydrogen chloride (STX1896, XDS04040)

To a solution of adamantan-1-yl bromomethyl ketone (256 mg, 1.0 mmol) in acetonitrile (10 mL) was added N-methyl(thiophen-2-yl)methanamine (128 mg, 1.0 mmol). The mixture was stirred at ambient temperature for 4 h, partitioned between ethyl acetate and brine. The organic phase was washed with brine, dried over sodium sulphate and concentrated in vacuo to give the crude product. Purification with flash column (DCM-ethyl acetate; gradient elution) yielded the free base of title compound, which was treated with HCl ether solution to give a white solid (65 mg, 19%). mp 201-203° C. (HCl salt); TLC single spot at R_{f}: 0.30 (20% EtOAc/DCM) (free base); ¹H NMR (free base) (270 MHz, CDCl₃) δ 1.63-2.00 (12H, m, 6×CH₂), 2.00 (3H, broad, 3×CH), 2.35 (3H, s, CH₃), 3.42 (2H, s, CH₂), 3.88 (2H, s, CH₂), 6.88 (1H, m, ArH), 6.93 (1H, dd, J=5.0, 3.5 Hz, ArH) and 7.23 (1H, dd, J=5.0, 1.3 Hz, ArH); LC/MS (Free base) (APCI) m/z 304 (M⁺+H); HPLC (HCl salt) tᵣ=6.39 min (>99%) in 20% water-acetonitrile.

### 1-Adamantan-1-yl-2-(furan-2-ylmethylsulfanyl)-ethanone (STX1897, XDS04041)

To a solution of adamantan-1-yl bromomethyl ketone (256 mg, 1.0 mmol) in acetonitrile (5 mL) was added 2-furfuryl mercaptan (0.1 mL, 1.0 mmol), followed by triethylamine (0.5 mL). The mixture was stirred at ambient temperature for 20 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as waxy-like semi solid (180 mg, 62%). TLC single spot at R_{f}: 0.59 (10% EtOAc/DCM); ¹H NMR (270 MHz, CDCl₃) δ 1.69-1.83 (6H, m, 3×CH₂), 1.82 (6H, d, J=2.2 Hz, 3×CH₂), 2.03 (3H, broad, 3×CH), 3.33 (2H, s, CH₂), 3.75 (2H, s, CH₂), 6.18-6.20 (1H, m, ArH), 6.29 (1H, dd, J=3.1, 1.7 Hz, ArH) and 7.35 (1H, dd, J=2.0, 1.0 Hz, ArH); LC/MS (APCI) m/z 291 (M⁺+H); HPLC tᵣ=3.58 min (>99%) in 20% water-acetonitrile.

### 1-Adamantan-1-yl-2-(1-methyl-1H-imidazol-2-ylsulfanyl)-ethanone (STX1898, XDS04042)

To a solution of adamantan-1-yl bromomethyl ketone (256 mg, 1.0 mmol) in acetonitrile (5 mL) was added 2-mecapto 1-methylimidazole (114 mg, 1.0 mmol), followed by triethylamine (0.5 mL). The mixture was stirred at ambient temperature for 20 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as off-white solid (230 mg, 79%). mp 65-69° C.; TLC single spot at R_{f}: 0.51 (50% EtOAc/hexane); ¹H NMR (270 MHz, CDCl₃) δ 1.63-1.81 (6H, m, 3×CH₂), 1.82 (6H, d, J=2.3 Hz, 3×CH₂), 2.03 (3H, broad, 3×CH), 3.64 (3H, s, CH₃), 4.23 (2H, s, CH₂), 6.89 (1H, d, J=1.2 Hz, ArH) and 7.01 (1H, d, J=1.2 Hz, ArH); LC/MS (APCI) m/z 291 (M⁺+H); HPLC tᵣ=3.00 min (>99%) in 10% water-acetonitrile.

### 1-Adamantan-1-yl-2-(thiophen-2-ylmethylsulfanyl)-ethanone (STX1899, XDS04043)

To a solution of adamantan-1-yl bromomethyl ketone (256 mg, 1.0 mmol) in acetonitrile (5 mL) was added 2-thienyl mercaptan (130 mg, 1.0 mmol), followed by triethylamine (0.5 mL). The mixture was stirred at ambient temperature for 20 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as waxy-like semi solid (160 mg, 52%). TLC single spot at R_{f}: 0.59 (50% hexane/DCM); ¹H NMR (270 MHz, CDCl₃) δ 1.62-1.81 (12H, m, 6×CH₂), 2.02 (3H, broad, 3×CH), 3.32 (2H, s, CH₂), 3.95 (2H, s, CH₂), 6.88-6.94 (2H, m, ArH) and 7.20 (1H, dd, J=5.0, 1.3 Hz, ArH); LC/MS (APCI) m/z 307 (M⁺+H); HPLC tᵣ=5.14 min (>97%) in 20% water-acetonitrile.

### Adamantan-1-yl-(6-benzyloxy-7-methoxy-3,4-dihydro-1H-isoquinolin-2-yl)-methanone (STX1900, XDS04044)

To a solution of 1-adarmantanecarbonyl chloride (179 mg, 0.901 mmol) in DCM (6 mL) was added triethylamine (0.20 mL), followed by 6-(benzyloxy)-7-methoxy-1,2,3,4-tetrahydroisoquinoline (230 mg, 0.86). The reaction mixture was stirred at ambient temperature under nitrogen overnight. PS-Trisamine (10-20 mg) was added. After stirred at ambient temperature for another 2 h, the mixture was filtered and evaporation of the solvent gave a residue that was purified by flash chromatography (Hexane-Ethyl acetate/hexane gradient elution) to give crystalline solid (350 mg, 94%). mp 144-145° C.; TLC single spot at R_{f}: 0.55 (50% ethyl acetate/hexane); ¹H NMR (270 MHz, CDCl₃) δ 1.73 (6H, s, 3×CH₂), 2.02 (9H, broad s, 3×CH and 3×CH₂), 2.73 (2H, t, J=5.7 Hz, CH₂), 3.85 (3H, s, CH₃), 3.86 (2H, t, J=5.5 Hz, CH₂), 4.69 (2H, s, CH₂), 5.10 (2H, s, CH₂), 6.61 (1H, s, ArH), 6.62 (1H, s, ArH) and 7.26-7.44 (5H, m, ArH); LC/MS (APCI) m/z 432 (M⁺+H); HPLC tᵣ=5.91 min (>99%) in 10% water-acetonitrile.

### 2-Adamantan-1-yl-1-(6-benzyloxy-7-methoxy-3,4-dihydro-1H-isoquinolin-2-yl)-ethanone (STX1901, XDS04045)

To a solution of 1-adamantyl acetic acid (175 mg, 0.90 mmol) in DCM (5 mL) were added EDCI (175 mg, 0.90 mmol), DMAP (20 mg, 0.31 mmol) and triethylamine (0.20 mL), followed by 6-(benzyloxy)-7-methoxy-1,2,3,4-tetrahydroisoquinoline (230 mg, 0.86). The mixture was stirred at ambient temperature for 24 h, partitioned between ethyl acetate and 2% HCl solution. The organic phase was washed with 5% sodium bicarbonate and brine, dried over sodium sulphate and concentrated in vacuo to give the crude product. Purification with flash column (ethyl acetate/hexane; gradient elution) yielded the title compound as white solid (285 mg, 74%). mp 133-135° C.; TLC single spot at R_{f}: 0.52 (50% ethyl acetate/hexane); ¹H NMR data indicated signals of rotamers in ˜1:1 ratio (270, CDCl₃) δ 1.57-1.69 (24H, m, 12×CH₂), 1.94 (6H, broad s, 6×CH), 2.20 (2H, s, CH₂), 2.21 (2H, s, CH₂), 2.69-2.73 (4H, m, 2×CH₂), 3.68 (2H, t, J=5.8 Hz, CH₂), 3.79 (2H, t, J=5.9 Hz, CH₂), 3.86 (6H, s, 2×CH₃), 4.58 (2H, s, CH₂), 4.66 (2H, s, CH₂), 5.11 (4H, s, 2×CH₂), 6.59-6.65 (4H, m, ArH) and 7.25-7.44 (10H, m, ArH); LC/MS (APCI) m/z 446 (b+H); HPLC tᵣ=6.00 min (>99%) in 10% water-acetonitrile.

### 1-Adamantan-1-yl-2-(thiophen-2-ylsulfanyl)-ethanone (STX1902, XDS04047)

To a solution of adamantan-1-yl bromomethyl ketone (256 mg, 1.0 mmol) in acetonitrile (5 mL) was added 2-mercapto thiophene (0.093 mL, 1.0 mmol), followed by triethylamine (0.5 mL). The mixture was stirred at ambient temperature for 20 h, partitioned between ethyl acetate and saturated sodium carbonate. The organic phase was washed with brine, dried over sodium sulphate and concentrated in vacuo to give the crude product. Purification with flash column (hexane-ethyl acetate; gradient elution) yielded the title compound as off-white solid (250 mg, 86%). mp 69-72° C.; TLC single spot at R_{f}: 0.59 (50% hexane/DCM); ¹H NMR (270 MHz, CDCl₃) δ 1.67-1.78 (12H, m, 6×CH₂), 2.02 (3H, broad, 3×CH), 3.84 (2H, s, CH₂), 6.94 (1H, dd, J=5.1, 3.2 Hz, ArH), 7.15 (1H, dd, J=3.3, 1.5 Hz, ArH) and 7.34 (1H, dd, J=5.2, 1.5 Hz, ArH); LC/MS (APCI) m/z 293 (M⁺+H); HPLC tᵣ=3.60 min (>99%) in 20% water-acetonitrile.

### Adamantan-1-yl-(6-hydroxy-7-methoxy-3,4-dihydro-1H-isoquinolin-2-yl)-methanone (STX1903, XDS04049)

The solution of adamantan-1-yl-(6-benzyloxy-7-methoxy-3,4-dihydro-1H-isoquinolin-2-yl)-methanone (280 mg, 0.65 mmol) in CH₃OH-THF (30:10 mL) was hydrogenated over 5% Pd/C (100 mg) at atmosphere for 3 h, filtered and evaporation of the solvent gave a residue that was purified by flash chromatography (Ethyl acetate/hexane gradient elution) to give white solid (178 mg, 80%). mp 224-228° C.; TLC single spot at R_{f}: 0.46 (50% ethyl acetate/hexane); ¹H NMR (270 MHz, CDCl₃) δ 1.73 (6H, s, 3×CH₂), 2.02 (9H, broad s, 3×CH and 3×CH₂), 2.76 (2H, t, J=5.5 Hz, CH₂), 3.85 (5H, m, CH₂ and CH₃), 4.67 (2H, s, CH₂), 5.51 (1H, s, OH), 6.56 (1H, s, ArH) and 6.66 (1H, s, ArH); LC/MS (APCI) m/z 342 (M⁺+H); HPLC tᵣ=3.00 min (92%) in 10% water-acetonitrile.

### 2-Adamantan-1-yl-1-(6-hydroxy-7-methoxy-3,4-dihydro-1H-isoquinolin-2-yl)-ethanone (STX1904, XDS04050)

The solution of 2-adamantan-1-yl-1-(6-benzyloxy-7-methoxy-3,4-dihydro-1H-isoquinolin-2-yl)-ethanone (220 mg, 0.49 mmol) in CH₃OH-THF (25:10 mL) was hydrogenated over 5% Pd/C (100 mg) at atmosphere for 3 h, filtered and evaporation of the solvent gave a residue that was purified by flash chromatography (Ethyl acetate/hexane gradient elution) to give white solid (155 mg, 89%). mp 169-172° C.; TLC single spot at R_{f}: 0.37 (45% ethyl acetate/hexane); ¹H NMR data indicated signals of rotamers in ˜1:1 ratio (270 MHz, CDCl₃) δ 1.62-1.75 (24H, m, 12×CH₂), 1.95 (6H, broad s, 6×CH), 2.21 (4H, s, 2×CH₂), 2.74 (4H, m, 2×CH₂), 3.68 (2H, t, J=5.5 Hz, CH₂), 3.79 (2H, t, J=5.5 Hz, CH₂), 3.85 (3H, s, OCH₃), 3.86 (3H, s, OCH₃), 4.57 (2H, s, CH₂), 4.65 (2H, s, CH₂), 5.52 (1H, s, OH), 5.53 (1H, s, OH), 6.55 (1H, s, ArH), 6.59 (1H, s, ArH), 6.67 (1H, s, ArH) and 6.70 (1H, s, ArH); LC/MS (APCI) m/z 356 (M⁺+H); HPLC tᵣ=3.11 min (94%) in 10% water-acetonitrile.

### Adamantan-1-yl-(6,7-dimethoxy-3,4-dihydro-1H-isoquinolin-2-yl)-methanone (STX1905, XDS04052)

To a solution of adamantan-1-yl-(6-hydroxy-7-methoxy-3,4-dihydro-1H-isoquinolin-2-yl)-methanone (120 mg, 0.35 mmol) in acetone (30 mL) was added potassium carbonate (200 mg), followed by CH₃I (0.13 mL, 2.1 mmol). The mixture was stirred at atmosphere for 18 h, concentrated in vacuo and extracted into DCM. The organic phase was washed with water, dried over sodium sulphate and concentrated in vacuo to give a yellow residue, which was purified with flash column (hexane-ethyl acetate; gradient elution) to yield the title compound as white solid (110 mg, 88%). mp 151-155° C.; TLC single spot at R_{f}: 0.47 (50% ethyl acetate/hexane); ¹H NMR (270 MHz, CDCl₃) δ 1.73 (6H, s, 3×CH₂), 2.03 (9H, broad s, 3×CH and 3×CH₂), 2.79 (2H, t, J=5.5 Hz, CH₂), 3.84-3.88 (8H, m, CH₂ and 2×OCH₃), 4.70 (2H, s, CH₂), 6.58 (1H, s, ArH) and 6.59 (1H, s, ArH); LC/MS (APCI) m/z 356 (M⁺+H); HPLC tᵣ=3.19 min (>99%) in 10% water-acetonitrile.

### 2-Adamantan-1-yl-1-(6,7-dimethoxy-3,4-dihydro-1H-isoquinolin-2-yl)-ethanone (STX1906, XDS04053)

The compound was synthesized according to the procedure for XDS04052. Purification with flash chromatography (Ethyl acetate/hexane gradient elution) gave white solid (90 mg, 70%). mp 165-167° C.; TLC single spot at R_{f}: 0.37 (45% ethyl acetate/hexane); 111 NMR data indicated signals of rotamers in ˜1:1 ratio (270 MHz, CDCl₃) δ 1.60-1.70 (24H, m, 12×CH₂), 1.95 (6H, broad s, 6×CH), 2.22 (4H, s, 2×CH₂), 2.77 (4H, m, 2×CH₂), 3.71 (2H, t, J=5.7 Hz, CH₂), 3.82 (2H, t, J=5.7 Hz, CH₂), 3.84 (6H, s, 2×OCH₃), 3.85 (3H, s, OCH₃), 3.86 (3H, s, OCH₃), 4.58 (2H, s, CH₂), 4.66 (2H, s, CH₂), 6.56 (1H, s, ArH), 6.60 (1H, s, ArH), 6.61 (1H, s, ArH) and 6.62 (1H, s, ArH); LC/MS (APCI) m/z 370 (M⁺+1); HPLC tᵣ=3.50 min (99%) in 10% water-acetonitrile.

### 1-Adamantan-1-yl-2-(furan-2-ylmethanesulfinyl)ethanone (STX1908, XDS04054)

To a solution of 1-adamantan-1-yl-2-(furan-2-ylmethylsulfanyl)-ethanone (120 mg, 0.41 mmol) in DCM (12 mL) was added m-CPBA (108 mg, purity 60-77%). The mixture was stirred at −5° C. for 35 min. partitioned between ethyl acetate and 5% sodium carbonate solution. The organic phase was washed with brine, dried over sodium sulphate and concentrated in vacuo to give an oily product. Purification with flash column (hexane-ethyl acetate; gradient elution) yielded the title compound as white solid (100 mg, 80%). mp 75-79° C.; TLC single spot at R_{f}: 0.23 (50% EtOAc/DCM); ¹H NMR (270 MHz, CDCl₃) δ 1.56-1.84 (12H, m, 6×CH₂), 2.06 (3H, broad, 3×CH), 3.67 (1H, d, J=15 Hz, CH₂), 3.93 (1H, d, J=15 Hz, CH₂), 4.20 (2H, AB, CH₂), 6.04-6.44 (2H, m, ArH) and 7.44 (1H dd, J=1.8, 0.7 Hz, ArH); LC/MS (APCI) m/z 307 (M⁺+H); HPLC tᵣ=2.54 min (>99%) in 20% water-acetonitrile.

### Adamantane-1-carboxylic acid methyl-2-thiophen-2-yl-ethyl)-amide (STX1910, CCM02003)

A solution of 1-methyl-cyclohexanecarboxylic acid (2-thiophen-2-yl-ethyl)-amide (145 mg, 0.50 mmol), sodium hydride (60 mg, 1.5 mmol) and iodomethane (94 μL, 1.5 mmol) in DMF (6 mL) is stirred at room temperature for 6 days. The reaction mixture is then poured in water. The aqueous layer is extracted with ethyl acetate, the combined organic layers are washed with brine and dried over MgSO₄. After evaporation, the crude product is purified by flash chromatography (MeOH/DCM 0/100 to 2/98 to 5/95) to give adamantane-1-carboxylic acid methyl-(2-thiophen-2-yl-ethyl)-amide as a white solid (10 mg, 0.03 mmol, 6%). mp 95-99° C.; ¹H NMR (270 MHz, CDCl₃) δ_{H} 1.67 (6H, bs, adamantane), 2.00 (9H, bs, adamantane), 3.02 (3H, s, N—CH₃), 3.06-3.09 (2H, m, CH₂-thiophene), 3.61 (2H, t, J=7.4 Hz, N—CH₂), 6.81-6.82 (1H, m, H_{Ar}), 6.92 (1H, dd, J=3.2, 5.0 Hz, H_{Ar}), 7.13 (1H, dd, J=1.0, 5.0 Hz, H_{Ar}); LC/MS (AP+) m/z 304.3 (M+H).

### 2-(2-Methylbenzo[d]thiazol-5-ylamino)-1-(1-methylcyclopropyl)ethanone (STX1911, CCM02012)

A solution of 2-methylbenzo[d]thiazol-5-amine (103 mg, 0.63 mmol), 2-bromo-1-(1-methylcyclopropyl)ethanone (128 mg, 0.72 mmol) and sodium carbonate (130 mg, 1.2 mmol) in EtOH (5 mL) is heated at reflux overnight. After cooling, EtOH is removed under reduced pressure and water and DCM are added. The aqueous layer is extracted with DCM. The combined organic layers are washed with brine, dried over MgSO₄ and filtered. After evaporation, the crude product is purified by flash chromatography three times (MeOH/DCM 0/100 to 5/95) to give 2-(2-methylbenzo[d]thiazol-5-ylamino)-1-(1-methylcyclopropyl)ethanone (15 mg, 0.06 mmol, 9%) as an orange solid. mp 116-120° C.; ¹H NMR (270 MHz, CDCl₃) δ_{H} 0.83 (2H, dd, J=4.0, 7.0 Hz, 2H cyclopropane), 1.35 (2H, dd, J=4.0, 7.0 Hz, 2H cyclopropane), 1.46 (3H, s, CH₃-Cq-CO), 2.78 (3H, s, CH₃ benzothiazole), 4.16 (2H, s, CH₂—CO), 4.84 (1H, bs, NH), 6.75 (1H, dd, J=2.3, 8.5 Hz, H_{Ar}), 7.09 (1H, d, J=2.3 Hz, H_{Ar}), 7.54 (1H, d, J=8.5 Hz, H_{Ar}); LC/MS (AP+) m/z 261.1 (M+H).

### 1-Adamantan-1-yl-2-(1-methyl-1H-imidazole-2-sulfinyl)-ethanone (STX1924, XDS04055)

To a solution of 1-adamantan-1-yl-2-(1-methyl-1H-imidazol-2-ylsulfanyl)-ethanone (180 mg, 0.62 mmol) in DCM (15 mL) was added m-CPBA (163 mg, purity 60-77%). The mixture was stirred at −5° C. for 40 min, partitioned between ethyl acetate and 5% sodium carbonate solution. The organic phase was washed with brine, dried over sodium sulphate and concentrated in vacuo to give the crude product. Purification with flash column (methanol-DCM; gradient elution) yielded the title compound as yellow solid (170 mg, 90%). mp 86-89° C.; TLC single spot at R_{f}: 0.15 (50% EtOAc/hexane); ¹H NMR (270 MHz, CDCl₃) δ 1.63-1.85 (12H, m, 6×CH₂), 2.04 (3H, broad, 3×CH), 3.93 (3H, s, CH₃), 4.51 (1H, d, J=17 Hz, CH₂), 4.91 (1H, d, J=17 Hz, CH₂), 6.98 (1H, d, J=1.0 Hz, ArH) and 7.15 (1H, d, J=1.0 Hz, ArH); LC/MS (APCI) m/z 307 (M⁺+H); HPLC t=2.61 min (>96%) in 10% water-acetonitrile.

### Adamantane-1-carboxylic acid 4-chloro-benzylamide (STX1925, XDS04056)

The title compound was synthesized with general amide formation method from 1-adamantane carbonyl chloride (400 mg, 2.0 mmol) and the amine (0.245 mL, 2.0 mmol). White solid (430 mg, 71%) was obtained. mp 150-153° C.; TLC single spot at R_{f}: 0.70 (40% EtOAc/hexane); ¹H NMR (270 MHz, CDCl₃) δ 1.65-1.76 (6H, m, 3×CH₂), 1.86 (6H, d, J=2.3 Hz, 3×CH₂), 2.04 (3H, s, 3×CH), 4.40 (2H, d, J=5.5 Hz, CH₂), 5.86 (1H, broad, NH) and 7.15-7.26 (4H, m, ArH); LC/MS (APCI) m/z 304 (M⁺+H); HPLC tᵣ=2.95 min (>99%) in 10% water-acetonitrile.

### Adamantane-1-carboxylic acid 4-methyl-benzylamide (STX1926, XDS04057)

The title compound was synthesized with general amide formation method from 1-adamantane carbonyl chloride (400 mg, 2.0 mmol) and the amine (0.255 mL, 2.0 mmol). White solid (400 mg, 71%) was obtained. mp 137-140° C.; TLC single spot at R_{f}: 0.72 (40% EtOAc/hexane); ¹H NMR (270 MHz, CDCl₃) δ 1.64-1.75 (6H, m, 3×CH₂), 1.87 (6H, d, J=2.2 Hz, 3×CH₂), 2.03 (3H, s, 3×CH), 2.33 (3H, s, CH₃), 4.38 (21, d, J=5.5 Hz, CH₂), 5.78 (1H, broad, NH) and 7.13 (4H, m, ArH); LC/MS (APCI) m/z 284 (M⁺+H); HPLC tᵣ=2.99 min (>99%) in 10% water-acetonitrile.

### 2-Adamantan-1-yl-N-(4-chloro-benzyl)-acetamide (STX1927, XDS04058)

The title compound was synthesized with general amide formation method from 1-adamantane acetic acid (388 mg, 2.0 mmol) and the amine (0.245 mL, 2.0 mmol). White solid (350 mg, 55%) was obtained. mp 166-168° C.; TLC single spot at R_{f}: 0.69 (40% EtOAc/hexane); ¹H NMR (270 MHz, CDCl₃) δ 1.60-1.72 (12H, m, 6×CH₂), 1.95 (2H, s, CH₂), 1.96 (3H, s, 3×CH), 4.39 (2H, d, J=5.6 Hz, CH₂), 5.59 (1H, broad, NH) and 7.22-7.30 (4H, m, ArH); LC/MS (APCI) m/z 318 (M⁺+H); HPLC tᵣ=3.12 min (>99%) in 10% water-acetonitrile.

### 2-Adamantan-1-yl-N-(4-methyl-benzyl)-acetamide (STX1928, XDS04059)

The title compound was synthesized with general amide formation method from 1-adamantane acetic acid (388 mg, 2.0 mmol) and the amine (0.255 mL, 2.0 mmol). White solid (310 mg, 52%) was obtained. mp 139-142° C.; TLC single spot at R_{f}: 0.72 (40% EtOAc/hexane); ¹H NMR (270 MHz, CDCl₃) δ 1.61-1.71 (12H, m, 3×CH₂), 1.93 (2H, s, CH₂), 1.95 (3H, s, 3×CM), 2.33 (3H, s, CH₃), 4.38 (2H, d, J=5.7 Hz, CH₂), 5.52 (1H, broad, NH) and 7.11-7.19 (4H, m, ArH); LC/MS (APCI) m/z 298 (M⁺+H); HPLC tᵣ=3.06 min (>99%) in 10% water-acetonitrile.

### 1-Adamantan-1-yl-2-(thiophen-2-ylmethanesulfinyl)-ethanone (STX1929, XDS04060)

To a solution of 1-adamantan-1-yl-2-(thiophen-2-ylmethylsulfanyl)-ethanone (125 mg, 0.41 mmol) in DCM (12 mL) was added m-CPBA (108 mg, purity 60-77%). The mixture was stirred at −5° C. for 35 min, partitioned between ethyl acetate and 5% sodium carbonate solution. The organic phase was washed with brine, dried over sodium sulphate and concentrated in vacuo to give crude product. Purification with flash column hexane-ethyl acetate; gradient elution) yielded the title compound as white solid (115 mg, 87%). mp 95-98° C.; TLC single spot at R_{f}: 0.50 (60% EtOAc/DCM); ¹H NMR (270 MHz, CDCl₃) δ 1.62-1.81 (12H, m, 6×CH₂), 2.05 (3H, broad, 3×CH), 3.57 (1H, d, J=16 Hz, CH₂), 3.86 (1H, d, J=16 Hz, CH₂), 4.36 (2H, AB, CH₂), 7.02-7.07 (2H, m, ArH) and 7.33 (1H, dd, J=5.2, 1.5 Hz, ArH); LC/MS (APCI) m/z 321 (M⁺−H); HPLC tᵣ=2.66 min (99%) in 20% water-acetonitrile.

### 1-Adamantan-1-yl-2-(thiophene-2-sulfinyl)-ethanone (STX1930, XDS04061)

To a solution of 1-adamantan-1-yl-2-(thiophen-2-ylsulfanyl)-ethanone (170 mg, 0.58 mmol) in DCM (12 mL) was added m-CPBA (152 mg, purity 60-77%). The mixture was stirred at −5° C. for 30 min, partitioned between ethyl acetate and 5% sodium carbonate solution. The organic phase was washed with brine, dried over sodium sulphate and concentrated in vacuo to give the crude product. Purification with flash column (methanol-DCM; gradient elution) yielded the title compound as yellow solid (160 mg, 89%). mp 114-115.5° C.; TLC single spot at R_{f}: 0.15 (30% EtOAc/hexane); ¹H NMR (270 MHz, CDCl₃) δ 1.56-1.75 (12H, m, 6×CH₂), 2.03 (3H, broad, 3×CH), 4.06 (1H, d, J=15 Hz, CH₂), 4.39 (1H, d, J=15 Hz, CH₂), 7.10 (1H, dd, J=5.1, 3.6 Hz, ArH), 7.48 (1H, dd, J=3.4, 1.2 Hz, ArH) and 7.65 (1H, dd, J=5.0, 1.3 Hz, ArH); LC/MS (APCI) m/z 307 (M⁺−H); HPLC tᵣ=2.68 min (>99%) in 10% water-acetonitrile.

### 1-(4-Chlorophenyl)-N-methyl-N-(2-(thiophen-2-yl)ethyl)cyclopentanecarboxamide (STX1945, CCM02034A)

A suspension of 1-(4-chloro-phenyl)-cyclopentanecarboxylic acid (2-thiophen-2-yl-ethyl)-amide (70 mg, 0.21 mmol) with potassium tert-butoxide (90 mg, 0.8 mmol) and iodomethane (50 μL, 0.8 mmol) in dry DMF (3 mL) is stirred at room temperature for 3 days. The reaction mixture is poured in water and the aqueous layer is extracted with EtOAc. The combined organic layers are then washed with brine and dried over MgSO₄. After filtration, the crude product is purified by flash chromatography on silica gel (EtOAc/hexane 0/10 to 3/7) to give 1-(4-chlorophenyl)-N-methyl-N-(2-(thiophen-2-yl)ethyl)cyclopentane carboxamide (45 mg, 0.13 mmol, 62% yield) as a waxy white powder. ¹H NMR (400 MHz, CDCl₃) δ_{H} 1.68-1.79 (2H, m, CH₂ cyclopentane), 1.90-1.94 (2H, m, CH₂ cyclopentane), 2.34-2.39 (2H, m, CH₂ cyclopentane), 2.45 (3H, s, CH₃), 2.95 and 3.16-3.18 (2H, s and m, CH₂ cyclopentane), 3.05 (2H, t, J=7.2 Hz, CH₂-thiophene), 3.59 (2H, pseudo q, J=7.2 Hz, CH₂—N), 6.82-6.91 (2H, m, H_{Ar}thiophene), 7.07-7.19 (3H, m, H_{Ar}+H_{Ar}thiophene), 7.21-7.25 (2H, m, H_{Ar}); ¹³C NMR (67.5 MHz, CDCl₃) δ_{C} 24.9 and 25.3 (CH₂, rotamers), 27.4 and 27.9 (CH₂, rotamers), 33.3 and 36.7 (N—CH₃, rotamers), 38.4 (CH₂ cyclopentane), 51.2 and 51.6 (CH₂, rotamers), 58.3 (Cq-CO), 123.8 (CHAR thiophene), 125.4 (CH_{Ar} thiophene), 126.7 (CH_{Ar}), 127.0 (CH_{Ar}), 128.9 (CH_{Ar}), 131.9 (Cq_{Ar}), 141.5 (Cq_{Ar}), 144.1 (Cq_{Ar}), 175.4 (CO); LC/MS (AP+) m/z 348.2 (M+H); HPLC t_{R}=3.3 min (98.5%).

### 1-(4-Fluorophenyl)-N-methyl-N-((thiophen-2-yl)methyl)cyclopentanecarboxamide (STX1956, CCM02046)

Reaction of 1-(4-fluorophenyl)-1-cyclopentanecarboxylic acid (210 mg, 1.0 mmol) in DCM (10 mL) with N-methyl(thiophen-2-yl)methanamine (152 mg, 1.2 mmol) in presence of triethylamine (170 μL, 1.2 mmol), EDCI (230 mg, 1.2 mmol) and DMAP (147 mg, 1.2 mmol) according to the general procedure gives 1-(4-fluorophenyl)-N-methyl-N-((thiophen-2-yl)methyl)cyclopentanecarboxamide (190 mg, 0.60 mmol, 60%) as a white solid after purification by flash chromatography on silica gel (EtOAc/hexane 0/10 to 2/8). mp 72-75° C.; ¹H NMR (270 MHz, CDCl₃) δ_{H} 1.63-1.79 (4H, m, CH₂ cyclopentane), 1.91-1.98 (2H, m, CH₂ cyclopentane), 2.40-2.45 (2H, m, CH₂ cyclopentane), 2.51 (3H, s, N—CH₃), 4.67 (2H, s, N—CH₂), 6.89-6.98 (4H, m, H_{Ar}), 7.12-7.21 (3H, m, H_{Ar}); ¹³C NMR (67.5 MHz, CDCl₃) δ_{C} 25.2 (CH₂), 35.5 (CH₃), 38.5 (CH₂), 47.3 (CH₂), 58.1 (Cq), 115.6 (d, ²J_{C-F}=20.2 Hz, CH_{Ar}), 125.6 (CHA thiophene), 126.4 (CH_{Ar} thiophene), 126.5 (CH_{Ar} thiophene), 126.8 (d, ³J_{C-F}=6.8 Hz, CH_{Ar}), 139.9 (C_{qAr}), 141.1 (CqAr), 161.3 (d, ¹J_{C-F}=243 Hz, C_{qAr}), 175.4 (CO); LC/MS (AP+) m/z 318.2 (M+H); HPLC t_{R}=3.2 min (99.9%).

### 1-(4-Chlorophenyl)-N-methyl-N-((thiophen-2-yl)methyl)cyclohexanecarboxamide (STX1957, CCM02047)

Reaction of 1-(4-chlorophenyl)-1-cyclohexanecarboxylic acid (240 mg, 1.0 mmol) in DCM (10 mL) with N-methyl(thiophen-2-yl)methanamine (152 mg, 1.2 mmol) in presence of triethylamine (170 μL, 1.2 mmol), EDCI (230 mg, 1.2 mmol) and DMAP (147 mg, 1.2 mmol) according to the general procedure gives 1-(4-fluorophenyl)-N-methyl-N-((thiophen-2-yl)methyl)cyclopentanecarboxamide (200 mg, 0.57 mmol, 57%) as a white powder after purification by flash chromatography on silica gel (EtOAc/hexane 0/10 to 2/8). mp 93-95° C.; ¹H NMR (270 MHz, CDCl₃) δ_{H} 1.56-1.77 (8H, m, CH₂ cyclohexane), 1.91-1.98 (2H, m, CH₂ cyclopentane), 2.33 (2H, bd, J=12.4 Hz, CH₂ cyclohexane), 2.55 (3H, s, N—CH₃), 4.56 (2H, bs, N—CH₂), 6.85-6.81 (2H, m, H_{Ar}), 7.14-7.26 (5H, m, H_{Ar}); ¹³C NMR (67.5 MHz, CDCl₃) δ_{C} 23.7 (CH₂), 25.9 (CH₂), 35.6 (CH₃), 36.8 (CH₂), 48.1 (Cq), 51.0 (CH₂), 125.6 (CH_{Ar}), 126.4 (CH_{Ar}), 126.5 (CH_{Ar}), 126.8 (CH_{Ar}), 129.1 (CH_{Ar}), 132.2 (C_{qAr}), 140.0 (C_{qAr}), 144.7 (C_{qAr}), 174.3 (CO); LC/MS (AP+) m/z 348.2 (M+H); HPLC t_{R}=4.1 min (99.9%).

### Adamantane-1-carboxylic acid (4-chloro-benzyl)-methyl-amide (STX2002, XDS04062)

To a solution of adamantane-1-carboxylic acid 4-chloro-benzylamide (175 mg, 0.58 mmol) in anhydrous DMF (5 mL) was added NaH (60% dispersion, 175 mg, 4.38 mmol), followed by CH₃I (0.36 mL, 5.80 mmol). The mixture was stirred at ambient temperature for 24 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (100 mg, 54%). mp 65-66.5° C.; TLC single spot at R_{f}: 0.50 (30% hexane/DCM); ¹H NMR (270 MHz, CDCl₃) δ 1.71 (6H, S, 3×CH₂), 2.03 (9H, s, 3×CH₂ and 3×CH), 2.98 (3H, s CH₃), 4.61 (2H, s, CH₂) and 7.20 (4H, AB, ArH); LC/MS (APCI) m/z 318 (M⁺+H); HPLC tᵣ=6.23 min (>99%) in 10% water-acetonitrile.

### Adamantane-1-carboxylic acid methyl-(4-methyl-benzyl)-amide (STX2003, XDS04063)

To a solution of adamantane-1-carboxylic acid 4-methyl-benzylamide (164 mg, 0.58 mmol) in anhydrous DMF (5 mL) was added NaH (60% dispersion, 175 mg, 4.38 mmol), followed by CH₃I (0.36 mL, 5.80 mmol). The mixture was stirred at ambient temperature for 24 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (150 mg, 87%). mp 93-95° C.; TLC single spot at R_{f}: 0.57 (30% hexane/DCM); ¹H NMR (270 MHz, CDCl₃) δ 1.70 (6H, S, 3×CH₂), 2.04 (9H, s, 3×CH₂ and 3×CH), 2.32 (3H, s CH₃), 2.93 (3H, s CH₃), 4.65 (2H, s, CH₂) and 7.10 (4H, AB, ArH); LC/MS (APCI) m/z 298 (M⁺+H); HPLC tᵣ=5.87 min (>99%) in 10% water-acetonitrile.

### 2-Adamantan-1-yl-N-(4-chloro-benzyl)-N-methyl-acetamide (STX2004, XDS04064)

To a solution of 2-adamantan-1-yl-N-(4-chloro-benzyl)-acetamide (184 mg, 0.58 mmol) in anhydrous DMF (5 mL) was added NaH (60% dispersion, 175 mg, 4.38 mmol), followed by CH₃I (0.36 mL, 5.80 mmol). The mixture was stirred at ambient temperature for 24 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (150 mg, 78%). mp 75-76° C.; TLC single spot at R_{f}: 0.45 (30% hexane/DCM); ¹H NMR (270 MHz, CDCl₃) δ 1.59-1.72 (12H, m, 6×CH₂), 1.96 (3H, s, 3×CH), 2.19 (2H, s CH₂), 2.92 (3H, s CH₃), 4.55 (2H, s, CH₂) and 7.09-7.38 (4H, m, ArH); LC/MS (APCI) m/z 332 (M⁺+H); HPLC tᵣ=6.47 min (>99%) in 10% water-acetonitrile.

### 2-Adamantan-1-yl-N-methyl-N-(4-methyl-benzyl)-acetamide (STX2005, XDS04065)

To a solution of adamantane-2-adamantan-1-yl-N-(4-methyl-benzyl)-acetamide (172 mg, 0.58 mmol) in anhydrous DMF (5 mL) was added NaH (60% dispersion, 175 mg, 4.38 mmol), followed by CH₃I (0.36 mL, 5.80 mmol). The mixture was stirred at ambient temperature for 24 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as clear oil (150 mg, 83%). TLC single spot at R_{f}: 0.52 (30% hexane/DCM); ¹H NMR (270 MHz, CDCl₃) δ 1.68 (12H, m, 3×CH₂), 1.95 (3H, s, 3×CH), 2.18 (2H, s CH₂), 2.32 (3H, s CH₃), 2.91 (3H, s CH₃), 4.55 (2H, s, CH₂) and 7.01-7.17 (4H, m, ArH); LC/MS (APCI) m/z 312 (M⁺+H); HPLC tᵣ=6.45 min (>99%) in 10% water-acetonitrile.

### Adamantane-1-carboxylic acid ethyl-thiophen-2-ylmethyl-amide (STX2062, XDS04068)

To a solution of adamantane-1-carboxylic acid (thiophen-2-ylmethyl)-amide (180 mg, 0.65 mmol) in anhydrous DMF (5 mL) was added NaH (60% dispersion, 156 mg, 3.9 mmol), followed by bromoethane (0.28 mL, 3.9 mmol). The mixture was stirred at ambient temperature for 48 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (120 mg, 61%). mp 103-105° C.; TLC single spot at R_{f}: 0.68 (20% EtOAc/hexane); ¹H NMR (270 MHz, CDCl₃) δ 1.49 (3H, t, J=6.9 Hz, CH₃), 1.71 (6H, s, 3×CH₂), 2.02 (9H, s, 3×CH₂ and 3×CH), 3.48 (2H, q, J=7.0 Hz, CH₂), 4.72 (2H, s, CH₂), 6.90-6.92 (2H, m, ArH) and 7.19 (1H, dd, J=4.2, 2.0 Hz, ArH); -LC/MS (APCI) m/z 304 (M⁺+H); HPLC tᵣ=5.50 min (>99%) in methanol.

### Adamantane-1-carboxylic acid benzyl-thiophen-2-ylmethyl-amide (STX2063, XDS04069)

To a solution of adamantane-1-carboxylic acid (thiophen-2-ylmethyl)-amide (180 mg, 0.65 mmol) in anhydrous DMF (5 mL) was added NaH (60% dispersion, 156 mg, 3.9 mmol), followed by benzyl bromide (0.46 mL, 3.9 mmol). The mixture was stirred at ambient temperature for 48 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (160 mg, 67%). mp 130-132° C.; TLC single spot at R_{f}: 0.72 (20% EtOAc/hexane); ¹H NMR (270 MHz, CDCl₃) δ 1.64-1.75 (6H, m, 3×CH₂), 2.02 (3H, broad s, 3×CH), 2.07 (6H, s, 3×CH₂), 4.64 (2H, s, CH₂), 4.71 (2H, s, CH₂), 6.81 (1H, d, J=2.7 Hz, ArH), 6.91 (1H, dd, J=5.0, 3.5 Hz, ArH) and 7.17-7.38 (6H, m, ArH); LC/MS (APCI) m/z 225 (M⁺−H); HPLC tᵣ=5.74 min (>99%) in methanol.

### Adamantane-1-carboxylic acid cyclohexylmethyl-thiophen-2-ylmethyl-amide (STX2064, XDS04071A)

To a solution of adamantane-1-carboxylic acid (thiophen-2-ylmethyl)-amide (180 mg, 0.65 mmol) in anhydrous DMF (5 mL) was added NaH (60% dispersion, 156 mg, 3.9 mmol), followed by bromomethyl cyclohexane (0.54 mL, 3.9 mmol). The mixture was stirred at ambient temperature for 48 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (35 mg, 33%). Starting material (100 mg) recovered. mp 112-115.5° C.; TLC single spot at R_{f}: 0.69 (40% EtOAc/hexane); ¹H NMR (270 MHz, CDCl₃) δ 0.88 (2H, m, CH₂), 1.20 (4H, m, 2×CH₂), 1.58-1.98 (1H, m, 5×CH₂ and CH), 2.03 (9H, s, 3×CH₂ and 3×CH), 3.18 (2H, d, J=7.2 Hz, CH₂), 4.86 (2H, s, CH₂), 6.88 (1H, dd, J=3.5, 1.2 Hz, ArH), 6.93 (1H, dd, J=5.0, 3.5 Hz, ArH) and 7.19 (1H, dd, J=4.9, 1.2 Hz, ArH); LC/MS (APCI) m/z 372 (M⁺+H); HPLC tᵣ=7.32 min (98%) in methanol.

### Adamantan-1-yl-(4-pyridin-2-yl-piperazin-1-yl)-methanone (STX2065, XDS04076)

The title compound was synthesized with general amide formation method from 1-adamantane carbonyl chloride (10 mg, 0.5 mmol) and the amine (0.073 mL, 0.5 mmol). White solid (142 mg, 87%) was obtained. mp 137-140° C.; TLC single spot at R_{f}: 0.49 (40% ethyl acetate/hexane); ¹H NMR (270 MHz, CDCl₃) δ 1.67-1.75 (6H, m, 3×CH₂), 2.09 (9H, s, 3×CH₂ and 3×CH), 3.50 (4H, dd, J=5.5, 3.2 Hz, 2×CH₂), 3.81 (4H, dd, J=5.5, 3.2 Hz, 2×CH₂), 6.63-6.68 (2H, m, ArH), 7.49 (1H, m, ArH) and 8.19 (1H, m, ArH); LC/MS (APCI) m/z 326 (M⁺+H); HPLC tᵣ=5.67 min (>99%) in 10% water-acetonitrile.

### 2-Adamantan-1-yl-1-(4-pyridin-2-yl-piperazin-1-yl)-ethanone (STX2066, XDS04077)

The title compound was synthesized with general amide formation method from 1-adamantane acetic acid (97 mg, 0.5 mmol) and the amine (0.073 mL, 0.5 mmol). White solid (150 mg, 88%) was obtained. mp 134-136° C.; TLC single spot at R_{f}: 0.42 (40% ethyl acetate/hexane); ¹H NMR (270 MHz, CDCl₃) δ 1.60-1.67 (12H, m, 6×CH₂), 1.96 (3H, s, 3×CH), 3.48 (2H, s CH₂), 3.62 (4H, m, 2×CH₂), 3.77 (4H, m, 2×CH₂), 6.63-6.67 (2H, m, ArH), 7.49 (1H, m, ArH) and 8.19 (1H, m, ArH); -LC/MS (APCI) m/z 340 (M⁺+H); HPLC tᵣ=5.68 min (>99%) in 10% water-acetonitrile.

### Adamantane-1-carboxylic acid methyl-(5-methyl-thiophen-2-ylmethyl)-amide (STX2067, XDS04078)

The title compound was synthesized with general amide formation method from 1-adamantane carbonyl chloride (100 mg, 0.5 mmol) and the amine (71 mg, 0.5 mmol). White solid (111 mg, 73%) was obtained. mp 83-85.5° C.; TLC single spot at R_{f}: 0.72 (30% ethyl acetate/hexane); ¹H NMR (270 MHz, CDCl₃) δ 1.70 (6H, s, 3×CH₂), 2.03 (9H, s, 3×CH₂ and 3×CH), 2.42 (3H, d, J=1.0 Hz, CH₃), 3.05 (3H, s, CH₃), 4.64 (2H, s, CH₂), 6.55 (1H, dq, J=3.3, 1.0 Hz, ArH) and 6.68 (1H, d, J=3.3 Hz, ArH); LC/MS (APCI) m/z 304 (M⁺+H); HPLC tᵣ=6.95 min (98.6%) in 10% water-acetonitrile.

### 2-Adamantan-1-yl-N-methyl-N-(5-methyl-thiophen-2-ylmethyl)-acetamide (STX2068, XDS04079)

The title compound was synthesized with general amide formation method from 1-adamantane acetic acid (97 mg, 0.5 mmol) and the amine (71 mg, 0.5 mmol). A clear oil (110 mg, 69%) was obtained. TLC single spot at R_{f}: 0.67 (30% ethyl acetate/hexane); ¹H NMR (270 MHz, CDCl₃) δ 1.60-1.67 (12H, s, 6×CH₂), 1.95 (3H, s, 3×CH), 2.14 (2H, s, CH₂), 2.42 (3H, s, CH₃), 2.98 (3H, s, CH₃), 4.61 (2H, s, CH₂), 6.55 (1H, m, ArH) and 6.72 (1H, d, J=3.3 Hz, ArH); LC/MS (APCI) m/z 318 (M+11); HPLC tᵣ=7.49 min (98%) in 10% water-acetonitrile.

### 1-Adamantan-1-yl-2-(2-methyl-benzothiazol-5-yloxy)-ethanone (STX2073, FPC01004)

The solution of 1-adamatyl bromomethyl ketone (150 mg, 0.58 mmol), 2-methyl-5-benzothiazolol (106 mg, 0.64 mmol) and potassium carbonate (160 mg, 1.16 mmol) in acetone (6 mL) was stirred at room temperature over 48 h and partitioned between ethyl acetate and a saturated sodium bicarbonate solution. The organic phase was washed with water then brine, dried over magnesium sulphate and concentrated in vacuo. Purification of the residue by flash chromatography (hexane/EtOAc; gradient elution) gave the titled compound as white solid (198 mg, 100%). TLC single spot at R_{f}: 0.23 (DCM/methanol 9:1); ¹H NMR (270 MHz, CDCl₃): δ 7.66 (1H, d, J=8.9 Hz), 7.28 (1H, d, J=2.5 Hz), 7.04 (1H, dd, J=2.5 Hz), 4.94 (2H, s), 2.78 (3H, s), 2.11-2.04 (3H, m) 1.93 (6H, broad d, J=3 Hz), 1.79-1.71 (6H, m); LC/MS (APCI) m/z 342.44 (M⁺+1); HPLC tᵣ=18.76 min (100%) in 10% water-acetonitrile.

### 1-Adamantan-1-yl-2-(4-tert-butyl-benzylsulfanyl)-ethanone (STX2081, FPC01005B)

To a solution of 1-adamatyl bromomethyl ketone (100 mg, 0.39 mmol) and 4-tertbutylbenzyl mercaptan (0.726 mL, 0.39 mmol) in CH₃CN (2.5 mL) was added triethylamine (0.108 mL, 0.78 mmol) at room temperature. When the staring materials were consumed, the reaction was partitioned between Ethyl acetate and water. The organic phase was washed with brine, dried over magnesium sulphate and concentrated in vacuo. Purification of the residue by flash chromatography (hexane/EtOAc gradient elution) gave the titled compound as white solid (131.6 mg, 95%). TLC single spot at R_{f} 0.41 (DCM/EtOAc 95:5); ¹H NMR (270 MHz, CDCl₃): δ 7.34-7.22 (4H, m), 3.68 (2H, s), 3.17 (2H, s), 2.01 (3H, br s), 1.80 (6H, m), 1.75-1.54 (6H, m), 1.29 (9H, s); LC/MS (APCI) m/z 357.62 (M⁺+H); HPLC tᵣ=14.86 nm in (100% purity) in 10% water-acetonitrile.

### 1-Adamantan-1-yl-2-(2,4-dichloro-benzylsulfanyl)-ethanone (STX2082, FPC01006B)

To a solution of 1-adamatyl bromomethyl ketone (100 mg, 0.39 mmol) and 2,4-dichlorobenzyl mercaptan (0.55 mL, 0.39 mmol) in CH₃CN (2.5 mL) was added triethylamine (0.108 mL, 0.78 mmol) at room temperature. When the staring materials were consumed, the reaction partitioned between ethyl acetate and water. The organic phase was with brine, dried over magnesium sulphate and concentrated in vacuo. Purification of the residue by flash chromatography (hexane/EtOAc gradient elution) gave the titled compound as transparent wax (127 mg, 88%). TLC single spot at R_{f} 0.16 (DCM/EtOAc 95:5); ¹H NMR (270 MHz, CDCl₃): δ 7.38 (1H, d, J=2.0 Hz), 7.33 (1H, d, J=8.0 Hz), 7.19 (1H, dd, J=2.0, 8.0 Hz), 3.79 (2H, s), 3.26 (2H, s), 2.03 (3H, br s), 1.83 (6H, d, J=3.0 Hz), 1.78-1.62 (6H, m); LC/MS (APCI) m/z 369.52 (M⁺+H); HPLC tᵣ=12.93 min (99.93% purity) in 10% water-acetonitrile.

### 1-Adamantan-1-yl-2-(4-tert-butyl-phenylmethanesulfinyl)-ethanone (STX2083, FPC01008)

To a solution of 1-adamantan-1-yl-2-(4-tert-butyl-benzylsulfanyl)-ethanone (0.29 mmol) in DCM (5 mL) was added m-CPBA (97 mg, 0.44 mmol, 77% max) at 0° C. and stirred vigorously for 45 min then partitioned between dichloromethane and a saturated solution of sodium carbonate. The organic phase was washed with water then brine, dried over magnesium sulphate and concentrated in vacuo. Purification of the residue by flash chromatography (DCM/EtOAc gradient elution) gave the titled compound as a white solid (21.2 mg, 20%). TLC single spot at R_{f} 0.15 (hexane/EtOAc 7:3); ¹H NMR (270 MHz, CDCl₃): δ 7.37 (1H, d, J=8.2 Hz), 7.21 (1H, d, J=8.4 Hz), 4.20 (1H, d, J=13.0 Hz), 4.05 (1H, d, J=13.0 Hz), 3.86 (1H, d, J=15.6 Hz), 3.58 (1H, d, J=15.6 Hz), 2.03 (3H, m), 1.79-1.61 (12H, m), 1.27 (9H, s); LC/MS (APCI) m/z 373.61 (M⁺+11); HPLC tᵣ=7.13 min (99.57% purity) in 10% water-acetonitrile.

### 1-Adamantan-1-yl-2-(2,4-dichloro-benzylsulfanyl)-ethanone (STX2084, FPC01009)

To a solution of 1-adamantan-1-yl-2-(2,4-dichloro-benzylsulfanyl)-ethanone (97.3 mg, 0.26 mmol) in DCM (5 mL) was added m-CPBA (90 mg, 0.40 mmol, 77% max) was added at 0° C. and stirred vigorously for 45 min then partitioned between dichloromethane and a saturated solution of sodium carbonate. The organic phase was washed with water then brine, dried over magnesium sulphate and concentrated in vacuo. Purification of the residue by flash chromatography (DCM/EtOAc, gradient elution) gave the expected titled compound as a white solid (56.4 mg, 56%). TLC single spot at R_{f} 0.15 (hexane/EtOAc 7:3); ¹H NMR (270 MHz, CDCl₃): δ 7.46 (1H, d, J=2.0 Hz), 7.36 (1H, d, J=8.4 Hz), 7.28 (1H, d, J=2.0 Hz), 4.42 (1H, d, J=12.9 Hz), 4.12 (1H, d, J=13.1 Hz), 3.95 (1H, d, J=15.6 Hz), 3.78 (1H, d, J=15.6 Hz), 2.07 (3H, m), 1.85-1.64 (12H, m); LC/MS (APCI) m/z 385.51 (M⁺); HPLC tᵣ=6.19 min (99.19% purity) in 10% water-acetonitrile.

### 1-Adamantan-1-yl-2-(6-methyl-pyridin-3-ylmethoxy)-ethanone (STX2094, FPC01023)

A mixture of 1-adamatyl bromomethyl ketone (150 mg, 0.58 mmol), 5-hydroxy-2-methylpyridine (63 mg, 0.58 mmol) and potassium carbonate (160 mg, 1.16 mmol) in acetone (6 mL) was stirred at room temperature over night. The reaction was partitioned between ethyl acetate and water. The organic phase was washed with brine, dried over magnesium sulphate and concentrated in vacuo. Purification of the residue by flash chromatography (hexane/EtOAc, gradient elution) gave the expected titled compound as yellow solid (132 mg, 80%). TLC single spot at R_{f} 0.08 (hexane/EtOAc 8:2); ¹H NMR (270 MHz, CDCl₃): δ 8.12 (1H, dd, J=1.2, 2.5 Hz), 7.10-7.00 (2H, m), 4.87 (2H, s), 2.47 (3H, s), 2.11-2.02 (3H, br s), 1.90 (6H, broad d, J=3 Hz), 1.82-1.67 (6H, m); LC/MS (APCI) m/z 286.39 (M⁺+H); HPLC tᵣ=6.15 min (95.57% purity) in 10% water-acetonitrile;

### 5-Chloro-3-methyl-benzo[b]thiophene-2-sulfonic acid (thiophen-2-ylmethyl)-amide (STX2095, FPC01014C)

Pyridine (0.058 mL, 0.72 mmol) was added to a solution of 5-Chloro-3-methyl-benzo[b]thiophene-2-sulfonyl chloride (100 mg, 0.36 mmol) and 2-thiophen methyl amine (0.037 mL, 0.36 mmol) in DCM (5 mL) at room temperature. The reaction was allowed to stir overnight and partitioned between ethyl acetate and water. The organic phase was washed with brine, dried over MgSO₄, filtrated and concentrated in vacuo. Purification of the residue by flash chromatography (DCM/methanol, gradient elution) gave the expected titled compound as a yellow solid (113 mg, 87%). TLC single spot at R_{f} 0.93 (DCM/MeOH 9:1); ¹H NMR (270 MHz, CDCl₃): δ 7.80-7.70 (2H, m), 7.45 (1H, dd, J=2.0, 8.4 Hz), 7.15 (1H, dd, J=1.7, 4.7 Hz), 6.85-6.81 (2H, m), 4.95 (1H, br d, J=5.2 Hz), 4.47 (2H, d, J=4.5 Hz), 2.61 (3H, s); LC/MS (APCI) m/z 356.38 (M⁺−H), 358.31 (M⁺+H); HPLC tᵣ=4.81 min (100% purity) in 10% water-acetonitrile.

### Adamantane-t-carboxylic acid isopropyl-thiophen-2-ylmethyl-amide (STX2115, XDS04082)

To a cold solution of adamantane-1-carboxylic acid (thiophen-2-ylmethyl)-amide (180 mg, 0.65 mmol) in anhydrous THF (10 mL) was added n-BuLi (2.5 MHz, 1.0 mL, 2.5 mmol). After stirred at −5° C. for 5 min, 2-bromoisopropane (0.235 mL, 2.5 mmol) was added. The mixture was stirred at ambient temperature for 40 min, quenched with 4N HCl, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (65 mg, 32%). mp 156-158° C.;. TLC single spot at R_{f}: 0.55 (15% EtOAc/hexane); ¹H NMR (270 MHz, CDCl₃) δ 0.92 (6H, d, J=6.8 Hz, 2×CH₃), 1.66-1.77 (6H, m, 3×CH₂), 1.86 (6H, d, J=3.0 Hz, 3×CH₂), 2.04 (3H, s, 3×CH), 2.10 (1H, septet, J=6.7 Hz, CH), 5.15 (1H, dd, J=8.5, 6.4 Hz, CH), 5.80 (1H, broad d, J=8.9 Hz, CH), 6.85-6.93 (1H, m, ArH) and 7.16 (1H, dd, J=4.9, 3.7 Hz, ArH), LC/MS (APCI) m/z 318 (M⁺+H); HPLC tᵣ=6.80 min (90%) in methanol.

### (Adamantan-1-ylsulfanyl)-acetic acid (XDS04074B)

The mixture of 1-bromoadamantane (2 g, 9.35 mmol) and 3-mercapto-propionic acid ethyl ester (2.2 g, 16.4 mmol) in acetic acid (25 mL) was refluxed for 18 h, cooled to room temperature and concentrated in vacuo. The crude oil was partitioned between ethyl acetate and brine. The organic phase was washed with brine, dried over sodium sulphate and concentrated in vacuo to give a residue, which was dissolved in methanol-THF (30 mL-10 mL). LiOH (550 mg) was added to the solution. After 4 h at ambient temperature, the mixture was neutralized to pH 6.5 with 4N HCl and extracted with ethyl acetate. The organic phase was washed with brine, dried over sodium sulphate and concentrated in vacuo to yield the title compound as white solid (0.5 g, 24%). mp 71-73° C.; TIC single spot at R_{f}: 0.29 (30% ethyl acetate/DCM); ¹H NMR (270 ME, CDCl₃) δ 1.62-1.68 (6H, m, 3×CH₂), 1.85 (6H, d, J=2.2 MHz, 3×CH₂), 2.05 (3H, broad s, 3×CH) and 3.30 (2H, s, CH₂); -LC/MS (APCI) m/z 227 (M⁺+H).

### 2-(Adamantan-1-ylsulfanyl)-N-methyl-N-thiophen-2-ylmethyl-acetamide (STX2116, XDS04086)

The title compound was synthesized with general amide formation method from (Adamantan-1-ylsulfanyl)-acetic acid (250 mg, 1.1 mmol) and the amine (134 mg, 1.05 mmol). The product (160 mg, 45%) was obtained as yellow solid. mp 51-53° C.; TLC single spot at R_{f}: 0.56 (30% ethyl acetate/hexane); ¹H NMR (270 MHz, CDCl₃) δ 1.67 (6H, m, 3×CH₂), 1.86 (6H, d, J=2.7 Hz, 3×CH₂), 2.03 (3H, s, 3×CH), 3.03 (3H, s, CH₃), 3.34 (2H, s, CH₂), 4.69 (2H, s, CH₂), 6.91-6.98 (2H, m, ArH) and 7.21 (1H, dd, J=5.0, 1.5 Hz, ArH); LC/MS (APCI) m/z 336 (H⁺+H); HPLC tᵣ=5.14 min (98%) in 10% water-acetonitrile.

### Adamantan-1-yl-[4-(4-chloro-benzyl)-piperazin-1-yl]-methanone (STX2118, XDS04090)

The title compound was synthesized with general amide formation method from 1-adamantane carbonyl chloride (100 mg, 0.5 mmol) and the amine (105 mg, 0.5 mmol). White solid (140 mg, 75%) was obtained. mp 141.5-143° C.; TLC single spot at R_{f}: 0.25 (30% ethyl acetate/hexane); ¹H NMR (270 MHz, CDCl₃) δ 1.64-1.68 (6H, m, 3×CH₂), 1.96 (6H, d, J=2.7 Hz, 3×CH₂), 2.01 (3H, s, 3×CH), 2.38 (4H, t, J=4.9 Hz, 2×CH₂), 3.44 (2H, s, CH₂), 3.67 (4H, t, J=4.9 Hz, 2×CH₂) and 7.20-7.26 (4H, m, ArH); LC/MS (APCI) m/z 373 (M⁺+H); HPLC tᵣ=8.39 min (>99%) in 10% water-acetonitrile.

### Adamantan-1-yl-[4-(3-methyl-benzyl)piperazin-1-yl]-methanone (STX2119, XDS04091)

The title compound was synthesized with general amide formation method from 1-adamantane carbonyl chloride (100 mg, 0.5 mmol) and the amine (95 mg, 0.5 mmol). White solid (140 mg, 75%) was obtained. mp 185-187° C.; TLC single spot at R_{f}: 0.26 (40% ethyl acetate/hexane); ¹H NMR (270 MHz, CDCl₃) δ 1.64-1.74 (6H, m, 3×CH₂), 1.97 (6H, s, 3×CH₂), 2.01 (3H, s, 3×CHI), 2.33 (3H, s, CH₃), 2.40 (4H, t, J=4.6 Hz, 2×CH₂), 3.45 (2H, s, CH₂), 3.68 (4H, t, J=4.6 Hz, 2×CH₂) and 7.04-7.22 (4H, m, ArH); LC/MS (APCI) m/z 353 (M⁺+H); HPLC tᵣ=7.91 min (>99%) in 10% water-acetonitrile.

### Adamantan-1-yl-[4-(4-chloro-phenyl)-piperazin-1-yl]-methanone (STX2120, XDS04092)

The title compound was synthesized with general amide formation method from 1-adamantane carbonyl chloride (100 mg, 0.5 mmol) and the amine HCl salt (135 mg, 0.5 mmol). White solid (140 mg, 78%) was obtained. mp 183-185° C.; TLC single spot at R_{f}: 0.50 (30% ethyl acetate/hexane); ¹H NMR (270 MHz, CDCl₃) δ 1.72 (6H, broad, 3×CH₂), 2.01 (6H, s, 3×CH₂), 2.03 (3H, s, 3×CH), 3.10 (4H, t, J=5.2 Hz, 2×CH₂), 3.83 (4H, t, J=5.2 Hz, 2×CH₂), 6.82 (2H, dt, J=9.1, 2.2 Hz, ArH) and 7.21 (2H, dt, J=9.1, 2.2 Hz, ArH); LC/MS (APCI) m/z 359 (M⁺+H); HPLC tᵣ=7.63 min (>99%) in 10% water-acetonitrile.

### Adamantane-1-carboxylic acid (5-chloro-thiophen-2-ylmethyl)-methyl-amide (STX2121, XDS04093)

The title compound was synthesized with general amide formation method from 1-adamantane carbonyl chloride (10 mg, 0.5 mmol) and the amine (81 mg, 0.5 mmol). White solid (120 mg, 74%) was obtained. mp 92-94° C.; TLC single spot at R_{f}: 0.69 (30% ethyl acetate/hexane); ¹H NMR (270 MHz, CDCl₃) δ 1.75 (6H, s, 3×CH₂), 2.01 (9H, s, 3×CH₂ and 3×CH), 3.10 (3H, s, CH₃), 4.55 (2H, s, CH₂) and 6.70 (2H, AB, ArH); LC/MS (APCI) m/z 324 (M⁺+H); HPLC tᵣ=9.69 min (>99%) in 10% water-acetonitrile.

### Adamantane-1-carboxylic acid (5-ethyl-thiophen-2-ylmethyl)-methyl-amide (STX2122, XDS04094)

The title compound was synthesized with general amide formation method from 1-adamantane carbonyl chloride (100 mg, 0.5 mmol) and the amine (78 mg, 0.5 mmol). White solid (138 mg, 87%) was obtained. mp 58-60° C.; TLC single spot at R_{f}: 0.72 (30% ethyl acetate/hexane); ¹H NMR (270 MHz, CDCl₃) δ 1.24 (3H, t, J=6.7 Hz, CH₃), 1.70 (6H, s, 3×CH₂), 2.03 (9H, s, 3×CH₂ and 3×CH), 2.77 (2H, q, J=6.7 Hz, CH₂), 3.05 (3H, s, CH₃), 4.65 (2H, s, CH₂), 6.59 (1H, d, J=3.4 Hz, ArH) and 6.71 (1H, d, J=3.4 Hz, ArH); LC/MS (APCI) m/z 318 (M⁺+H); HPLC tᵣ=10.2 min (>99%) in 10% water-acetonitrile.

### Adamantane-1-carboxylic acid methyl-(5-methylsulfanyl-thiophen-2-ylmethyl)-amide (STX2123, XDS04095)

The title compound was synthesized with general amide formation method from 1-adamantane carbonyl chloride (300 mg, 1.5 mmol) and the amine (260 mg, 1.5 mmol). White solid (320 mg, 64%) was obtained. mp 73-75.5° C.; TLC single spot at R_{f}: 0.62 (30% ethyl acetate/hexane); ¹H NMR (270 MHz, CDCl₃) δ 1.71 (6H, s, 3×CH₂), 2.02 (9H, s, 3×CH₂ and 3×CH), 2.45 (3H, s, CH₃), 3.09 (3H, s, CH₃), 4.63 (2H, s, CH₂), 6.75 (1H, d, J=3.4 Hz, ArH) and 6.88 (1H, d, J=3.4 Hz, ArH); LC/MS (APCI) m/z 336 (M⁺+H); HPLC tᵣ=9.38 min (>99%) in 10% water-acetonitrile.

### (Adamantane-1-sulfonyl)-acetic acid (XDS04048)

To a solution of (adamantan-1-ylsulfanyl)-acetic acid (860 mg, 3.8 mmol) in DCM (50 mL) was added m-CPBA (2 g, purity 60-77%). The mixture was stirred at ambient temperature for 20 h, partitioned between ethyl acetate and brine. The organic phase was washed with brine, dried over sodium sulphate and concentrated in vacuo to give the crude product. Purification with flash column (methanol-DCM; gradient elution) yielded the title compound as white solid (900 mg, 96%). mp 158-162° C.; TLC single spot at R_{f}: 0.16 (10% methanol/DCM); ¹H NMR (270 MHz, CDCl₃) δ 1.60-1.69 (6H, m, 3×CH₂), 1.94 (6H, d, J=2.2 Hz, 3×CH₂), 2.08 (3H, broad s, 3×CH) and 3.70 (2H, s, CH₂); LC/MS (APCI) m/z 257 (M⁺−H).

### 2-(Adamantane-1-sulfonyl)-N-methyl-N-thiophen-2-ylmethyl-acetamide (STX2124, XDS04097)

The title compound was synthesized with general amide formation method from (adamantane-1-sulfonyl)-acetic acid (90 mg, 0.36 mmol) and the amine (90 mg, 0.70 mmol). The product (25 mg, 19%) was obtained as white solid. mp 120-122° C.; TLC single spot at R_{f}: 0.28 (30% ethyl acetate/hexane); ¹H NMR (270 MHz, CDCl₃) δ 1.72 (6H, m, 3×CH₂), 2.09 (6H, d, J=2.6 Hz, 3×CH₂), 2.18 (3H, s, 3×CH), 3.17 (3H, s, CH₃), 4.05 (2H, s, CH₂), 4.74 (2H, s, CH₂), 6.91-6.94 (2H, m, ArH) and 7.22 (1H, dd, J=5.0, 1.3 Hz, ArH); LC/MS (APCI) m/z 366 (M⁺−H); HPLC tᵣ=4.44 min (>99%) in 10% water-acetonitrile.

### 2-(Adamantane-1-sulfonyl)N-(6-methyl-pyridin-2-yl)acetamide (STX2125, XDS04098)

The title compound was synthesized with general amide formation method from (adamantane-1-sulfonyl)-acetic acid (90 mg, 0.36 mmol) and the amine (90 mg, 0.83 mol). The product (28 mg, 22%) was obtained as clear oil, which turned into white solid upon treatment with ether. mp 126-129° C.; TLC single spot at R_{f}: 0.36 (30% ethyl acetate/hexane); ¹H NMR (270 MHz, CDCl₃) δ 1.71 (6H, m, 3×CH₂), 2.07 (6H, s, 3×CH₂), 2.17 (3H, s, 3×CH), 2.43 (3H, s, CH₃), 3.93 (2H, s, CH₂), 6.91 (1H, d, J=8.1 Hz, ArH), 7.57 (1H, t, J=8.1 Hz, ArH), 7.89 (1H, d, J=8.1 Hz, ArH) and 9.09 (1H, broad, NH); LC/MS (APCI) m/z 349 (M⁺+H); HPLC tᵣ=4.70 min (97%) in 10% water-acetonitrile.

### 5-{[(Adamantane-1-carbonyl)methyl-amino]-methyl}-thiophene-2-carboxylic acid methylamide (STX2126, XDS04102P)

The title compound was synthesized with general amide formation procedure from 1-adamantane carbonyl chloride (200 mg, 1.0 mmol) and the amine (185 mg, 1.0 mmol). White solid (280 mg, 81%) was obtained. mp 211-211.5° C.; TLC single spot at R_{f}: 0.69 (6% methanol/DCM); ¹H NMR (270 MHz, CDCl₃) δ 1.66 (6H, s, 3×CH₂), 1.97 (9H, s, 3×CH₂ and 3×CH), 2.89 (3H, d, J=4.7 Hz, CH₃), 3.08 (3H, s, CH₃), 4.62 (2H, s, CH₂), 6.69 (1H, q, J=4.7 Hz, NH), 6.80 (1H, d, J=3.7 Hz, ArH) and 6.38 (1H, d, J=3.7 Hz, ArH); LC/MS (APCI) m/z 347 (M⁺+H); HPLC tᵣ=4.57 min (>99%) in 10% water-acetonitrile.

### 1-(Adamantane-1-carbonyl)-piperidine-4-carboxylic acid ethyl ester (XDS04103P)

The title compound was synthesized with general amide formation method from 1-adamantane carbonyl chloride (1.6 g, 8.0 mmol) and the amine (1.24 mL, 8.0 mmol). White solid (2.55 g, 99%) was obtained. The analytical sample (150 mg) was obtained by flash column chromatography of 200 mg crude sample. mp 112-114° C.; TLC single spot at R_{f}: 0.52 (30% ethyl acetate/hexane); ¹H NMR (270 MHz, CDCl₃) δ 1.24 (3H, t, J=7.1 Hz, CH₃), 1.56-1.66 (2H, m, CH₂), 1.70 (6H, s, 3×CH₂), 1.90 (2H, m, CH₂), 1.98 (6H, s, 3×CH₂), 2.02 (3H, s, 3×CH), 2.52 (1H, tt, J=10.9, 4.2 Hz, 2×CH₂), 2.95 (1H, td, J=13.6, 2.5 Hz, 2×CH), 4.14 (2H, q, J=7.1 Hz, CH₂) and 4.37 (2H, dt, J=13.4, 3.3 Hz, 2×CH); LC/MS (APCI) m/z 320 (M⁺+H); HPLC tᵣ=5.95 min (>99%) in 10% water-acetonitrile.

### 1-(5-chloro-3-methylbenzo[b]thiophen-2-yl)-2-(4-chlorobenzylthio)ethanone (STX2128, XDS04104P)

The solution of 2-bromo-1-(5-chloro-3-methylbenzo[b]thiophen-2-yl)ethanone (340 mg, 1.12 mmol) and (4-chloro-phenyl)-methanethiol (213 mg, 1.34 mmol) in acetonitrile/triethylamine (6 mL: 1 mL) was stirred at ambient temperature for 12 h, and then at 70° C. for 24 h. After cooling to room temperature, the mixture was partitioned between ethyl acetate and 1N HCl solution. The organic phase was washed with 1N NaOH and brine, dried over sodium sulphate and concentrated in vacuo to give the crude product. Purification with flash column (hexane-EtOAc; gradient elution) yielded the title compound as off-white crystalline solid (280 mg, 66%). mp 123-124° C.; TLC single spot at R_{f}: 0.71 (10% EtOAc/DCM); ¹H NMR (300 MHz, CDCl₃) δ 2.71 (3H, s, CH₃), 3.54 (2H, s, CH₂), 3.75 (2H, s, CH₂), 7.25-7.32 (4H, m, ArH), 7.45 (1H, dd, J=8.6, 1.9 Hz, ArH), 7.74 (1H, d, J=8.7 Hz, ArH) and 7.83 (1H, d, J=1.9 Hz, ArH); LC/MS (APCI) m/z 379 (MN-H); HPLC tᵣ=12.2 min (>98%) in 10% water-acetonitrile.

### Adamantane-1-carboxylic acid methyl-(3-methyl-thiophen-2-ylmethyl)-amide (STX2130, XDS04109)

The title compound was synthesized with general amide formation procedure from 1-adamantane carbonyl chloride (10 mg, 0.50 mmol) and the amine (106 mg, 0.75 mmol). White solid (140 mg, 92%) was obtained. mp 72-73.5° C.; TLC single spot at R_{f}: 0.70 (30% EtOAc/hexane); ¹H NMR (270 MHz, CDCl₃) δ 1.71 (6H, s, 3×CH₂), 2.04 (9H, s, 3×CH₂ and 3×CH), 2.20 (3H, s, CH₃), 3.05 (3H, s, CH₃), 4.68 (2H, s, CH₂), 6.77 (1H, d, J=5.2 Hz, ArH) and 7.10 (1H, d, J=5.2 Hz, ArH); LC/MS (APCI) m/z 304 (M⁺+H); HPLC tᵣ=8.06 min (>99%) in 10% water-acetonitrile.

### Adamantane-1-carboxylic acid (4-bromo-thiophen-2-ylmethyl)-methyl-amide (STX2131, XDS04110)

The title compound was synthesized with general amide formation procedure from 1-adamantane carbonyl chloride (100 mg, 0.50 mmol) and the amine (155 mg, 0.75 mmol). White solid (160 mg, 87%) was obtained. mp 107-109° C.; TLC single spot at R_{f}: 0.70 (30% EtOAc/hexane); ¹H NMR (270 MHz, CDCl₃) δ 1.71 (6H, s, 3×CH₂), 2.02 (9H, s, 3×CH₂ and 3×CH), 3.11 (3H, s, CH₃), 4.64 (2H, s, CH₂), 6.83 (1H, m, ArH) and 7.10 (1H, d, J=1.5 Hz, ArH); LC/MS (APCI) m/z 368 (M⁺+H); HPLC tᵣ=8.90 min (>99%) in 10% water-acetonitrile.

### Adamantane-1-carboxylic acid methyl-thiophen-3-ylmethyl-amide (STX2132, XDS04111)

The title compound was synthesized with general amide formation procedure from 1-adamantane carbonyl chloride (100 mg, 0.50 mmol) and the amine (95 mg, 0.75 mmol). The product (125 mg, 86%) was obtained as clear oil. TLC single spot at R_{f}: 0.55 (30% EtOAc/hexane); ¹H NMR (270 MHz, CDCl₃) δ 1.70 (6H, s, 3×CH₂), 2.03 (9H, s, 3×CH₂ and 3×CH), 3.00 (3H, s, CH₃), 4.63 (2H, s, CH₂), 6.95 (1H, dd, J=4.9, 1.2 Hz, ArH), 7.05 (1H, dd, J=3.0, 1.2 Hz, ArH) and 7.27 (1H, dd, J=4.9, 3.0 Hz, ArH); LC/MS (APCI) m/z 290 (M⁺+H); HPLC tᵣ=6.87 min (>99%) in 10% water-acetonitrile.

### Adamantane-1-carboxylic acid methyl-(1-methyl-1H-pyrrol-2-ylmethyl)-amide (STX2133, XDS04112)

The title compound was synthesized with general amide formation procedure from 1-adamantane carbonyl chloride (100 mg, 0.50 mmol) and the amine (93 mg, 0.75 mmol). The product (125 mg, 87%) was obtained as white solid. mp 104-105.5° C.; TLC single spot at R_{f}: 0.63 (30% EtOAc/hexane); ¹H NMR (270 MHz, CDCl₃) δ 1.76 (6H, s, 3×CH₂), 2.02 (9H, s, 3×CH₂ and 3×CH), 3.02 (3H, s, CH₃), 3.50 (3H, s, CH₃), 4.61 (2H, s, CH₂), 6.03 (2H, d, J=2.3 Hz, ArH) and 6.58 (1H, t, J=2.2 Hz, ArH); LC/MS (APCI) m/z 287 (M⁺+H); HPLC tᵣ=6.58 min (>99%) in 10% water-acetonitrile.

### Adamantane-1-carboxylic acid methyl-(6-methyl-pyridin-2-ylmethyl)-amide (STX2134, XDS04113)

The title compound was synthesized with general amide formation procedure from 1-adamantane carbonyl chloride (100 mg, 0.50 mmol) and the amine (102 mg, 0.75 mmol). The product (120 mg, 80%) was obtained as white solid. mp 76-77.5° C.; TLC single spot at R_{f}: 0.70 (10% methanol/DCM); ¹H NMR (270 MHz, CDCl₃) δ 1.69 (6H, s, 3×CH₂), 2.03 (9H, s, 3×CH₂ and 3×CH), 2.53 (3H, s, CH₃), 3.06 (3H, s, CH₃), 4.76 (2H, s, CH₂), 6.91 (1H, d, J=7.6 Hz, ArH), 7.02 (1H, d, J=7.6 Hz, ArH) and 7.53 (1H, t, J=7.7 Hz, ArH); LC/MS (APCI) m/z 299 (M⁺+H); HPLC tᵣ=5.98 min (>99%) in 10% water-acetonitrile.

### Adamantane-1-carboxylic acid methyl-pyridin-3-ylmethyl-amide (STX2135, XDS04114)

The title compound was synthesized with general amide formation procedure from 1-adamantane carbonyl chloride (100 mg, 0.50 mmol) and the amine (92 mg, 0.75 mmol). The product (130 mg, 91%) was obtained as white solid. mp 57-59.5° C.; TLC single spot at R_{f}: 0.62 (10% methanol/DCM); ¹H NMR (270 MHz, CDCl₃) δ 1.70 (6H, s, 3×CH₂), 2.03 (9H, s, 3×CH₂ and 3×CM), 3.05 (3H, s, CH₃), 4.62 (2H, s, CH₂), 7.25 (1H, dd, J=7.9, 5.0 Hz, ArH), 7.53 (R₁, dt, J=7.9, 1.7 Hz, ArH), 8.46 (1H, d, J=1.7 Hz, ArH) and 8.50 (1H, dd, J=4.9, 1.5 Hz, ArH); LC/MS (APCI) m/z 285 (+H); HPLC tᵣ=5.40 min (>99%) in 10% water-acetonitrile.

### Adamantan-1-yl-[4-(furan-2-carbonyl)-piperazin-1-yl]-methanone (STX2136, XDS04115)

The title compound was synthesized with general amide formation method from 1-adamantane carbonyl chloride (100 mg, 0.5 mmol) and the amine (118 mg, 0.6 mmol). White solid (160 mg, 89%) was obtained. mp 182-183.5° C.; TLC single spot at R_{f}: 0.28 (20% ethyl acetate/DCM); ¹H NMR (270 MHz, CDCl₃) δ 1.72 (6H, s, 3×CH₂), 2.00 (6H, s, 3×CH₂), 2.05 (3H, broad s, 3×CH), 3.76 (8H, s, 4×CH₂), 6.48 (1H, dd, J=3.5, 1.8 Hz, ArH), 7.03 (1H, dd, J=3.5, 0.8 Hz, ArH) and 7.48 (1H, dd, J=2.0, 1.0 Hz, ArH); LC/MS (APCI) m/z 341-H); HPLC tᵣ=4.91 min (>99%) in 10% water-acetonitrile.

### Adamantane-1-carboxylic acid (pyridin-4-ylmethyl)-amide (STX2137, XDS04116)

The title compound was synthesized with general amide formation procedure from 1-adamantane carbonyl chloride (100 mg, 0.50 mmol) and the amine (65 mg, 0.60 mmol). The product (100 mg, 74%) was obtained as white solid. mp 173.5-175° C.; TLC single spot at R_{f}: 0.55 (10% methanol/DCM); ¹H NMR (270 MHz, CDCl₃) δ 1.67-1.78 (6H, m, 3×CH₂), 1.89 (6H, d, J=2.9 Hz, 3×CH₂), 2.06 (3H, broad s, 3×CH), 4.44 (2H, d, J=5.9H, CH₂), 6.02 (1H, broad, NH), 7.14 (21H, m, ArH) and 8.54 (2H, m, ArH); LC/MS (APCI) m/z 271 (M⁺+H); HPLC tᵣ=4.55 min (>99%) in 10% water-acetonitrile.

### 5-{[(Adamantane-1-carbonyl)-methyl-amino]-methyl}-thiophene-2-carboxylic acid dimethylamide (STX2153, XDS04118)

To a solution of 5-{[(adamantane-1-carbonyl)-methyl-amino]-methyl}-thiophene-2-carboxylic acid methylamide (150 mg, 0.43 mmol) in DMF (5 mL) was added NaH (60% dispersion, 52 mg, 1.29 mmol), followed by CH₃I (0.27 mL, 4.3 mmol). The mixture was stirred at ambient temperature overnight, partitioned between DCM and 1N HCl solution. The organic phase was washed with brine, dried over magnesium sulphate and concentrated in vacuo to give the crude product. Purification with flash column (methanol-DCM; gradient elution) yielded the title compound as white solid (75 mg, 48%). mp 145-146° C.; TLC single spot at R_{f}: 0.68 (10% methanol/DCM); ¹H NMR (270 MHz, CDCl₃) δ 1.66 (6H, s, 3×CH₂), 2.02 (9H, s, 3×CH₂ and 3×CH), 3.12 (3H, s, CH₃), 3.15 (6H, s, 2×CH₃), 4.69 (2H, s, CH₂), 6.85 (1H, d, J=3.7 Hz, ArH) and 7.19 (1H, d, J=3.7 Hz, ArH); LC/MS (APCI) m/z 361 (M⁺+H); HPLC tᵣ=4.87 min (>99%) in 10% water-acetonitrile.

### 1-Adamantan-1-yl-2-(6-methyl-pyridin-3-ylmethoxy)-ethanone (STX2154, FPC01027)

A mixture of 1-adamatyl bromomethyl ketone (150 mg, 0.58 mmol), 2-hydroxy-6-methylpyridine (63 mg, 0.58 mmol) and K₂CO₃ (160 mg, 1.16 mmol) in acetone (6 mL) was stirred at room temperature over night. The reaction was partitioned between ethyl acetate and water. The organic phase was washed with brine, dried over magnesium sulphate and concentrated in vacuo. Purification of the residue by flash chromatography (hexane/EtOAc gradient elution) gave the expected titled compound as white solid (114 mg, 69%). TLC single spot at R_{f} 0.48 (hexane/EtOAc 8:2); ¹H NMR (270 MHz, CDCl₃): δ 7.42 (1H, t, J=7.7 Hz), 6.45 (2H, dd app, J=3.9, 7.2 Hz), 2.32 (3H, s), 2.05 (3H, br s), 1.94 (6H, br s), 1.81-1.68 (6H, m); LC/MS (APCI) m/z 286.56 (M⁺+H); HPLC tᵣ=8.94 min (100% purity) in 10% water-acetonitrile.

### 1-Adamantan-1-yl-2-(4-methyl-benzyloxy)-ethanone (STX2155, FPC01028B)

4-Methyl benzylalcohol (93 mg, 0.76 mmol) was added in THF (0.4 mL) to a suspension of NaH (22 mg, 60% in mineral oil, 0.84 mmol) in DMF (0.4 mL) at 0° C. and was stirred at for 30 min. 1-Adamatyl bromomethyl ketone (200 mg, 0.78 mmol) was added in THF (0.2 mL×2) to the solution at 0° C. and stirred for 3 h. The reaction was partitioned between ether and water. The organic phase was washed with brine, dried over magnesium sulphate and concentrated in vacuo. Purification of the residue by flash chromatography (hexane/EtOAc, gradient elution) gave the expected titled compound as white crystal (33.3 mg, 28%). TLC single spot at R_{f} 0.50 (hexane/EtOAc 8:2); ¹H NMR (270 MHz, CDCl₃): δ 7.25 (2H, m), 7.19-7.11 (2H, m), 4.53 (2H, s), 4.27 (2H, s), 2.33 (3H, s), 2.01 (3H, br s), 1.98-1.59 (12H, m); LC/MS (APCI) m/z 299.44 (M⁺+H, 1), 141.0 (100); HPLC tᵣ=9.40 min (99.65% purity) in 10% water-acetonitrile.

### 1-Adamantan-1-yl-2-(4-methoxy-benzyloxy)ethanone (STX2163, FPC01031A)

4-Methoxy benzylalcohol (0.066 mL, 0.53 mmol) was added neat to a suspension of NaH (14.5 mg, 60% in mineral oil, 0.58 mmol) in DMF (0.4 mL) at 0° C. then was stirred at for 30 min. 1-Adamatyl bromomethyl ketone (150 mg, 0.58 mmol) was added in DMF (0.4 mL) to the solution at 0° C. and stirred for 2 h. The reaction was partitioned between ether and water. The organic phase was washed with brine, dried over magnesium sulphate and concentrated in vacuo. Purification of the residue by flash chromatography (hexane/EtOAc, gradient elution) gave the expected titled compound as transparent oil (69.2 mg, 38%). TLC single spot at R_{f}: 0.30 (hexane/EtOAc 8:2); ¹H NMR (270 MHz, CDCl₃): δ 7.32-7.24 (2H, m), 6.90-6.82 (2H, m), 4.49 (2H, s), 4.25 (2H, s), 3.78 (3H, s), 2.00 (3H, br s), 1.85-1.62 (12H, m); LC/MS (APCI) m/z 337.5 (M⁺+Na); HPLC tᵣ=7.00 min (100% purity) in 10% water-acetonitrile.

### 1-Adamantan-1-yl-2-furan-2-ylmethoxy)-ethanone (STX2164, FPC01033)

Furfuryl alcohol (0.046 mL, 0.53 mmol) was added neat to a suspension of NaH (14.5 mg, 60% in mineral oil, 0.58 mmol) in dry THF (2.5 mL) at 0° C. The suspension was stirred for 30 min at 0° C. then 1-adamatyl bromomethyl ketone (150 mg, 0.58 mmol) was added in dry THF (2.5 mL). The reaction was stirred 2 hours at a temperature between 0° C. and 5° C., and then was partitioned between ether and water. The organic phase was washed with brine, dried over magnesium sulphate and concentrated in vacuo. Purification of the residue by flash chromatography (hexane/EtOAc, gradient elution) gave the expected titled compound as yellow solid (64.2 mg, 44%). TLC single spot at R_{f}: 0.34 (hexane/EtOAc 8:2); ¹H NMR (270 MHz, CDCl₃): δ 7.39 (1H, t, J=1.5 Hz), 6.31 (2H, d, J=1.2 Hz), 4.52 (2H, s), 4.29 (2H, s), 2.00 (3H, br s), 1.78-1.61 (12H, m); LC/MS (APCI) m/z 310.45 (M⁺+H); HPLC tᵣ=7.20 min (100% purity) in 10% water-acetonitrile;

### Adamantane-1-carboxylic acid (5-methanesulfinyl-thiophen-2-ylmethyl)-methyl-amide (STX2238, XDS04128)

To a cold solution of adamantane-1-carboxylic acid methyl-(5-methylsulfanyl-thiophen-2-ylmethyl)-amide (300 mg, 0.858 mmol) in DCM (20 mL) was added m-CPBA (171 mg, purity 60-77%). The mixture was stirred at −5° C. for 30 min, partitioned between DCM and 5% sodium carbonate solution. The organic phase was washed with brine, dried over magnesium sulphate and concentrated in vacuo to give crude product. Purification with flash column (ethyl acetate-DCM; gradient elution) yielded the title compound as white solid (270 mg, 90%). mp 91.5-93.5° C.; TLC single spot at R_{f}: 0.38 (30% EtOAc/DCM); ¹H NMR (270 MHz, CDCl₃) δ 1.66 (6H, s, 3×CH₂), 2.02 (9H, s, 3×CH₂ and 3×CH), 2.90 (3H, s, CH₃), 3.15 (3H, s, CH₃), 4.58 (1H, d, J=15 Hz, CH), 4.82 (1H, d, J=15 Hz, CH), 6.91 (1H, d, J=3.6 Hz, ArH) and 7.33 (1H, d, J=3.7 Hz, ArH); LC/MS (APCI) m/z 352 (M⁺+H); HPLC tᵣ=4.88 min (>99%) in 10% water-acetonitrile.

### 2-Adamantane-1-sulfinyl)-N-methyl-N-thiophen-2-ylmethyl-acetamide (STX2239, XDS04130)

To a cold solution of 2-(adamantan-1-ylsulfanyl)-N-methyl-N-thiophen-2-ylmethyl-acetamide (90 mg, 0.268 mmol) in DCM (3 mL) was added m-CPBA (72 mg, purity 60-77%). The mixture was stirred at −5° C. for 20 min, partitioned between DCM and 5% sodium carbonate solution. The organic phase was washed with brine, dried over magnesium sulphate and concentrated in vacuo to give crude product. Purification with flash column (ethyl acetate-DCM; gradient elution) yielded the title compound as clear oil (80 mg, 85%). TLC single spot at R_{f}: 0.65 (10% methanol/DCM); ¹H NMR (270 MHz, CDCl₃) indicated 4 sets of signals from stereo isomers: δ 1.60-1.82 (48H, m, 24×CH₂), 2.19 (12H, s, 12×CH), 3.05 (6H, s, 2×CH₃), 3.11 (6H, s, 2×CH₃), 3.45 (2H, d, J=13 Hz, 2×CH), 3.52 (2H, d, J=13 Hz, 2×CH), 3.68 (2H, d, J=13 Hz, 2×CH), 3.78 (2H, d, J=13 Hz, 2×CH), 4.52 (2H, d, J=17 Hz, 2×CH), 4.65 (2H, d, J=15 Hz, 2×CH), 4.86 (2H, d, J=15 Hz, 2×CH), 5.20 (2H, d, J=17 Hz, 2×CH), 6.91-6.99 (8H, m, ArH) and 7.24 (4H, dd, J=5.7, 1.2 Hz, ArH); LC/MS (APCI) m/z 352 (M⁺+H); HPLC tᵣ=4.20 min (>99%) in 10% water-acetonitrile.

### N-[4-(2-Adamantan-1-yl-2-oxo-ethylamino)-phenyl]-acetamide (STX2241, XDS04132)

To a solution of 1-adamantyl bromomethyl ketone (330 mg, 1.28 mmol) in acetonitrile (10 mL) was added diisopropylethylamine (0.5 mL), followed by N-(4-aminophenyl)acetamide (192 mg, 1.28 mmol). The mixture was stirred at ambient temperature for 24 h, partitioned between DCM and 5% sodium bicarbonate solution. The organic phase was washed with brine, dried over sodium sulphate and concentrated in vacuo to give the crude product as white solid. Purification with flash column (ethyl acetate/DCM; gradient elution) yielded the title compound as white solid (255 mg, 61%). mp 156-158° C.; TLC single spot at R_{f}: 0.68 (5% methanol/DCM); ¹H NMR (270 MHz, CDCl₃) δ 1.66-1.75 (611, n, 3×CH₂), 1.87 (6H, d, J=2.8 Hz, 3×CH₂), 2.07 (3H, broad s, 3×CH), 2.12 (3H, s, CH₃), 4.04 (2H, s, CH₂), 4.63 (1H, broad, NH), 6.57 (2H, dd, J=7.1, 1.9 Hz, ArH), 6.99 (1H, broad, NH) and 7.28 (2H, dd, J=7.2, 1.9 Hz, ArH); LC/MS (APCI) m/z 327 (M⁺+H); HPLC tᵣ=4.93 min (96.8%) in 10% water-acetonitrile.

### N-[4-(2-Adamantan-1-yl-2-oxo-ethylsulfanyl)-phenyl]-acetamide (STX2242, XDS04133)

To a solution of adamantan-1-yl bromomethyl ketone (500 mg, 1.94 mmol) in acetonitrile (15 mL) was added N-(4-mercaptophenyl)acetamide (356 mg, 2.13 mmol), followed by triethylamine (1.5 mL). The mixture was stirred at ambient temperature overnight 2-Chloro-tritylchloride resin (400 mg, 1.6 mmol/g) was added and the mixture was stirred for 2 h, filtered and concentrated in vacuo to give the crude product. Purification with flash column (ethyl acetate/DCM; gradient elution) yielded the title compound as white solid (520 mg, 78%). TLC single spot at R_{f}: 0.20 (20% EtOAc/DCM); ¹H NMR (270 MHz, CDCl₃) δ 1.62-1.75 (6H, m, 3×CH₂), 1.82 (6H, d, J=2.8 Hz, 3×CH₂), 2.03 (3H, broad, 3×CH), 2.16 (3H, s, CH₃), 3.86 (2H, s, CH₂), 7.21 (1H, broad, NH), 7.34 (2H, dd, J=7.0, 2.0 Hz, ArH) and 7.42 (2H, dd, J=7.0, 2.0 Hz, ArH); LC/MS (APCI) m/z 342 (M⁺−H); HPLC t=5.25 min (>99%) in 10% water-acetonitrile.

### Adamantane-1-carboxylic acid methyl-4-methyl-thiazol-2-ylmethyl)-amide (STX2243, XDS04135)

The title compound was synthesized with general amide formation procedure from 1-adamantane carbonyl chloride (10 mg, 0.50 mmol) and the amine (106.5 mg, 0.75 mmol). The product (135 mg, 88%) was obtained as clear oil. TLC single spot at R_{f}: 0.36 (25% EtOAc/DCM); ¹H NMR (270 MHz, CDCl₃) δ 1.71 (6H, s, 3×CH₂), 2.03 (9H, s, 3×CH₂ and 3×CH), 2.41 (3H, s, CH₃), 3.19 (3H, s, CH₃), 4.80 (2H, s, CH₂) and 6.81 (1H, s, ArH); -LC/MS (APCI) m/z 305 (M⁺+H); HPLC tᵣ=6.02 min (98.9%) in 10% water-acetonitrile.

### Adamantane-1-carboxylic acid (2,5-dimethyl-2H-pyrazol-3-ylmethyl)-methyl-amide (STX2244, XDS04136)

The title compound was synthesized with general amide formation procedure from 1-adamantane carbonyl chloride (10 mg, 0.50 mmol) and the amine (104 mg, 0.75 mmol). The product (136 mg, 90%) was obtained as white solid. mp 102-103.5° C.; TLC single spot at R_{f}: 0.20 (25% EtOAc/DCM); ¹H NMR (270 MHz, CDCl₃) δ 1.71 (6H, s, 3×CH₂), 2.01 (9H, s, 3×CH₂ and 3×CH), 2.20 (3H, s, CH₃), 3.08 (3H, s, CH₃), 3.70 (3H, s, CH₃), 4.58 (2H, s, CH₂) and 5.87 (1H, s, ArH); LC/MS (APCI) m/z 302 (M⁺+H); HPLC tᵣ=5.28 min (>99%) in 10% water-acetonitrile.

### Adamantane-1-carboxylic acid (4-acetylamino-benzyl)-methyl-amide (STX2245, XDS04137)

The title compound was synthesized with general amide formation procedure from 1-adamantane carbonyl chloride (100 mg, 0.50 mmol) and the amine (133 mg, 0.75 mmol). The product (25 mg, 15%) was obtained as white solid. mp 109-111° C.; TLC single spot at R_{f}: 0.20 (25% EtOAc/DCM); ¹H NMR (270 MHz, CDCl₃) δ 1.70 (6H, s, 3×CH₂), 2.03 (9H, s, 3×CH₂ and 3×CH), 2.16 (3H, s, CH₃), 2.95 (3H, s, CH₃), 4.63 (2H, s, CH₂), 7.12 (2H, d, J=8.7 Hz, ArH), 7.45 (2H, d, J=8.7 Hz, ArH) and 7.51 (1H, s, NH); LC/MS (APCI) m/z 339 (M⁺−H); HPLC tᵣ=4.80 min (>99%) in 10% water-acetonitrile.

### Adamantane-1-carboxylic acid (4-acetylamino-phenyl)-amide (STX2246, XDS04138)

The title compound was synthesized with general amide formation procedure from 1-adamantane carbonyl chloride (100 mg, 0.50 mmol) and the amine (113 mg, 0.75 mmol). The product (38 mg, 24%) was obtained as white solid. mp>300° C.; TLC single spot at R_{f}: 0.21 (25% EtOAc/DCM); ¹H NMR (270 MHz, DMSO) δ 1.69 (6H, s, 3×CH₂), 1.89 (6H, d, J=2.8 Hz, 3×CH₂), 2.00 (3H, s, 3×CH), 2.01 (3H, s, CH₃), 7.51 (4H, AB, ArH), 9.05 (1H, s, NH) and 9.85 (1H, s, NH); LC/MS (APCI) m/z 311 (M⁺−H); HPLC tᵣ=4.25 min (>99%) in 10% water-acetonitrile.

### Adamantane-1-carboxylic acid furan-2-ylmethyl-methyl-amide (STX2247, XDS04139)

The title compound was synthesized with general amide formation procedure from 1-adamantane carbonyl chloride (10 mg, 0.50 mmol) and the amine (83 mg, 0.75 mmol). The product (130 mg, 95%) was obtained as white solid. mp 53.5-54.5° C.; TLC single spot at R_{f}: 0.68 (30% EtOAc/hexane); ¹H NMR (270 MHz, CDCl₃) δ 1.70 (6H, s, 3×CH₂), 2.02 (9H, s, 3×CH₂ and 3×CH), 3.06 (3H, s, CH₃), 4.59 (2H, s, CH₂), 6.19 (1H, dd, J=3.2, 0.7 Hz, ArH), 6.31 (1H, dd, J=3.2, 1.7 Hz, ArH) and 7.33 (1H, dd, J=1.7, 0.7 Hz, ArH); LC/MS (APCI) m/z 274 (M⁺+H); HPLC tᵣ=6.14 min (>99%) in 10% water-acetonitrile.

### Adamantane-1-carboxylic acid (4-methyl-thiophen-2-ylmethyl)-amide (STX2248, XDS04140)

The title compound was synthesized with general amide formation procedure from 1-adamantane carbonyl chloride (205 mg, 1.03 mmol) and the amine (125 mg, 0.98 mmol). The product (270 mg, 95%) was obtained as white solid. mp 127-128.5° C.; TLC single spot at R_{f}: 0.60 (30% EtOAc/hexane); ¹H NMR (270 MHz, CDCl₃) δ 1.65-1.91 (12H, m, 6×CH₂), 2.03 (3H, broad, 3×CH), 2.20 (3H, d, J=1.0 Hz, CH₃), 4.53 (2H, d, J=5.4 Hz, CH₂), 5.86 (1H, broad, NH) and 6.74-6.77 (2H, m, ArH); LC/MS (APCI) m/z 288 (M⁺−H); HPLC tᵣ=5.52 min (>99%) in 10% water-acetonitrile.

### Adamantane-1-carboxylic acid (3-methyl-3H-imidazol-4-ylmethyl)-amide (STX2249, XDS04141)

The title compound was synthesized with general amide formation procedure from 1-adamantane carbonyl chloride (234 mg, 1.18 mmol) and the amine (125 mg, 1.12 mmol). The product (255 mg, 83%) was obtained as white solid. mp 197.5-199° C.; TLC single spot at R_{f}: 0.39 (10% methanol/DCM); 111 NMR (270 MHz, CDCl₃) δ 1.64-1.75 (6H, m, 3×CH₂), 1.83 (6H, d, J=2.7 Hz, 3×CH₂), 2.03 (3H, broad, 3×CM), 3.55 (3H, s, CH₃), 4.45 (2H, d, J=5.5 Hz, CH₂), 5.66 (1H, broad, NM, 6.89 (1H, s, ArH) and 7.41 (1H, s, ArH); LC/MS (APCI) m/z 272 (M⁺−H); HPLC tᵣ=15.0 min (97.5%) in 100% methanol.

### Adamantane-1-carboxylic acid (5-methanesulfonyl-thiophen-2-ylmethyl)-methyl-amide (STX2250, XDS04143)

To a solution of adamantane-1-carboxylic acid (5-methanesulfinyl-thiophen-2-ylmethyl)-methyl-amide (144 mg, 0.39 mmol) in DCM (5 mL) was added m-CPBA (132 mg, purity 60-77%). The mixture was stirred at ambient temperature for 7 h, partitioned between DCM and 5% sodium carbonate solution. The organic phase was washed with brine, dried over magnesium sulphate and concentrated in vacuo to give crude product. Purification with flash column (ethyl acetate-DCM; gradient elution) yielded the title compound as white solid (136 mg, 95%). mp 114.5-115.5° C.; TLC single spot at R_{f}: 0.69 (25% EtOAc/DCM); ¹H NMR (270 MHz, CDCl₃) δ 1.71 (6H, s, 3×CH₂), 2.02 (9H, s, 3×CH₂ and 3×CH), 3.14 (3H, s, CH₃), 3.19 (3H, s, CH₃), 4.67 (2H, s, CH₂), 6.93 (1H, d, J=4.0 Hz, ArH) and 7.55 (1H, d, J=4.0 Hz, ArH); LC/MS (APCI) m/z 366 (M⁺−H); HPLC tᵣ=4.78 min (>99%) in 10% water-acetonitrile.

### 1-(5-Chloro-3-methyl-benzo[b]thiophen-2-yl)-2-(4-chloro-phenylmethanesulfinyl)-ethanone (STX2251, XDS04145)

To a cold solution of 1-(5-chloro-3-methylbenzo[b]thiophen-2-yl)-2-(4-chlorobenzylthio)ethanone (160 mg, 0.42 mmol) in DCM (8 mL) was added m-CPBA (113 mg, purity 60-77%). The mixture was stirred at −5° C. for 20 min, partitioned between DCM and 5% sodium carbonate solution. The organic phase was washed with brine, dried over magnesium sulphate and concentrated in vacuo to give crude product. Purification with flash column (ethyl acetate-DCM; gradient elution) yielded the title compound as white solid (125 mg, 75%). mp 168.5-170° C.; TLC single spot at R_{f}: 0.59 (30% EtOAc/DCM); ¹H NMR (300 MHz, CDCl₃) δ 2.73 (3H, s, CH₃), 4.07 (1H, d, J=14.9 Hz, CU), 4.11 (1H, d, J=132 Hz, CH), 4.19 (1H, d, J=14.9 Hz, CH), 4.30 (1H, d, J=13.1 Hz, CU), 7.26-7.37 (4H, m, ArH), 7.48 (1H, dd, J=8.7, 1.8 Hz, ArH), 7.77 (1H, d, J=8.7 Hz, ArH) and 7.86 (1H, d, J=1.8 Hz, ArH); LC/MS (APCI) m/z 395 (M⁺+H); HPLC tᵣ=5.37 min (>99%) in 10% water-acetonitrile.

### N-[4-(2-Adamantan-1-yl-2-oxo-ethanesulfinyl)-phenyl]-acetamide (STX2253, XDS04147)

To a cold solution of N-[4-(2-adamantan-1-yl-2-oxo-ethylsulfanyl)-phenyl]-acetamide (250 mg, 0.729 mmol) in DCM (10 mL) was added m-CPBA (195 mg, purity 60-77%). The mixture was stirred at −5° C. for 20 min, partitioned between DCM and 5% sodium carbonate solution. The organic phase was washed with brine, dried over magnesium sulphate and concentrated in vacuo to give crude product. Purification with flash column (ethyl acetate-DCM; gradient elution) yielded the title compound as white solid (200 mg, 76%). mp 173.5-175.5° C.; TLC single spot at R_{f}: 0.22 (40% EtOAc/DCM); ¹H NMR (270 MHz, CDCl₃) δ 1.59-1.73 (12H, m, 6×CH₂), 2.01 (3H, broad, 3×CM), 2.19 (3H, s, CH₃), 3.78 (1H, d, J=5.6 Hz, CM), 4.15 (1H, d, J=5.6 Hz, CH) and 7.65 (5H, m, NH and ArH); LC/MS (APCI) m/z 358 (M⁺−H); HPLC tᵣ=4.35 min (>99%) in 10% water-acetonitrile.

### Adamantane-1-carboxylic acid methyl-(4-methyl-thiophen-2-ylmethyl)-amide (STX2254, XDS04149)

To a solution of adamantane-1-carboxylic acid (4-methyl-thiophen-2-ylmethyl)-amide (150 mg, 0.52 mmol) in anhydrous DMF (5 mL) was added NaH (60% dispersion, 48 mg, 1.04 mmol), followed by CH₃I (0.32 mL, 5.2 mmol). The mixture was stirred at ambient temperature for 24 h, partitioned between ethyl acetate and brine. The organic phase was washed with brine, dried over sodium sulphate and concentrated in vacuo to give the crude product. Purification with flash column (hexane-ethyl acetate; gradient elution) yielded the title compound as white solid (130 mg, 82%). mp 87-88° C.; TLC single spot at R_{f}: 0.50 (30% hexane/DCM); ¹H NMR (270 MHz, CDCl₃) δ 1.70 (6H, S, 3×CH₂), 2.03 (9H, s, 3×CH₂ and 3×CM), 2.20 (3H, d, J=1.0 Hz, CH₃), 3.06 (3H, s, CH₃), 4.67 (2H, s, CH₂), 6.71 (1H, s, ArH) and 6.77 (1H, q, J=1.2 Hz, ArH); LC/MS (APCI) m/z 304 (M⁺+H); HPLC tᵣ=8.34 min (>99%) in 10% water-acetonitrile.

### 1-(Adamantane-1-carbonyl)-piperidine-4-carboxylic acid (XDS04144)

To a solution of 1-adamantane-1-carbonyl)-piperidine-4-carboxylic acid ethyl ester (1.2 g, 3.76 mmol) in methanol (30 mL) was added LiOH.H₂O (510 mg, 12 mmol). The was stirred at ambient temperature under nitrogen for 20 h, concentrated in vacuo and acidified with 4N HCl to pH 3. The precipitate was collected and washed with water, dried in vacuo to yield the crude product (1.05 g, 96%). Purification of 100 mg crude product with flash column (ethyl acetate-DCM; gradient elution) gave the analytical sample. mp 2055-207.5° C.; TLC single spot at R_{f}: 0.50 (10% methanol/DCM); ¹H NMR (270 MHz, CDCl₃) δ 1.60-1.75 (8H, m, 3×CH₂ and 2×CH), 1.91-1.92 (2H, m, 2×CH), 1.98 (6H, s, 3×CH₂), 2.03 (3H, s, 3×CH), 2.59 (1H, m, CH), 3.00 (2H, td, J=13.6, 2.5 Hz, 2×CH) and 4.37 (2H, td, J=13.6, 2.3 Hz, 2×CH); LC/MS (APCI) m/z 290 (M⁺−H); HPLC t=11.8 min (>99%) in 100% methanol.

### 1-Adamantan-1-yl-2-(pyridin-2-ylmethoxy)-ethanone (STX2286, FPC01037B)

2-Pyridylcarbinol (0.051 mL, 0.53 mmol) was added neat to a suspension of NaH (20 mg, 60% in mineral oil, 0.80 mmol) in dry THF (2.5 mL) at 0° C. The suspension was stirred 30 min at 0° C. then 1-adamatyl bromomethyl ketone (150 mg, 0.58 mmol) was added in dry THF (2.5 mL) at 0° C. then the reaction was allowed to warm up to room temperature over night. The reaction was partitioned between dichloromethane and water; the organic layer was washed with brine, dried over magnesium sulphate, filtered and concentrated in vacuo. Purification of the residue by flash chromatography (DCM/MeOH, gradient elution) gave the expected titled compound as yellow oil (95.5 mg, 63.5%). TLC single spot at R_{f} 0.4 (DCM/MeOH 95:5); ¹H NMR (270 MHz, CDCl₃): δ 8.53 (1H, dq, J=0.8, 5.0 Hz), 7.69 (1H, td, J=1.7, 7.6 Hz), 7.51 (1H, d, J=7.6 Hz), 7.21-7.13 (1H, m), 4.67 (2H, s), 4.43 (2H, s), 2.01 (3H, br s), 1.81 (6H, d, J=2.7 Hz), 1.80-1.60 (6H, m); LC/MS (APCI) m/z 286.35 (M⁺+H); HPLC tᵣ=5.57 min (100% purity) in 10% water-acetonitrile.

### 1-Adamantan-1-yl-2-(pyridin-3-ylmethoxy)-ethanone (STX2287, FPC01038-2)

3-Pyridylcarbinol (0.051 mL, 0.53 mmol) was added neat to a suspension of NaH (20 mg, 60% in mineral oil, 0.80 mmol) in dry THF (2.5 mL) at 0° C. The suspension was stirred 30 min at 0° C. then 1-adamatyl bromomethyl ketone (150 mg, 0.58 mmol) was added in dry THF (2.5 mL) at 0° C. then the reaction was allowed to warm up to room temperature over night. The reaction was partitioned between ethyl acetate and water; the organic layer was washed with brine, dried over magnesium sulphate, filtered and concentrated in vacuo. Purification of the residue by flash chromatography (DCM/MeOH gradient elution) gave the expected titled compound as yellow crystal (72.6 mg, 48%). TLC single spot at R_{f}: 0.21 (DCM/MeOH 95:5); ¹H NMR (270 MHz, CDCl₃): δ 8.56 (2H, m), 7.74 (1H, td, J=2.0, 7.9 Hz), 7.27 (1H, dd, J=4.9, 7.9 Hz), 4.56 (2H, s), 4.34 (2H, s), 2.01 (3H, br s), 1.80 (6H, d app, J=3.0 Hz), 1.78-1.58 (6H, m); LC/MS (APCI) m/z 286.35 (M⁺+H); HPLC tᵣ=5.59 min (100% purity) in 10% water-acetonitrile.

### 1-Adamantan-1-yl-2-(6-methyl-pyridin-2-ylmethoxy)-ethanone (STX2288, FPC01039)

6-Methyl-2-pyridine-methanol (65 mg, 0.53 mmol) was added in dry THF (0.5 mL) to a suspension of NaH (20 mg, 60% in mineral oil, 0.80 mmol) in dry THF (2.5 mL) at 0° C. The suspension was stirred 30 min at 0° C. then 1-adamatyl bromomethyl ketone (150 mg, 0.58 mmol) was added in dry THF (2 mL) at 0° C. then the reaction was allowed to warm up to room temperature over night. The reaction was partitioned between ethyl acetate and water; the organic layer was washed with brine, dried over magnesium sulphate, filtered and concentrated in vacuo. Purification of the residue by flash chromatography (DCM/MeOH gradient elution) gave the expected titled compound as yellow wax (68.6 mg, 43%). TLC single spot at R_{f} 0.37 (DCM/MeOH 95:5); ¹H NMR (270 MHz, CDCl₃): δ 7.58 (1H, t, J=7.7 Hz), 7.31 (1H, d app, J=7.7 Hz), 7.42 (1H, d app, J=7.4 Hz), 4.64 (2H, s), 4.42 (2H, s), 2.52 (3H, s), 2.02 (3H, br s), 1.81 (6H, d app, J=3.0 Hz), 1.80-1.60 (6H, m); LC/MS (APCI) m/z 300.46 (M⁺+H); HPLC tᵣ=5.94 min (100% purity) in 10% water-acetonitrile.

### 1-Adamantan-1-yl-2-(biphenyl-4-ylmethoxy)-ethanone (STX2289, FPC01041A)

4-Biphenylmethanol (98 mg, 0.53 mmol) was added in dry THF (2 mL) to a suspension of NaH (20 mg, 60% in mineral oil, 0.80 mmol) in dry THF (0.5 mL) at 0° C. The suspension was stirred 30 min at 0° C. then 1-adamatyl bromomethyl ketone (150 mg, 0.58 mmol) was added in dry THF (2.5 mL) at 0° C. then the reaction was allowed to warm up to room temperature over night. The reaction was partitioned between ethyl acetate and water; the organic layer was washed with brine, dried over magnesium sulphate, filtered and concentrated in vacuo. Purification of the residue by flash chromatography (hexanes/EtOAc, gradient elution) gave the expected titled compound as a white crystal (81.4 mg, 43%). TLC single spot at R_{f} 0.45 (hexanes/EtOAc 80:20); ¹H NMR (270 MHz, CDCl₃): δ 7.62-7.54 (4H, m), 7.47-7.39 (1H, m), 7.37-7.29 (1H, m), 4.62 (2H, s), 4.34 (2H, s), 2.02 (3H, br s), 1.82 (6H, d app, J=3.0 Hz), 1.79-1.61 (6H, m); LC/MS (APCI) m/z 383.58 (M⁺+Na); HPLC tᵣ=3.99 min (100% purity) in 10% water-acetonitrile.

### 1-Adamantan-1-yl-2-(toluene-4-sulfinyl)-ethanone (STX2290, FPC01045)

m-CPBA (110 mg, 0.49 mmol, 77% max) was added to a solution of 1-adamantan-1-yl-2-p-tolylsulfanyl-ethanone (99 mg, 0.33 mmol) in DCM (5 mL) at 0° C. After 45 min of vigorous stirring, the reaction was partitioned between dichloromethane and a saturated solution of NaHCO₃. The organic phase was washed with water then brine, dried over MgSO₄ and concentrated in vacuo. Purification of the residue by flash chromatography (hexanes/EtOAc, gradient elution) gave the expected titled compound as a white solid (70.2 mg, 67%). TLC single spot at R_{f} 0.22 (hexane/EtOAc 6:4); ¹H NMR (270 MHz, CDCl₃): δ 7.56 (2H, d app, J=8.1 Hz), 7.30 (2H, d app, J=7.9 Hz), 4.13 (1H, d, J=15.3 Hz), 3.75 (1H, d, J=15.1 Hz), 2.39 (3H, s), 1.99 (3H, br s), 1.74-1.57 (6H, m, containing d app, J=3.0 Hz); LC/MS (APCI) m/z 315.38 (M⁺−H); HPLC tᵣ=5.42 min (98.93% purity) in 10% water-acetonitrile;

### 1-Adamantan-1-yl-2-(4-chloro-benzenesulfinyl)-ethanone (STX2291, FPC01046)

m-CPBA (103 mg, 0.46 mmol, 77% max) was added to a solution of 1-adamantan-1-yl-2-(4-chloro-phenylsulfanyl)-ethanone (98.5 mg, 0.31 mmol) in DCM (5 mL) at 0° C. After 45 min of vigorous stirring, the reaction was partitioned between dichloromethane and a saturated solution of NaHCO₃. The organic phase was washed with water then brine, dried over MgSO₄ and concentrated in vacuo. Purification of the residue by flash chromatography (hexanes/EtOAc, gradient elution) gave the expected titled compound as a white solid (46 mg, 44%). TLC single spot at R_{f} 0.24 (hexane/EtOAc 6:4); ¹H NMR (270 MHz, CDCl₃): δ 7.64 (2H, dt, J=2.5, 8.6 Hz), 7.49 (2H, dt, J=2.2, 8.9 Hz), 4.15 (1H, d, J=15.3 Hz), 3.80 (1H, d, J=15.3 Hz), 2.01 (3H, br s), 1.78-1.55 (12H, m); LC/MS (APCI) m/z 335.47 (M⁺−H); HPLC tᵣ=5.74 min (100% purity) in 10% water-acetonitrile;

### 1-Adamantan-1-yl-2-(thiophen-3-ylmethoxy)-ethanone (STX2292, FPC01047)

3-Thiophene methanol (0.10 mL, 1.06 mmol) was added neat to suspension of NaH (40 mg, 60% in mineral oil, 1.60 mmol) in THF (4 mL) at 0° C. The suspension was stirred 30 min at 0° C. and 1-adamatyl bromomethyl ketone (300 mg, 1.06 mmol) was added in THF (5 mL) at 0° C. then the reaction was allowed to warm up to room temperature overnight. The reaction was partitioned between dichloromethane and water; the organic layer was washed with brine, dried over magnesium sulphate, filtered and concentrated in vacuo. Purification of the residue by flash chromatography (hexanes/EtOAc, gradient elution) gave the expected titled compound as a white solid (67.5 mg, 20%). TLC single spot at R 0.59 (hexanes/EtOAc 60:40); ¹H NMR (270 MHz, CDCl₃): δ 7.30 (1H, dd, J=3.0, 5.0 Hz), 7.24-7.19 (1H, m), 7.10 (1H, dd, J=1.5, 5.0 Hz), 4.58 (2H, s), 4.28 (2H, s), 2.01 (3H, br s), 1.80 (6H, d app, J=3.0 Hz), 1.79-1.63 (6H, m); LC/MS (APCI) m/z 291 (M⁺+H), 313 (M⁺+Na); HPLC tᵣ=7.26 min (94.22% purity) in 10% water-acetonitrile;

### 1-Adamantan-1-yl-2-(2,4-dichloro-phenylmethanesulfonyl)-ethanone (STX2293, FPC01051)

m-CPBA (35 mg, 0.16 mmol, 77% max) was added to a solution of 1-adamantan-1-yl-2-(2,4-dichloro-benzylsulfanyl)-ethanone (40 mg, 0.10 mmol) in DCM (2.5 mL) at room temperature. After 17 h, the reaction was partitioned between dichloromethane and a saturated solution of NaHCO₃. The organic phase was washed with water then brine, dried over MgSO₄ and concentrated in vacuo. Purification of the residue by flash Chromatography (hexanes/EtOAc, gradient elution) gave the expected titled compound as a white solid (7.8 mg, 19%). TLC single spot at R_{f} 0.42 (hexane/EtOAc 5:5); ¹H NMR (270 MHz, CDCl₃): δ 7.49 (1H, d, J=8.4 Hz), 7.47 (1H, d, J=2.2 Hz), 7.28 (1H, dd, J=2.2, 8.1 Hz), 4.77 (2H, s), 4.08 (2H, s), 2.08 (3H, br s), 1.81 (6H, d, J=2.7 Hz), 1.79-1.60 (6H, m); LC/MS (APCI) m/z 399.32 (M⁺−H), 402.40 (M⁺+H); HPLC t=6.75 min (97.97% purity) in 10% water-methanol.

### 1-Adamantan-1-yl-2-(1-methyl-1H-imidazole-2-sulfonyl)-ethanone (STX2294, XDS04151)

To a solution of 1-adamantan-1-yl-2-(1-methyl-1H-imidazole-2-sulfinyl)-ethanone (98 mg, 0.32 mmol) in DCM (5 mL) was added m-CPBA (112 mg, purity 60-77%). The mixture was stirred at ambient temperature for 7 h, partitioned between DCM and 5% sodium carbonate solution. The organic phase was washed with brine, dried over magnesium sulphate and concentrated in vacuo to give crude product. Purification with flash column (ethyl acetate-hexane; gradient elution) yielded the title compound as white solid (66 mg, 64%). mp 124-125° C.; TLC single spot at R_{f}: 0.22 (30% EtOAc/hexane); ¹H NMR (270 MHz, CDCl₃) δ 1.61-1.74 (12H, m, 6×CH₂), 2.04 (3H, broad, 3×CH), 4.01 (3H, s, CH₃), 4.58 (2H, s, CH₂) and 7.14 (1H, d, J=1.0 Hz, ArH); LC/MS (APCI) m/z 321 (M⁺−H); HPLC tᵣ=2.00 min (>99%) in 10% water-acetonitrile.

### 1-Adamantan-1-yl-2-(thiophene-2-sulfonyl)-ethanone (STX2295, XDS04152)

To a solution of 1-adamantan-1-yl-2-(thiophene-2-sulfinyl)-ethanone (103 mg, 0.33 mmol) in DCM (5 mL) was added m-CPBA (112 mg, purity 60-77%). The mixture was stirred at ambient temperature for 7 h, partitioned between DCM and 5% sodium carbonate solution. The organic phase was washed with brine, dried over magnesium sulphate and concentrated in vacuo to give crude product. Purification with flash column (ethyl acetate-hexane; gradient elution) yielded the title compound as white solid (90 mg, 84%). mp 117.5-119° C.; TLC single spot at R_{f}: 0.70 (30% EtOAc/hexane); ¹H NMR (270 MHz, CDCl₃) δ 1.61-1.74 (6H, m, 3×CH₂), 1.75 (6H, d, J=2.7 Hz, 3×CH₂), 2.04 (3H, broad, 3×CH), 4.36 (2H, s, CH₂), 7.14 (1H, dd, J=5.0, 3.7 Hz, ArH) and 7.72-7.76 (2H, m, ArH); LC/MS (APCI) m/z 323 (M⁺−H); HPLC tᵣ=2.25 min (>99%) in 10% water-acetonitrile.

### 1-(5-Chloro-3-methyl-benzo[b]thiophen-2-yl)-2-(4-chloro-phenylmethanesulfonyl)-ethanone (STX2296, XDS04153)

To a solution of 1-(5-chloro-3-methyl-benzo[b]thiophen-2-yl)-2-(4-chloro-phenylmethanesulfinyl)-ethanone (70 mg, 0.18 mmol) in DCM (5 mL) was added m-CPBA (59 mg, purity 60-77%). The mixture was stirred at ambient temperature for 7 h, partitioned between DCM and 5% sodium carbonate solution. The organic phase was washed with brine, dried over magnesium sulphate and concentrated in vacuo to give crude product. Purification with flash column (ethyl acetate-hexane; gradient elution) yielded the title compound as white solid (36 mg, 48%). mp 204.5-205.5° C.; TLC single spot at R_{f}: 0.68 (5% EtOAc/DCM); ¹H NMR (270 MHz, CDCl₃) δ 2.79 (3H, s, CH₃), 4.30 (2H, s, CH₂), 4.55 (2H, s, CH₂), 7.38 (2H, dt, J=8.6, 2.2 Hz, ArH), 7.48-7.53 (3×m, ArH), 7.78 (1H, d, J=8.7 Hz, ArH) and 7.89 (1H, d, J=1.9 Hz, ArH); LC/MS (APCI) m/z 411 (M⁺−H); HPLC tᵣ=2.43 min (98%) in 10% water-acetonitrile.

### N-[4-(2-Adamantan-1-yl-2-oxo-ethanesulfonyl)-phenyl]-acetamide (STX2298, XDS04155)

To a solution of N-[4-(2-adamantan-1-yl-2-oxo-ethanesulfinyl)-phenyl]-acetamide (95 mg, 0.26 mmol) in DCM (5 mL) was added m-CPBA (88 mg, purity 60-77%). The mixture was stirred at ambient temperature for 7 h, partitioned between DCM and 5% sodium carbonate solution. The organic phase was washed with brine, dried over magnesium sulphate and concentrated in vacuo to give crude product. Purification with flash column (ethyl acetate/DCM; gradient elution) yielded the title compound as white solid (79 mg, 81%). mp 151-151.5° C.; TLC single spot at R_{f}: 0.25 (10% EtOAc/DCM); ¹H NMR (270 MHz, CDCl₃) δ 1.56-1.70 (6H, m, 3×CH₂), 1.73 (6H, d, J=2.7 Hz, 3×CH₂), 2.04 (3H, broad, 3×CH), 2.22 (3H, s, CH₃), 4.27 (2H, s, CH₂), 7.43 (1H, broad, NH), 7.69 (2H, dt, J=8.7, 2.2 Hz, ArH) and 7.86 (2H, dt, J=8.7, 2.2 Hz, ArH); LC/MS (APCI) m/z 374 (M⁺−H); HPLC tᵣ=1.85 min (>99%) in 10% water-acetonitrile.

### 1-Adamantan-1-yl-2-(thiophen-2-ylmethanesulfonyl)-ethanone (STX2299, XDS04156)

To a solution of 1-adamantan-1-yl-2-(thiophen-2-ylmethanesulfinyl)-ethanone (75 mg, 0.23 mmol) in DCM (5 mL) was added m-CPBA (78 mg, purity 60-77%). The mixture was stirred at ambient temperature for 7 h, partitioned between DCM and 5% sodium carbonate solution. The organic phase was washed with brine, dried over magnesium sulphate and concentrated in vacuo to give crude product. Purification with flash column (ethyl acetate-hexane; gradient elution) yielded the title compound as white solid (68 mg, 87%). mp 95.5-98° C.; TLC single spot at R_{f}: 0.72 (35% EtOAc/hexane); ¹H NMR (270 MHz, CDCl₃) δ 1.63-1.72 (6H, m, 3×CH₂), 1.78 (6H, d, J=2.7 Hz, 3×CH₂), 2.07 (3H, broad, 3×CH), 3.95 (2H, s, CH₂), 4.73 (2H, s, CH₂), 7.03 (1H, dd, J=5.2, 3.4 Hz, ArH), 7.20 (1H, dd, J=3.4, 1.3 Hz, ArH) and 7.36 (1H, dd, J=5.2, 1.3 Hz, ArH); LC/MS (APCI) m/z 337 (M⁺−H); HPLC tᵣ=2.40 min (98.8%) in 10% water-acetonitrile.

### 1-Adamantan-1-yl-2-(2-methyl-benzothiazol-5-ylamino)propan-1-one (STX2303, FPC01053)

NaH (5 mg, 0.18 mmol) then CH₃I (0.011 mL, 0.18 mmol) were added neat to a solution of 1-adamantan-1-yl-2-(2-methyl-benzothiazol-5-ylamino)-ethanone (20 mg, 0.059 mmol) in DMF (1 mL) at room temperature. The reaction was stirred for 36 hours then partitioned between ethyl acetate and water. The organic phase was washed with water then brine, dried over MgSO₄ and concentrated in vacuo. Purification of the residue by flash chromatography (DCM/EtOAc, gradient elution) gave the expected titled compound as orange wax (12 mg, 57%). TLC single spot at R_{f} 0.51 (DCM/EtOAc 9:1); ¹H NMR (270 MHz, CDCl₃): δ 7.52 (1H, d, J=8.6 Hz), 7.14 (1H, d, J=2.2 Hz), 6.69 (1H, dd, J=2.2, 8.4 Hz), 4.68 (1H, q, J=6.2 Hz), 4.37 (1H, br s, NH), 2.77 (3H, s), 2.03 (3H, br s), 1.88-1.55 (2H, m), 1.30 (3H, d, J=6.4 Hz); LC/MS (APCI) m/z 353.48 (M⁺−H), 354.49 (M⁺); HPLC tᵣ=2.91 min (94.67% purity) in 10% water-acetonitrile.

### 1-(Adamantane-1-carbonyl)-piperidine-4-carboxylic acid methyl-thiophen-2-ylmethyl-amide (STX2304, XDS04157)

The title compound was synthesized with general amide formation method from 1-(adamantane-1-carbonyl)-piperidine-4-carboxylic acid (116 mg, 0.4 mmol) and the amine (56 mg, 0.44 mmol). The title compound (140 mg, 87%) was obtained as foamy powder. TLC single spot at R_{f}: 0.66 (10% methanol/DCM); ¹H NMR (270 MHz, CDCl₃) δ 1.70-1.80 (10H, m, 2×CH₂), 1.99 (6H, s, 3×CH₂), 2.02 (3H, broad, 3×CH), 2.67-2.90 (3H, m, 3×CH), 3.02 (3H, s, CH₃), 4.53 (2H, dt, J=13.1, 3.4 Hz, 2×CH), 4.70 (2H, s, CH₂), 6.89-6.99 (2H, m, ArH) and 7.21 (1H, dd, J=4.4, 1.9 Hz, ArH); LC/MS (APCI) m/z 401 (M⁺+H); HPLC tᵣ=1.92 min (99%) in 10% water-acetonitrile.

### 1-(Adamantane-1-carbonyl)-piperidine-4-carboxylic acid methyl-pyridin-3-ylmethyl-amide (STX2305, XDS04158)

The title compound was synthesized with general amide formation method from 1-(adamantane-1-carbonyl)-piperidinecarboxylic acid (116 mg, 0.4 mmol) and the amine (54 mg, 0.44 mmol). The title compound (120 mg, 76%) was obtained as foamy powder. TLC single spot at R_{f}: 0.23 (10% methanol/DCM); ¹H NMR (270 MHz, CDCl₃) δ 1.70-1.80 (10H, m, 2×CH₂), 1.99 (6H, s, 3×CH₂), 2.01 (3H, broad, 3×CH), 2.72-2.93 (3H, m, 3×CH), 3.00 (3H, s, CH₃), 4.57 (2H, d, J=13.5 Hz, 2×CH), 4.58 (2H, s, CH₂), 7.25 (1H, m, ArH), 7.57 (1H, d, J=7.6 Hz, ArH) and 8.47-8.57 (2H, m, ArH); LC/MS (APCI) m/z 396 (M⁺+H); HPLC t=1.51 min (98.5%) in 10% water-acetonitrile.

### 1-(Adamantane-1-carbonyl)-piperidine-4-carboxylic acid 4-chloro-benzylamide (STX2306, XDS04159)

The title compound was synthesized with general amide formation method from 1-(adamantane-1-carbonyl)-piperidine-4-carboxylic acid (116 mg, 0.4 mmol) and the amine (62 mg, 0.44 mmol). The title compound (96 mg, 58%) was obtained as white solid. mp 150-152° C.; TLC single spot at R_{f}: 0.51 (10% methanol/DCM); ¹H NMR (270 MHz, CDCl₃) δ 1.60-1.88 (110H, m, 2×CH₂), 1.96 (6H, s, 3×CH₂), 2.02 (3H, broad, 3×CH), 2.36 (1H, ft, J=11.3, 4.2 Hz, CH), 2.82 (2H, td, J=13.8, 2.2 Hz, 2×CM), 4.39 (2H, d, J=5.7 Hz, 2×CH), 4.52 (2H, d, J=13.4 Hz, 2×CH), 5.77 (1H, broad, NH), 7.17 (2H, dt, J=8.5, 2.0 Hz, ArH) and 7.28 (2H, dt, J=8.5, 2.0 Hz, ArH); LC/MS (APCI) m/z 413 (M⁺−H); HPLC tᵣ=1.89 min (96.1%) in 10% water-acetonitrile.

### 1-(Adamantane-1-carbonyl)-piperidine-4-carboxylic acid 4-methyl-benzylamide (STX307, XDS04160)

The title compound was synthesized with general amide formation method from 1-(adamantane-1-carbonyl)-piperidine-4-carboxylic acid (116 mg, 0.4 mmol) and the amine (53 mg, 0.44 mmol). The title compound (90 mg, 57%) was obtained as white solid. mp 155.5-157° C.; TLC single spot at R_{f}: 0.55 (10% methanol/DCM); ¹H NMR (270 MHz, CDCl₃) δ 1.63-1.89 (10H, m, 2×CH₂), 1.97 (6H, s, 3×CH₂), 2.02 (3H, broad, 3×CH), 2.32 (3H, s, CH₃), 2.33 (1H, m, CM), 2.81 (2H, td, J=13.5, 2.2 Hz, 2×CH), 4.39 (2H, d, J=5.7 Hz, 2×CH), 4.53 (2H, d, J=13.2 Hz, 2×CH), 5.66 (1H, broad, NH) and 7.13 (4H, s, ArH); LC/MS (APCI) m/z 393 (M⁺−H); HPLC tᵣ=1.86 min (96.9%) in 10% water-acetonitrile.

### 1-Adamantan-1-yl-2-(4-tert-butyl-phenylmethanesulfonyl)-ethanone (STX2314, FPC 01056A)

m-CPBA (529 mg, 2.36 mmol, 77% max) was added to a solution of 1-Adamantan-1-yl-2-(4-tert-butyl-benzylsulfanyl)-ethanone (420 mg, 1.18 mmol) in DCM (20 mL) at 0° C. and the reaction was stirred for 17 h. The reaction was quenched with NaHCO₃ (10 mL). The aqueous phase was extracted twice with DCM (2×), washed with water then brine, dried over MgSO₄ and concentrated under reduced pressure. The residue was purified by flash chromatography (gradient hexanes/EtOAc 0-10-20-30-40-50% but problems of crystalisation in the column so eluent changed to DCM/EtOAc 20-30%). The first fraction isolated gave the expected sulfone FPC 01056A (261.8 mg, 57%) as a white solid. R_{f} 0.56 (hexane/EtOAc 5:5); Mp=127.7° C.; ¹H NMR (270 MHz, CDCl₃): δ 7.38 (4H, s app), 4.48 (2H, s), 3.88 (2H, s), 2.08 (3H, br s), 1.77 (6H, d, J=2.9 Hz), 1.75-1.60 (6H, m), 1.30 (9H, s); LC/MS (APCI) tᵣ=1.81 min, m/z 387.42 (M⁺−1H, 100), 388.43 (M⁺, 30); HPLC tᵣ=3.10 min (98.55% purity).

### 1-Adamantan-1-yl-2-m-tolylmethanesulfonyl-ethanone (STX2315, FPC 01057A)

m-CPBA (443 mg, 1.97 mmol, 77% max) was added to a solution of 1-Adamantan-1-yl-2-(3-methyl-benzylsulfanyl)-ethanone (310.6 mg, 0.99 mmol) in DCM (15 mL) at 0° C. and the reaction was stirred for 17 h. The reaction was quenched with NaHCO₃ (10 mL). The aqueous phase was extracted twice with DCM (2×), washed with water then brine, dried over MgSO₄ and concentrated under reduced pressure. The residue was purified by flash chromatography (gradient DCM/EtOAc 10-20-30%). The first fraction isolated gave the expected sulfone FPC 01057A (277 mg, 81%) as a white solid. R_{f} 0.68 (hexane/EtOAc 5:5); Mp=132.8° C.; ¹H NMR (270 MHz, CDCl₃): δ 7.31-7.13 (4H, m), 4.47 (2H, s), 3.88 (2H, s), 2.34 (3H, s), 2.06 (3H, br s), 1.77 (6H, d, J=2.7 Hz), 1.75-1.60 (6H, m); LC/MS (APCI) tᵣ=1.48 min. m/z 345.42 (M⁺−1H, 100), 346.43 (M⁺, 25); HPLC tᵣ=2.21 min (95.76% purity).

### 1-Adamantan-1-yl-2-(3-methyl-benzylsulfanyl)-ethanone (FPC01048, STX2316)

Et₃N (0.362 mL, 2.60 mmol) was then added to a solution of 1-Adamatyl bromomethyl ketone (335 mg, 1.30 mmol) and 3-(methylphenyl)methanethiol (180 mg, 1.30 mmol) in acetonitrile (10 mL). After 20 h of vigorous stirring at room temperature, the reaction was partitioned between ethyl acetate and water; the organic layer was washed with brine, dried over magnesium sulphate, filtered and concentrated in vacuo. Purification of the residue by flash chromatography (hexane/EtOAc, gradient elution) gave the expected titled compound as transparent oil (110 mg, 27%). TLC single spot at R_{f} 0.52 (DCM/EtOAc 95:5); ¹H NMR (270 MHz, CDCl₃): δ 7.21-7.03 (4H, m), 3.68 (2H, s), 3.22 (2H, s), 2.32 (3H, s), 2.02 (3H, br s), 1.81 (6H, d, J=2.9 Hz), 1.78-1.62 (6H, m); LC/MS (APCI) m/z 337.5 (M⁺+Na); HPLC tᵣ=9.56 min (96.90% purity) in 10% water-acetonitrile.

### 4-(2-Adamantan-1-yl-2-oxo-ethoxy)-benzoic acid methyl ester (STX2317, FPC 01058B)

Methyl 4-hydroxybenzoate (195 mg, 1.28 mmol) was added to a suspension of NaH (44 mg, 1.76 mmol, 60% in oil) in dry THF (4 mL) at 0° C. After 30 min, 1-adamatyl bromomethyl ketone (300 mg, 1.17 mmol) was added in dry THF (3.5 mL) then the reaction was allowed to slowly warm up over the week end. A major part of the solvent evaporated over that time, but the reaction was quenched with water, extracted with EtOAc (×2), washed with water then brine, then dried over MgSO₄ and concentrated under reduced pressure. The crude was purified by flash chromatography (gradient hexane/EtOAc 5-10-20-30%). The second fraction was isolated as the expected ether FPC 01058B (121.6 mg, 32%) as a white solid. R_{f} 0.30 (hexane/EtOAc 8:2); Mp=128.7° C.; ¹H NMR (270 z, CDCl₃): δ 7.96 (2H, dt, J=2.7, 8.9 Hz), 6.85 (dt, J=2.7, 9.1 Hz), 4.90 (2H, s), 3.87 (3H, s), 2.08 (3H, br s), 1.91 (6H, d, J=2.7 Hz), 1.83-1.68 (6H, m); LC/MS (APCI) tᵣ=1.58 min, m/z 328.54 (M⁺−1H, 10), 151.05 (benzoate-1H, 100); HPLC t, 2.39 min (97.78% purity).

### 4-(2-Adamantan-1-yl-2-oxo-ethoxy)benzoic acid (STX2318, FPC 01060)

Grined LiOH (38 mg, 0.91 mmol) was added in water (0.5 mL) to a suspension of 4-(2-Adamantan-1-yl-2-oxo-ethoxy)-benzoic acid methyl ester (100 mg, 0.30 mmol) in MeOH (2.5 mL) at room temperature. After 40 h, the starting material was gone and the clear solution was concentrated in vacuo. The aqueous phase was acidified from PH=12-14 to PH=2-0 with a solution of HCl 2N, then extracted with EtOAc twice, washed with water then brine, dried over magnesium sulphate and concentrated in vacuo. The purification of the crude by flash Chromatography (DCM/MeOH 5-10-15-20%) gave the titled compound FPC01060 as white solid (74.9 mg, 79%). R_{f} 0.37 (DCM/MeOH 9:1); Mp=185.2° C.; ¹H NMR (270 MHz, CDCl₃): δ 8.03 (2H, d, J=8.9 Hz), 6.87 (d, J=8.9 Hz), 4.93 (2H, s), 2.08 (3H, br s), 1.92 (6H, d, J=2.7 Hz), 1.85-1.69 (6H, m); LC/MS (APCI) tᵣ=1.07 min, m/z 313.49 (M⁺−1H, 100), 314.54 (M>, 25); HPLC tᵣ=1.28 min (97.98% purity).

### 3-(2-Adamantan-1-yl-2-oxo-ethoxy)-benzoic acid methyl ester (STX2319, FPC 01061)

Methyl 3-hydroxybenzoate (414 mg, 2.72 mmol) was added to a suspension of K₂CO₃ (752 mg, 5.44 mmol) in acetone (10 mL) at room temperature After 30 min, 1-adamatyl bromomethyl ketone (700 mg, 2.72 mmol) was added acetone (5+6 mL) and the reaction was stirred over night. In the morning, the starting material has disappeared and the reaction was quenched with water, extracted with EtOAc (×2), washed with brine, dried over MgSO₄ and concentrated under reduced pressure. The crude did not require any purification and gave the expected acid FPC01061 (907 mg, >99%) as white solid. R_{f} 0.31 (hexane/EtOAc 8:2); Mp=84.5° C.; ¹H NMR (270 MHz, CDCl₃): δ 7.63 (1H, dt, J=1.2, 6.5 Hz), 7.46 (1H, dd, J=1.5, 2.7 Hz), 7.33 (1H, t app, J=7.9 Hz), 7.11 (1H, ddd, J=1.0, 2.7, 8.4 Hz), 4.90 (2H, s), 3.89 (3H, s), 2.08 (3H, br s), 1.92 (6H, d, J=2.9 Hz), 1.82-1.68 (6H, m); LC/MS (APCI) tᵣ=1.52 min, m/z 327.63 (M⁺−1H, 100), 328 (M⁺, 30); HPLC tᵣ=3.05 min (99.58% purity).

### [4-(2-Adamantan-1-yl-2-oxo-ethoxy)-phenyl]-acetic acid methyl ester (STX2320, FPC01062)

Methyl 4-hydroxyphenylacetate (549 mg, 3.31 mmol) was added to a suspension of K₂CO₃ (915 mg, 6.62 mmol) in acetone (15 mL) at room temperature. After 30 min, 1-adamatyl bromomethyl ketone (850 mg, 3.31 mmol) was added acetone (10 mL) and the reaction was stirred over night. In the morning, the starting material has disappeared and the reaction was quenched with water, extracted with EtOAc (×2), washed with brine, dried over MgSO₄ and concentrated under reduced pressure. The crude was purified by flash chromatography (DCM/EtOAc 0-5-10%) and gave the expected ester FPC01062 (977.1 mg, 86%) as white solid. R_{f} 0.26 (hexane/EtOAc 8:2); Mp=71.9° C.; ¹H NMR (270 MHz, CDCl₃): δ 7.16 (2H, dt, J=2.9, 8.7 Hz), 6.80 (2H, dt, J=2.9, 8.7 Hz), 4.82 (2H, s), 3.66 (3H, s), 3.54 (2H, s), 2.06 (3H, br s), 1.90 (6H, d, J=2.9 Hz), 1.83-1.65 (6H, m); LC/MS (APCI) tᵣ=1.46 min, m/z 341.69 (M⁺−1H, 100); HPLC tᵣ=2.23 min (98.45% purity).

### 3-(2-Adamantan-1-yl-2-oxo-ethoxy)-benzoic acid (STX2361, FPC01064-2)

Grained LiOH (337 mg, 8.02 mmol) was added in water (4 mL) to a suspension of 3-(2-Adamantan-1-yl-2-oxo-ethoxy)-benzoic acid methyl ester (880.1 mg, 2.67 mmol) in MeOH (23 mL) at room temperature. After 5 days, the starting material was gone and the solution was concentrated in vacuo. The aqueous phase was acidified from pH=10 to pH=1-0 with a solution of HCl 2N, then extracted with EtOAc twice, washed with water then brine, dried over magnesium sulphate and concentrated in vacuo. The crude material did not need any purification and gave the titled compound FPC01064 (824.7 mg, 98%). The purification of the crude by flash Chromatography (DCM/MeOH 5-10%) gave the expected compound as white solid (796.8 mg, 95%). R_{f} 0.38 (DCM/MeOH 9:1); Mp=156.2° C.; ¹H NMR (270 MHz, CDCl₃): δ 7.72 (1H, dt, J=1.2, 7.9 Hz), 7.51 (1H, dd, J=1.5, 2.7 Hz), 7.37 (1H, t app, J=8.2 Hz), 7.17 (1H, ddd, J=1.0, 2.7, 8.4 Hz), 4.91 (2H, s), 2.09 (3H, br s), 1.93 (6H, d, J=2.9 Hz), 1.85-1.67 (6H, m); LC/MS (APCI) tᵣ=1.11 min, m/z 313.56 (M⁺−1H, 100); HPLC tᵣ=1.21 min (93.15% purity).

### 4-(2-Adamantan-1-yl-2-oxo-ethoxy)-N-benzyl-benzamide (STX2362, FPC01065A)

EDCI (43 mg, 0.23 mmol), DMAP (28 mg, 0.23 mmol) and Et₃N (32 μL, 0.23 mmol) were added to a solution of 4-(2-Adamantan-1-yl-2-oxo-ethoxy)-benzoic acid (59.4 mg, 0.19 mmol) in DCM (5 mL) at room temperature. After 30 min, benzylamine (25 μL, 0.23 mmol) was added neat and the reaction was allowed to stir over night. When the staring material was consumed, the reaction was quenched with a saturated solution of NH₄Cl, extracted with DCM twice then washed with a saturated solution of NaHCO₃ then brine. The organic phases were dried over magnesium sulphate, filtrated then concentrated in vacuo. The crude was purified by flash chromatography hexanes/EtOAc 8:2 then DCM/MeOH 5-10%) and the first fraction was isolated as the expected amide FPC01065A (30.4 mg, 40%) as a brownish crystal. R_{f} 0.76 (DCM/MeOH 9:1); Mp=145.6° C.; ¹H NMR (270 MHz, CDCl₃): δ 7.72 (2H, d app, J=8.9 Hz), 7.34-7.24 (5H, m), 6.84 (2H, d app, J=8.9 Hz), 6.35 (1H, t, J=5.2 Hz, NH), 4.88 (2H, s), 4.60 (2H, d, J=5.7 Hz), 2.03 (3H, br s), 1.90 (6H, d, J=2.7 Hz), 1.85-1.62 (6H, m); LC/MS (APCI) tᵣ=1.32 min, m/z 402.63 (M⁺−1H, 100), 403.47 (M⁺, 15); HPLC tᵣ=2.11 min (99.25% purity).

### 4-(2-Adamantan-1-yl-2-oxo-ethoxymethyl)-benzoic acid methyl ester (STX2363, FPC01066)

Methyl 4-(hydroxymethyl)benzoate (194 mg, 1.17 mmol) was added to a suspension of K₂CO₃ (323 mg, 2.34 mmol) in acetone (4 mL) at room temperature. After 30 min, 1-adamatyl bromomethyl ketone (300 mg, 1.17 mmol) was added acetone (5 mL) and the reaction was stirred for 48 h. The starting material had not disappeared so the reaction was quenched with water, extracted with EtOAc (×2), washed with brine, dried over MgSO₄ and concentrated under reduced pressure. The crude was resubmitted by dissolution in dry THF (9 mL) and addition of NaH neat (59 mg, 60% in oil, 2.34 mmol). After 24 h at room temperature, the reaction was quenched with water, extracted with EtOAc (×2), washed with brine, dried over MgSO₄ and concentrated under reduced pressure. The crude was purified by flash chromatography (hexanes/EtOAc 0-10-20-30%) and gave the expected ester FPC01066A (60.9 mg, 15%). Re-purification of FPC01066A (89% purity by HPLC, same conditions) gave the fraction FPC01066 as white solid. R_{f} 0.47 (hexane/EtOAc 7:3); Mp=88.7° C.; ¹H NMR (270 MHz, CDCl₃): δ 8.00 (2H, d app, J=8.4 Hz), 7.42 (2H, d, J=8.2 Hz), 4.61 (2H, s), 4.32 (2H, s), 3.90 (3H, s), 2.061 (3H, br s), 1.80 (6H, d, J=2.7 Hz), 1.79-1.60 (6H, m); LC/MS (APCI) tᵣ=1.63 min, m/z 365.48 (M⁺+Na, 45), 148.94 (100); HPLC tᵣ=2.20 min (94.89% purity).

### [4-(2-Adamantan-1-yl-2-oxo-ethoxy)-phenyl]-acetic acid (STX2364, FPC01067cr)

Grined LiOH (355 mg, 8.45 mmol) was added in water (4 mL) to a suspension of [4-(2-Adamantan-1-yl-2-oxo-ethoxy)-phenyl]-acetic acid methyl ester (965.1 mg, 2.82 mmol) in MeOH (24 mL) at room temperature. The reaction was vigorously stirred over night and the starting material was consumed in 15 h. The solution was concentrated in vacuo, the aqueous phase was acidified from pH=14 to pH=1-0 with a solution of HCl 2N, then extracted with EtOAc twice, washed with water then brine, dried over magnesium sulphate and concentrated in vacuo. The crude material did not need any purification and gave the titled compound FPC01064 as white solid (885.6 mg, 96%). No need of further purification for preliminary testing. R_{f} 0.46 (DCM/MeOH 9:1); Mp=153.4° C.; ¹H NMR (270 MHz, CDCl₃): δ 7.17 (2H, d, J=8.9 Hz), 6.81 (2H, d, J=8.9 Hz), 4.82 (2H, s), 3.56 (2H, s), 2.07 (3H, br s), 1.90 (6H, d, J=2.7 Hz), 1.82-1.65 (6H, m); LC/MS (APCI) tᵣ=1.12 min, m/z 327.56 (M⁺−1H, 100), 328.61 (20); HPLC tᵣ=1.23 min (96.97% purity).

### 4-(2-Adamantan-1-yl-2-oxo-ethoxy)-N-phenyl-benzamide (STX2365, FPC01068A)

EDCI (73 mg, 0.38 mmol), DMAP (46 mg, 0.38 mmol) and Et₃N (563 μL, 0.38 mmol) were added to a solution of 4-(2-Adamantan-1-yl-2-oxo-ethoxy)-benzoic acid (100 mg, 0.32 mmol) in DCM (8.5 mL) at room temperature. After 30 min, aniline (35 μL, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01068A (54.8 mg, 44%) as white solid. R_{f} 0.79 (DCM/MeOH 9:1); Mp=184.3° C.; ¹H NMR (270 MHz, CDCl₃): δ 7.80 (2H, d app, J=8.7 Hz), 7.71 (1H, br s), 7.60 (2H, d, J=8.6 Hz), 7.35 (2H, t, J=7.4 Hz), 7.13 (1H, t, J=7.2 Hz), 6.91 (2H, d app, J=8.9 Hz), 4.93 (2H, s), 2.09 (3H, br s), 1.93 (6H, s app), 1.90-1.65 (6H, m); LC/MS (APCI) tᵣ=1.37 min, m/z/z 388.50 (M⁺−1H, 100); HPLC tᵣ=2.18 min (98.83% purity).

### 4-(2-Adamantan-1-yl-2-oxo-ethoxy)-N-ethyl-benzamide (STX2366, FPC01069B1)

EDCI (73 mg, 0.38 mmol), DMAP (46 mg, 0.38 mmol) and Et₃N (563 μL, 0.38 mmol) were added to a solution of 4-(2-Adamantan-1-yl-2-oxo-ethoxy)benzoic acid (100 mg, 0.32 mmol) in DCM (8.5 mL) at room temperature. After 30 min, ethylamine (19 μL, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01069B1 (31.4 mg, 44%) as white solid. R_{f} 0.50 (DCM/MeOH 9:1); ¹H NMR (270 MHz, CDCl₃): δ 7.68 (2H, d app, J=8.9 Hz), 6.83 (2H, d app, J=8.9 Hz), 6.11 (1H, br s, NE, 4.88 (2H, s), 3.50-3.38 (2H, m), 2.07 (3H, br s), 1.90 (6H, d, J=2.7 Hz), 1.82-1.64 (6H, m), 1.20 (3H, t, J=7.4 Hz); LC/MS (APCI) tᵣ=1.29 min. m/z 341.41 (M⁺, 10), 340.43 (M⁺−1H, 38), 147.89 (100); HPLC (FPC01069B1) tᵣ=1.88 min (96.43% purity).

### 4-(2-Adamantan-1-yl-2-oxo-ethoxy)-N-isopropyl-benzamide (STX2367, FPC01070)

EDCI (73 mg, 0.38 mmol), DMAP (46 mg, 0.38 mmol) and Et₃N (563 μL, 0.38 mmol) were added to a solution of 4-(2-Adamantan-1-yl-2-oxo-ethoxy)-benzoic acid (100 mg, 0.32 mmol) in DCM (8.5 mL) at room temperature. After 30 min, isopropyl amine (32 mL, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01070 (57.2 mg, 50%) as white solid. R_{f} 0.59 (DCM/MeOH 9:1); ¹H NMR (270 MHz, CDCl₃): δ 7.65 (2H, d app, J=8.9 Hz), 6.82 (2H, d app, J=8.9 Hz), 5.87 (1H, d, J=7.4 Hz, NH), 4.87 (2H, s), 4.30-4.16 (1H, m), 2.06 (3H, br s), 1.90 (6H, d, J=2.7 Hz), 1.85-1.64 (6H, m); LC/MS (APCI) tᵣ=1.33 min, m/z 355.48 (M⁺, 25), 354.36 (M⁺−1H, 85), 161.98 (100); HPLC tᵣ=1.95 min (97.92% purity).

### 4-(2-Adamantan-1-yl-2-oxo-ethoxy)-N-methyl-benzamide (STX2368, FPC01071A)

EDCI (73 mg, 0.38 mmol), DMAP (46 mg, 0.38 mmol) and Et₃N (563 μL, 0.38 mmol) were added to a solution of 4-(2-Adamantan-1-yl-2-oxo-ethoxy)-benzoic acid (100 mg, 0.32 mmol) in DCM (8.5 mL) at room temperature. After 30 min, methylamine (0.19 μL, 2M solution in MeOH, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01071A (39.1 mg, 37%) as white solid. R_{f} 0.81 (DCM/MeOH 9:1); Mp=127.1° C.; ¹H NMR (270 MHz, CDCl₃): δ 7.68 (2H, d app, J=9.1 Hz), 6.83 (2H, d app, J=8.9 Hz), 6.08 (1H, br s, NH), 4.88 (2H, s), 2.96 (3H, d, J=−4.9 Hz), 2.07 (3H, br s), 1.90 (6H, d, J=2.7 Hz), 1.83-1.66 (6H, m); LC/MS (APCI) tᵣ=1.55 min, m/z 351.56 (M⁺+Na, 100); HPLC tᵣ=2.55 min (97.51% purity).

### 4-(2-Adamantan-1-yl-2-oxo-ethoxy)-N,N-diethyl-benzamide (STX2375, FPC01072,)

EDCI (73 mg, 0.38 mmol), DMAP (46 mg, 0.38 mmol) and Et₃N (563 μL, 0.38 mmol) were added to a solution of 4-(2-Adamantan-1-yl-2-oxo-ethoxy)-benzoic acid (100 mg, 0.32 mmol) in DCM (8.5 mL) at room temperature. After 30 min, diethylamine (39 mL, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the main first fraction was isolated as the expected amide FPC01072 (44.1 mg, 37%) as white solid. R_{f} 0.73 (DCM/MeOH 9:1); Mp=103.9° C.; ¹H NMR (270 MHz, CDCl₃): δ 7.30 (2H, d app, J=8.9 Hz), 6.83 (2H, d app, J=8.9 Hz), 4.86 (2H, s), 3.37 (4H, br s), 2.07 (3H, br s), 1.91 (6H, d, J=2.7 Hz), 1.84-1.64 (6H, m), 1.15 (6H, br s); LC/MS (APCI) tᵣ=1.35 min, m/z 392.70 (M⁺+Na, 100); HPLC tᵣ=2.26 min (>96.71% purity).

### 4-(2-Adamantan-1-yl-2-oxo-ethoxy)-N-furan-2-ylmethyl-benzamide (STX2376, FPC01073)

EDCI (73 mg, 0.38 mmol), DMAP (46 mg, 0.38 mmol) and Et₃N (563 mL, 0.38 mmol) were added to a solution of 4-(2-Adamantan-1-yl-2-oxo-ethoxy)-benzoic acid (100 mg, 0.32 mmol) in DCM (8.5 mL) at room temperature. After 30 min, furfurylamine (34 μL, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01073 (70.4 mg, 56%) as white solid. R_{f} 0.73 (DCM/MeOH 9:1); Mp=162.2° C.; ¹H NMR (270 MHz, CDCl₃): δ 7.71 (2H, d app, J=8.9 Hz), 7.34 (1H, dd, J=1.0, 2.0 Hz), 6.83 (2H, d app, J=8.9 Hz), 6.44 (1H, t, J=4.9 Hz, NH), 6.27 (1H, ddd, J=2.0, 3.0, 15.0 Hz), 4.87 (2H, s), 4.58 (2H, d, J=5.5 Hz), 2.06 (3H, br s), 1.90 (6H, d, J=2.7 Hz), 1.83-1.63 (6H, m); LC/MS (APCI) tᵣ=1.26 min, m/z 392.63 (M⁺−1H, 100), 393.54 (M⁺, 12); HPLC tᵣ=1.89 min (>95.06% purity).

### 4-(2-Adamantan-1-yl-2-oxo-ethoxy)-N-tert-butyl-benzamide (STX2377, FPC01074)

EDCI (73 mg, 0.38 mmol), DMAP (46 mg, 0.38 mmol) and Et₃N (563 μL, 0.38 mmol) were added to a solution of 4-(2-Adamantan-1-yl-2-oxo-ethoxy)-benzoic acid (100 mg, 0.32 mmol) in DCM (8.5 mL) at room temperature. After 30 min, tert-butylamine (40 μL, 0.38 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01074 (41 mg, 35%) as white solid. R_{f} 0.78 (DCM/MeOH 9:1); Mp=179.3° C.; ¹H NMR (270 MHz, CDCl₃): δ 7.64 (2H, d app, J=8.9 Hz), 6.83 (2H, d app, J=8.9 Hz), 5.84 (1H, br s, NM, 4.87 (2H, s), 2.07 (3H, br s), 1.90 (6H, d, J=2.7 Hz), 1.82-1.65 (6H, m), 1.41 (9H, s); LC/MS (APCI) tᵣ=1.35 min, m/z 368.49 (M⁺−1H, 100), 369.54 (M⁺, 15); HPLC tᵣ=2.29 min (>97.45% purity).

### 2-Adamantan-1-yl-N-(5-chloro-thiophen-2-ylmethyl)-N-methyl-acetamide (STX2393, XDS04163)

The title compound was synthesized with general amide formation method from adamantan-1-yl-acetyl chloride (0.5 mmol) and the amine (123 mg, 0.75 mmol). The title compound (140 mg, 83%) was obtained as white solid. mp 73.5-75° C.; TLC single spot at R_{f}: 0.39 (30% EtOAc/hexane); ¹H NMR (270 MHz, CDCl₃) indicated signals from rotamers in 4:1 ratio: δ 1.55-1.71 (12H, m, 6×CH₂), 1.95 (3H, broad, 3×CH), 2.14 (2H, s, CH₂), 2.99 (3H, s, CH₃), 4.57 (2H, s, CH₂) and 6.71 (2H, s, ArH); LC/MS (APCI) m/z 338 (M⁺+H); HPLC tᵣ=3.81 min (>99%) in 10% water-acetonitrile.

### 2-Adamantan-1-yl-N-5-ethyl-thiophen-2-ylmethyl)-N-methyl-acetamide (STX2394, XDS04164)

The title compound was synthesized with general amide formation method from adamantan-1-yl-acetyl chloride (0.5 mmol) and the amine (117 mg, 0.75 mmol). The title compound (140 mg, 84%) was obtained as white solid. mp 54-55° C.; TLC single spot at R_{f}: 0.51 (30% EtOAc/hexane); ¹H NMR (270 MHz, CDCl₃) indicated signals from rotamers in 2:1 ratio: δ 1.26 (3H, t, J=7.6 Hz, CH₃), 1.55-1.71 (12H, m, 6×CH₂), 1.95 (3H, broad, 3×CM), 2.14 (2H, s, CH₂), 2.79 (2H, q, J=7.6 Hz, CH₂), 2.98 (3H, s, CH₃), 4.63 (2H, s, CH₂), 6.58 (1H, d, J=3.4 Hz, ArH) and 6.57 (1H, d, J=3.4 Hz, ArH); δ 1.27 (3H, t, J=7.6 Hz, CH₃), 1.55-1.71 (12H, m, 6×CH₂), 1.95 (3H, broad, 3×CH), 2.23 (2H, s, CH₂), 2.79 (2H, q, J=7.6 Hz, CH₂), 2.92 (3H, s, CH₃), 4.60 (2H, s, CH₂), 6.62 (1H, d, J=3.4 Hz, ArH) and 6.68 (1H, d, J=3.4 Hz, ArH); LC/MS (APCI) m/z 332 (M⁺+H), tᵣ=2.02 min (97%) in 5% water-methanol; HPLC t=4.00 min (>99%) in 10% water-acetonitrile.

### 2-Adamantan-1-yl-N-methyl-N-(3-methyl-thiophen-2-ylmethyl)-acetamide (STX2395, XDS04165)

The title compound was synthesized with general amide formation method from adamantan-1-yl-acetyl chloride (0.5 mmol) and the amine (106 mg, 0.75 mmol). The title compound (140 mg, 88%) was obtained as clear oil. TLC single spot at R_{f}: 0.50 (30% EtOAc/hexane); ¹H NMR (270 MHz, CDCl₃) δ 1.55-1.68 (12H, m, 6×CH₂), 1.95 (3H, broad, 3×CH), 2.15 (2H, s, CH₂), 2.20 (3H, s, CH₃), 2.96 (3H, s, CH₃), 4.69 (2H, s, CH₂), 6.78 (1H, d, J=4.9 Hz, ArH) and 7.10 (1H, d, J=4.9 Hz, ArH); LC/MS (APCI) m/z 318 (M⁺+H), tᵣ=1.78 min (97%) in 5% water-methanol; HPLC tᵣ=3.24 min (>99%) in 10% water-acetonitrile.

### 2-Adamantan-1-yl-N-(4-bromo-thiophen-2-ylmethyl)-N-methyl-acetamide (STX2396, XDS04166)

The title compound was synthesized with general amide formation method from adamantan-1-yl-acetyl chloride (0.5 mmol) and the amine (155 mg, 0.75 mmol). The title compound (140 mg, 73%) was obtained as white solid. mp 86-88.5° C.; TLC single spot at R_{f}: 0.49 (30% EtOAc/hexane); ¹H NMR (270 MHz, CDCl₃) indicated signals from rotamers in 5:1 ratio: δ 1.55-1.71 (12H, m, 6×CH₂), 1.95 (3H, broad, 3×CM), 2.15 (2H, s, CH₂), 3.00 (3H, s, CH₃), 4.64 (2H, s, CH₂), 6.87 (1H, d, J=1.5 Hz, ArH) and 7.11 (1H, d, J=1.5 Hz, ArH); LC/MS (APCI) m/z 382 (M⁺+H), tᵣ=1.80 min (94%) in 5% water-methanol; HPLC tᵣ=3.53 min (95%) in 10% water-acetonitrile.

### N-(4-hydroxy-2-methylphenyl)acetamide (XDS04167)

To a cold solution (0° C.) of 4-acetamido-3-methylphenyl acetate (1000 mg, 4.8 mmol) in methanol (50 mL) was added K₂CO₃ (730 mg, 5.3 mmol). The mixture was stirred at 0° C. for 40 min, neutralized with 4N HCl to pH 3, then concentrated in vacuo, partitioned between EtOAc and brine solution. The organic phase was washed with brine, dried over magnesium sulfate and concentrated in vacuo. The product (680 mg, 86%) was obtained as yellow solid. mp 126.5-127° C.; TLC single spot at R_{f}: 0.61 (10% methanol/DCM); ¹H NMR (270 MHz, DMSO) δ 1.98 (3H, s, CH₃), 2.07 (3H, s, CH₃), 6.52 (1H, dd, J=8.4, 2.7 Hz, ArH), 6.58 (1H, d, J=2.7 Hz, ArH), 7.01 (1H, d, J=8.4 Hz, ArH), 9.08 (1H, s, OH) and 9.18 (1H, s, NH); LC/MS (APCI) m/z 164 (M⁺−H); HPLC tᵣ=1.13 min (95%) in 10% water-acetonitrile.

### N-(2-chloro-4-hydroxyphenyl)acetamide (XDS04168)

To a cold solution (0° C.) of 4-amino-3-chlorophenol (1000 mg, 5.5 mmol) in DCM (50 mL) was added triethylamine (2.2 mL, 15.8 mmol), followed by Ac₂O (1.15 mL, 12.1 mmol). The mixture was stirred at 0° C. for 3 h, then at room temperature overnight. Methanol (20 mL) was added and the mixture was concentrated in vacuo, partitioned between EtOAc and brine solution. The organic phase was washed with 2N HCl, saturated NaHCO₃ and brine, dried over magnesium sulfate and concentrated in vacuo to give the crude intermediate, which was dissolved in methanol (50 mL). K₂CO₃ (730 mg, 5.3 mmol) was added. The mixture was stirred at 0° C. for 1.5 h, neutralized with 4N HCl to pH 3, then concentrated in vacuo, partitioned between EtOAc and brine solution. The organic phase was washed with brine, dried over magnesium sulfate and concentrated in vacuo. The product (690 mg, 68%) was obtained as yellow solid. mp 123-124° C.;-TLC single spot at R_{f}: 0.68 (10% methanol/DCM); ¹H NMR (270 MHz, CDCl₃) δ 2.21 (3H, s, CH₃), 5.35 (1H, s, OH), 6.70 (11H, dd, J=8.8, 2.8 Hz, ArH), 6.86 (11H, d, J=2.8 Hz, ArH), 7.32 (1H, s, NH) and 7.39 (1H, d, J=8.4 Hz, ArH); LC/MS (APCI) m/z 184 (M⁺−H); HPLC tᵣ=1.24 min (>98%) in 10% water-acetonitrile.

### N-(2-fluoro-4-hydroxyphenyl)acetamide (XDS04169)

The title compound was synthesized as above. The product (670 mg, 50%) was obtained as off-white solid. mp 148.5-150° C.; TLC single spot at R_{f}: 0.69 (10% methanol/DCM); ¹H NMR (270 MHz, CDCl₃) δ 2.19 (3H, s, CH₃), 5.19 (1H, s, OH), 6.54-6.63 (2H, m, ArH), 6.62 (1H, s, NH) and 7.98 (1H, t, J=8.7 Hz, ArH); LC/MS (APCI) m/z 168 (M⁺−H); HPLC tᵣ=1.23 min (94%) in 10% water-acetonitrile.

### N-[4-(2-Adamantan-1-yl-2-oxo-ethoxy)-2-methyl-phenyl]-acetamide (STX2397, XDS04171)

To a solution of N-(4-hydroxy-2-methylphenyl)acetamide (165 mg, 1.0 mmol) in DMF (5 mL) were added K₂CO₃ (200 mg) and tetrabutylammonium bromide (10 mg). The mixture was stirred at room temperature for 10 min, 1-adamantyl bromomethyl ketone (258 mg, 1.0 mmol) was added. After 3 h, the mixture was diluted with water and the precipitate was collected and washed with water. Purification with flash column (DCM-methanol gradient elution) gave product (158 mg, 46%) as off-white amorphous solid. mp 201-204° C.; TLC single spot at R_{f}: 0.51 (10% methanol/DCM); ¹H NMR (270 MHz, CDCl₃) δ 1.68-1.82 (6H, m, 3×CH₂), 1.90 (6H, d, J=2.7 Hz, 3×CH₂), 2.07 (3H, broad, 3×CH), 2.16 (3H, s, CH₃), 2.20 (3H, s, CH₃), 4.80 (2H, s, CH₂), 6.65 (1H, dd, J=8.8, 2.9 Hz, ArH), 6.72 (1H, d, J=3.0 Hz, ArH), 6.83 (1H, broad, NH), and 7.17 (1H, d, J=8.7 Hz, ArH); LC/MS (APCI) m/z 440 (M⁺−H); tᵣ=1.20 min (93%) in 5% water-methanol; HPLC tᵣ=1.78 min (99%) in 10% water-acetonitrile.

### Adamantane-1-carboxylic acid (4-chloro-phenyl)-amide (STX2398, XDS04173)

The title compound was synthesized with general amide formation method. The title compound (40 mg, 66%) was obtained as white crystals. mp 208-209° C.; TLC single spot at R_{f}: 0.70 (40% EtOAc/Hexane); ¹H NMR (270 MHz, CDCl₃) δ 1.69-1.80 (6H, m, 3×CH₂), 1.94 (6H, d, J=2.6 Hz, 3×CH₂), 2.09 (3H, broad, 3×CM), 7.25 (2H, m, ArH) and 7.48 (2H, dt, J=7.2, 2.2 Hz, ArH); LC/MS (APCI) m/z 288 (M⁺−H), tᵣ=1.52 min (99%) in 5% water-methanol; HPLC tᵣ=2.76 min (99%) in 10% water-acetonitrile.

### Adamantane-1-carboxylic acid pyridin-3-ylamide (STX2399, XDS04174)

The title compound was synthesized with general amide formation method. The title compound (40 mg, 78%) was obtained as white solid. mp 129-131° C.; TLC single spot at R_{f}: 0.66 (10% methanol/DCM); ¹H NMR (270 MHz, CDCl₃) δ 1.66-1.81 (6H, m, 3×CH₂), 1.91-2.02 (6H, m, 3×CH₂), 2.11 (3H, broad, 3×CH), 7.25 (1H, m, ArH), 7.34 (1H, br, NM, 8.22 (1H, dq, J=5.2, 1.5 Hz, ArH), 8.33 (1H, dd, J=4.9, 1.4 Hz, ArH) and 8.54 (1H, d, J=2.5 Hz, ArH); LC/MS (APCI) m/z 255 (M⁺−H), tᵣ=1.28 min (98%) in 5% water-methanol; HPLC tᵣ=1.83 min (99%) in 10% water-acetonitrile.

### Adamantane-1-carboxylic acid (6-methyl-pyridin-2-yl)-amide (STX2400, XDS04175)

The title compound was synthesized with general amide formation method. The title compound (35 mg, 65%) was obtained as white solid. mp 122-123° C.; TLC single spot at R_{f}: 0.75 (20% EtOAc/DCM); ¹H NMR (270 MHz, CDCl₃) δ 1.66-1.79 (6H, m, 3×CH₂), 1.96 (6H, d, J=2.7 Hz, 3×CH₂), 2.07 (3H, broad, 3×CH), 2.43 (3H, s, CH₃), 6.86 (1H, d, J=7.4 Hz, ArH), 7.55 (1H, t, J=7.9 Hz, ArH), 7.86 (1H, br, NH) and 8.05 (1H, d, J=8.4 Hz, ArH); LC/MS (APCI) m/z 269 (M⁺−H), tᵣ=1.61 min (98%) in 5% water-methanol; HPLC tᵣ=2.75 min (98%) in 10% water-acetonitrile.

### Adamantane-1-carboxylic acid thiazol-2-ylamide (STX2401, XDS04176)

The title compound was synthesized with general amide formation method. The title compound (38 mg, 73%) was obtained as white solid. mp 200-201° C.; TLC single spot at R_{f}: 0.70 (20% EtOAc/DCM); ¹H NMR (270 MHz, CDCl₃) δ 1.68-1.80 (6H, m, 3×CH₂), 1.95 (6H, d, J=2.8 Hz, 3×CH₂), 2.09 (3H, broad, 3×CH), 6.95 (1H, d, J=3.5 Hz, ArH), 7.43 (1H, d, J=3.5 Hz, ArH) and 8.98 (1H, br, NH); LC/MS (APCI) m/z 261 (M⁺−H), tᵣ=1.42 min (99%) in 5% water-methanol; HPLC tᵣ=2.07 min (98%) in 10% water-acetonitrile.

### 2-Adamantan-1-yl-1-[4-(4-chloro-benzyl)-piperazin-1-yl]-ethanone (STX2402, XDS04178)

The title compound was synthesized with general amide formation method from adamantan-1-yl-acetyl chloride (0.5 mmol) and the amine (106 mg, 0.5 mmol). The title compound (189 mg, 98%) was obtained as off-white solid. mp 119-121° C.; TLC single spot at R_{f}: 0.78 (10% methanol/DCM); ¹H NMR (270 MHz, CDCl₃): δ 1.59-1.70 (12H, m, 6×CH₂), 1.95 (3H, broad, 3×CH), 2.12 (2H, s, CH₂), 2.39 (4H, m, 2×CH₂), 3.46 (2H, s, CH₂), 3.49 (2H, t, J=5.0 Hz, 2×CH), 3.63 (2H, t, J=5.0 Hz, 2×CH) and 7.22-7.32 (4H, m, ArH); LC/MS (APCI) m/z 387 (<+H), tᵣ=2.09 min (99%) in 5% water-methanol; HPLC tᵣ=4.38 min (99%) in 10% water-acetonitrile.

### 2-Adamantan-1-yl-1-[4-(3-methyl-benzyl)-piperazin-1-yl]-ethanone (STX2403, XDS04179)

The title compound was synthesized with general amide formation method from adamantan-1-yl-acetyl chloride (0.5 mmol) and the amine (95 mg, 0.5 mmol). The title compound (180 mg, 98%) was obtained as off-white solid. mp 86-88° C.; TLC single spot at R_{f}: 0.69 (10% methanol/DCM); ¹H NMR (270 MHz, CDCl₃): δ 1.58-1.71 (12H, m, 6×CH₂), 1.95 (3H, broad, 3×CH), 2.12 (2H, s, CH₂), 2.33 (3H, s, CH₃), 2.40 (4H, m, 2×CH₂), 3.46 (2H, s, CH₂), 3.50 (2H, t, J=5.2 Hz, 2×CH), 3.64 (2H, t, J=5.2 Hz, 2×CH), 7.05-7.11 (3H, m, ArH) and 7.20 (1H, t, J=7.2 Hz, ArH); LC/MS (APCI) m/z 367 (M⁺+H), tᵣ=2.13 min (99%) in 5% water-methanol; HPLC tᵣ=4.31 min (99%) in 10% water-acetonitrile.

### 2-Adamantan-1-yl-1-[4-(4-chloro-phenyl)-piperazin-1-yl]-ethanone (STX2404, XDS04180)

The title compound was synthesized with general amide formation method from adamantan-1-yl-acetyl chloride (0.5 mmol) and the amine HCl salt (135 mg, 0.5 mmol). The title compound (106 mg, 57%) was obtained as off-white solid. mp 112.5-115° C.; TLC single spot at R_{f}: 0.69 (30% EtOAc/DCM); ¹H NMR (270 MHz, CDCl₃): δ 1.58-1.75 (12H, m, 6×CH₂), 1.96 (3H, broad, 3×CH), 2.18 (2H, s, CH₂), 3.11 (4H, m, 2×CH₂), 3.66 (2H, t, J=5.2 Hz, 2×CH), 3.79 (2H, t, J=5.2 Hz, 2×CH), 6.83 (2H, dt, J=9.1, 2.2 Hz, ArH) and 7.21 (2H, dt, J=9.1, 2.2 Hz, ArH); LC/MS (APCI) m/z 395 (M⁺+Na), tᵣ=1.98 min (99%) in 5% water-methanol; HPLC tᵣ=3.76 min (99%) in 10% water-acetonitrile.

### 2-Adamantan-1-yl-1-[4-(furan-2-carbonyl)-piperazin-1-yl]-ethanone (STX2405, XDS04181)

The title compound was synthesized with general amide formation method from adamantan-1-yl-acetyl chloride (0.5 mmol) and the amine (90 mg, 0.5 mmol). The title compound (79 mg, 44%) was obtained as off-white solid. mp 98-100° C.; TLC single spot at R_{f}: 0.72 (10% methanol/DCM); ¹H NMR (270 MHz, CDCl₃): δ 1.58-1.75 (12H, m, 6×CH₂), 1.96 (3H, broad, 3×CH), 2.18 (2H, s, CH₂), 3.61 (2H, m, 2×CH), 3.73 (2H, m, 2×CH), 3.79 (4H, br, 2×CH₂), 6.49 (1H, dd, J=3.4, 1.7 Hz, ArH), 7.04 (1H, dd, J=3.4, 0.7 Hz, ArH) and 7.48 (1H, dd, J=1.7, 0.7 Hz, ArH); LC/MS (APCI) m/z 379 (M⁺+Na), tᵣ=1.27 min (99%) in 5% water-methanol; HPLC tᵣ=1.94 min (99%) in 10% water-acetonitrile.

### 2-Adamantan-1-yl-N-methyl-N-thiophen-3-ylmethyl-acetamide (STX2406, XDS04182)

The title compound was synthesized with general amide formation method from adamantan-1-yl-acetyl chloride (0.5 mmol) and the amine (95 mg, 0.75 mmol). The title compound (75 mg, 49%) was obtained as white solid. mp 69-71° C.; TLC single spot at R_{f}: 0.75 (30% EtOAc/DCM); ¹H NMR (270 MHz, CDCl₃) indicated signals from rotamers in 2:1 ratio: δ 1.55-1.75 (12H, m, 6×CH₂), 1.95 (3H, broad, 3×CH), 2.16 (2H, s, CH₂), 2.95 (3H, s, CH₃), 4.57 (2H, s, CH₂), 7.02 (1H, m, ArH), 7.11 (1H, m, ArH) and 7.26 (1H, m, ArH); δ 1.55-1.75 (12H, m, 6×CH₂), 1.95 (3H, broad, 3×CM), 2.19 (2H, s, CH₂), 2.92 (3H, s, CH₃), 4.54 (2H, s, CH₂), 6.91 (1H, m, ArH), 7.02 (1H, m, ArH) and 7.33 (1H, m, ArH); LC/MS (APCI) m/z 304 (M⁺+H), tᵣ=1.60 min (99%) in 5% water-methanol; HPLC tᵣ=2.90 min (>99%) in 10% water-acetonitrile.

### 2-Adamantan-1-yl-N-methyl-N-(1-methyl-1H-pyrrol-2-ylmethyl)-acetamide (STX2407, XDS04183)

The title compound was synthesized with general amide formation method from adamantan-1-yl-acetyl chloride (0.5 mmol) and the amine (93 mg, 0.75 mmol). The title compound (75 mg, 50%) was obtained as white solid. mp 75-76.5° C.; TLC single spot at R_{f}: 0.78 (30% EtOAc/DCM); ¹H NMR (270 MHz, CDCl₃): δ 1.60-1.72 (12H, m, 6×CH₂), 1.95 (3H, broad, 3×CM), 2.14 (2H, s, CH₂), 2.88 (3H, s, CH₃), 3.57 (3H, s, CH₃), 4.59 (2H, s, CH₂), 6.03-6.07 (2H, m, ArH) and 6.59 (1H, t, J=2.1 Hz, ArH); LC/MS (APCI) m/z 323 (M⁺+Na), tᵣ=1.62 min (97%) in 5% water-methanol; HPLC tᵣ=2.81 min (98%) in 10% water-acetonitrile.

### 2-Adamantan-1-yl-N-methyl-N-(6-methyl-pyridin-2-ylmethyl)-acetamide (STX2408, XDS04184)

The title compound was synthesized with general amide formation method from adamantan-1-yl-acetyl chloride (0.5 mmol) and the amine (102 mg, 0.75 mmol). The title compound (82 mg, 53%) was obtained as clear oil. TLC single spot at R_{f}: 0.68 (10% methanol/DCM); ¹H NMR (270 MHz, CDCl₃) indicated signals from rotamers in 1:1 ratio: δ 1.57-1.75 (12H, m, 6×CH₂), 1.96 (3H, broad, 3×CH), 2.21 (2H, s, CH₂), 2.51 (3H, s, CH₃), 3.05 (3H, s, CH₃), 4.69 (2H, s, CH₂), 7.02 (1H, d, J=7.8 Hz, Arm), 7.07 (1H, d, J=7.7 Hz, ArH), and 7.52 (1H, d, J=7.8 Hz, ArH); δ 1.57-1.75 (12H, m, 6×CH₂), 1.96 (31, broad, 3×CH), 2.13 (2H, s, CH₂), 2.54 (3H, s, CH₃), 2.98 (3H, s, CH₃), 4.65 (2H, s, CH₂), 6.89 (1H, d, J=7.8 Hz, ArH), 7.07 (1H, d, J=7.7 Hz, ArH), and 7.57 (1H, d, J=7.8 Hz, ArH); LC/MS (APCI) m/z 313 (M⁺+H), tᵣ=5.52 min (98%) in 5% water-methanol; HPLC tᵣ=4.92 min (98%) in 10% water-acetonitrile.

### 2-Adamantan-1-yl-N-methyl-N-pyridin-3-ylmethyl-acetamide (STX2409, XDS04185)

The title compound was synthesized with general amide formation method from adamantan-1-yl-acetyl chloride (0.5 mmol) and the amine (61 mg, 0.50 mmol). The title compound (78 mg, 52%) was obtained as off-white solid. mp 67-68.5° C.; TLC single spot at R_{f}: 0.52 (10% methanol/DCM); ¹H NMR (270 MHz, CDCl₃) δ 1.58-1.72 (12H, m, 6×CH₂), 1.96 (3H, broad, 3×CH), 2.19 (2H, s, CH₂), 2.97 (3H, s, CH₃), 4.60 (2H, s, CH₂), 7.24 (1H, m, ArH), 7.65 (1H, dt, J=8.2, 1.7 Hz, ArH) and 8.47-8.57 (2H, m, ArH); LC/MS (APCI) m/z 299 (+H), tᵣ=1.34 min (97%) in 5% water-methanol; HPLC tᵣ=2.04 min (96%) in 10% water-acetonitrile.

### 2-Adamantan-1-yl-N-methyl-N-(5-methylsulfanyl-thiophen-2-ylmethyl)-acetamide (STX2410, XDS04186)

The title compound was synthesized with general amide formation method from adamantan-1-yl-acetyl chloride (1.0 mmol) and the amine (173 mg, 1.0 mmol). The title compound (158 mg, 45%) was obtained as clear oil. TLC single spot at R_{f}: 0.81 (30% EtOAc/DCM); ¹H NMR (270 MHz, CDCl₃) indicated signals from rotamers in 4:1 ratio: δ 1.56-1.70 (12H, m, 6×CH₂), 1.95 (3H, broad, 3×CH), 2.15 (2H, s, CH₂), 2.45 (3H, s, CH₃), 3.00 (3H, s, CH₃), 4.62 (2H, s, CH₂), 6.78 (1H, d, J=3.5 Hz, ArH) and 6.87 (1H, d, J=3.5 Hz, ArH); δ 1.56-1.70 (12H, m, 6×CH₂), 1.95 (3H, broad, 3×CH), 2.21 (2H, s, CH₂), 2.46 (3H, s, CH₃), 2.93 (3H, s, CH₃), 4.61 (2H, s, CH₂), 6.72 (1H, d, J=3.5 Hz, ArH) and 6.92 (1H, d, J=3.5 Hz, ArH); LC/MS (APCI) m/z 350 (+11), tᵣ=1.79 min (99%) in 5% water-methanol; HPLC tᵣ=3.72 min (>99%) in 10% water-acetonitrile.

### Adamantane-1-carboxylic acid 3-nitro-benzylamide (STX2411, XDS04187)

The title compound was synthesized with general amide formation method from adamantan-1-yl-carbonyl chloride (400 mg, 2.0 mmol) and the amine HCl salt (396 mg, 2.1 mmol). The title compound (550 mg, 87%) was obtained as white solid. mp 145.5-147° C.; TLC single spot at R_{f}: 0.72 (15% EtOAc/DCM); ¹H NMR (270 MHz, CDCl₃): δ 1.68-1.75 (6H, m, 3×CH₂), 1.89 (6H, d, J=2.8 Hz, 3×CH₂), 2.05 (3H, broad, 3×CH), 4.53 (2H, d, J=5.9 Hz, CH₂), 6.10 (1H, br, NH), 7.48 (1H, t, J=7.9 Hz, ArH), 7.59 (1H, t, J=8.0 Hz, ArH) and 8.07-8.12 (2H, m, ArH); LC/MS (APCI) m/z 315 (M⁺+11), tᵣ=1.26 min (99%) in 5% water-methanol; HPLC tᵣ=1.89 min (>99%) in 10% water-acetonitrile.

### N-[4-(2-Adamantan-1-yl-2-oxo-ethoxy)-2-chloro-phenyl]-acetamide (STX2412, XDS04188)

To a solution of N-(2-chloro-4-hydroxyphenyl)acetamide (186 mg, 1.0 mmol) in DMF (5 mL) were added K₂CO₃ (200 mg) and tetrabutylammonium bromide (10 mg). The mixture was stirred at room temperature for 5 min, 1-adamantyl bromomethyl ketone (258 mg, 1,0 mmol) was added. After 4 h, the mixture was diluted with water and the precipitate was collected and washed with water. Purification with flash column (DCM-EtOAc gradient elution) gave product (280 mg, 78%) as white solid. mp 204-206° C.; TLC single spot at R_{f}: 0.39 (15% EtOAc/DCM); ¹H NMR (270 MHz, CDCl₃) δ 1.65-1.80 (6H, m, 3×CH₂), 1.90 (6H, d, J=2.8 Hz, 3×CH₂), 2.07 (3H, broad, 3×CH), 2.20 (3H, s, CH₃), 4.81 (2H, s, CH₂), 6.76 (1H, dd, J=9.0, 3.0 Hz, ArH), 7.24 (1H, d, J=2.9 Hz, ArH), 7.38 (1H, broad, NH), and 8.17 (1H, d, J=9.0 Hz, ArH); LC/MS (APCI) m/z 360 (M⁺−H), tᵣ=1.24 mm (98%) in 5% water-methanol; HPLC t=1.92 min (99%) in 10% water-acetonitrile.

### N-[4-(2-Adamantan-1-yl-2-oxo-ethoxy)-2-fluoro-phenyl]-acetamide (STX2413, XDS04189)

To a solution of N-(2-fluoro-4-hydroxyphenyl)acetamide (169 mg, 1.0 mmol) in DMF (5 mL) were added K₂CO₃ (200 mg) and tetrabutylammonium bromide (10 mg). The mixture was stirred at room temperature for 5 min, 1-adamantyl bromomethyl ketone (258 mg, 1.0 mmol) was added. After 4 h, the mixture was diluted with water and the precipitate was collected and washed with water. Purification with flash column (DCM-EtOAc gradient elution) gave product (230 mg, 67%) as white solid. mp 191-192.5° C.; TLC single spot at R_{f}: 0.42 (15% EtOAc/DCM); ¹H NMR (270 MHz, CDCl₃) δ 1.65-1.80 (6H, m, 3×CH₂), 1.90 (6H, d, J=2.8 Hz, 3×CH₂), 2.07 (3H, broad, 3×CH), 2.18 (3H, s, CH₃), 4.81 (2H, s, CH₂), 6.55-6.70 (2H, m, ArH), 7.14 (1H, broad, NH), and 8.08 (1H, t, J=8.9 Hz, ArH); LC/MS (APCI) m/z 344 (M⁺−H), tᵣ=1.20 min (98%) in 5% water-methanol; HPLC tᵣ=1.79 min (99%) in 10% water-acetonitrile.

### 2-[4-(2-Adamantan-1-yl-2-oxo-ethoxy)-phenyl]-N-benzyl-acetamide (STX2415, FPC01075)

EDCI (69 mg, 0.36 mmol), DMAP (44 mg, 0.36 mmol) and Et₃N (50 μL, 0.36 mmol) were added to a solution of [4-(2-Adamantan-1-yl-2-oxo-ethoxy)-phenyl]-acetic acid (100 mg, 0.30 mmol) in DCM (8.5 mL) at room temperature. After 30 min, benzylamine (39 μL, 0.36 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01075 (89 mg, 71%) as cream solid. Mp=131.4° C.; ¹H NMR (270 MHz, CDCl₃): δ 7.31-7.20 (3H, m), 7.78-7.10 (4H, m), 5.90 (2H, d app, J=8.7 Hz), 5.88 (1H, br s, NH), 4.81 (2H, s), 4.36 (2H, d, J=6.0 Hz), 3.51 (2H, s), 2.06 (3H, br s), 1.89 (6H, d, J=2.7 Hz), 1.82-1.64 (6H, m); LC/MS (APCI) tᵣ=1.37 min, m/z 416.55 (M⁺−1H, 100); HPLC tᵣ=2.07 min (>98.66% purity).

### 2-[4-(2-Adamantan-1-yl-2-oxo-ethoxy)-phenyl]-N-phenyl-acetamide (STX2416, FPC01076C)

EDCI (69 mg, 0.36 mmol), DMAP (44 mg, 0.36 mmol) and Et₃N (50 μL, 0.36 mmol) were added to a solution of [4-(2-Adamantan-1-yl-2-oxo-ethoxy)-phenyl]-acetic acid (100 mg, 0.30 mmol) in DCM (8 mL) at room temperature. After 30 min, aniline (0.180 mL, 2M solution in THF, 0.36 mmol) was added neat and the reaction was allowed to stir over night. When the starting material seemed to be consumed, the reaction was quenched with a saturated solution of NH₄Cl, extracted with DCM twice then washed with a saturated solution of NaHCO₃ then water and brine. The organic phases were dried over magnesium sulphate, filtrated then concentrated in vacuo. The crude was purified by flash chromatography (hexanes/EtOAc 9:1 and 8:2 then DCM/MeOH 95:5 and 90:10) and the first fraction was isolated as the expected amide FPC01076C (55.8 mg, 46%) as cream solid. Mp=153.4° C.; ¹H NMR (270 MHz, CDCl₃): δ 7.43-7.37 (2H, m), 7.30-7.15 (4H, m), 7.60 (1H, tt, J=1.2, 7.2 Hz), 6.52 (2H, d app, J=8.7 Hz), 4.86 (2H, s), 3.64 (2H, s), 2.07 (3H, br s), 1.91 (6H, d, J=2.9 Hz), 1.84-1.66 (6H, m); LC/MS (APCI) tᵣ=1.38 min, m/z 402.42 (M⁺−1H, 100); HPLC tᵣ=2.21 min (>98.41% purity).

### Biological Assays

### 11β-HSD1 HTRF Assay Protocol

The 11β-HSD Type 1 assay was carried out at room temperature in 96 well microtitre plates in a total volume of 40 μl. Assay buffer contained 30 mM Tris.HCl, 1 mM EDTA, pH 7.2. Inhibitor compounds were tested at varying concentrations at a final Dimethyl Sulphoxide concentration of 1%. Compounds were usually tested at 10 μM. The substrate mixture contained cortisone, NADPH and glucose-6-phosphate at final concentrations of 1 μM, 180 μM and 1 mM respectively. Assays were started by the addition of 10 μl human liver microsome fractions, 20 mg per ml protein, containing 1113-HSD Type 1, diluted 1 to 40 prior to use. Following mixing microtitre plates were shaken for 30 minutes. Reactions were stopped by the addition of 10 μl 1 mM Glyccheritinnic acid solution. Production of Cortisol was measured by Homogenous Time Resolved Fluorescence using a cortisol assay kit; Cisbio International Cortisol HTRF kit (62CORPEB), following the manufacturer's protocol.

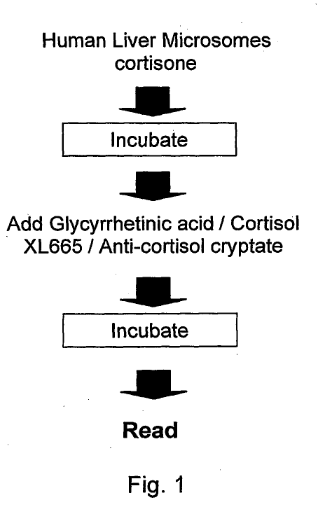

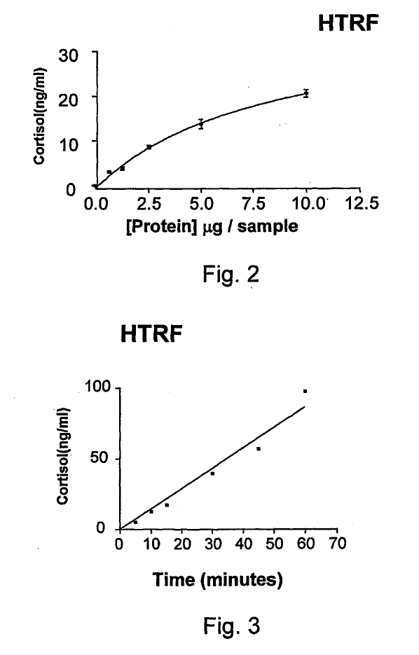

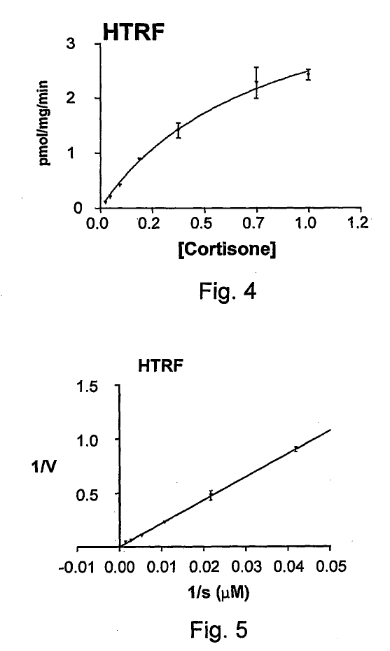

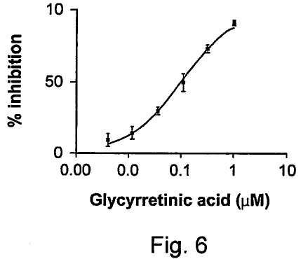

The nature and verification of the assay is shown in FIGS. 1 to 6.

### 11β-HSD1 HEK Assay Protocol (Cell Based Assay)

### Introduction

11β-HSD1 activity is measured in whole HEK 293 cells stably transfected with the HSD11B1 gene. Cells are incubated in 96-well microplates in the presence of tritiated substrate. Enzyme activity is determined by measuring the amount of tritiated product by scintillation proximity assay (SPA). Assay plates contain internal high and low controls to allow calculation of percentage inhibition.

### Method

1. Each well of a 96-well culture plate is seeded with HEK 293/HSD11B1 cells in 100 μl medium.
2. When the cells are 80% confluent the medium is removed from each well. 100 μl fresh, serum-free, medium containing ³H-cortisone and test compound in 1% DMSO is added to each well*. Control wells are also dispensed. High control wells do not contain compound, while low controls do not contain cells.
3. The plate is incubated at 37° C. for the required time period.
4. 50 μl of media is removed from each well and transferred to a microplate containing 100 μl of a pre-incubated mixture of anti-cortisol antibody and SPA bead. The mixture is incubated with gentle shaking until equilibrium is reached, before transferring to a scintillation counter to establish the enzyme activity in each sample.

### *Preparation of Samples

10 μl of compound is dispensed into each well of a 96-well microplate in 10% DMSO at 100 μM concentration. 90 μl media containing ³H-cortisone is added to each well. The compound/media/substrate mixture is then transferred to the assay plate containing the cells. The final concentration of compound and DMSO is 10 μM and 1% respectively.

### Inhibition Data

The structures of the above synthesised compounds and the data obtained are given in the table below

Compounds showing >60% inhibition of 11β-HSD1 when tested at 10 μM using the above protocol have been designated (a) in the table below, those showing from 20 to 60% inhibition of 11β-HSD1 when tested at 10 μM using the same protocol have been designated (b) in the table below, and those showing less than 20% inhibition of 11β-HSD1 when tested at 10 μM using the same protocol have been designated (c) in the table below

| | | % INHIBITION | % INHIBITION |
|---|---|---|---|
| | | of Human 11 β- | of Human 11β- |
| STX | | HSD1 at 10 μM | HSD1 at 10 μM |
| CODE | STRUCTURE | (HTRF) | (HEK) |
| 1341 | 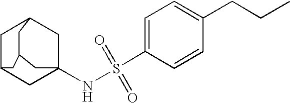 | b | b |
| 1342 | 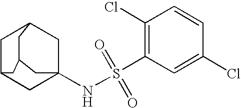 | — | b |
| 1343 | 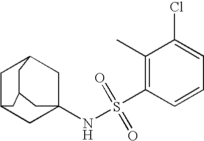 | — | b |
| 1344 | 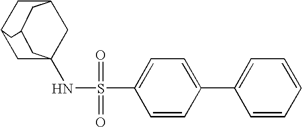 | b | b |
| 1368 | 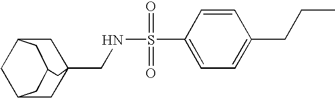 | a | b |
| 1370 | 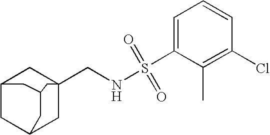 | — | b |
| 1371 | 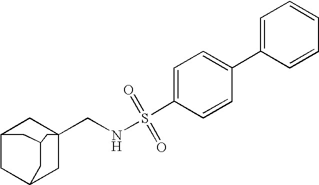 | — | b |
| 1372 | 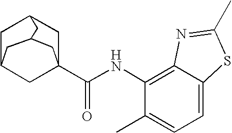 | b | b |
| 1373 | 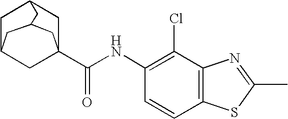 | b | a |
| 1374 | 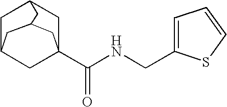 | a | a |
| 1375 | 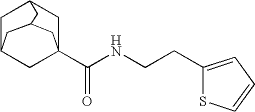 | a | a |
| 1487 | 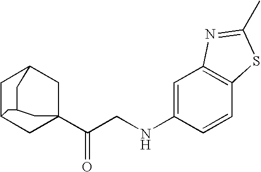 | a | a |
| 1499 | 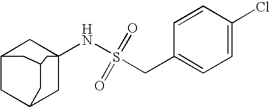 | b | c |
| 1500 | 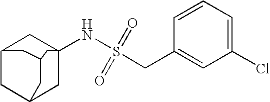 | b | c |
| 1535 | 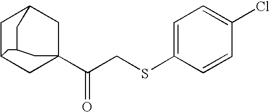 | a | a |
| 1537 | 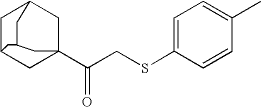 | a | a |
| 1538 | 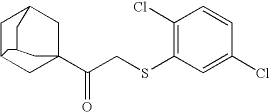 | a | a |
| 1539 | 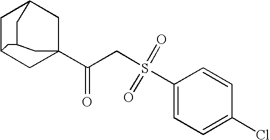 | a | b |
| 1540 | 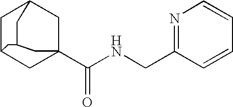 | a | b |
| 1541 | 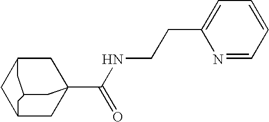 | a | a |
| 1542 | 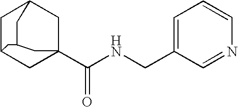 | a | a |
| 1543 | 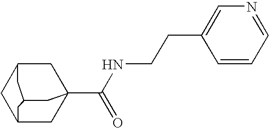 | a | c |
| 1544 | 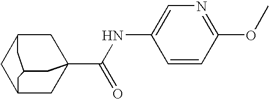 | a | b |
| 1545 | 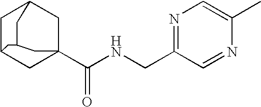 | a | b |
| 1562 | 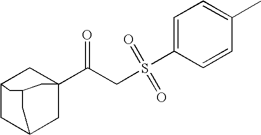 | a | a |
| 1563 | 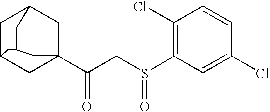 | a | a |
| 1564 | 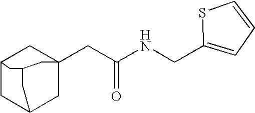 | a | a |
| 1565 | 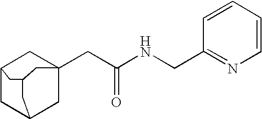 | a | b |
| 1566 | 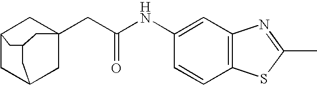 | — | b |
| 1567 | 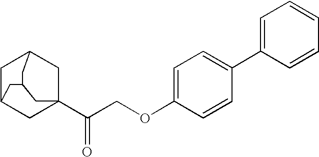 | a | a |
| 1568 | 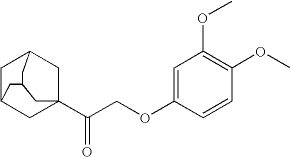 | a | a |
| 1569 | 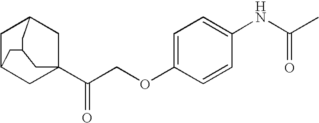 | a | a |
| 1570 | 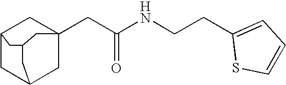 | a | a |
| 1572 | 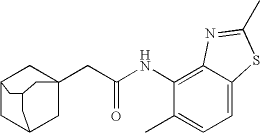 | a | c |
| 1573 | 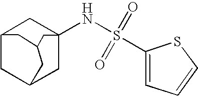 | a | — |
| 1574 | 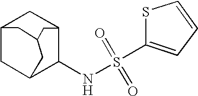 | a | b |
| 1575 | 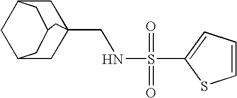 | a | c |
| 1576 | 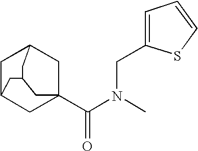 | a | a |
| 1577 | 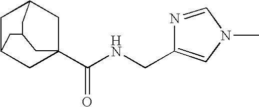 | a | a |
| 1578 | 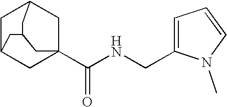 | a | a |
| 1579 | 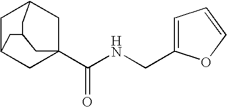 | a | a |
| 1580 | 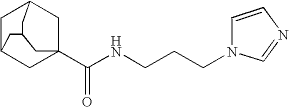 | b | b |
| 1581 | 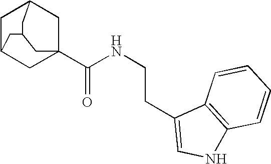 | a | a |
| 1597 | 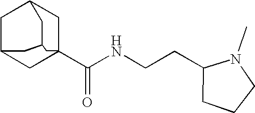 | a | c |
| 1598 | 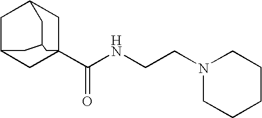 | a | a |
| 1599 | 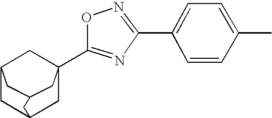 | b | b |
| 1610 | 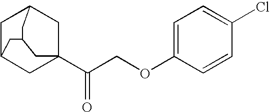 | a | a |
| 1618 | 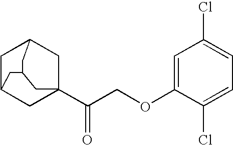 | a | a |
| 1619 | 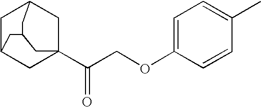 | a | a |
| 1621 | 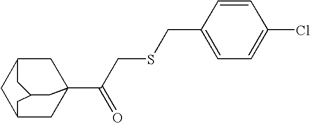 | a | a |
| 1626 | 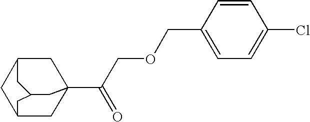 | a | b |
| 1627 | 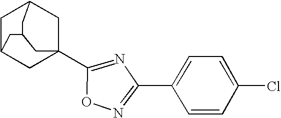 | b | — |
| 1628 | 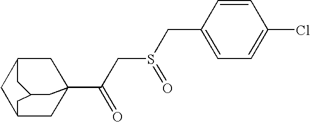 | a | a |
| 1632 | 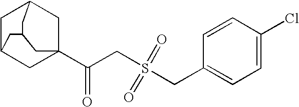 | a | a |
| 1638 | 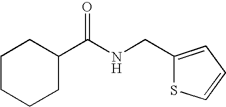 | — | b |
| 1639 | 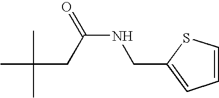 | — | b |
| 1640 | 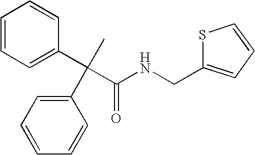 | — | b |
| 1641 | 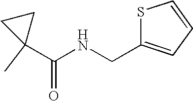 | — | b |
| 1642 | 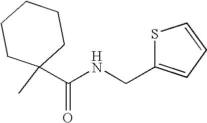 | — | b |
| 1648 | 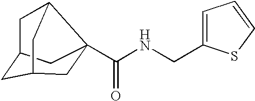 | — | a |
| 1650 | 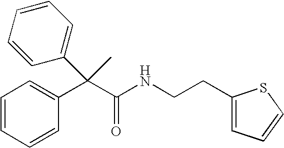 | — | c |
| 1651 | 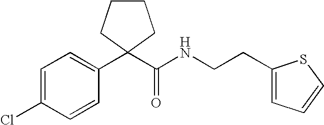 | | c |
| 1652 | 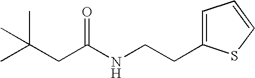 | — | b |
| 1653 | 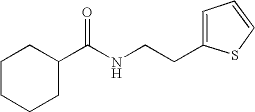 | — | b |
| 1654 | 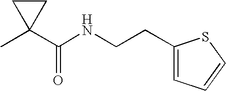 | — | b |
| 1655 | 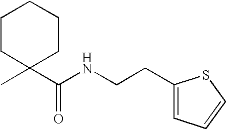 | — | a |
| 1656 | 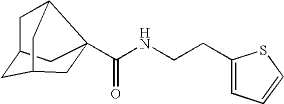 | | a |
| 1660 | 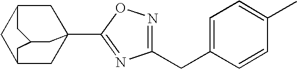 | — | b |
| 1661 | 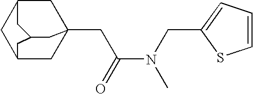 | — | a |
| 1662 | 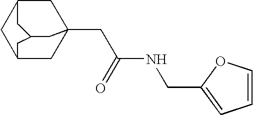 | — | a |
| 1663 | 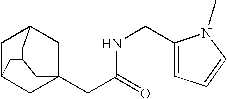 | — | a |
| 1664 | 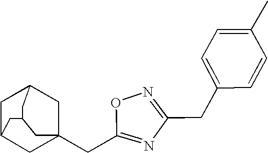 | — | b |
| 1671 | 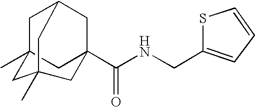 | — | a |
| 1672 | 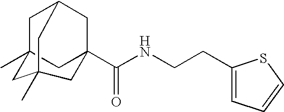 | — | a |
| 1673 | 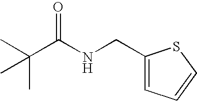 | — | b |
| 1674 | 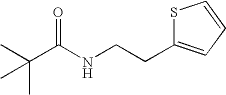 | — | b |
| 1675 | 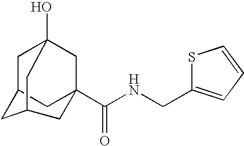 | — | c |
| 1676 | 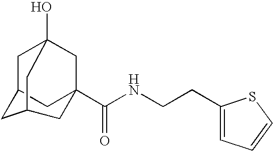 | — | a |
| 1687 | 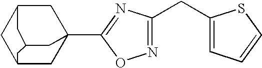 | — | b |
| 1688 | 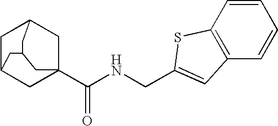 | — | c |
| 1689 | 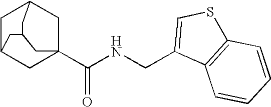 | — | b |
| 1690 | 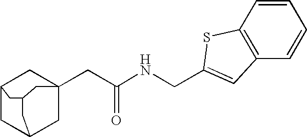 | — | b |
| 1691 | 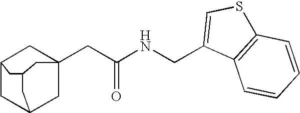 | — | b |
| 1692 | 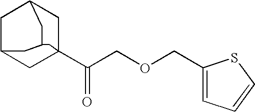 | — | a |
| 1693 | 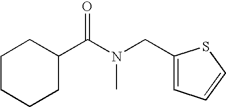 | — | a |
| 1694 | 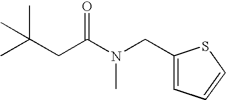 | — | a |
| 1695 | 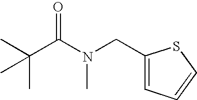 | — | c |
| 1696 | 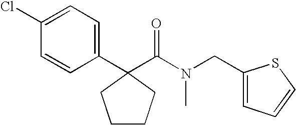 | — | a |
| 1697 | 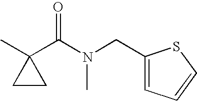 | — | c |
| 1698 | 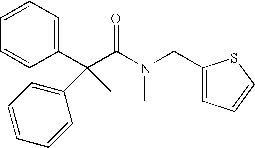 | — | b |
| 1699 | 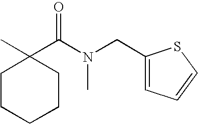 | — | a |
| 1700 | 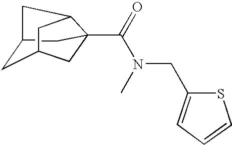 | — | a |
| 1706 | 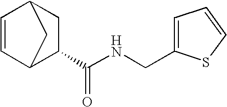 | — | b |
| 1707 | 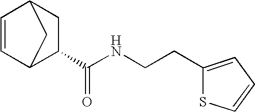 | — | b |
| 1708 | 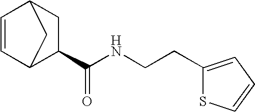 | — | b |
| 1709 | 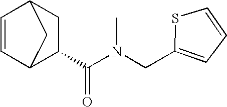 | — | a |
| 1710 | 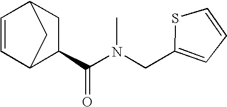 | — | a |
| 1711 | 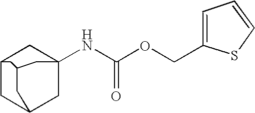 | — | a |
| 1712 | 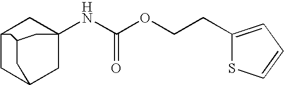 | — | a |
| 1713 | 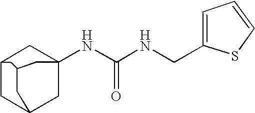 | — | b |
| 1714 | 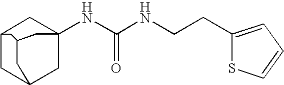 | — | a |
| 1721 | 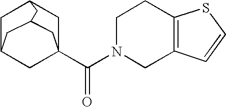 | — | a |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

### REFERENCES

1. Hammond, G H (1990): Molecular properties of corticosteroid binding globulin and sex-steroid binding proteins. Endocr. Rev. 11, 65-79.
2. Gomez-Sanchez E P, Gomex-Sanchez C E (1997): First there was one, then two why not more 11β-Hydroxysteroid Dehydrogenases? Endocrinology vol. 138, 12.
3. Krozowski Z S, Funder J W (1983): Renal mineralocorticosterone receptors and hippocampal corticosterone binding species have identical intrinsic steroid specificity. Proc. Natl. Sci. USA 80: 6056-60
4. Ulick S, Levine L S, Gunczler P, Zanconato G, Rarnirez L C, Rauh W, Rosler A, Bradlow H L, Mew M I (1979): A syndrome of apparent mineralocorticoid excess associated with defects in the peripheral metabolism of cortisol. J. Clin. Endo. And Metab. 49: 757-64.
5. Edwards C R W, Stewart P M, Burt D, Brett L, McIntyre M A, Sutanto W S, Kloet E R, Monder C (1998): Localisation of 11β-HSD-tissue specific protector of the mineralocorticoid receptor. Lancet 2: 986-989.
6. Moore C C D, Melloh S H, Murai I, Siiteri P K, Miller W L (1993): Structure and function of the hepatic form of 11β-HSD in the squirrel monkey, an animal model of glucocorticoid resistance. Endocrinology 133: 368-375.
7. Kotelevtsev Y V, Iarnieson P M, Best R, Stewart F, Edwards C R W, Seckl J R, Mullins I I (1996): Inactivation of 11β-HSD type 1 by gene targeting in mice. Endocrinology Res. 22: 791-792.
8. Ricketts M L, Verhaeg J M, Bujalska I, Howie A J, Rainey W E, Stewart P M (1998): Immunohistochemicallocalisation of type 1 11β-HSD in human tissues. I. Clin. Endoc. Metab. 83: 1325-35.
9. Stewart P M, Sheppard M C (1992): Novel aspects of hormone action: intracellular ligand supply and its control by a series of tissue specific enzymes. Molecular and Cellular Endocrinology 83: C13-C18.
10. Secki J R, Chapman K E (1997): The 11β-HSD system, a determinant of glucocorticoid and mineralocorticoid action. Medical and physiological aspects. European L Biochem. 249: 361-364.
11. Maser E (1998): 11β-HSD responsible for carbonyl reduction of the tobacco-specific nitrosoamine in mouse lung microsomes. Cancer Res. 58: 2996-3003.
12. Walker B R, Stewart P M, Shackleton C H L, Padfield P L, Edwards C R W (1993): Deficient inactivation of cortisol by 11β-HSD in essential hypertension. Clin. Endocr. 38: 221-227.
13. Daynes R A, Araneo B A (1998): Contrasting effects of glucocorticoids on the capacity of T-cells to produce the growth factors interleukin-2 and interleukin-4. Eur. J. Immunol. 19: 2319-2324.
14. Barf, T. et al., (2002), Arylsulfonamidothiazoles as a new class of potential antidiabetic drugs. Discovery of potent and selective inhibitors of the 111-Hydroxysteroid Dehydrogenase Type 1. J. Med. Chem., 45, 3813-3815.
15. Matassa, Victor G. et. al. J. Med. Chem.; 33(9); 1990; 2621.
16. This compound is synthesized in the literature and the NMR spectrum is reported, however the spectrum obtained here differs from that in the literature. Baraldi, Pier Giovanni et. al.; Bioorg. & Med. Chem. Lett.; 10; 2002, 1611.
17. Horaguchi, Takaaki; Matsuda, Shinichi; Tanemura, Kiyoshi; Suzuki, Tsuneo. J. Heterocyclic Chem.; 24; 1987; 965.
18. Plé, Patrick A., Marnett, Lawrence J.; J. Heterocyclic Chem.; 25; 1988; 1271.
19. Rao, U. and Balasubramanian, K. K.; Tetrahedron Lett.; 24; 1983; 5023.
20. Bordwell, F. G. and Stange, Hugo; J. Amer. Chem. Soc.; 77; 1955; 5939.
21. Elderfield, Robert C.; Williamson, Thurmond A.; Gensler, Walter J.; Kremer, Chester B.; J. Org. Chem; 12; 1947; 405.
22. For 6-nitro-2,3-dimethylquinoxaline see: Barluenga, Jose; Aznar, Fernando; Liz, Ramon; Cabal, Maria-Paz; Synthesis; 3; 1985; 313., then for 6-amino-2,3-dimethylquinoxaline: Salon, Jozef; Milata, Viktor; Pronayova, Nadezda; Lesko, Jan; Collect. Czech. Chem. Commun.; 66; 11; 2001; 1691.
23. Klicnar, J.; and Kosek, F.; Collect. Czech. Chem. Commun.; 30; 1965; 3102.
24. Gloster, Daniel F.; Cincotta, Louis; Foley, James W.; J. Heterocyclic Chem.; 36; 1999; 25.
25. The same reduction was carried out using SnCl₂ by: Case et al.; J. Amer. Chem. Soc.; 81; 1959; 6297.
26. Modified procedure from similar compound described in U.S. Pat. No. 6,355,796 (Example 20)
27. Hollfelder, F.; Kirby, A. J.; Tawfik, D. S.; Kikuchi, K.; Hilvert, D.; J. Amer. Chem. Soc.; 122 (6); 2000; 1022-1029
28. Fujimoto, M.; Okabe, K.; Chem. Pharm. Bull.; 10; 1962; 572-575.
29. Kawamura, T.; Yagi, N.; Sugawara, H.; Yamahata, K.; Takada, M.; Chem. Pharm. Bull.; 28; 1; 1980; 268-276.
30. Stewart, P. M. and Mason, J. I., (1995), Cortisol to cortisone: Glucocorticoid to mineralocortcoid. Steriods, 60, 143-146.
31. Escher, G. et al., (1995), Furosemide inhibits 11β-Hydroxysteroid Dehydrogenase in vitro and in vivo. Endocrinology, 136, 1759-1765.
32. Hult, M. et. al., (1998), Selective inhibition of human type 1 11β-hydroxysteroid dehydrogenase by synthetic steroids and xenobiotics. FEBS Letters, 441, 2528.
33. Diederich S, Grossmann C, Hanke B, Quinkler M, Herrrnann M, Bahr V, Oelkers W (2000): In the search for specific inhibitors of human 11β-HSD: chenodeoxycholic acid selectively inhibits 11β-HSD type 1. Europ. J. Endocrin. 142: 200-207.

1. A compound having Formula I
R₁-Z-R₂ Formula I
wherein
R₁ is a group selected from optionally substituted fused polycyclic groups, substituted alkyl groups, branched alkyl groups, and optionally substituted cycloalkyl groups
Z is a linker which is or comprises a carbonyl group or a isostere of a carbonyl group
R₂ is selected from optionally substituted aromatic rings and optionally substituted heterocyclic rings wherein
a) R₂ is a 2-substituted thiophene group, and/or
(b) Z is a group of the formula —C(—O)—CR₃R₄—X—(CR₅R₆)n-, wherein X is selected from NR₇, S, O, S═O, and S(═O)₂, wherein n is 0 or 1 and/or
(c) R₁ is an adamantyl group and Z is or comprises an amide group, and/or
(d) R₁ is an adamantyl group and Z is or comprises a group of the formula —(CR₈R₉)p-NR₁₀—SC═O)₂—(CR₁₁R₁₂)q-, wherein p is 0 or 1 and q is 0 or 1 and/or
(e) R₁ is an adamantyl group and Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is 0 or 1 and w is 0 or 1;
wherein each of R₃, R₄, R₅, R₆, R₈, R₉, R₁₁, R₁₂, R₁₃, R₁₄, R₁₅ and R₁₆, are independently selected from H, hydrocarbyl and halogen, wherein each of R₇ and R₁₀ are independently selected from H and hydrocarbyl.

2. A compound according to claim 1 wherein R₁ is a group selected from unsubstituted fused polycyclic groups, substituted alkyl groups, branched alkyl groups, and optionally substituted cycloalkyl groups.

3. A compound according to claim 1 or 2 wherein the fused polycyclic group comprises three fused rings.

4. A compound according to claim 1 or 2 wherein the fused polycyclic group comprises only carbocyclic fused rings.

5. A compound according to claim 1 wherein the fused polycyclic group is non-aromatic.

6. A compound according to claim 1 wherein the fused polycyclic groups are selected from adamantyl group and a noradamantyl group.

7. A compound according to claim 1 wherein the fused polycyclic groups is an adamantyl group.

8. A compound according to claim 1 wherein the substituted alkyl group is an alkyl group substituted with at least one aryl group.

9. A compound according to claim 1 wherein the substituted alkyl group is an alkyl group substituted with at least one of an phenyl group.

10. A compound according to claim 1 wherein the substituted alkyl group is di-substituted.

11. A compound according to claim 1 wherein the substituted alkyl group is a substituted C₁₋₁₀ alkyl group.

12. A compound according to claim 1 wherein the substituted alkyl group is a substituted C₁₋₅ alkyl group.

13. A compound according to claim 1 wherein the substituted alkyl group is a substituted ethyl group.

14. A compound according to claim 1 wherein the substituted alkyl group is a disubstituted ethyl group.

15. A compound according to claim 1 wherein the substituted alkyl group is —C(Ph)₂-CH₃.

16. A compound according to claim 1 wherein the branched alkyl group is a branched C₁₋₁₀ alkyl group.

17. A compound according to claim 1 wherein the branched alkyl group is a branched C₁₋₅ alkyl group.

18. A compound according to claim 1 wherein the branched alkyl group is a branched C₅ alkyl group.

19. A compound according to claim 1 wherein the branched alkyl group is or comprises a —C(CH₃)₃ group.

20. A compound according to claim 1 wherein the branched alkyl group is a —CH₂C(CH₃)₃ group.

21. A compound according to claim 1 wherein the optionally substituted cycloalkyl group is an optionally substituted C₃₋₁₀ cycloalkyl group.

22. A compound according to claim 1 wherein the optionally substituted cycloalkyl group is an optionally substituted C₃₋₆ cycloalkyl group.

23. A compound according to claim 1 wherein the optionally substituted cycloalkyl group is an optionally substituted C₃, C₅ or C₆ cycloalkyl group.

24. A compound according to claim 1 wherein the optionally substituted cycloalkyl group is substituted at the carbon attaching the cycloalkyl group to Z.

25. A compound according to claim 1 wherein the optionally substituted cycloalkyl group is substituted only at the carbon attaching the cycloalkyl group to Z.

26. A compound according to claim 1 wherein the optionally substituted cycloalkyl group is mono-substituted.

27. A compound according to claim 1 wherein the or each optional substituent of the optionally substituted cycloalkyl group is independently selected from hydrocarbyl groups, halogens, hydroxyl, amines, and amides.

28. A compound according to claim 1 wherein the or each optional substituent of the optionally substituted cycloalkyl group is independently selected from hydrocarbon groups, oxyhydrocarbon groups, hydroxyl, amines, and halogens.

29. A compound according to claim 1 wherein the or each optional substituent of the optionally substituted cycloalkyl group is independently selected from alkyl groups and optionally substituted aryl groups.

30. A compound according to claim 1 wherein the or each optional substituent of the optionally substituted cycloalkyl group is independently selected from C₁₋₁₀ alkyl groups.

31. A compound according to claim 1 wherein the or each optional substituent of the optionally substituted cycloalkyl group is independently selected from C₁₋₅ alkyl groups.

32. A compound according to claim 1 wherein the or each optional substituent of the optionally substituted cycloalkyl group is independently selected from C₁₋₃ alkyl groups.

33. A compound according to claim 1 wherein the or each optional substituent of the optionally substituted cycloalkyl group is a methyl group.

34. A compound according to claim 29 wherein the optionally substituted aryl group is an optionally substituted phenyl group.

35. A compound according to claim 29 wherein the optionally substituted aryl group is a substituted phenyl group.

36. A compound according to claim 35 wherein the substituted phenyl group is substituted with at least one halogen.

37. A compound according to claim 35 wherein the substituted phenyl group is substituted with at least one chloro group.

38. A compound according to claim 1 wherein the optionally substituted cycloalkyl group is selected from

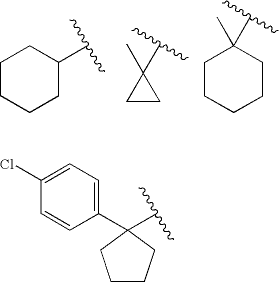

39. A compound according to claim 1 wherein R₁ is selected from an adamantyl group, a —C(Ph)₂-CH₃ group, a —CH₂C(CH₃)₃ 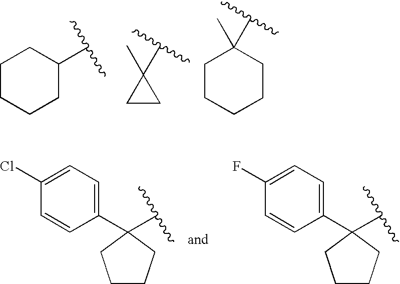
group.

40. A compound according to claim 1 wherein Z is a linker which is or comprises a carbonyl group.

41. A compound according to claim 1 wherein the isostere of a carbonyl group is a group selected from CON, C—OH, C═C, C═NOH, C═NOC₁₋₅ alkyl, C═NNH₂, C═NNHC₁₋₅ alky, C═NN(C₁₋₅ alky)₂, C≡N, C═NCN, C═NNO₂, C═S, S═O, S(—O)₂, S═NH, S═NC₁₋₅ alky, P═O, P(═O)_{z}, P—OH, P═S, P—SH, P═NH, and P═NC₁₋₅ alky.

42. A compound according to claim 1 wherein Z is a group of the formula —C(═O)—CR₃R₄—X—(CR₅R₆)n-, wherein X is selected from NR₇, S, O, S═O, and S(═O)₂, wherein n is 0 or 1.

43. A compound according to claim 1 wherein Z is selected from —C(═O)CH₂NH—, —C(═O)CH₂NMe-, —C(═O)CH₂NHCH₂—, —C═O)CH₂NMeCH₂—(.HCI), —C(═O)CH₂—S—, —C(═O)CH₂—S(═O)₂—, —C(—O)CH₂—S(═O)—, —C(═O)CH₂—O—, —C(═O)—CH₂—S—CH₂—, —C(═O)CH₂—O—CH₂—, —C(═O)CH₂—S(═O)₂—CH₂—, and —C(═O)CH₂—S(═O)—CH₂—.

44. A compound according to claim 1 wherein Z is or comprises an amide group.

45. A compound according to claim 1 wherein Z is selected from —(CH₂)₀₋₆—C(═O)NH—(CH₂)₀₋₆—.

46. A compound according to claim 1 wherein Z is selected from —C(═O)NH—, —C(═O)NH—CH₂—, —C(═O)NH—(CH₂)₂—, —CH₂—C(═O)NH—CH₂—, —CH₂—C(═O)NH—, —CH₂—C(═O)NH—(CH₂)₂—, —C(═O)NMe-CH₂—, —C(═O)NH—(CH₂)₃—, and —CH₂—C(—O)NMe-CH₂—.

47. A compound according to claim 1 wherein Z is or comprises a group of the formula —(CR₈R₉)p-NR₁₀—S(═O)₂—(CR₁₁R₁₂)q-, wherein p is 0 or 1 and q is 0 or 1.

48. A compound according claim 1 wherein Z is selected from —NH—S(═O)₂—, —CH₂—NH—S(═O)₂—, and —NH—S(═O)₂—CH₂—.

49. A compound according to claim 1 wherein Z is or comprises a group of the formula —(CR₁₃R₁₄)v-Y—(CR₁₅R₁₆)w- where Y is a heteroaryl group in which a bond in the heteroaryl ring is a isostere of a carbonyl group, wherein v is 0 or 1 and w is 0 or 1.

50. A compound according to claim 1 wherein Z is selected from groups of the formula —CH₂—Y—CH₂—, —Y—CH₂—, —CH₂—Y— and —Y—.

51. A compound according to claim 1 wherein Y is an oxadiazole group or 1H-1,2,3-triazole group.

52. A compound according to claim 1 wherein Z is or comprises a group of the formula 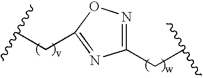
wherein v is 0 or 1 and w is 0 or 1.

53. A compound according to claim 1 wherein R₂ is selected from substituted carbocyclic aromatic rings and unsubstituted heterocyclic rings.

54. A compound according to claim 1 wherein R₂ is selected from optionally substituted rings

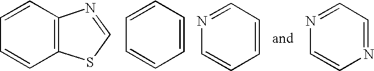

55. A compound according to claim 1 wherein R₂ is selected from optionally substituted heterocyclic rings.

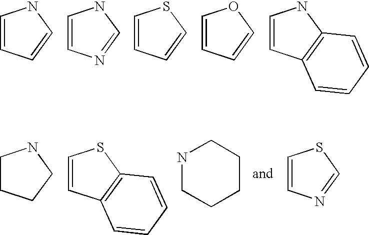

56. A pharmaceutical composition comprising a compound according to claim 1 optionally admixed with a pharmaceutically acceptable carrier, diluent, excipient or adjuvant.

57. (canceled)

58. A method of treating or preventing a condition or disease associated with 11β-HSD comprising administering an effective amount of a compound according to claim 1 to an individual in need thereof.

59. The method according to claim 58 wherein the condition or disease is selected from the group consisting of metabolic disorders such as diabetes and obesity; cardiovascular disorders such as hypertension; glaucoma; inflammatory disorders such as arthritis or asthma; immune disorders; bone disorders such as osteoporosis; cancer; intra-uterine growth retardation; apparent mineralocorticoid excess syndrome (AME); polycystic ovary syndrome (PCOS); hirsutism; acne; oligo- or amenorrhea; adrenal cortical adenoma and carcinoma; Cushing's syndrome; pituitary tumours; invasive carcinomas; breast cancer; and endometrial cancer.

60. The method according to claim 58 wherein the condition or disease is associated with adverse 11β-HSD levels.

61. The method according to claim 58 further comprising modulating 11β-HSD activity.

62. The method according to claim 58 further comprising inhibiting 11β-HSD activity.

63. A method comprising (a) performing a 11β-HSD assay with one or more candidate compounds having the formula as defined in claim 1; (b) determining whether one or more of said candidate compounds is/are capable of modulating 11β-HSD activity; and (c) selecting one or more of said candidate compounds that is/are capable of modulating 11β-HSD activity.

64. A method comprising (a) performing a 11β-HSD assay with one or more candidate compounds having the formula as defined in claim 1; (b) determining whether one or more of said candidate compounds is/are capable of inhibiting 11β-HSD activity; and (c) selecting one or more of said candidate compounds that is/are capable of inhibiting 11β-HSD activity.

65. A compound identified by the method according to claim 63 or claim 64.

66. (canceled)

67. A pharmaceutical composition comprising the compound according to claim 65 optionally admixed with a pharmaceutically acceptable carrier, diluent, excipient or adjuvant.

68. A method of treating or preventing a condition or disease associated with 11β-HSD comprising administering an effective amount of a compound according to claim 65 to an individual in need thereof.

69. The method according to claim 68 wherein the condition or disease is selected from the group consisting of metabolic disorders such as diabetes and obesity; cardiovascular disorders such as hypertension; glaucoma; inflammatory disorders such as arthritis or asthma; immune disorders; bone disorders such as osteoporosis; cancer; intra-uterine growth retardation; apparent mineralocorticoid excess syndrome (AME); polycystic ovary syndrome (PCOS); hirsutism; acne; oligo- or amenorrhea; adrenal cortical adenoma and carcinoma; Cushing's syndrome; pituitary tumours; invasive carcinomas; breast cancer; and endometrial cancer.

70. The method according to claim 65 wherein the condition or disease is associated with adverse 11β-HSD levels.

71. The invention of any one of claims 58, 63 or 64 wherein 11β-HSD is 11β-HSD Type 1.

72. The invention of any one of claims 58, 63 or 64 wherein 11β-HSD is 11β-HSD Type 2.

73-76. (canceled)
